(19) United States
(12) Patent Application Publication
(10) Pub. No.: US 2025/0136981 A1
(43) Pub. Date: May 1, 2025

(54) COMPOSITIONS AND METHODS COMPRISING ENGINEERED SHORT NUCLEAR RNA (SNRNA)

(71) Applicant: Regeneron Pharmaceuticals, Inc., Tarrytown, NY (US)

(72) Inventors: Rea LARDELLI MARKMILLER, San Diego, CA (US); Ranjan BATRA, San Diego, CA (US); Rachel A. ADAMS, San Diego, CA (US); Daniela ROTH, San Diego, CA (US); Daniel A. KNOWLAND, San Diego, CA (US)

(21) Appl. No.: 18/733,477

(22) Filed: Jun. 4, 2024

Related U.S. Application Data
(63) Continuation of application No. 18/447,651, filed on Aug. 10, 2023, now Pat. No. 12,037,588.
Application No. 18/447,651 is a continuation of application No. 18/179,110, filed on Mar. 6, 2023.
(60) Provisional application No. 63/379,983, filed on Oct. 18, 2022.
(60) Provisional application No. 63/316,659, filed on Mar. 4, 2022.

(51) Int. Cl. C12N 15/113; A61P 21/00; A61P 25/28; C12N 15/86

(52) U.S. Cl. CPC C12N 15/113; A61P 21/00; A61P 25/28; C12N 15/86; C12N 2310/14; C12N 2310/531; C12N 2750/14143

(57) ABSTRACT

SnRNA systems comprising engineered stem loops are disclosed herein.

### RELATED APPLICATIONS

This application is a continuation of U.S. application Ser. No. 18/447,651, filed Aug. 10, 2023, which is a continuation of U.S. application Ser. No. 18/179,110, filed Mar. 6, 2023, which claims priority to, and the benefit of U.S. Provisional Application No. 63/316,659 filed Mar. 4, 2022 and U.S. Provisional Application No. 63/379,983 filed Oct. 18, 2022. The contents of each of these applications are hereby incorporated by reference in their entireties.

### REFERENCE TO AN ELECTRONIC SEQUENCE LISTING

The contents of the electronic sequence listing (LOCN_020_C02US_SeqList_ST26.xml; Size: 289,775 bytes; and Date of Creation: Jun. 4, 2024) is herein incorporated by reference in its entirety.

### FIELD OF THE DISCLOSURE

The disclosure is directed to molecular biology, gene therapy, and compositions and methods for modifying expression and activity of RNA molecules.

### BACKGROUND

There are long-felt but unmet needs in the art for providing effective therapies for correcting dysfunctional messenger RNA.

Small nuclear RNA (snRNA) is one of the smallest types of RNA with an average size of about 150 nucleotides. snRNAs are functional non-coding RNAs. Eucaryotic genomes code for a variety of non-coding RNA such as snRNA, a class of highly abundant RNA, localized in the nucleus with important functions in intron splicing and RNA processing. snRNA, in the pre-mRNA splicing process, are capable of forming ribonucleoprotein particles (snRNPs) along with other proteins. These snRNPs and additional proteins form a large particulate complex (spliceosome) bound to the unspliced pre-mRNA transcripts. In addition to splicing, snRNAs function in nuclear maturation of nascent transcripts, gene expression regulation, as a splice donor in non-canonical systems, and in 3′ end processing of replication-dependent histone mRNAs. U7 snRNA can be programmed to bind and modulate mRNA without exogenous protein expression but there still exists a need to develop a highly specific mRNA-targeting therapeutic that minimizes immunogenic risk. Furthermore, the small size of these programmed snRNAs creates an opportunity to develop single vector, highly specific (allele-specific), single target and multi-targeting gene therapy approaches.

Accordingly, the disclosure provides compositions and methods comprising a new therapeutic RNA-targeting platform comprised of engineered snRNAs.

### SUMMARY

The disclosure provides gene therapy compositions comprising a new therapeutic RNA-targeting platform comprised of engineered snRNA (esnRNA) comprising an engineered snRNA stem loop (eSL).

Disclosed herein are compositions comprising nucleic acid molecules, and vectors comprising the engineered snRNA (esnRNA).

Disclosed herein is an RNA-targeting nucleic acid molecule comprising an engineered snRNA (esnRNA) system, wherein the esnRNA system comprises an engineered stem loop (eSL).

In one embodiment, the esnRNA comprises a 5′ interaction stabilizer domain (5′ISD).

In one embodiment, the esnRNA comprises a targeting sequence (TS) (or spacer) that targets an RNA of interest. In another embodiment, the target RNA of interest is a microsatellite repeat RNA or a non-repeat RNA. In another embodiment, the microsatellite repeat RNA of interest is selected from the group consisting of CUG, CAG, GGGGCC, and CCCCGG. In one embodiment, the esnRNA comprises a targeting sequence (TS) that targets two target RNAs of interest which are GGGGCC and CCCCGG. In another embodiment, the two target RNAs of interest are a microsatellite repeat RNA and a non-repeat RNA. In another embodiment, the non-repeat RNA is a flanking sequence to the microsatellite repeat RNA. In another embodiment, the esnRNA comprises a targeting sequence (TS) that targets two or more RNAs of interest. In another embodiment the esnRNA comprises two or more targeting sequence (TS) that targets two or more RNAs of interest. In another embodiment, the esnRNA comprises a fusion of the two or more TSs.

In one embodiment, the esnRNA comprises an Sm binding domain (SmBD). In one embodiment, the SmBD is selected from the group consisting of U1, U2, U4, and U5 snRNAs. In another embodiment, the SmBD is derived from a pseudo snRNA.

In one embodiment, the esnRNA is operably linked to an snRNA promoter. In another embodiment, the snRNA promoter is a U7 promoter. In another embodiment, the U7 promoter is an endogenous U7 promoter. In another embodiment, the U7 promoter is a human U7 promoter (hU7) or a mouse U7 promoter (mU7). In another embodiment, the U7 promoter is an endogenous human U7 promoter of SEQ ID NO: 43. In one embodiment, the snRNA promoter is a U1 promoter. In another embodiment, the U1 promoter is a human U1 promoter or a mouse U1 promoter. In another embodiment, the snRNA promoter is selected from the group consisting of: a human U7 promoter, a human U1 promoter, a human U2 promoter, a human U4 promoter, a human U5 promoter, a human U6 promoter, a human 7sk promoter, a tRNA(Val) promoter, a mouse U1 promoter, and a mouse U7 promoter. In another embodiment, the vector comprises one or more esnRNAs, or one or more copies of the esnRNA driven by one or more snRNA promoters. In one embodiment, the vector comprises two copies (2×) of the esnRNA. In one embodiment, the vector comprises two snRNA promoters, wherein each promoter drives a copy of the 2×esnRNA. In another embodiment, the two snRNA promoters are the U7 promoter and the U1 promoter. In another embodiment, the two snRNA promoters are selected from the group consisting of: a human U7 promoter, a human U1 promoter, a human U2 promoter, a human U4 promoter, a human U5 promoter, a human U6 promoter, a human 7sk promoter, a tRNA(Val) promoter, a mouse U1 promoter, a mouse U2 promoter, a mouse U5 promoter, a mouse U6 promoter, a mouse U7 promoter, and a mouse H1 promoter. In another embodiment the two snRNA promoters are a mouse U7 promoter and a mouse U1 promoter.

In one embodiment, the esnRNA is operably linked to an snRNA downstream terminator (DT). In another embodiment, the snRNA DT is a U7 DT.

In one embodiment, the esnRNA comprises the eSL, the U7 promoter, the TS, the SmBD, the 5′ISD, and the DT. In another embodiment, the esnRNA comprises the eSL, the U7 promoter, the U1 promoter, the TS, the SmBD, the 5′ISD, and the DT.

In one embodiment, the esnRNA comprises the eSL, the U7 promoter, the TS, the SmBD, the 5′ISD, and the DT. In another embodiment, the esnRNA comprises the eSL, the U7 promoter, the U1 promoter, the TS, the SmBD, the 5′ISD, and the DT.

Also disclosed herein is a vector comprising the esnRNA. In some embodiments, the vector comprises multiple copies of the esnRNA. In some embodiments, the multiple copies of the esnRNA is 2, 3, or 4 copies (2×, 3×, or 4×) of the esnRNA. In some embodiments, the multiple copies of the esnRNA is 4 or more copies (4× or more) of the esnRNA. In some embodiments, each esnRNA of the multiple copies of esnRNA is separated by a nucleic acid buffer sequence derived from human non-coding genomic sequences downstream of an snRNA. In one embodiment, the buffer sequence is derived from human genomic sequences downstream of U7. In one embodiment, the buffer sequence is selected from the group consisting of: buffer 1 (30 bp, 100 bp, or 500 bp), buffer 2 (30 bp, 100 bp, or 500 bp), buffer 3 (30 bp, 100 bp, or 500 bp), and a combination thereof. In one embodiment, the vector is a viral vector or a non-viral vector. In another embodiment, the viral vector is an AAV vector. In another embodiment, the AAV vector is an scAAV vector or a ssAAV vector. In another embodiment, the AAV vector is an AAV9 vector. In another embodiment, the AAV9 vector is an ssAAV9 vector or an scAAV9 vector.

Also disclosed herein is a method of targeting one or more target RNAs of interest and blocking, knocking down, editing, exon-skipping, exon inclusion or splicing the one or more target RNAs, comprising contacting an esnRNA of the disclosure with a cell comprising the one or more target RNAs.

The disclosure provides aa RNA-targeting nucleic acid molecule comprising an engineered snRNA esnRNA, wherein the esnRNA system comprises an engineered stem loop (eSL) comprising one or more nucleic acid sequences selected from SEQ ID NO: 1-SEQ ID NO: 11, SEQ ID NO: 146-SEQ ID NO: 148, SEQ ID NO: 163, or SEQ ID NO: 186-205.

In some embodiments, the esnRNA comprises a targeting sequence (TS) that targets a target RNA of interest.

In some embodiments, the target RNA is a pre-mRNA or mRNA sequence. In some embodiments, the target RNA of interest is a microsatellite repeat RNA. In some embodiments, the microsatellite repeat RNA is selected from the group consisting of CUG, CAG, and GGGGCC+CCCCGG.

In some embodiments, the target RNA is a sequence encoding DMD. In some embodiments, the targeting sequence is selected from SEQ ID NO: 206 or SEQ ID NO: 207. In some embodiments, the targeting sequence comprises one or more nucleic acid sequences set forth in SEQ ID NO: 208-SEQ ID NO: 227.

In some embodiments, the esnRNA comprises two targeting sequences that target two RNAs of interest. In some embodiments, the two TSs are a fusion sequence.

In some embodiments, the esnRNA comprises an Sm binding domain (SmBD) selected from the group consisting of a U1, U2, U4, and U5 SmBD.

In some embodiments, the SmBD comprises a nucleic acid sequence set forth in any one of SEQ ID NO: 31-SEQ ID NO: 38, or SEQ ID NO: 164.

In some embodiments, the esnRNA comprises a 5′ interaction stabilizer domain (5′ISD) comprising a nucleotide sequence selected any one of SEQ ID NO: 12-SEQ ID NO: 23.

In some embodiments, the esnRNA comprises a nucleic acid sequence set forth in any one of SEQ ID NO: 65-SEQ ID NO: 119 or SEQ ID NO: 179-SEQ ID NO: 185.

The disclosure provides a vector comprising one or more esnRNA of the disclosure. In some embodiments, the viral vector is an AAV vector. In some embodiments, the esnRNA is operably linked to a promoter.

In some embodiments, the esnRNA is operably linked to a U7 promoter or a U1 promoter.

In some embodiments, the esnRNA is operably linked to a downstream terminator (DT). In some embodiments, the esnRNA is operably linked to a U7 downstream terminator or a U1 downstream terminator.

In some embodiments, the vector comprises at least one, at least two, at least three, at least four, or at least five esnRNA. In some embodiments, the least one, at least two, at least three, at least four, or at least five esnRNA each target the same target RNA sequences.

In some embodiments, the least one, at least two, at least three, at least four, or at least five esnRNA target two or more target RNA sequences In some embodiments, each esnRNA is separated by a buffer sequence. In some embodiments, the buffer sequence comprises a nucleic acid sequence set forth in any one SEQ ID NO: 24-SEQ ID NO: 30.

In some embodiments, the vector comprises a nucleic acid sequence set forth in any one of SEQ ID NO: 123-SEQ ID NO: 143, SEQ ID NO: 168-SEQ ID NO: 178, or SEQ ID NO: 231-SEQ ID NO: 233.

A DMD exon 51 RNA-targeting nucleic acid molecule comprising a spacer sequence set forth in any one of SEQ ID NO: 206-SEQ ID NO: 230.

The disclosure provides a method of treating a disease or disorder in a subject comprising administering an RNA-targeting nucleic acid molecule of the disclosure or an AAV vector of the disclosure.

In some embodiments, the disease or disorder is associated with a toxic repeat RNA sequence. In some embodiments, the toxic repeat RNA sequence is a CAG, CUG, GGCCCC, CCGGG, or GGCCC+CCGGGG RNA repeat.

In some embodiments, the disease or disorder is myotonic dystrophy (DM1) or Huntington's disease (HD).

In some embodiments, the disease or disorder is Duchenne Muscular Dystrophy. In some embodiments, the RNA-targeting nucleic acid molecule or AAV vector targets an RNA sequence encoding dystrophin (DMD).

In some embodiments, the RNA sequence encoding DMD comprises an intronic or exonic sequence. In some embodiments, the exonic sequence comprises exon 51, or a flanking region thereof, of DMD. In some embodiments, the administration is administration is intravenous, intramuscular, subpial, intrathecal, intraparenchymal, intrathecal, intrastriatal, subcutaneous, intradermal, intraperitoneal, intratumoral, intravenous, intraocular, and/or parenteral administration.

### BRIEF DESCRIPTION OF THE DRAWINGS

FIG. 1A-D. FIG. 1A shows a tape station image of PCR products which increase in abundance for the engineered SL indicating increased stability of an esnRNA disclosed herein. Predicted PCR product size is shown below the image. FIG. 1B illustrates a schematic of the engineered snRNA (esnRNA) comprising the engineered stem loop (eSL) disclosed herein alongside non-engineered stem loops. FIG. 1C shows alternative mechanisms to protect the 5′ end of the snRNA which forego the use of an interaction stabilizer domain (ISD). FIG. 1D shows the approximate size of an snRNA cassette.

FIG. 2A-C. FIG. 2A-2B shows FISH quantification and images of CUG RNA Foci of an esnRNA targeting CUG disclosed herein compared to NT (non-targeting) esnRNA. Y axis depicts percentage average foci per cell normalized to NT, and x axis depicts treatment. Cell nuclei was stained with DAPI and CUG RNA foci labeled with a specific labeled probe. FIG. 2C shows a schematic of the esnRNA MOA targeting and blocking CUG repeat expansions releasing MBLN and restoring splicing defects characteristic in DM1 disease.

FIG. 3A-C. FIG. 3A shows qPCR results of a dose-dependent knockdown of CAG80 reporter mRNA to baseline levels. Y axis depicts CAG repeat RNA expression levels relative to reference gene GAPDH, and x axis depicts snRNA treatment CAG targeting (CUG15) or non-targeting (nt) (using different doses 250 ng, 500 ng or 1000 ng). FIG. 3B shows a Western blot for CAG80 reporter poly-Q protein expression. Top lanes show CAG reporter poly-Q expression and bottom lane GAPDH loading control. FIG. 3C shows CAG repeat expansions in mutant HTT in Huntington's disease and the esnRNA MOA blocking the CAG repeats.

FIG. 4A-D. FIG. 4A shows a tape station image of the PCR product to detect USH2A exon 13 inclusion (top band) or exclusion (bottom band) after treatment with esnRNA. FIG. 4C shows the quantification of percentage of exon 13 exclusion after esnRNA treatment compared to NT esnRNA control (shown in A). FIG. 4B shows a schematic of the engineered snRNA comprising the eSL compared to the non-engineered snRNAs. FIG. 4D shows a schematic of splicing modulation in Usher's Syndrome and DMD (Duchenne Muscular Dystrophy).

FIG. 5A-F shows PCR results for restoration of splicing isoforms for BIN1, DMD and LBD3 (mispliced in DM1 patient cells) after treatment with esnRNAs to target CUG repeats in DMlpatient derived myotubes. Figures A-C show quantitation of the percentage restoration to the healthy splice isoform from the tape station images shown in Figures D-F. Y axis depicts percentage of correction, and x axis depicts treatment (knt, refers to non-targeting negative control; CAGx15 refers to CUG targeting esnRNA; unt refers to untreated).

FIG. 6A-6B shows quantification of efficacy of esnRNA with alternative 5′ interaction stabilizing domain (ISD) in knocking down CUG RNA foci, synthetic snRNA transfection in CTG480 HeLa cells followed by fluorescent in-situ hybridization for CUG RNA foci. (Bars sR220109, sR220110, sR220111, sR220112, sR220113, sR220114, sR220115, sR220116, sR220117, sR220118, sR220119, sR220120, sR2200092, sR2200093, sR2200094, sR2200095, and sR2200096=alternative engineered stem loops with corresponding 5′ ISDs. Bar sR220010=selected engineered stem loop and corresponding 5′ ISD. sR220004=non-targeting snRNA). Synthetic snRNAs were transfected at 33 nM (A) or 11 nM (B) dose.

FIG. 7A-7B shows that different ISDs lead to varying levels of stability over time. FIGS. 7A and B show the relative abundance over time of synthetic engineered snRNAs with alternative 5′ stabilizing domains. RNA was collected at 24 hr, 48 hr, and 72 hr time points after transfection of synthetic snRNAs with varying stabilizing domains to determine the relative stability of the snRNA.

FIG. 8 shows levels of snRNA expression by qPCR in CTG480 HeLa cells when snRNAs are expressed under distinct PolII and PolII promoters. Y axis depicts snRNA expression levels relative to GAPDH reference gene, and x axis depicts the different snRNAs promoters used to express snRNAs (Pol II U1 and U7 promoters or PolIII: U6, tRNA and 7SK). Plasmids with varying promoters were transfected at two different doses (250 and 500 ng).

FIG. 9A-9B shows quantification of CUG foci by RNA FISH in CTG480 HeLa cells after transfection of 500 ng (A) or 250 ng (B) engineered snRNA constructs with varying Pol III and Pol II promoters and respective DT sequences. Constructs with Pol II promoters (P02225 and P03557) driving expression of engineered snRNAs are more effective at depleting microsatellite repeat-containing foci than constructs with Pol III promoters driving snRNAs. Bars P03491, P03463, P03466, P03561, and P02313 represent NT esnRNA and bars P03490, P03462, P03465, P03557, and P02225 represent CUG targeting esnRNAs.

FIG. 10 shows levels of snRNA expression by qPCR in HEK293T cells when snRNAs are expressed under PolIII (U6, tRNA and 7SK) or PolII promoters (U7 and U1) and contain the SmOpt or the U1 Sm binging sites.

FIG. 11A-11B shows levels of snRNA expression relative to GAPDH reference gene after transfection into HEK293 cells. Primers specific for each snRNA (z) were used for qPCR to determine relative promoter strength. Y axis depicts snRNA expression levels relative to GAPDH and x axis depicts the snRNA spacer znt (non-targeting control, in A) and z38 (targeting spacer, B) when they are expressed under snRNA distinct promoters (U1, U2, U4, U5 and U7; unt refers to untreated). Primer sets specific to z-nt (FIG A) or z-38 (FIG B) were used.

FIG. 12A-12B shows results for exon 51 skipping after transduction of human delta exon 52 myotubes with snRNA disclosed herein (A03980: 2×z38/42 fusion snRNA cassettes; A03981: 2 snRNA cassettes, one with z42 TS and the other with z38 TS). FIG. 12A shows a tape station image of PCR products representing the exon 51 included and excluded isoforms and a schematic showing expected fragment sizes. FIG. 12B shows the quantification of the band intensities in FIG. 12A semi-quantitative PCR. Y axis depicts percentage of exon 51 skipping and x axis the treatment (either synthetic snRNA or AAV transduced for A03980 of A03981 with different MOIs).

FIG. 13A-13B shows dystrophin staining and restoration of human delta exon 52 myotubes after treatment with snRNAs for exon 51 skipping. FIG. 13A shows immunofluorescence for desmin or dystrophin in untreated myotubes, myotubes treated with synthetic snRNAs or myotubes treated with MOI of 1e6 or 5e6 of A03980 (2×z38/z42 fusion snRNA cassettes). FIG. 13B shows immunofluorescence for desmin or dystrophin in myotubes transduced with MOI of 1e6 or 5e6 of A03981 (2 snRNA cassettes with z42 TS and z38 TS).

FIG. 14A illustrates the vector genome of A04569, which expresses 2 fusion snRNAs of identical sequence, as depicted in FIG. 14B. FIG. 14C shows that exon 51 Exon Splicing Enhancer (ESE)-targeting fusion snRNAs can be constructed such z38-targeting sequence is in the 5′-most position in the antisense sequence with z42-targeting sequence downstream of the z38 sequence (z73) or in the opposite orientation with z42-targeting sequence in the 5′-most position and z38 downstream (z187). In both cases, the antisense sequence of the snRNAs is composed of 2 targeting sequences, each of which targets a separate and unique ESE in exon 51 (FIG. 14C bottom diagram).

FIG. 15A-D show the activity of exon 51 ESE-targeting fusion snRNAs in human skeletal myotubes with exon 52 deleted and del52hDMD/mdx mice. (A) tape station image and quantification of exon 51 skipping in human skeletal myotubes with DMD exon 52 deleted 24 hours after transfection of a low dose of synthetic snRNAs carrying the fusion antisense sequences z73 (z38/z42) and z187 (z42/z38). At a low dose, transfection of synthetic snRNA z187 induces higher levels of exon 51 skipping than z73. (FIG. 15B) shows quantification of exon 51 skipping in human skeletal myotubes with DMD exon 52 deleted after transduction with AAV9-A04526 (carrying two esnRNA cassettes each expressing z73) and A04569 (carrying two cassettes each expressing z187) for 7 days with MOIs of 2.5e4, 1e5 and 5e5. (FIG. 15C) shows quantification of exon 51 skipping in the humanized exon 52-deleted DMD (del52hDMD/mdx) mouse model. Mice we injected retro-orbitally with 3e12 vector genomes of A04526 and A04569. Following 3 week survival, RNA was extracted from the tibialis anterior muscle (TA), the Gastrocnemius (Gc) and the heart. Semi-quantitative RT-PCR was performed to detect exon 51 included and excluded bands. (FIG. 15D) shows the quantification of dystrophin positive fibers 4 weeks after intramuscular delivery of a 3e11 vg/muscle dose of A04526 or an ESE-targeting Vivo-Morpholino (ViM) to the Gastrocnemius of del52hDMD/mdx mice. hDMD/mdx mice (gray bar) which expresses wildtype human dystrophin serves as a positive control for dystrophin expression. U=untreated.

FIG. 16A-D show splicing reversal of DMD and LBD3 splice isoforms and knockdown of DMPK after treatment with snRNA targeting CUG repeats in patient derived myotubes (containing 1700 CUG repeats). DM1 patient fibroblasts were differentiated into myotubes by transduction with myoD Adenovirus vector (for 5 days). Then cells were transduced with multiple MOIs of A04233 and A04234 (scAAV9 dual snRNAs targeting CUG repeats) and harvested 7 days post-transduction. FIG. 16A shows a tape station image of RT-PCR products using specific primers to detect DMD exon 78 inclusion (top band) and LBD3 exon 11 exclusion (bottom band) which are markers of splicing correction seem in normal WT cells. Quantification of DMD exon 78 inclusion (FIG. 16B) and LBD3 exon 11 exclusion (FIG. 16C). The x-axis depicts the treatment (vector and MOI). The y-axis depicts the percentage of splice correction, either exon inclusion for DMD exon 78 or exclusion for LBD3 exon 11. FIG. 16D shows levels of DMPK RNA expression by ddPCR after treatment with snRNAs (AAV9 A04233 and AAV9 A04234) with different MOIs. The x-axis depicts the treatment. The y-axis depicts DMPK copies per 1000 copies of GAPDH reference gene normalized to UNT (untreated cells).

FIG. 17A-B shows images and quantification of nuclear RNA foci from patient-derived myotubes transduced with different MOIs of AAV9 A04233 and AAV9 A04234. DM1 patient fibroblasts were differentiated into myotubes by transduction with myoD Adenovirus vector (for 5 days). Then cells were transduced with multiple MOIs of A04233 and A04234 (scAAV9 dual snRNAs targeting CUG repeats) and harvested 7 days post-transduction for RNA FISH (to detect CUG Foci). FIG. 17A shows images of CUG RNA foci obtained from untreated and treated myotubes with AAV9 snRNA vectors A04233 and A04234. Cell nuclei was stained with DAPI and CUG FISH was performed with a CUG labeled probe. Cells were processed 7 days post-transduction. FIG. 17B shows quantification for the average number of CUG foci per cell. The x-axis depicts the treatment and MOI used. The y-axis depicts average of CUG foci per cell normalized to UNT (untreated cells).

FIG. 18A-C depicts the treatment, timeline and quantification of HTT mutant soluble protein after treatment with AAV-snRNA targeting CAG repeats. FIG. 18A shows the experimental design for the vector used AAV-A04390 (2×hU7/hU1 snRNA targeting CAG repeats), the 2 MOIs and the end point assay to detect mutant (mut) HTT soluble protein. FIG. 18B depicts the timeline for this experiment. HD patient iPSc (containing 66 CAG repeats) were differentiated into cortical neurons for 2 weeks and then transduced with AAV-A04390 snRNA vector for an additional 2 weeks. Then cortical neurons were harvested and mutant soluble HTT protein quantified by Meso scale discovery immunoassay (MSD, shown in FIG. 18C). The y-axis depicts fmol of mutant soluble HTT per mg of protein lysate, and the x-axis the treatment and MOI.

FIG. 19A-B depicts the in vivo study design to target CAG repeats on R6/2 HD mouse model and quantification of mutant HTT soluble protein after treatment with AAV-snRNA targeting CAG repeats (A03081). FIG. 19A diagram shows dosage (2e10 vector genomes) of AAVrh10-snRNA (4×CAGx15 targeting snRNAs vector) administered by intrastriatal injection. Untreated or uninjected contralateral striatal side was used as a control. (FIG. 19B) shows quantification of mutant soluble HTT protein by Meso scale discovery immunoassay (MSD) on untreated and snRNA treated mice (n=10). The y-axis depicts fmol of mutant soluble HTT per mg of total protein lysate, and the x-axis the treatment.

FIG. 20A depicts the differences between the snRNAs expressed from A04527 and A04526 with A04526 expressing engineered snRNAs that have a 5′ISD and engineered mouse SL and A04527 which has no 5′ISD and a mouse native SL. FIG. 20B shows a bar graph of the expression of z38/42 (in snRNA copies/nanogram) 2 days after transduction of CHO-Lec2 cells at an MOI of 1e6 for with AAV9-A04527 or A04526 virus.

### DETAILED DESCRIPTION

The disclosure provides gene therapy compositions comprising a new therapeutic RNA-targeting platform comprised of engineered snRNA (esnRNA) comprising an engineered snRNA stem loop (eSL).

Disclosed herein are compositions comprising nucleic acid molecules, and vectors comprising the engineered snRNA (esnRNA).

Small nuclear ribonucleic acids (snRNAs) are essential components of small nuclear ribonucleoprotein complexes (snRNPs) which, when assembled with additional proteins, form the large ribonucleoprotein complex known as the spliceosome, the cell machinery appointed to mediate the entire mRNA maturation process. The spliceosome is responsible for precursor mRNA splicing; the process that removes introns from RNA transcripts before protein production. An individual snRNA is generally about 250 nucleotides or less in size. For example, U1 snRNA is 164 nucleotides in length and is encoded by genes that occur in several copies within the human genome. U1 snRNA represents the ribonucleic component of the nuclear particle U1 snRNP. The U1 snRNA has a stem and loop tridimensional structure and within the 5′ region there is a single-stranded sequence, generally about 9 nucleotides in length, capable of binding by complementary base pairing to the splicing donor site on the pre-mRNA molecule. (Horowitz et al., 1994, Trends Genet., 10(3):100-6.) The various spliceosomal snRNAs have been designated as U1, U2, U4, U5, U6, U4ATAC, U6ATAC, U7, U11 and U12, due to the generous amount of uridylic acid they contain. (Mattaj et al., 1993, FASEB J, 15, 7:47-53.)

snRNA systems can be used for treating toxic mutations. For example, antisense oligonucleotides that interfere with splice sites and regulatory elements within an exon containing toxic mutations can induce skipping of specific exons at the pre-RNA level. Such antisense sequences can be packaged in an snRNA sequence delivered using viral vectors carrying a nucleic acid sequence from which the snRNA can be transcribed. U7 snRNA is endogenously involved in histone pre-mRNA 3′-end processing, but can be converted into a versatile tool for splicing modulation by a small change in the binding site for Sm/Lsm proteins. One such therapeutic strategy for treating Duchenne muscular dystrophy has used modified U7 snRNA to convert an out-of-frame mutation into an in-frame mutation, which gives rise to internally deleted toxic RNA, but still functional dystrophin. (Goyenvalle et al., 2009, 17(7): 1234-1240.)

Most U-rich snRNPs are complexes that mediate the splicing of pre-mRNAs. U7 snRNP is an exception. U7 is not involved in splicing but rather is a key factor in the unique 3′-end processing of replication-dependent histone mRNAs. By modifying the U7 snRNA histone binding sequence and the Sm motif, U7 can no longer be involved in processing the histone pre-mRNA and instead targets pre-mRNAs or smRNA for blocking or splicing modulation. In this manner, U7 snRNA can be used as an effective gene therapy platform. A U7 snRNA platform also has the additional advantages of being a compact size, having the capability to accumulate in the nucleus without causing cellular toxicity, and possesses little to no immunoreactivity. (Gadgil et al., 2021, J Gene Med, 23(4): e3321.)

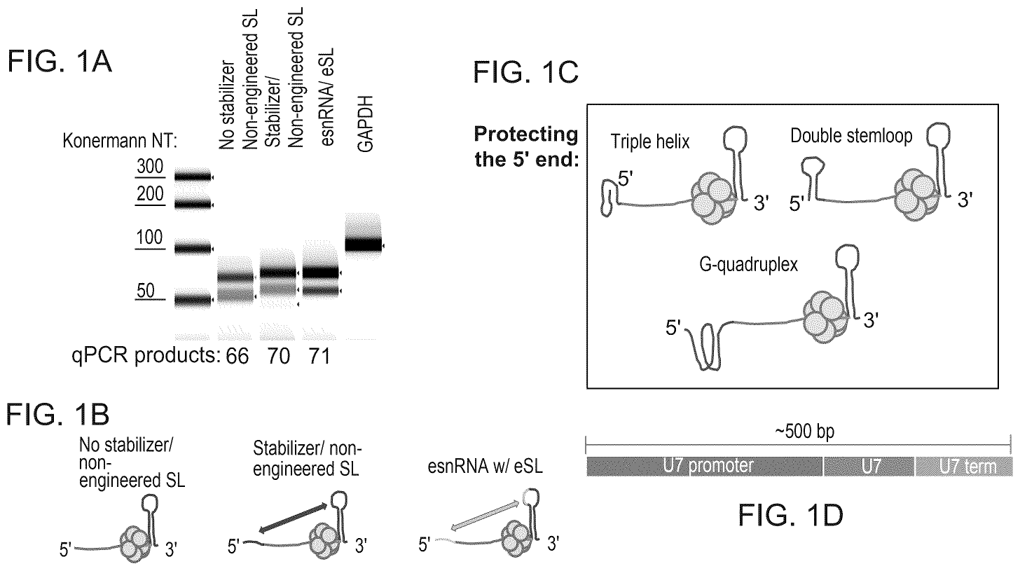

Disclosed herein is a newly engineered and redesigned snRNA platform (or known herein as an esnRNA platform) comprising an 1) engineered stem loop (eSL). Compensatory modifications made to the native stem loop sequence create an engineered stem loop (eSL) which more effectively communicates (folds and anneals) with the snRNA interaction stabilization domain (ISD) which in turn creates a snRNA platform with increased stability. See FIG. 1. U7 snRNAs have been previously shown to be programmable to modulate mRNAs. Disclosed herein are programmed engineered snRNA improvements which are capable of being used as a gene therapy tool. These engineered snRNA systems are shown herein to lead to blocking microsatellite repeat expansions (shown herein for treating myotonic dystrophy (DM1) or Huntington's disease (HD)), exon skipping (for treating DMD) and splicing modulation (shown herein for treating USH2A (Usher Syndrome type 2). In one embodiment, these snRNAs are human snRNAs. In another embodiment, these snRNAs are mouse snRNAs. In another embodiment, the snRNAs are a combination of human and mouse snRNAs. In one embodiment the U7 is a human U7 or a mouse U7. In another embodiment disclosed herein, engineered snRNA comprises varying types of snRNAs (U1-U12, etc.) by combining domains of endogenous snRNAs to fine tune stabilization of the platform and/or to reduce off-target effects. For example, in one embodiment, the engineered snRNA system comprises a combination of human or mouse U7 and human or mouse U1 snRNA components.

Additional elements that can tune the processing and abundance of the RNA can be further engineered into the esnRNAs comprising eSLs. See FIG. 1C. In one embodiment, additional elements that can tune the processing, stability, and abundance of the esnRNA can be further engineered into the esnRNAs at the 5′ or 3′ ends. In another embodiment, such elements may include but are not limited to stem loops, hairpins, G-C clamps, kissing loops, triplexes, quadruplexes, and protein binding sites.

The esnRNA platform and portions thereof disclosed herein can be used in any therapeutic setting and context so long as a suitable spacer(s) (or TS(s)) is included in the design of the esnRNA therapeutic composition. In certain embodiments, a therapeutic esnRNA composition is used to treat a disease selected from the group consisting of Duchenne Muscular Dystrophy, DM1, and HD. In another embodiment, the esnRNA composition is used to treat DMD caused by exon 51 mutations

### Engineered Stem Loops

The engineered snRNA (esnRNA) system disclosed herein comprises an engineered stem loop (eSL) which includes compensatory modifications to a native snRNA stem loop. These modifications result in increased stability of the esnRNP compared to snRNP comprising an unmodified stem loop. An eSL disclosed herein can be derived from any snRNP such as U1-U12. In one embodiment, the eSL is a human or mouse U7 eSL. In one embodiment, the eSL is a human or mouse eSL. In some embodiments, the eSL is a human and mouse eSL. In some embodiments, the eSL is a non-human eSL selected from the group consisting of mouse, pig, sheep, goat, cow, dog, cat, horse, or a combination thereof. In some embodiments, the eSL is an eSL selected from the group consisting of human, mouse, pig, sheep, goat, cow, dog, cat, horse, or a combination thereof. In some embodiments, the eSL sequence is not a native stem loop sequence. In some embodiments, the nucleic acid sequence of the eSL is at least 65%, 70%, 75%, 80%, 85%, 90%, 95%, 96%, 97%, 98%, 99% or 100% (or any percentage in between) is not a native stem loop sequence.

In some embodiments, a human eSL comprises, consists essentially of, or consists of a nucleic acid sequence at least 65%, 70%, 75%, 80%, 85%, 90%, 95%, 96%, 97%, 98%, 99% or 100% (or any percentage in between) identical to one or more of the following nucleotide sequences:

(SEQ ID NO: 1))
ggctttctggctccttaccggaaagcc,
(SEQ ID NO: 2)
ggctttctgggaggttaccggaaagcc,
(SEQ ID NO: 3)
ggctttctggcctccttaccggaaagcc,
(SEQ ID NO: 4)
ggctttctggggaggttaccggaaagcc,
(SEQ ID NO: 5)
ggctttctggctggctaccggaaagcc,
(SEQ ID NO: 6)
ggctttctggcttccccggaaagcc,
(SEQ ID NO: 7)
ggctttctggcttcttcccggaaagcc,
(SEQ ID NO: 8)
ggctttctggcaacttaccggaaagcc,
(SEQ ID NO: 9)
ggctttctggttcggtaccggaaagcc,
(SEQ ID NO: 10)
ggctttctggaagccttaccggaaagcc,
(SEQ ID NO: 11)
ggctttctggcttcttaccggaaagcc,
or
(SEQ ID NO: 186)
GGCTTTCTGGCCTCCGCCGGAAAGCCCCT.

In some embodiments, a murine eSL comprises, consists essentially of, or consists of a nucleic acid sequence at least 65%, 70%, 75%, 80%, 85%, 90%, 95%, 96%, 97%, 98%, 99% or 100% (or any percentage in between) identical to one or more of the following nucleotide sequences:

(SEQ ID NO: 163)
ggctttctggctccttaccggaaagcccct
(SEQ ID NO: 146)
Ggttttctgacctccgtcggaaaacccct,
(SEQ ID NO: 147)
ggttttctgacctccttcggtcggaaaacccct,
(SEQ ID NO: 148)
Ggttttctgacctccgtcggaaaacc,
(SEQ ID NO: 228)
GGTTTTCTGACACTCCGTCGGAAAACCCCT,
(SEQ ID NO: 229)
GGTTTTCTGATCTCCATCGGAAAACCCCT,
or
(SEQ ID NO: 230)
GGTTTTCCGACCTCCGTCGGAAAACCCCT.

In some embodiments, a human or murine eSL comprises, consists essentially of, or consists of a nucleic acid sequence at least 65%, 70%, 75%, 80%, 85%, 90%, 95%, 96%, 97%, 98%, 99% or 100% (or any percentage in between) identical to one or more of the following nucleotide sequences:

(SEQ ID NO: 190)
GGCTTTCTGGCACTCCACCGGAAAGCCCCT,
(SEQ ID NO: 191)
GGCTTTCTGGCACTCCGCCGGAAAGCCCCT,
or
(SEQ ID NO: 192)
GGCTTTCTGGCCTCCACCGGAAAGCCCCT.

In some embodiments, a dog or cat eSL comprises, consists essentially of, or consists of a nucleic acid sequence at least 65%, 70%, 75%, 80%, 85%, 90%, 95%, 96%, 97%, 98%, 99% or 100% (or any percentage in between) identical to one or more of the following nucleotide sequences:

(SEQ ID NO: 193)
GGTTTTCCGGTCTCCACCGGAAAGCCCCC.

In some embodiments, a cow, sheep, or goat eSL comprises, consists essentially of, or consists of a nucleic acid sequence at least 65%, 70%, 75%, 80%, 85%, 90%, 95%, 96%, 97%, 98%, 99% or 100% (or any percentage in between) identical to one or more of the following nucleotide sequences:

(SEQ ID NO: 194)
GGCTTTCCGGTCTCCACCGGAAAGCCCCT,
or
(SEQ ID NO: 195)
GGCTTTCCGGCCTCCGCCGGAAAGCCCCT.

In some embodiments, a pig eSL comprises, consists essentially of, or consists of a nucleic acid sequence at least 65%, 70%, 75%, 80%, 85%, 90%, 95%, 96%, 97%, 98%, 99% or 100% (or any percentage in between) identical to one or more of the following nucleotide sequences:

(SEQ ID NO: 196)
GGTTTTCCGGTCTCCACCGGAAAACCCTT,
(SEQ ID NO: 197)
GGTTTTCCGTGCTCCCACGGAAAACCCTT,
(SEQ ID NO: 198)
GGTTTTCCGGCCTCCGCCGGAAAACCCTT,
(SEQ ID NO: 199)
GGTTTTCCGTGACTCCCACGGAAAACCCTT,
or
(SEQ ID NO: 200)
GGTTTTCCGGCACTCCGCCGGAAAACCCTT.

In some embodiments, a horse eSL comprises, consists essentially of, or consists of a nucleic acid sequence at least 65%, 70%, 75%, 80%, 85%, 90%, 95%, 96%, 97%, 98%, 99% or 100% (or any percentage in between) identical to one or more of the following nucleotide sequences:

(SEQ ID NO: 201)
GGTCTTCCGGTCTCCTCCGGAAGGCCCCC,
or
(SEQ ID NO: 202)
GGTCTTCCGGCTCCCCGGAAGGCCCCC.

In some embodiments, a sheep eSL comprises, consists essentially of, or consists of a nucleic acid sequence at least 65%, 70%, 75%, 80%, 85%, 90%, 95%, 96%, 97%, 98%, 99% or 100% (or any percentage in between) identical to one or more of the following nucleotide sequences:

(SEQ ID NO: 203)
GGCTTTCCGTGCTCCCACGGAAAGCCCCT,
(SEQ ID NO: 204)
GGCTTTCCGTGACTCCCACGGAAAGCCCCT,
or
(SEQ ID NO: 205)
GGCTTTCCGGCACTCCGCCGGAAAGCCCCT.

In some embodiments, engineered stem loops provide for enhanced stability of an snRNA relative to an snRNA comprising a native stem loop. In some embodiments is a native snRNA stem loop comprises, consists essentially of, or consists of a nucleic acid sequence at least 65%, 70%, 75%, 80%, 85%, 90%, 95%, 96%, 97%, 98%, 99% or 100% (or any percentage in between) identical to one or more of the following nucleotide sequences:

(SEQ ID NO: 145)
Ggttttctgacttcggtcggaaaacccct,
(SEQ ID NO: 206)
ggttttctgacttcggtcggaaaacc,
(SEQ ID NO: 235)
Ggctttctggctttttaccggaaagcc,
or
(SEQ ID NO: 236)
ggctttctggctttttaccggaaagccCCT.

### 5′ Interaction Stability Domain

The eSL disclosed herein possesses more effective folding and annealing properties with a 5′ interaction stability domain (5′ISD) and this in turn results in increased stability of the esnRNA compared to a non-engineered snRNA. The 5′ ISD has nucleotides that are complementary to the nucleotides within the engineered SL, and without wishing to be bound by theory, an interaction between the 5′ISD and eSL is predicted to form secondary structure that protects the 5′ end of an snRNA. In some aspects, the 5′ ISD anneals and/or hybridizes to an eSL of the disclosure. In some aspects, the 5′ISD is a sequence having complementarity and/or reverse complementarity to a sequence present in an eSL of the disclosure. In some aspects, a 5′ISD disclosed herein can be one of the 5′ISDs selected from the following nucleotide sequences:

(SEQ ID NO: 12)
ggagt,
(SEQ ID NO: 13)
cctct,
(SEQ ID NO: 14)
ggaggt,
(SEQ ID NO: 15)
cctcct,
(SEQ ID NO: 16)
agccag,
(SEQ ID NO: 17)
ggaag,
(SEQ ID NO: 18)
gaagaag,
(SEQ ID NO: 19)
gttg,
(SEQ ID NO: 20)
ccgaa,
(SEQ ID NO: 21)
taaggag,
(SEQ ID NO: 22)
gaag,
or
(SEQ ID NO: 23)
ggctt.

### Targeting Sequences

The esnRNA systems can be programmed to comprise a targeting sequence (TS) (also termed spacer). In some aspects, the targeting sequence is a 5′ targeting sequence (5′TS) (also termed spacer) that targets one or more RNAs of interest. In this context, 5′ is in reference to the snRNA insert's 5′ end and not necessarily to the overall vector configuration comprising the snRNA insert or inserts. And the TS can be located in or near the 5′ end of the snRNA. In an alternative embodiment, the targeting sequence(s) (TS) can be located in or near a 3′ position in the snRNA construct, thereby generating a 3′ targeting sequence (3′ TS), particularly if the snRNA construct is not a U7-based snRNA.

Targeting sequences of the disclosure, including 5′ TS, and 3′TS can be between about 1 and about 200 nucleotides in length. In some aspects, targeting sequences of the disclosure are between about 10 and about 150 nucleotides in length. In some aspects, targeting sequences of the disclosure are between about 10 and about 100 nucleotides in length. In some aspects, targeting sequences of the disclosure are between about 20 and about 60 nucleotides in length. In some aspects, targeting sequences of the disclosure are at least about 10, 20, 30, 40, 50, 60, or about 70 nucleotides in length.

In one example, U7 snRNA can be programmed by replacing the histone mRNA binding sequence with a sequence complementary to a target of interest. In some aspects, esnRNA systems of the disclosure bind a target mRNA or pre-mRNA sequence of interest. The exemplary esnRNA systems shown herein lead to blocking or knocking down microsatellite repeat expansions (for treating myotonic dystrophy (DM1) or Huntington's disease (HD), hexanucleotide repeat expansion (for treating C9/ALS), splicing modulation (for treating USH2A (Usher Syndrome type 2)), or targeting one or more exon splicing enhancers (ESE) to induce exon skipping (for treating DMD, e.g., DMD exon 51 skipping). In one embodiment, the target RNA of interest is a microsatellite or hexanucleotide repeat RNA or a non-repeat RNA. In another embodiment, the repeat RNA of interest is selected from the group consisting of CUG, CAG, GGGGCC, and CCCCGG. In one embodiment, the esnRNA comprises a targeting sequence (TS) that targets two target RNAs of interest are GGGGCC and CCCCGG. In another embodiment, the two target RNAs of interest are a microsatellite repeat RNA and a non-repeat RNA. In another embodiment, the non-repeat RNA is a flanking sequence to the repeat RNA.

In some embodiments, esnRNA of the disclosure target a pre-mRNA or mRNA sequence encoding the DMD gene. DMD is a gene encoding the protein dystrophin. Mutations in DMD are associated with Duchene muscular dystrophy. In some embodiments, the DMD RNA sequence targeted by esnRNA compositions of the disclosure is an exon 51 DMD RNA sequence.

Target sequences that bind DMD can comprise, consist essentially of, or consist of a nucleic acid sequence at least 65%, 70%, 75%, 80%, 85%, 90%, 95%, 96%, 97%, 98%, 99% or 100% (or any percentage in between) identical to one or more of the following nucleotide sequences set forth in the table which follows:

### DMD Exon 51 RNA Targeting Sequences

| | | SEQ |
|---|---|---|
| | | ID |
| UID | Targeting Sequences | NO: |
| z38 | TACCTCCAACATCAAGGAAGATGGCA | 206 |
| z42 | GTAACCACAGGTTGTGTCACCAGAGTAACA | 207 |

Fusion Sequences

| | | |
|---|---|---|
| z70 | catacCTTCTGCTTGATGATCATCTCGCACC | 208 |
| | AGAGTAACAGTCTGAGTAGGAGctaaa | |
| z71 | catacCTTCTGCTTGATGATCATCTCGTGCC | 209 |
| | GCTGCCCAATGCCATCCTGGAG | |
| z72 | catacCTTCTGCTTGATGATCATCTCGGCCC | 210 |
| | AATGCCATCCTGGAGTTCctg | |
| z73 | TACCTCCAACATCAAGGAAGATGGCAGTAAC | 211 |
| (z38/z42) | CACAGGTTGTGTCACCAGAGTAACA | |
| z74 | AACATCAAGGAAGATGGCATTTCTAGGTAAC | 212 |
| | CACAGGTTGTGTCACCAGAGTAACA | |
| z75 | AACATCAAGGAAGATGGCATTTCTAGTGCCG | 213 |
| | CTGCCCAATGCCATCCTGGAG | |
| z76 | TTTTTCTCATACCTTCTGCTTGATGTCTGAG | 214 |
| | TAGGAGCTAAAATATTTTGGG | |
| Z187 | GTAACCACAGGTTGTGTCACCAGAGTAACAT | 215 |
| (z42/z38) | ACCTCCAACATCAAGGAAGATGGCA | |
| z230 | CGAGATGATCATCAAGCAGAAGgtatgCACC | 216 |
| | AGAGTAACAGTCTGAGTAGGAGctaaa | |
| z231 | CACCAGAGTAACAGTCTGAGTAGGAGctaaa | 217 |
| | TACCTCCAACATCAAGGAAGATGGCA | |
| z232 | TACCTCCAACATCAAGGAAGATGGCACACCA | 218 |
| | GAGTAACAGTCTGAGTAGGAGctaaa | |
| z233 | GTCTGAGTAGGAGctaaaatattttgggTAC | 219 |
| | CTCCAACATCAAGGAAGATGGCA | |
| z234 | TACCTCCAACATCAAGGAAGATGGCAGTCTG | 220 |
| | AGTAGGAGctaaaatattttggg | |
| z235 | GTCTGAGTAGGAGctaaaatattttgggttt | 221 |
| | ttctcatacCTTCTGCTTGAT | |
| z237 | cCTTCTGCTTGATGATCATCTCGGAGTAACA | 222 |
| | GTCTGAGTAGGAGc | |
| z238 | cCTTCTGCTTGATGATCATCTCGCCAGAGTA | 223 |
| | ACAGTCTGAGTAGGAGc | |
| z239 | cCTTCTGCTTGATGATCATCTCGCAGTCTGA | 224 |
| | GTAGGAGctaaa | |
| z240 | ctcatacCTTCTGCTTGATGGAGTAACAGTC | 225 |
| | TGAGTAGGAGc | |
| z241 | ctcatacCTTCTGCTTGATGCCAGAGTAACA | 226 |
| | GTCTGAGTAGGAGc | |
| z242 | ctcatacCTTCTGCTTGATGCAGTCTGAGTA | 227 |
| | GGAGctaaa | |

In one embodiment, TSs which are a fusion as in the above table can be a single TS sequence comprised within the fusion sequence.

In another embodiment, the targeting sequences (TSs) that target two or more RNAs of interest. In another embodiment, the TSs which target two or more RNAs of interest are different sequences which target the same pre-mRNA molecule. In one embodiment, the spacers or TSs are a fusion sequence. In another embodiment, the fusion sequence is a spacer targeting DMD exon 51, wherein the fusion sequence is set forth in the table above.

In some embodiments, CUG repeat targeting sequences comprise the nucleic acid sequence: CAGCAGCAGCAGCAGCAGCAGCAGCAGCAGCAGCAGCAGCAGCAG (SEQ ID NO: 165).

In some embodiments, CAG repeat targeting sequences comprise the nucleic acid sequence: CTGCTGCTGCTGCTGCTGCTGCTGCTGCTGCTGCTGCTGCTGCTG (SEQ ID NO: 167).

### Sm Binding Domains

The esnRNA systems disclosed herein utilize an Sm binding domain (SmBD). The Sm protein ring that assembles around the Sm binding domain (SmBD) to form an snRNP includes SmB/B′, SmD1, SmD2, SmD3, SmE, SmF, and SmG. The U7 Sm binding site recruits endogenous RNA binding factors and can be replaced with a non-U7 snRNA to make the esnRNA more stable. In one embodiment, the SmBD is selected from the group consisting of U1, U2, U4, and U5 snRNAs. In another embodiment, the SmBD is derived from a pseudo snRNA. In another embodiment, the SmBD is a nucleotide sequence comprising SEQ ID NO: 31 (ATTTTT). In another embodiment, the SmBD comprises a nucleotide sequence selected from the group consisting of SEQ ID NO: 32 (AATTTTTGG), SEQ ID NO: 33 (AATTTGTGG), SEQ ID NO: 34 (AATTTGTGG), SEQ ID NO: 35 (AATTTCTGG), SEQ ID NO: 36 (GATTTTTGG), SEQ ID NO: 37 (AATTTTTGA), SEQ ID NO: 38 (AATTTTTTG), SEQ ID NO: 161 (AATTTTTGGAGCA), and SEQ ID NO: 163 (AATTTTTGGAGTA).

### Promoter Sequences

The esnRNA systems disclosed herein comprise an snRNA promoter from any of U1-U12. In one embodiment, the snRNA promoter is a U7 promoter. In another embodiment, the U7 promoter is a human U7 promoter (hU7) or a mouse U7 promoter (mU7). In another embodiment, the U7 promoter is an endogenous human U7 promoter comprising SEQ ID NO: 39:

TACTGCCGAATCCAGGTCTCCGGGCTTAACAACAACGAAGGGGCTGTGAC
TGGCTGCTTTCTCAACCAATCAGCACCGAACTCATTTGCATGGGCTGAGA
ACAAATGTTCGCGAACTCTAGAAATGAATGACTTAAGTAAGTTCCTTAGA
ATATTATTTTTCCTACTGAAAGTTACCACATGCGTCGTTGTTTATACAGT
AATAGGAACAAGAAAAAAGTCACCTAAGCTCACCCTCATCAATTGTGGAG
TTCCTTTATATCCCATCTTCTCTCCAAACACATACGCA.

In one embodiment, the snRNA promoter is a U1 promoter. In another embodiment, the U1 promoter is a human U1 promoter or a mouse U1 promoter.

In another embodiment, an snRNA promoter drives a copy of an snRNA insert. In another embodiment, each copy of an snRNA insert is the same. In another embodiment, different snRNA promoters drive each copy of an snRNA insert. In one embodiment, a 2×snRNA comprises a mouse U7 promoter driving one copy of an snRNA insert and a mouse U1 promoter drives the other copy of an snRNA insert.

In other aspects, the snRNA promoter is a PolII promoter or a PolII promoter. In other aspects, the snRNA promoter comprises, consists essentially of, or consists of a nucleic acid sequence at least 65%, 70%, 75%, 80%, 85%, 90%, 95%, 96%, 97%, 98%, 99% or 100% (or any percentage in between) identical to a promoter and/or promoter sequence listed in the Exemplary Promoter Table which follows:

| Exemplary | |
|---|---|
| Promoter | Promoter Sequence |
| hU1 | AAGGACCAGCTTCTTTGGGAGAGAACAGACGCAGGGGCGGGAGGGA |
| | AAAAGGGAGAGGCAGACGTCACTTCCCCTTGGCGGCTCTGGCAGCAG |
| | ATTGGTCGGTTGAGTGGCAGAAAGGCAGACGGGGACTGGGCAAGGC |
| | ACTGTCGGTGACATCACGGACAGGGCGACTTCTATGTAGATGAGGCA |
| | GCGCAGAGGCTGACGTCTTCGCCACTTGCTGCTTCACCACGAAGGAG |
| | TTCCCGTGCCCTGGGAGCGGGTTCAGGACCGCTGATCGGAAGTGAGA |
| | ATCCCAGCTGTGTGTCAGGGCTGGAAAGGGCTCGGGAGTGCGCGGGG |
| | CAAGTGACCGTGTGTGTAAAGAGTGAGGCGTATGAGGCTGTGTCGGG |
| | GCAGAGGCacaacgtttc |
| | (SEQ ID NO: 40) |
| hU2 | CCACGCCCTCTGTGAAAGGGCGGGGCATGCAAATTCGAAATGAAAGC |
| | CCGGGAACGCCGGAAGAAGCACGGGTGTAAGATTTCCCTTTTCAAAG |
| | GCAGAGAATAAGAAATCAGCCCGAGAGTGTAAGGGCGTCAATAGCG |
| | CTGTGGACGAGACAGAGGGAATGGGGCAAGGAGCGAGGCTGGGGCT |
| | CTCACCGCGACTTGAATGTGGATGAGAGTGGGACGGTGACGGCGGGC |
| | GCGAAGGCGAGCGC |
| | (SEQ ID NO: 41) |
| hU4 | TTCGCAGTCTCTGAATTAAGTCTATTAGCATGTTCCTCCCATAGTGCT |
| | TTGCTTCATATCAACAAAAACCTAGCTAAGTGAAATCAGCAACGATA |
| | TGCAGAAACCACCTACGCAGGTCACAAACATCTTTCTATGATTGTATA |
| | ATTTTCAAGCAAGCAATAAGTGAAGATTTTTCCATAGGCCCTAAACTC |
| | ACCTTTGCGAAATAGGAAGCTGGTTTATTGGGAGTGATGAGCAGGGG |
| | GCGTAACAAATT |
| | (SEQ ID NO: 42) |
| hU5 | GCAGCAAGGCCTCCACTTCACCCCCTAAAGGTTGCCCCAAGAGCACC |
| | GTGTGACTGCTAAGGTATTTCCGGAGTCTAAAGACGATTATTCAGGTC |
| | TCATTTGCATACCCATAATACACTGCAAACAGTATTTTTTTCGGAAAA |
| | ACATTTATATATTGCTTGACATTTTTAAGTATGAGAATTTTGCATGCA |
| | GAATTTTTTTGTATAAACTTTCTCAGGTAGTAACCCTTGGGATTAGTA |
| | GACACCATCAGTGTACTAGGAATTGCAGTTACCCGAAAATTGAGTTA |
| | CAGAAGTAACTGGT |
| | (SEQ ID NO: 43) |
| hU6 | Gtttattacagggacagcagagatccagtttggttaattaaggtaccgaggg |
| | cctatttcccatgattccttcatatttgcatatacgatacaaggctgttaga |
| | gagataattagaattaatttgactgtaaacacaaagatattagtacaaaata |
| | cgtgacgtagaaagtaataatttcttgggtagtttgcagttttaaaattatg |
| | ttttaaaatggactatcatatgcttaccgtaacttgaaagtatttcgatttc |
| | ttggctttatatatcttgtggaaaggacgaaacacc |
| | (SEQ ID NO: 44) |
| h7sk | CTGCAGTATTTAGCATGCCCCACCCATCTGCAAGGCATTCTGGATAGT |
| | GTCAAAACAGCCGGAAATCAAGTCCGTTTATCTCAAACTTTAGCATTT |
| | TGGGAATAAATGATATTTGCTATGCTGGTTAAATTAGATTTTAGTTAA |
| | ATTTCCTGCTGAAGCTCTAGTACGATAAGTAACTTGACCTAAGTGTAA |
| | AGTTGAGATTTCCTTCAGGTTTATATAGCTTGTGCGCCGCCTGGGTAC |
| | CTC |
| | (SEQ ID NO: 45) |
| tRNA(Val) | CAGGACTAGTCTTTTAGGTCAAAAAGAAGAAGCTTTGTAACCGTTGG |
| | TTTCCGTAGTGTAGTGGTTATCACGTTCGCCTAACACGCGAAAGGTCC |
| | CCGGTTCGAAG |
| | (SEQ ID NO: 46) |
| mU1 | TTGTTCCTCTTAGTGTTAATTCACACTAAAGACTGTGCATCCGACTCC |
| | TACATTTATGAAAGTAAATGCCTGTTGTTAGAACAAAAAAGGCTACA |
| | GAACAAAAAACAAAGCGAAATACCATCTGCTTTAGGTTCAGTGTGGT |
| | ATTTTCCCGCTGACAGGGAGGCGGGTTTTTGGGTACAGGAAACGAGT |
| | CACTATGGAGGCGGTACTATGTAGATGAGAATTCAGGTGCAAACTGG |
| | GAAAAGCAACTGCTTCCAAATATTTGTGATTTTTACAGTGTAGTTTTG |
| | GAAAAACTCTTAGCCTACCAATTCTTCTAAGTGTTTTAAAATGTGGGA |
| | GCCAGTACACATGAAGTTATAGAGTGTTTTAATGAGGCTTAAATATTT |
| | ACCGTAACTATGAAATGCTACGCATATCATGCTGTTCAGGCTCCGTGG |
| | CCACGCAACTC |
| | (SEQ ID NO: 47) |
| mU7+ | TaacaacataggagctgtgattggctgttttcagccaatcagcactgActcattt |
| cxtra 3′ | gcatagcctttacaagcggtcacaaactcaagaaacgagcggttttaatagtctt |
| | ttagaatattgtttatcgaaccgaataaggaactgtgctttgtgattcacatatc |
| | agtggaggggtgtggaaatggcaccttgatctcaccctcatcgaaagtggagttg |
| | atgtcctTccctggctcgctacagacgcacttccgcaa |
| | (SEQ ID NO: 48) |
| mU7 | taacaacataggagctgtgattggctgttttcagccaatcagcactgActcattt |
| | gcatagcctttacaagcggtcacaaactcaagaaacgagcggttttaatagtctt |
| | ttagaatattgtttatcgaaccgaataaggaactgtgctttgtgattcacatatc |
| | agtggaggggtgtggaaatggcaccttgatctcaccctcatcgaaagtggagttg |
| | atgtcctTccctggctcgctacagacgcacttccgc |
| | (SEQ ID NO: 151) |
| hU7 | TACTGCCGAATCCAGGTCTCCGGGCTTAACAACAACGAAGGGGCTGT |
| | GACTGGCTGCTTTCTCAACCAATCAGCACCGAACTCATTTGCATGGGC |
| | TGAGAACAAATGTTCGCGAACTCTAGAAATGAATGACTTAAGTAAGT |
| | TCCTTAGAATATTATTTTTCCTACTGAAAGTTACCACATGCGTCGTTG |
| | TTTATACAGTAATAGGAACAAGAAAAAAGTCACCTAAGCTCACCCTC |
| | ATCAATTGTGGAGTTCCTTTATATCCCATCTTCTCTCCAAACACATAC |
| | GCA |
| | (SEQ ID NO: 39) |
| mU5 | TGCCAACAGCTTTGAAATCCTCTGTGCTCTTGTGCCAATCCCACCCTC |
| | AGAACAGGGAGTCTGTCGGTTATGTTTTCGTCCTTTACAGTTCTTCGC |
| | CGTCTAATCCATTGGAAACCTCCTTTATTTAAAGGAAACCGTGTCCAA |
| | ATTCAGTGTACAGAGGTGCAAACCTCCGCCCCGCTCCTTGTAGGAAG |
| | CCCACGGGAACTGGGCCAATCGGACGGCGCTTACTCGGCTCATTTAC |
| | ATACCCATAACACACCGCGGCTAATGCAAATATTTTCGTGAAAAGAT |
| | CTTCCATTACTCGAAGTTTTAAAACTAAAAACAAAATATTATTTCAAA |
| | AGATCATGAAGAAATTTCTGTCGGTAACAGCAGTTTCAATTGATAAA |
| | TCACCATCACTTATCTAGGAGGTTCTGTTACTCTAGAAGTGAATTAAG |
| | CAGGACAGCTGT (SEQ ID NO: 152) |
| mU2 | Gggcggggcatgcaaataactgctctgtggaactctgggagcaaaaacaaaaaac |
| | tgcaaccaaaacttctcggcctccttgaatcttacaggcttttcgtggcgtaaag |
| | gtggtgtactcaatgaagaggagagtctgtgttggctgcatgtttgagtcggttg |
| | gttggtgactgtgaatTAAAGGTGTGGtcggtgttgagtgtatggggcgtgtggg |
| | cgtagttcggt |
| | (SEQ ID NO: 153) |
| mU6 | cgacgccgccatctctaggcccgcgccggccccctcgcacagacttgtgggagaa |
| | gctcggctactcccctgccccggttaatttgcatataatatttcctagtaactat |
| | agaggcttaatgtgcgataaaagacagataatctgttctttttaatactagctac |
| | attttacatgataggcttggatttctataagagatacaaatactaaattattatt |
| | ttaaaaaacagcacaaaaggaaactcaccctaactgtaaagtaattgtgtgtttt |
| | gagactataaatatcccttggagaaaagccttgttt |
| | (SEQ ID NO: 154) |
| mH1 | CATgCAAATTACGCGCTgTGCTTTGTGGGAAATCACCCTAAACGTAAA |
| | ATTTATTCCTCTTTcGAGCCTTATAGTGGcGGCCGGTCTACACCCTAAA |
| | (SEQ ID NO: 155) |

### Terminator Sequences

The esnRNA systems disclosed herein comprise an snRNA downstream terminator (DT). Downstream terminators define the end of a transcriptional unit, such as an esnRNA or snRNA. In another embodiment the snRNA DT is a U7 DT comprising:

(SEQ ID NO: 49)
CCTCTTATGATGTTTGTTGCCAATGATAGATTGTTTTCACTGTGCAAAAA
TTATGGGTAGTTTTGGTGGTCTTGATGCAGTTGTAAGCTTGGAG.

In one embodiment, the esnRNA comprises the eSL, one or more promoters, the TS, the SmBD, the 5′ISD, and the DT. In one aspect, promoter and DT combinations are be mixed and matched.

In another embodiment, the DT is selected from the exemplary DTs and/or DT sequences listed in the Exemplary DT Table below:

| Exemplary | |
|---|---|
| DT | DT Sequence |
| hU1 | ACTTTCTGGAGTTTCAAAAACAGACTGTACGCCA |
| | AGGGTCATATCTTTTT |
| | (SEQ ID NO: 50) |
| hU2 | CCGGGGATACAACGTGTTTCCTAAAAGTAGAGGG |
| | AGGTAAGAGACGGTAG |
| | (SEQ ID NO: 51) |
| hU4 | CTGAATTTTCTTGCAGTTGAACAACAGAGGCTTT |
| | TTTTGTGTGTGTGGGG |
| | (SEQ ID NO: 52) |
| hU5 | ATATGTGGTAATCCAACAATAGAAATTATTTTTA |
| | AGTTTGTGTGTTCCTT (SEQ ID NO: 53) |
| hU6 | TTTTTT |
| | (SEQ ID NO: 54) |
| h7sk | TTTTTT |
| | SEQ ID NO: 55) |
| tRNA(Val) | TTTTTT |
| | (SEQ ID NO: 56) |
| mU1 | GTTTACTTGGTTTTAAAAATAGCTTGCACTAGCG |
| | ATACGGAATATGGTTATTAGGTTTGTTAGGCATC |
| | ATGTCGTGTCTTACTATAGAAAAATAACGTAGTG |
| | TCATTTTAGCCTGCCTGTATGTGTTAATTTGTCC |
| | TTATTGCGCATTGTTCTTGTTAAGTCTTCTGTAA |
| | GGAGTTGCGGGTTTCAAACTGTCAGTCTGAGAGC |
| | A (SEQ ID NO: 57) |
| mU7+ | Cccaatttcactggtctacaatgaaagcaaaaca |
| | gttctcttccccgctccccggtgtgtgagagggg |
| | ctttgatccttctctggtttcctaggaaacgcgt |
| | atgtgtac (SEQ ID NO: 58) |
| mU7 | Cccaatttcactggtctacaatgaaagcaaaaca |
| | gttctcttccccgctccccggtgtgtgagagggg |
| | ctttgatccttctctggtttcctaggaaacgcgt |
| | atgtg (SEQ ID NO: 156) |
| hU7 | CCTCTTATGATGTTTGTTGCCAATGATAGATTGT |
| | TTTCACTGTGCAAAAATTATGGGTAGTTTTGGTG |
| | GTCTTGATGCAGTTGTAAGCTTGGAG |
| | (SEQ ID NO: 49) |
| hU7 | CTTATGATGTTTGTTGCCAATGATAGATTGTTTT |
| | CACTGTGCAAAAATTATGGGTAGTTTTGGTGGTC |
| | TTGATGCAGTTGTAAGCTTGGAG |
| | (SEQ ID NO: 234) |
| mU5 | CCACATTTTGTGTTTAAAAATAGAAATATTTAAG |
| | TGAGATCAGTTTAAATCTGCTTTATCTAGGGTGT |
| | CTAACTGCTTGCATCTTTTTAACTTTTCCTTATT |
| | TGTGAGAAGGTCTGTAAAACTTATTATATGTTAG |
| | TACACTGTAGCTGTCTTCAGACACTCCAGAAGAG |
| | GGATTCAGATCACCTTACAGATGGTTTTGA |
| | (SEQ ID NO: 157) |
| mU2 | CCCTCTGGGGAgtaaagttggttttaaagtcaga |
| | gcatggtgattgtagggcagtccaacttttttaa |
| | atatgctgtg (SEQ ID NO: 158) |
| mU6 | TTTTTT (SEQ ID NO: XX) |
| mH1 | TTTTTT (SEQ ID NO: XX) |

In one embodiment, the esnRNA is delivered in an AAV vector.

In some embodiments, the AAV vector comprises multiple copies of the esnRNA. In some embodiments, the multiple copies of the esnRNA are 2, 3, or 4 copies (2×, 3×, or 4×) of the esnRNA. In some embodiments, the multiple copies of the esnRNA are 4 or more copies of the esnRNA.

In some embodiments, each esnRNA of the multiple copies of esnRNA is separated by a nucleic acid buffer sequence derived from human non-coding genomic sequences downstream of an snRNA. In one embodiment, the buffer sequence is derived from human genomic sequences downstream of U7.

In one embodiment, the buffer sequence is selected from the group consisting of the following nucleic acid sequence:

buffer 1 (30 bp)
(SEQ ID NO: 24)
CAAACTACAGAGCCAAGTGCTATCCACAGA,
buffer 2 (30 bp)
(SEQ ID NO: 25)
GAGCTTTCTGGGTTGCCATCTCAAGCAGAC,
buffer 3 (30 bp)
(SEQ ID NO: 26)
TACAAGGCCATCAGCTCATACTCACAATTG,
and a combination thereof.

In another embodiment, the buffer sequence is selected from the group consisting of the following nucleic acid sequence:

buffer 1 (100 bp)
(SEQ ID NO: 27)
CAAACTACAGAGCCAAGTGCTATCCACAGAGAGCTTTCTGGGTTGCCAT
CTCAAGCAGACTACAAGGCCATCAGCTCATACTCACAATTGACTTTGAG
AG,
buffer 2 (100 bp)
(SEQ ID NO: 28)
TTGACCACATACGTGCTCTTTCAAAGTTCTGTGTTTGAAGTTATGTTAG
TAACAACTGATGCCCATCCTGCAATGACAAATCCAATTCTCAGTGCAGC
TC,
and a combination thereof.

In another embodiment, the buffer sequence is selected from the group consisting of the following nucleic acid sequence:

buffer 1 (500 bp)
(SEQ ID NO: 29)
CAAACTACAGAGCCAAGTGCTATCCACAGAGAGCTTTCTGGGTTGCCAT
CTCAAGCAGACTACAAGGCCATCAGCTCATACTCACAATTGACTTTGAG
AGTCATTTTCCAATGCTCCTACACACCCCTTCTTCACAATCCCCAACAA
ATCTGAGGCTGGAACTTGGTACCATAACAATCATTACATTATTTCACCA
GAAGTACACCTTGCCTGGAAGATTGGCATTATAGCATCTTCTAACATTG
TGAAAGTTAGTGACCAATGAGGAGATCCAAGTCAGTTCCAGTTGGATTT
CTCTATACTCTATAATAAATATATATGGTGTCTTCAACAATAGGACTTT
GCCATCCAGTGATGCTAAAAATCAATAACAATGGCAATAACCTGCCCTG
TTTGGAAAGCCTCTGGCTTCCATGACTAACAATTCAAGGCAGGTCTCCT
ATACCTAGTACTGAGATTTTTATTTGATAAACTATATCTTCTGGGAGGA
GAAGC,
buffer 2 (500 bp)
(SEQ ID NO: 30)
TTGACCACATACGTGCTCTTTCAAAGTTCTGTGTTTGAAGTTATGTTAG
TAACAACTGATGCCCATCCTGCAATGACAAATCCAATTCTCAGTGCAGC
TCTCTGAAATAGTTTTGCTTTCTCTCTCTAGGTCTGTTCTATACTCCTA
ACTCTCCAGGAGTTTACAAGGAATAAAATCTCTTCCAAATGCTTTCTGT
TGCAACAACTGGACCATACTGAAAGCTGAGGCCCACAATTGCAATCTAG
GTTAGCAGGTAATCATTGTTGGTGAGGTCCTCCCTTTCCCCAGGCTCGT
GTTTGTATTGGGGAGCAGGAAATTTTTGCTAGAGCAGCACTGCCATCTC
TCTACACTCCACCTGATTGGTGGGATGGACCAGAGAAATGGACATTCCC
AACACAGTCCCTCCTTTCACATCTGCTCACCTGCCCACAGGATACTTTC
CACCATGCATACTGGGCTCTGCACCAACCATTCAGCAGTGATGAAGAGG
AAACTTGAAC,
and/or
a combination thereof.

The 100 bp and 500 bp buffer 1 sequences are derived from a sequence starting 100 bp downstream of the Mus musculus U7 pseudogene 8 (Location Chromosome 14: 4,409,359-4,409,421 reverse strand. GRCm39:CM001007.3). The 100 bp and 500 bp buffer 2 sequences are derived from the sequence starting 130 bp downstream of human U7 pseudogene 5 (Chromosome X: 140,451,148-140,451,208 forward strand. GRCh38:CM000685.2). Both 100 bp buffers are the first 100 bp of the corresponding 500 bp buffer. The 30 bp buffers 1, 2, and 3, are sequential 30 bp sequences within “100 bp buffer 1”, downstream of the Mus musculus U7 pseudogene 8. These downstream sequences were selected due to the lack of any known regulatory sites or genes within or nearby to the sequence (using Gencode/Ensembl), in addition to lack of repetitive sequence, 40-60% GC content for total buffer, 40-60% GC content in the 20 bp region at both ends of the buffer, and minimal sequence complexity.

esnRNA Sequences

Exemplary esnRNA sequences of the disclosure can comprise any combination of esnRNA features described herein. In some aspects, the esnRNA comprises, consists essentially of, or consists of a nucleic acid sequence at least 65%, 70%, 75%, 80%, 85%, 90%, 95%, 96%, 97%, 98%, 99% or 100% (or any percentage in between) identical to an esnRNA listed in the esnRNA Table which follows:

esnRNA sequences of the disclosure

| | | Target | | |
|---|---|---|---|---|
| | | Repeat or | SEQ | |
| | | Gene | ID | |
| ID | Description | Symbol | NO: | Sequence |
| | u7 snRNA CUG | CUG | 65 | ggagtCAGCAGCAGCAGCAGCAGCAGCAGCAGCAGC |
| | | | | AGCAGCAGCAGCAGAATTTTTGGAGTAggctttctggctc |
| | | | | cttaccggaaagcc |
| | u7 snRNA NT | non- | 66 | ggagtTCACCAGAAGCGTACCATACTCACGAAATTTT |
| | | targeting | | TGGAGTAggctttctggctccttaccggaaagcc |
| | u7 snRNA CAG | CAG | 67 | ggagtCTGCTGCTGCTGCTGCTGCTGCTGCTGCTGCT |
| | | | | GCTGCTGCTGCTGAATTTTTGGAGTAggctttctggctcctta |
| | | | | ccggaaagcc |
| | z38/42 esnRNA | DMD | 68 | ggagtTACCTCCAACATCAAGGAAGATGGCAGTAAC |
| | targeting | exon 51 | | CACAGGTTGTGTCACCAGAGTAACAAATTTTTGGA |
| | dystrophin exon | | | GTAggctttctggctccttaccggaaagcc |
| | 51 | | | |
| | z42 esnRNA | DMD | 69 | ggagtGTAACCACAGGTTGTGTCACCAGAGTAACAA |
| | | exon 51 | | ATTTTTGGAGTAggctttctggctccttaccggaaagcc |
| | z38 esnRNA | DMD | 70 | ggagtTACCTCCAACATCAAGGAAGATGGCAAATTTT |
| | | exon 51 | | TGGAGTAggctttctggctccttaccggaaagcc |
| SR2 | U7CUGx15-U1 | CUG | 71 | ggagtCAGCAGCAGCAGCAGCAGCAGCAGCAGCAGC |
| 20031 | Sm site_U7 term | | | AGCAGCAGCAGCAGAATTTGTGGAGTAggctttctggctc |
| | opt sl | | | cttaccggaaagcc |
| SR2 | U7CUGx15-Sm | CUG | 72 | ggagtCAGCAGCAGCAGCAGCAGCAGCAGCAGCAGC |
| 20032 | mut 1_U7 term | | | AGCAGCAGCAGCAGTCGAGTTGGAGTAggctttctggctc |
| | opt sl | | | cttaccggaaagcc |
| SR2 | U7CAGx15-no | CUG | 73 | ggagtCAGCAGCAGCAGCAGCAGCAGCAGCAGCAGC |
| 20033 | Sm_U7 term opt | | | AGCAGCAGCAGCAGAGTAggctttctggctccttaccggaaagcc |
| | sl | | | |
| SR2 | U7knt-U1 Sm | non- | 74 | ggagtTCACCAGAAGCGTACCATACTCACGAAATTTG |
| 20034 | site_U7 term opt | targeting | | TGGAGTAggctttctggctccttaccggaaagcc |
| | sl | | | |
| SR2 | U7knt-Sm mut | non- | 75 | ggagtTCACCAGAAGCGTACCATACTCACGATCGAGT |
| 20035 | 1_U7 term opt sl | targeting | | TGGAGTAggctttctggctccttaccggaaagcc |
| SR2 | U7knt-no | non- | 76 | ggagtTCACCAGAAGCGTACCATACTCACGAAGTAgg |
| 20036 | Sm_U7 term opt | targeting | | ctttctggctccttaccggaaagcc |
| | sl | | | |
| SR2 | U7CAGx15-U1 | CAG | 77 | ggagtCTGCTGCTGCTGCTGCTGCTGCTGCTGCTGCT |
| 20037 | Sm site_U7 term | | | GCTGCTGCTGCTGAATTTGTGGAGTAggctttctggctcctta |
| | opt sl | | | ccggaaagcc |
| SR2 | U7CAGx15-Sm | CAG | 78 | ggagtCTGCTGCTGCTGCTGCTGCTGCTGCTGCTGCT |
| 20038 | mut 1_U7 term | | | GCTGCTGCTGCTGTCGAGTTGGAGTAggctttctggctcctt |
| | opt sl | | | accggaaagcc |
| SR2 | U7CAGx15-no | CAG | 79 | ggagtCTGCTGCTGCTGCTGCTGCTGCTGCTGCTGCT |
| 20039 | Sm_U7 term opt | | | GCTGCTGCTGCTGAGTAggctttctggctccttaccggaaagcc |
| | sl | | | |
| SR2 | U7CAGx5-U1 | CAG | 80 | ggagtCTGCTGCTGCTGCTGAATTTGTGGAGTAggctttct |
| 20040 | Sm site_U7 term | | | ggctccttaccggaaagcc |
| | opt sl | | | |
| SR2 | U7CAGx5-Sm | CAG | 81 | ggagtCTGCTGCTGCTGCTGTCGAGTTGGAGTAggctttct |
| 20041 | mut 1_U7 term | | | ggctccttaccggaaagcc |
| | opt sl | | | |
| SR2 | U7CAGx5-no | CAG | 82 | ggagtCTGCTGCTGCTGCTGAGTAggctttctggctccttaccgga |
| 20042 | Sm_U7 term opt | | | aagcc |
| | Sl | | | |
| SR2 | U7-z38-delSm | DMD | 83 | ggagtTACCTCCAACATCAAGGAAGATGGCAggctttctg |
| 20056 | | | | gctccttaccggaaagcc |
| SR2 | U7 5′SL with | non- | 84 | GTTCCCGAAGTAACCCTTCGGGAACggagtTCACCAG |
| 20087 | ISD; non- | targeting | | AAGCGTACCATACTCACGAAATTTTTGGAGTAggcttt |
| | targeting NT (not | | | ctggctccttaccggaaagcc |
| | made) | | | |
| SR2 | U7 5′SL with no | non- | 85 | GTTCCCGAAGTAACCCTTCGGGAACTCACCAGAAG |
| 20088 | ISD; non- | targeting | | CGTACCATACTCACGAAATTTTTGGAGTAggctttctggc |
| | targeting NT (not | | | tccttaccggaaagcc |
| | made) | | | |
| SR2 | U7 5′Kissing SL | non- | 86 | GTTCCCGAAGggagtCTTCGGGAACTCACCAGAAGC |
| 20089 | with no ISD; | targeting | | GTACCATACTCACGAAATTTTTGGAGTAggctttctggctc |
| | non-targeting NT | | | cttaccggaaagcc |
| | (not made) | | | |
| SR2 | U7 5′GC clamp | non- | 87 | GGGCCCTCACCAGAAGCGTACCATACTCACGAAAT |
| 20090 | with no ISD; | targeting | | TTTTGGAGTAggctttctggctccttaccggaaagcc |
| | non-targeting NT | | | |
| | (not made) | | | |
| SR2 | U7 5′Triple helix | non- | 88 | GAGAAGATTCGTCTTCTCTCTCTCTCTTCTTCACCA |
| 20091 | with no ISD; | targeting | | GAAGCGTACCATACTCACGAAATTTTTGGAGTAggc |
| | non-targeting NT | | | tttctggctccttaccggaaagcc |
| | (not made) | | | |
| SR2 | U7 5′SL with | CUG | 89 | GTTCCCGAAGTAACCCTTCGGGAACggagtCAGCAG |
| 20092 | ISD; CUGx15 | | | CAGCAGCAGCAGCAGCAGCAGCAGCAGCAGCAGC |
| | | | | AGCAGAATTTTTGGAGTAggctttctggctccttaccggaaagcc |
| SR2 | U7 5′SL with no | CUG | 90 | GTTCCCGAAGTAACCCTTCGGGAACCAGCAGCAGC |
| 20093 | ISD; CUGx15 | | | AGCAGCAGCAGCAGCAGCAGCAGCAGCAGCAGCA |
| | | | | GAATTTTTGGAGTAggctttctggctccttaccggaaagcc |
| SR2 | U7 5′Kissing SL | CUG | 91 | GTTCCCGAAGggagtCTTCGGGAACCAGCAGCAGCA |
| 20094 | with no ISD; | | | GCAGCAGCAGCAGCAGCAGCAGCAGCAGCAGCAG |
| | CUGx15 | | | AATTTTTGGAGTAggctttctggctccttaccggaaagcc |
| SR2 | U7 5′GC clamp | CUG | 92 | GGGCCCCAGCAGCAGCAGCAGCAGCAGCAGCAGC |
| 20095 | with no ISD; | | | AGCAGCAGCAGCAGCAGAATTTTTGGAGTAggctttct |
| | CUGx15 | | | ggctccttaccggaaagcc |
| SR2 | U7 5′Triple helix | CUG | 93 | GAGAAGATTCGTCTTCTCTCTCTCTCTTCTCAGCAG |
| 20096 | with no ISD; | | | CAGCAGCAGCAGCAGCAGCAGCAGCAGCAGCAGC |
| | CUGx15 | | | AGCAGAATTTTTGGAGTAggctttctggctccttaccggaaagcc |
| SR2 | U7-nt | non- | 94 | ggagtTCACCAGAAGCGTACCATACTCACGAAATTTT |
| 20004 | | targeting | | TGGAGTAggctttctggctccttaccggaaagcc |
| SR2 | U7 alt ISD | non- | 95 | cctctTCACCAGAAGCGTACCATACTCACGAAATTTT |
| 20097 | SEQID 16 non- | targeting | | TGGAGTAggctttctgggaggttaccggaaagcc |
| | targeting nt1 | | | |
| SR2 | U7 alt ISD | non- | 96 | ggaggtTCACCAGAAGCGTACCATACTCACGAAATTT |
| 20098 | SEQID17 non- | targeting | | TTGGAGTAggctttctggcctccttaccggaaagcc |
| | targeting nt1 | | | |
| SR2 | U7 alt ISD | non- | 97 | cctcctTCACCAGAAGCGTACCATACTCACGAAATTTT |
| 20099 | SEQID18 non- | targeting | | TGGAGTAggctttctggggaggttaccggaaagcc |
| | targeting nt1 | | | |
| SR2 | U7 alt ISD | non- | 98 | agccagTCACCAGAAGCGTACCATACTCACGAAATTT |
| 20100 | SEQID19 non- | targeting | | TTGGAGTAggctttctggctggctaccggaaagcc |
| | targeting nt1 | | | |
| SR2 | U7 alt ISD | non- | 99 | ggaagTCACCAGAAGCGTACCATACTCACGAAATTTT |
| 20101 | SEQID20 non- | targeting | | TGGAGTAggctttctggcttccccggaaagcc |
| | targeting nt1 | | | |
| SR2 | U7 alt ISD | non- | 100 | gaagaagTCACCAGAAGCGTACCATACTCACGAAATT |
| 20102 | SEQID21 non- | targeting | | TTTGGAGTAggctttctggcttcttcccggaaagcc |
| | targeting nt1 | | | |
| SR2 | U7 alt ISD | non- | 101 | gttgTCACCAGAAGCGTACCATACTCACGAAATTTTT |
| 20103 | SEQID22 non- | targeting | | GGAGTAggctttctggcaacttaccggaaagcc |
| | targeting nt1 | | | |
| SR2 | U7 alt ISD | non- | 102 | ccgaaTCACCAGAAGCGTACCATACTCACGAAATTTT |
| 20104 | SEQID23 non- | targeting | | TGGAGTAggctttctggttcggtaccggaaagcc |
| | targeting nt1 | | | |
| SR2 | U7 alt ISD | non- | 103 | taaggagTCACCAGAAGCGTACCATACTCACGAAATT |
| 20105 | SEQID24 non- | targeting | | TTTGGAGTAggctttctggctccttaccggaaagcc |
| | targeting nt1 | | | |
| SR2 | U7 alt ISD | non- | 104 | gaagTCACCAGAAGCGTACCATACTCACGAAATTTT |
| 20106 | SEQID25 non- | targeting | | TGGAGTAggctttctggcttcttcccggaaagcc |
| | targeting nt1 | | | |
| SR2 | U7 alt ISD | non- | 105 | ggcttTCACCAGAAGCGTACCATACTCACGAAATTTT |
| 20107 | SEQID26 non- | targeting | | TGGAGTAggctttctggaagccttaccggaaagcc |
| | targeting nt1 | | | |
| SR2 | U7 alt ISD | non- | 106 | gaagTCACCAGAAGCGTACCATACTCACGAAATTTT |
| 20108 | SEQID27 non- | targeting | | TGGAGTAggctttctggcttcttaccggaaagcc |
| | targeting nt1 | | | |
| SR2 | U7-CUGx15 | CUG | 107 | ggagtCAGCAGCAGCAGCAGCAGCAGCAGCAGCAGC |
| 20010 | | | | AGCAGCAGCAGCAGAATTTTTGGAGTAggctttctggctc |
| | | | | cttaccggaaagcc |
| SR2 | U7 alt ISD | CUG | 108 | cctctCAGCAGCAGCAGCAGCAGCAGCAGCAGCAGC |
| 20109 | SEQID16 | | | AGCAGCAGCAGCAGAATTTTTGGAGTAggctttctgggag |
| | CUGx15 | | | gttaccggaaagcc |
| SR2 | U7 alt ISD | CUG | 109 | ggaggtCAGCAGCAGCAGCAGCAGCAGCAGCAGCAG |
| 20110 | SEQID17 | | | CAGCAGCAGCAGCAGAATTTTTGGAGTAggctttctggcc |
| | CUGx15 | | | tccttaccggaaagcc |
| SR2 | U7 alt ISD | CUG | 110 | cctcctCAGCAGCAGCAGCAGCAGCAGCAGCAGCAGC |
| 20111 | SEQID18 | | | AGCAGCAGCAGCAGAATTTTTGGAGTAggctttctgggga |
| | CUGx15 | | | ggttaccggaaagcc |
| SR2 | U7 alt ISD | CUG | 111 | agccagCAGCAGCAGCAGCAGCAGCAGCAGCAGCAG |
| 20112 | SEQID19 | | | CAGCAGCAGCAGCAGAATTTTTGGAGTAggctttctggct |
| | CUGx15 | | | ggctaccggaaagcc |
| SR2 | U7 alt ISD | CUG | 112 | ggaagCAGCAGCAGCAGCAGCAGCAGCAGCAGCAG |
| 20113 | SEQID20 | | | CAGCAGCAGCAGCAGAATTTTTGGAGTAggctttctggct |
| | CUGx15 | | | tccccggaaagcc |
| SR2 | U7 alt ISD | CUG | 113 | gaagaagCAGCAGCAGCAGCAGCAGCAGCAGCAGCA |
| 20114 | SEQID21 | | | GCAGCAGCAGCAGCAGAATTTTTGGAGTAggctttctgg |
| | CUGx15 | | | cttcttcccggaaagcc |
| SR2 | U7 alt ISD | CUG | 114 | gttgCAGCAGCAGCAGCAGCAGCAGCAGCAGCAGCA |
| 20115 | SEQID22 | | | GCAGCAGCAGCAGAATTTTTGGAGTAggctttctggcaact |
| | CUGx15 | | | taccggaaagcc |
| SR2 | U7 alt ISD | CUG | 115 | ccgaaCAGCAGCAGCAGCAGCAGCAGCAGCAGCAGC |
| 20116 | SEQID23 | | | AGCAGCAGCAGCAGAATTTTTGGAGTAggctttctggttcg |
| | CUGx15 | | | gtaccggaaagcc |
| SR2 | U7 alt ISD | CUG | 116 | taaggagCAGCAGCAGCAGCAGCAGCAGCAGCAGCA |
| 20117 | SEQID24 | | | GCAGCAGCAGCAGCAGAATTTTTGGAGTAggctttctgg |
| | CUGx15 | | | ctccttaccggaaagcc |
| SR2 | U7 alt ISD | CUG | 117 | gaagCAGCAGCAGCAGCAGCAGCAGCAGCAGCAGC |
| 20118 | SEQID25 | | | AGCAGCAGCAGCAGAATTTTTGGAGTAggctttctggcttc |
| | CUGx15 | | | ttcccggaaagcc |
| SR2 | U7 alt ISD | CUG | 118 | ggcttCAGCAGCAGCAGCAGCAGCAGCAGCAGCAGC |
| 20119 | SEQID26 | | | AGCAGCAGCAGCAGAATTTTTGGAGTAggctttctggaag |
| | CUGx15 | | | ccttaccggaaagcc |
| SR2 | U7 alt ISD | CUG | 119 | gaagCAGCAGCAGCAGCAGCAGCAGCAGCAGCAGC |
| 20120 | SEQID27 | | | AGCAGCAGCAGCAGAATTTTTGGAGTAggctttctggcttc |
| | CUGx15 | | | ttaccggaaagcc |
| | DMD z42/38 | DMD | 179 | ggagtGTAACCACAGGTTGTGTCACCAGAGTAACAT |
| | reverse fusion | exon 51 | | ACCTCCAACATCAAGGAAGATGGCAaATTTTTGGA |
| | | | | Gcaggttttctgacctccgtcggaaaacccct |
| | DMD z38/42 | DMD | 180 | ggagtTACCTCCAACATCAAGGAAGATGGCAGTAAC |
| | forward fusion | exon 51 | | CACAGGTTGTGTCACCAGAGTAACAaATTTTTGGA |
| | | | | Gcaggttttctgacctccgtcggaaaacccct |
| | z42/38 reverse | DMD | 181 | ggagtGTAACCACAGGTTGTGTCACCAGAGTAACAT |
| | fusion no CCU | exon 51 | | ACCTCCAACATCAAGGAAGATGGCAaATTTTTGGA |
| | tail | | | Gcaggttttctgacctccgtcggaaaacc |
| | z38/42 for fusion | DMD | 182 | TACCTCCAACATCAAGGAAGATGGCAGTAACCAC |
| | no ISD no eSL | exon 51 | | AGGTTGTGTCACCAGAGTAACAaATTTTTGGAGcagg |
| | | | | ttttctgacttcggtcggaaaacccct |
| | z38/42 forward | DMD | 183 | ggagtTACCTCCAACATCAAGGAAGATGGCAGTAAC |
| | fusion human | exon 51 | | CACAGGTTGTGTCACCAGAGTAACAAATTTTTGGA |
| | loop with ISD | | | GTAggctttctggctccttaccggaaagcc |
| | and eSL | | | |
| | z38/42 forward | DMD | 184 | ggagtTACCTCCAACATCAAGGAAGATGGCAGTAAC |
| | fusion human | exon 51 | | CACAGGTTGTGTCACCAGAGTAACAAATTTTTGGA |
| | loop with ISD | | | GTAggctttctggctccttaccggaaagcccct |
| | and eSL and | | | |
| | added CCU tail | | | |
| | Z38/z42 forward | DMD | 185 | ggagtTACCTCCAACATCAAGGAAGATGGCAGTAAC |
| | fusion long | exon 51 | | CACAGGTTGTGTCACCAGAGTAACAaATTTTTGGA |
| | mouse loop with | | | GcaGgttttctgacctccttcggtcggaaaacccct |
| | 5′ ISD and eSL | | | |

### Vectors

Within the context of a recombinant expression vector, the terminology “operably linked” is intended to mean that the hybrid promoter is linked to an NOI in a manner permitting expression of the nucleotide sequence in, for example, a host cell when the vector is introduced into (or in contact with) the host cell.

In some embodiments of the compositions and methods of the disclosure, a vector comprises the engineered snRNA. In some embodiments, the therapeutic snRNA is in a single or unitary vector.

In some embodiments of the compositions and methods of the disclosure, the RNA-binding snRNA systems are capable of targeting toxic CAG, CUG, GGCCCC, CCGGG, or GGCCC+CCGGGG RNA repeats (or flanking sequences thereof) are in a single vector. In some embodiments of the compositions and methods of the disclosure, the RNA-targeting systems are capable of targeting a non-repeat RNA of interest. In some embodiments of the compositions and methods of the disclosure, the RNA-targeting systems are capable of targeting one or more sequences of DMD. In some aspects, the snRNA systems are capable of targeting multiple (i.e., two or more) RNAs of interest. In some embodiments, the two or more RNAs of interest can be the same pre-mRNA molecule but different sequences within the pre-mRNA molecule.

One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses). Viral vectors also include polynucleotides carried by a virus for transfection into a host cell. In some embodiments, the vector is a lentivirus (such as an integration-deficient lentiviral vector) or adeno-associated viral (AAV) vector. Vectors are capable of autonomous replication in a host cell into which they are introduced such as e.g., bacterial vectors having a bacterial origin of replication and episomal mammalian vectors and other vectors such as, e.g., non-episomal mammalian vectors, are integrated into the genome of a host cell upon introduction into the host cell, and thereby are replicated along with the host genome.

In some embodiments, vectors such as e.g., expression vectors, are capable of directing the expression of genes to which they are operatively-linked. Common expression vectors are often in the form of plasmids. In some embodiments, recombinant expression vectors comprise a nucleic acid provided herein such as e.g., an esnRNA in a form suitable for expression of a protein in a host cell. Recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence such as e.g., in an in vitro transcription/translation system or in a host cell when the vector is introduced into the host cell. Certain embodiments of a vector depend on factors such as the choice of the host cell to be transformed, and the level of expression desired. A vector can be introduced into host cells to thereby produce transcripts, proteins, or peptides, including fusion proteins or peptides, encoded by nucleic acids as described herein such as, e.g., snRNAs, CRISPR transcripts, proteins, enzymes, mutant forms thereof, fusion proteins thereof, etc.

### Viral Vectors

In some embodiments of the compositions and methods of the disclosure, a vector of the disclosure is a viral vector. In some embodiments, the viral vector comprises a sequence isolated or derived from a retrovirus. In some embodiments, the viral vector comprises a sequence isolated or derived from a lentivirus. In some embodiments, the viral vector comprises a sequence isolated or derived from an adenovirus. In some embodiments, the viral vector comprises a sequence isolated or derived from an adeno-associated virus (AAV). In some embodiments, the viral vector is replication incompetent. In some embodiments, the viral vector is isolated or recombinant. In some embodiments, the viral vector is self-complementary.

### Adeno-Associated Virus Vectors

An “AAV vector” as used herein refers to a vector comprising, consisting essentially of, or consisting of one or more nucleic acid molecules and one or more AAV inverted terminal repeat sequences (ITRs). In some aspects, the nucleic acid molecule encodes for an esnRNA of the disclosure. Such AAV vectors can be replicated and packaged into infectious viral particles when present in a host cell that provides the functionality of rep and cap gene products, for example, by transfection of the host cell. In some aspects, AAV vectors contain a promoter, at least one nucleic acid that may encode at least one protein or RNA, and/or an enhancer and/or a terminator within the flanking ITRs that is packaged into the infectious AAV particle. The encapsidated nucleic acid portion may be referred to as the AAV vector genome. Plasmids containing AAV vectors may also contain elements for manufacturing purposes, e.g., antibiotic resistance genes, origin of replication sequences etc., but these are not encapsidated and thus do not form part of the AAV particle.

In some aspects, an AAV vector can comprise at least one nucleic acid encoding an esnRNA composition of the disclosure. In some aspects, an AAV vector can comprise at least one regulatory sequence. In some aspects, an AAV vector can comprise at least one AAV inverted terminal (ITR) sequence. In some aspects, an AAV vector can comprise a first ITR sequence and a second ITR sequence. In some aspects, an AAV vector can comprise at least one promoter sequence. In some aspects, an AAV vector can comprise at least one enhancer sequence. In some aspects, an AAV vector can comprise at least one terminator sequence. In some aspects, an AAV vector can comprise at least one polyA sequence. In some aspects, an AAV vector can comprise at least one linker sequence. In some aspects, an AAV vector can comprise at least one buffer sequence. In some aspects, an AAV vector of the disclosure can comprise at least one nuclear localization signal, or nuclear export signal and/or both.

In some aspects, an AAV vector can comprise a first AAV ITR sequence, a promoter sequence, an esnRNA sequence, a terminator sequence and a second AAV ITR sequence. In some aspects, an AAV vector can comprise, in the 5′ to 3′ direction, a first AAV ITR sequence, a promoter sequence, an esnRNA sequence, a terminator sequence, and a second AAV ITR sequence.

In some aspects, an AAV vector can comprise a first AAV ITR sequence, a first promoter sequence, a first esnRNA sequence, a second promoter sequence, second esnRNA sequence, and a second AAV ITR sequence. In some aspects, an AAV vector can comprise a first AAV ITR sequence, a first promoter sequence, a first esnRNA sequence, a second promoter sequence, a second esnRNA sequence, a third promoter sequence, a third esnRNA sequence, and a second AAV ITR sequence. In some aspects, an AAV vector can comprise a first AAV ITR sequence, a first promoter sequence, a first esnRNA sequence, a second promoter sequence, second esnRNA sequence, and a second AAV ITR sequence. In some aspects, an AAV vector can comprise a first AAV ITR sequence, a first promoter sequence, a first esnRNA sequence, a second promoter sequence, a second esnRNA sequence, a third promoter sequence, a third esnRNA sequence, a fourth promoter sequence, a fourth esnRNA sequence, and a second AAV ITR sequence.

The term “adeno-associated virus” or “AAV” as used herein refers to a member of the class of viruses associated with this name and belonging to the genus Dependoparvovirus, family Parvoviridae. Adeno-associated virus is a single-stranded DNA virus that grows in cells in which certain functions are provided by a co-infecting helper virus. General information and reviews of AAV can be found in, for example, Carter, 1989, Handbook of Parvoviruses, Vol. 1, pp. 169-228, and Berns, 1990, Virology, pp. 1743-1764, Raven Press, (New York). It is fully expected that the same principles described in these reviews will be applicable to additional AAV serotypes characterized after the publication dates of the reviews because it is well known that the various serotypes are quite closely related, both structurally and functionally, even at the genetic level. (See, for example, Blacklowe, 1988, pp. 165-174 of Parvoviruses and Human Disease, J. R. Pattison, ed.; and Rose, Comprehensive Virology 3: 1-61 (1974)). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control. Multiple serotypes of this virus are known to be suitable for gene delivery; all known serotypes can infect cells from various tissue types.

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is inserted as cloned DNA in plasmids, which makes construction of recombinant genomes feasible. Furthermore, because the signals directing AAV replication and genome encapsidation are contained within the ITRs of the AAV genome, some or all of the internal approximately 4.3 kb of the genome (encoding replication and structural capsid proteins, rep-cap) may be replaced with foreign DNA to generate AAV vectors. The rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

Recombinant AAV (rAAV) genomes of the invention comprise, consist essentially of, or consist of a nucleic acid molecule encoding at least one esnRNA and one or more AAV ITRs flanking the nucleic acid molecule. Production of pseudotyped rAAV is disclosed in, for example, WO2001083692. Other types of rAAV variants, for example rAAV with capsid mutations, are also contemplated. See, e.g., Marsic et al., Molecular Therapy, 22(11): 1900-1909 (2014). The nucleotide sequences of the genomes of various AAV serotypes are known in the art.

In some embodiments of the compositions and methods of the disclosure, the viral vector comprises a sequence isolated or derived from an adeno-associated virus (AAV). In some embodiments, the viral vector comprises an inverted terminal repeat sequence or a capsid sequence that is isolated or derived from an AAV of serotype AAV1, AAV2, AAV3, AAV4, AAV5, AAV6, AAV7, AAV8, AAV9, AAV10, AAVrh10, AAV11 or AAV12. In some embodiments, the AAV serotype is AAVrh.74. In one embodiment, the AAV vector comprises a modified capsid. In one embodiment the AAV vector is an AAV2-Tyr mutant vector. In one embodiment the AAV vector comprises a capsid with a non-tyrosine amino acid at a position that corresponds to a surface-exposed tyrosine residue in position Tyr252, Tyr272, Tyr275, Tyr281, Tyr508, Tyr612, Tyr704, Tyr720, Tyr730 or Tyr673 of wild-type AAV2. See also WO 2008/124724 incorporated herein in its entirety. In some embodiments, the AAV vector comprises an engineered capsid. AAV vectors comprising engineered capsids include without limitation, AAV2.7m8, AAV9.7m8, AAV2 2tYF, and AAV8 Y733F). In some embodiments, the capsid is a ubiquitination resistant capsid. In another embodiment, the ubiquitination capsid is an AAV2 capsid comprising tyrosine (Y) and serine (S) mutations. In another embodiment, the AAV2 capsid comprises Y, S and threonine (T) mutations. In another embodiment, the AAV2 capsid includes, without limitation, AAV2 capsid mutants such as T455V, T491V, T550V, T659V, Y444+500+730F, and Y444+500+730F+T491V. In some embodiments, the viral vector is replication incompetent. In some embodiments, the viral vector is isolated or recombinant (rAAV). In some embodiments, the viral vector is self-complementary (scAAV). In some embodiments, the viral vector is single-stranded (ssAAV).

### AAV ITR Sequences

In some embodiments of the compositions and methods of the disclosure, an AAV inverted terminal repeat sequence can comprise any AAV ITR sequence known in the art. In some aspects, an AAV ITR sequence can comprise or consist of an AAV1 ITR sequence, an AAV2 ITR sequence, an AAV3 ITR sequence, an AAV4 ITR sequence, an AAV5 ITR sequence, an AAV6 ITR sequence, an AAV7 ITR sequence, an AAV8 ITR sequence, an AAV9 ITR sequence, an AAV10 ITR sequence, an AAVrh10 ITR sequence, an AAV11 ITR sequence, an AAV12 ITR sequence, an AAV13 ITR sequence, or an AAVrh74 ITR sequence.

In some aspects, the ITR sequence can comprise a modified AAV ITR sequence.

In some aspects, an AAV ITR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 59, SEQ ID NO: 60, SEQ ID NO: 61, SEQ ID NO: 162, or SEQ ID NO: 166.

In some aspects, a first AAV ITR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 59, SEQ ID NO: 60, SEQ ID NO: 61, SEQ ID NO: 162, or SEQ ID NO: 166 and a second AAV ITR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 59, SEQ ID NO: 60, SEQ ID NO: 61, SEQ ID NO: 162, or SEQ ID NO: 166. In some aspects, the first AAV ITR sequence is positioned at the 5′ of a AAV vector. In some aspects, the second AAV ITR sequence is positioned at the 3′ of a AAV vector.

In some embodiments of the compositions and methods of the disclosure, the viral vector comprises a sequence isolated or derived from an adeno-associated virus (AAV).

In some embodiments of the compositions and methods of the disclosure, a vector of the disclosure is a non-viral vector. In some embodiments, the vector comprises or consists of a nanoparticle, a micelle, a liposome or lipoplex, a polymersome, a polyplex or a dendrimer. In some embodiments, the vector is an expression vector or recombinant expression system. As used herein, the term “recombinant expression system” refers to a genetic construct for the expression of certain genetic material formed by recombination.

esnRNA Vector Constructs

Exemplary esnRNA AAV vectors of the disclosure can comprise one or more esnRNA sequences of the disclosure. In some aspects, the esnRNA AAV vector comprises, consists essentially of, or consists of a nucleic acid sequence at least 65%, 70%, 75%, 80%, 85%, 90%, 95%, 96%, 97%, 98%, 99% or 100% (or any percentage in between) identical to a an esnRNA AAV vector listed in the esnRNA AAV vector Table which follows:

| | | SEQ ID |
|---|---|---|
| ID | Description | NO: |
| A02962 | ss pAAV-4X U7(15XCUG) (derivative of P00369) | 120 |
| | targeting CUG repeats | |
| A02963 | ss pAAV-4X U7(NT1) (derivative of P00369) | 121 |
| A02967 | ss pAAV-4X U7(15XCUG) targeting CUG repeats | 122 |
| A03079 | sc pAAV-4X U7(15XCUG) (derivative of P00369) | 123 |
| | targeting CAG repeats | |
| A03080 | sc pAAV-4X U7(NT1) (derivative of P00369) | 124 |
| A03081 | sc pAAV-4X U7(15XCTG) targeting CUG repeats | 125 |
| A02888 | pAAV_1xU7(15xCUG)_CMV_GFP (no buffer) targeting | 126 |
| | CUG repeats | |
| A02889 | pAAV_1xU7(NT1)_CMV_GFP (no buffer) | 127 |
| A02925 | pAAV_1xU7(15xCAG)_CMV_GFP (no buffer) targeting | 128 |
| | CAG repeats | |
| A02894 | pAAV_3xU7(15xCUG)_CMV_GFP (100 bp buffer) | 129 |
| | targeting CUG repeats | |
| A02895 | pAAV_3xU7(NT1)_CMV_GFP (100 bp buffer) | 130 |
| A02968 | pAAV_3xU7(15XCAG)_CMV_GFP (100 bp buffer) | 131 |
| | targeting CAG repeats | |
| A02896 | pAAV_3xU7(15xCUG)_CMV_GFP (500 bp buffer) | 132 |
| A02897 | PAAV_3xU7(NT1)_CMV_GFP (500 bp buffer) | 133 |
| A02969 | pAAV_3xU7(15xCAG)_CMV_GFP (500 bp buffer) | 134 |
| | targeting CAG repeats | |
| A03980 | pAAV(ssAAV) U7promoter-esnRNA-z38/42-U7 termination- | 135 |
| | U1promoter-esnRNAz38/z42-U1 termination | |
| A03981 | pAAV(ssAAV)_U7promoter-esnRNA-z42-U7termination- | 136 |
| | U1promoter-esnRNA-z38-U1 termination | |
| A04229 | pAAV(scAAV) U7promoter-esnRNA-z38/42-U7 termination- | 137 |
| | U1promoter-esnRNAz38/z42-U1 termination-mut ITR | |
| A04184 | pAAV(ssAAV)-U7promoter-z38/42-U7term_U1promoter- | 138 |
| | z38/42-U1term_buffer combination sequencestuffer | |
| A03681 | pAAV(ssAAV)-U7 promoter-zCUGx15-U7 term_U1 promoter- | 139 |
| | zCUGx15-U1 term-eCMV-eGFP | |
| A03682 | pAAV(ssAAV)-U1 promoter-zCUGx15-U1 term_U7 promoter- | 140 |
| | zCUGx15-U7 term-eCMV-eGFP | |
| A03683 | pAAV(ssAAV)-U7 promoter-znt-U7 term_U1 promoter-znt-U1 | 141 |
| | term-eCMV-eGFP | |
| A03864 | pAAV(ssAAV)-U7 promoter-zCUGx15-U7 term_U1 promoter- | 142 |
| | zCUGx15-U1 term (minus GFP − derivative of 3681) | |
| A04233 | pAAV(scAAV)-U7promoter-zCUGx15-U7termination- | 143 |
| | U1promoter-zCUGx15-U1termination (derivative of | |
| | A03681 no GFP in scAAV) | |
| A04569 | scAAVmU7p-z42/38 rev fus-mU7term_mU1p-z42/38 rev fus- | 168 |
| | mU1term; mouse loop with5′ ISD and eSL | |
| A04526 | scAAVmU7p-z38/42 Forward fus-mU7term_mU1p-z38/42 | 169 |
| | forward fus-mU1term; mouseloop with5′ ISD and eSL | |
| A04771 | scAAVmU7p-z42/38 reverse fus-mU7term_mU1p-z42/38 | 170 |
| | reverse fus-mU1term; mouseloop with 5′ ISD and eSL no | |
| | CCU tail | |
| A04525 | scAAV-mU7p-z38/42-mU7term_mU7p-z38/42-mU7term; | 171 |
| | mouse loop no ISD no eSL | |
| A04527 | scAAV-mU7p-z38/42-mU7term_mU1p-z38/42-mU1term; | 172 |
| | mouse loop no ISD no eSL | |
| A04528 | scAAV-mU7p-z38/42-mU7term_mU1p-z38/42-mU1term; | 173 |
| | human loop with ISD and eSL | |
| A04529 | scAAV-mU7p-z38/42-mU7term_mU1p-z38/42-mU1term; | 174 |
| | human loop with ISD and eSL with added CCU tail | |
| A04537 | scAAV-mU7p-z38/42-mU7term_mU1prom- p-z38/42-mU1term; | 175 |
| | long mouse loop with5′ ISD and eSL | |
| A04233 | scAAV- hU7-CUGx15-hU1-CUGx15 (3′mut ITR) | 176 |
| A04234 | scAAV- hU7-CUGx15-hU1-CUGx15 (5′ mut ITR) | 177 |
| A04390 | scAAV 5′mITR hU7-CAG15X-hU1-CAGx15 | 178 |
| A04530 | scAAV- mU7p-CUGx15-mU1p-CUGx15; mouseloop with | 231 |
| | ISD and eSL | |
| A04940 | scAAV- mU7p-CUGx10-mU1p-CUGx10; mouseloop with ISD | 232 |
| | and eSL no CCU | |
| A04533 | scAAV- mU7p-CAGx15-mU1p-CAGx15; mouseloop with ISD | 233 |
| | and eSL | |

An exemplary AAV vector of the disclosure comprises from 5′ to 3′: a first inverted terminal repeat (ITR) sequence, a U7 promoter sequence, a DMD exon 51 targeting U7 esnRNA sequence, a U7 downstream terminator sequence, a U1 promoter sequence, a DMD exon 51 targeting U7 esnRNA sequence, a U1 downstream terminator sequence, and a second ITR sequence. In some aspects, a nucleic acid sequence encoding AAV vector A04569 encoding DMD exon 51 targeting snRNA sequences comprises SEQ ID NO: 168.

A04569: scAAVmU7p-z42/38 rev fus-mU7term_mU1p-z42/38 rev fus-mU1term; mouse loop with 5′ ISD and eSL

| Plasmid Element | Nucleotide Sequence 5′ to 3′ |
|---|---|
| 5′ ITR | ctgcgcgctcgctcgctcactgaggccgcccgggcaaagcccgggcgtcgggcgacctttggtcgcccggcctcagtgagcgag |
| | cgagcgcgcagagagggagtggggtt (SEQ ID NO: 162) |
| Mouse U7 | taacaacataggagctgtgattggctgttttcagccaatcagcactgActcatttgcatagcctttacaagcggtcacaaactca |
| promoter | agaaacgagcggttttaatagtcttttagaatattgtttatcgaaccgaataaggaactgtgctttgtgattcacatatcagtgg |
| | aggggtgtggaaatggcaccttgatctcaccctcatcgaaagtggagttgatgtcctTccctggctcgctacagacgcacttcc |
| | gcaa (SEQ ID NO: 48) |
| ISD | ggagt (SEQ ID NO: 12) |
| Z42/z38 reverse | GTAACCACAGGTTGTGTCACCAGAGTAACATACCTCCAACATCAAGGAAGATGG |
| fusion spacer | CA (SEQ ID NO: 149) |
| SMOPT | aATTTTTGGAGca (SEQ ID NO: 161) |
| Mousc stem loop | ggttttctgacctccgtcggaaaacccct (SEQ ID NO: 146) |
| (eSL) short | |
| loop | |
| Mouse U7 | cccaatttcactggtctacaatgaaagcaaaacagttctcttccccgctccccggtgtgtgagaggggctttgatccttctctgg |
| terminator | tttcctaggaaacgcgtatgtg (SEQ ID NO: 156) |
| Mousc U1 | TTGTTCCTCTTAGTGTTAATTCACACTAAAGACTGTGCATCCGACTCCTACATTTA |
| promoter | TGAAAGTAAATGCCTGTTGTTAGAACAAAAAAGGCTACAGAACAAAAAACAAA |
| | GCGAAATACCATCTGCTTTAGGTTCAGTGTGGTATTTTCCCGCTGACAGGGAGGC |
| | GGGTTTTTGGGTACAGGAAACGAGTCACTATGGAGGCGGTACTATGTAGATGAG |
| | AATTCAGGTGCAAACTGGGAAAAGCAACTGCTTCCAAATATTTGTGATTTTTACA |
| | GTGTAGTTTTGGAAAAACTCTTAGCCTACCAATTCTTCTAAGTGTTTTAAAATGT |
| | GGGAGCCAGTACACATGAAGTTATAGAGTGTTTTAATGAGGCTTAAATATTTACC |
| | GTAACTATGAAATGCTACGCATATCATGCTGTTCAGGCTCCGTGGCCACGCAACT |
| | C (SEQ ID NO: 47) |
| ISD | ggagt (SEQ ID NO: 12) |
| Z42/z38 reverse | GTAACCACAGGTTGTGTCACCAGAGTAACATACCTCCAACATCAAGGAAGATGG |
| fusion spacer | CA (SEQ ID NO: 149) |
| SMOPT | aATTTTTGGAGca (SEQ ID NO: 161) |
| Mouse stem loop | ggttttctgacctccgtcggaaaacccct (SEQ ID NO: 146) |
| (eSL) short | |
| loop | |
| Mouse U1 | GTTTACTTGGTTTTAAAAATAGCTTGCACTAGCGATACGGAATATGGTTATTAGG |
| terminator | TTTGTTAGGCATCATGTCGTGTCTTACTATAGAAAAATAACGTAGTGTTCATTTT |
| | AGCCTGCCTGTATGTGTTAATTTGTCCTTATTGCGCATTGTTCTTGTTAAGTCTTC |
| | TGTAAGGAGTTGCGGGTTTCAAACTGTCAGTCTGAGAGCA (SEQ ID NO: 57) |
| 3′ ITR | Aggaacccctagtgatggagttggccactccctctctgcgcgctcgctcgctcactgaggccgggcgaccaaaggtcgcccgacg |
| | cccgggctttgcccgggggcctcagtgagcgagcgagcgcgcagctgcctgcagg (SEQ ID NO: 60) |

An exemplary AAV vector of the disclosure comprises from 5′ to 3′: a first inverted terminal repeat (ITR) sequence, a U7 promoter sequence, a DMD exon 51 targeting U7 esnRNA sequence, a U7 downstream terminator sequence, a U1 promoter sequence, a DMD exon 51 targeting U7 esnRNA sequence, a U1 downstream terminator sequence, and a second ITR sequence. In some aspects, a nucleic acid sequence encoding AAV vector A04526 encoding DMD exon 51 targeting snRNA sequences comprises SEQ ID NO: 169.

A04526: scAAVmU7p-z82/42 Forward Fus-mU7term_mU1p-z38/42 Forward Fus-mU1term; Mouseloop with 5′ ISD and eSL

| Plasmid Element | Nucleotide Sequence 5′ to 3′ |
|---|---|
| 5′ ITR | ctgcgcgctcgctcgctcactgaggccgcccgggcaaagcccgggcgtcgggcgacctttggtcgcccggcctcagtgagcgag |
| | cgagcgcgcagagagggagtggggtt (SEQ ID NO: 162) |
| Mouse U7 | taacaacataggagctgtgattggctgttttcagccaatcagcactgActcatttgcatagcctttacaagcggtcacaaactc |
| promoter | aagaaacgagcggttttaatagtcttttagaatattgtttatcgaaccgaataaggaactgtgctttgtgattcacatatcagt |
| | ggaggggtgtggaaatggcaccttgatctcaccctcatcgaaagtggagttgatgtcctTccctggctcgctacagacgcact |
| | tccgcaa (SEQ ID NO: 48) |
| ISD | ggagt (SEQ ID NO: 12) |
| Z38/z42 forward | TACCTCCAACATCAAGGAAGATGGCAGTAACCACAGGTTGTGTCACCAGAGTAA |
| fusion spacer | CA (SEQ ID NO: 150) |
| SMOPT | aATTTTTGGAGca (SEQ ID NO: 161) |
| Mouse stem loop | ggttttctgacctccgtcggaaaacccct (SEQ ID NO: 146) |
| (eSL) short | |
| loop | |
| Mouse U7 | cccaatttcactggtctacaatgaaagcaaaacagttctcttccccgctccccggtgtgtgagaggggctttgatccttctctg |
| terminator | gtttcctaggaaacgcgtatgtg (SEQ ID NO: 156) |
| Mouse U1 | TTGTTCCTCTTAGTGTTAATTCACACTAAAGACTGTGCATCCGACTCCTACATTTA |
| promoter | TGAAAGTAAATGCCTGTTGTTAGAACAAAAAAGGCTACAGAACAAAAAACAAA |
| | GCGAAATACCATCTGCTTTAGGTTCAGTGTGGTATTTTCCCGCTGACAGGGAGGC |
| | GGGTTTTTGGGTACAGGAAACGAGTCACTATGGAGGCGGTACTATGTAGATGAG |
| | AATTCAGGTGCAAACTGGGAAAAGCAACTGCTTCCAAATATTTGTGATTTTTACA |
| | GTGTAGTTTTGGAAAAACTCTTAGCCTACCAATTCTTCTAAGTGTTTTAAAATGT |
| | GGGAGCCAGTACACATGAAGTTATAGAGTGTTTTAATGAGGCTTAAATATTTACC |
| | GTAACTATGAAATGCTACGCATATCATGCTGTTCAGGCTCCGTGGCCACGCAACT |
| | C (SEQ ID NO: 47) |
| ISD | ggagt (SEQ ID NO: 12) |
| Z38/z42 forward | TACCTCCAACATCAAGGAAGATGGCAGTAACCACAGGTTGTGTCACCAGAGTAA |
| fusion spacer | CA (SEQ ID NO: 150) |
| SMOPT | aATTTTTGGAGca (SEQ ID NO: 161) |
| Mouse stem loop | ggttttctgacctccgtcggaaaacccct (SEQ ID NO: 146) |
| (eSL) short | |
| loop | |
| Mousc U1 | GTTTACTTGGTTTTAAAAATAGCTTGCACTAGCGATACGGAATATGGTTATTAGG |
| terminator | TTTGTTAGGCATCATGTCGTGTCTTACTATAGAAAAATAACGTAGTGTTCATTTT |
| | AGCCTGCCTGTATGTGTTAATTTGTCCTTATTGCGCATTGTTCTTGTTAAGTCTTC |
| | TGTAAGGAGTTGCGGGTTTCAAACTGTCAGTCTGAGAGCA (SEQ ID NO: 57) |
| 3′ ITR | Aggaacccctagtgatggagttggccactccctctctgcgcgctcgctcgctcactgaggccgggcgaccaaaggtcgcccgacg |
| | ccgggctttgcccgggcggcctcagtgagcgagcgagcgcgcagctgcctgcagg (SEQ ID NO: 60) |

An exemplary AAV vector of the disclosure comprises from 5′ to 3′: a first inverted terminal repeat (ITR) sequence, a U7 promoter sequence, a DMD exon 51 targeting U7 esnRNA sequence, a U7 downstream terminator sequence, a U1 promoter sequence, a DMD exon 51 targeting U7 esnRNA sequence, a U1 downstream terminator sequence, and a second ITR sequence. In some aspects, a nucleic acid sequence encoding AAV vector A04771 encoding DMD exon 51 targeting snRNA sequences comprises SEQ ID NO: 170.

A04771: scAAVmU7p-z42/38 Reverse Fus-mU7term_mU1p-z42/38 Reverse Fus-mU1term; Mouseloop with 5 ISD and eSL No CCU Tail

| Plasmid Element | Nucleotide Sequence 5′ to 3′ |
|---|---|
| 5′ ITR | ctgcgcgctcgctcgctcactgaggccgcccgggcaaagcccgggcgtcgggcgacctttggtcgcccggcctcagtgagcgagc |
| | gagcgcgcagagagggagtggggtt (SEQ ID NO: 162) |
| Mouse U7 | taacaacataggagctgtgattggctgttttcagccaatcagcactgActcatttgcatagcctttacaagcggtcacaaactca |
| promoter | agaaacgagcggttttaatagtcttttagaatattgtttatcgaaccgaataaggaactgtgctttgtgattcacatatcagtgg |
| | aggggtgtggaaatggcaccttgatctcaccctcatcgaaagtggagttgatgtcctTccctggctcgctacagacgcacttcc |
| | gcaa (SEQ ID NO: 48) |
| ISD | ggagt (SEQ ID NO: 12) |
| Z42/z38 reverse | GTAACCACAGGTTGTGTCACCAGAGTAACATACCTCCAACATCAAGGAAGATGGC |
| fusion spacer | A (SEQ ID NO: 149) |
| SMOPT | aATTTTTGGAGca (SEQ ID NO: 161) |
| Mouse stem loop | ggttttctgacctccgtcggaaaacc (SEQ ID NO: 148) |
| (eSL) short | |
| loop no CCU | |
| Mouse U7 | cccaatttcactggtctacaatgaaagcaaaacagttctcttccccgctccccggtgtgtgagaggggctttgatccttctctg |
| terminator | gtttcctaggaaacgcgtatgtg (SEQ ID NO: 156) |
| Mousc U1 | TTGTTCCTCTTAGTGTTAATTCACACTAAAGACTGTGCATCCGACTCCTACATTTAT |
| promoter | GAAAGTAAATGCCTGTTGTTAGAACAAAAAAGGCTACAGAACAAAAAACAAAGC |
| | GAAATACCATCTGCTTTAGGTTCAGTGTGGTATTTTCCCGCTGACAGGGAGGCGG |
| | GTTTTTGGGTACAGGAAACGAGTCACTATGGAGGCGGTACTATGTAGATGAGAAT |
| | TCAGGTGCAAACTGGGAAAAGCAACTGCTTCCAAATATTTGTGATTTTTACAGTGT |
| | AGTTTTGGAAAAACTCTTAGCCTACCAATTCTTCTAAGTGTTTTAAAATGTGGGAG |
| | CCAGTACACATGAAGTTATAGAGTGTTTTAATGAGGCTTAAATATTTACCGTAACT |
| | ATGAAATGCTACGCATATCATGCTGTTCAGGCTCCGTGGCCACGCAACTC (SEQ ID |
| | NO: 47) |
| ISD | ggagt (SEQ ID NO: 12) |
| Z42/z38 reverse | GTAACCACAGGTTGTGTCACCAGAGTAACATACCTCCAACATCAAGGAAGATGGC |
| fusion spacer | A (SEQ ID NO: 149) |
| SMOPT | aATTTTTGGAGca (SEQ ID NO: 161) |
| Mouse stem loop | ggttttctgacctccgtcggaaaacc (SEQ ID NO: 148) |
| (eSL) short | |
| loop no CCU | |
| Mouse U1 | GTTTACTTGGTTTTAAAAATAGCTTGCACTAGCGATACGGAATATGGTTATTAGGT |
| terminator | TTGTTAGGCATCATGTCGTGTCTTACTATAGAAAAATAACGTAGTGTTCATTTTAG |
| | CCTGCCTGTATGTGTTAATTTGTCCTTATTGCGCATTGTTCTTGTTAAGTCTTCTGT |
| | AAGGAGTTGCGGGTTTCAAACTGTCAGTCTGAGAGCA (SEQ ID NO: 57) |
| 3′ ITR | Aggaacccctagtgatggagttggccactccctctctgcgcgctcgctcgctcactgaggccgggcgaccaaaggtcgcccgacg |
| | gggctttgcccgggggcctcagtgagcgagcgagcgcgcagctgcctgcagg (SEQ ID NO: 60) |

An exemplary AAV vector of the disclosure comprises from 5′ to 3′: a first inverted terminal repeat (ITR) sequence, a U7 promoter sequence, a DMD exon 51 targeting U7 esnRNA sequence, a U7 downstream terminator sequence, a U7 promoter sequence, a DMD exon 51 targeting U7 esnRNA sequence, a U7 downstream terminator sequence, and a second ITR sequence. In some aspects, a nucleic acid sequence encoding AAV vector A04525 encoding DMD exon 51 targeting snRNA sequences comprises SEQ ID NO: 171.

A04525: scAAV-mU7p-z38/42-mU7term_U7p-z38/42-mU7term; mouse loop no ISD no eSL

| Plasmid Element | Nucleotide Sequence 5′ to 3′ |
|---|---|
| 5′ ITR | ctgcgcgctcgctcgctcactgaggccgcccgggcaaagcccgggcgtcgggcgacctttggtcgcccggcctcagtgagcgag |
| | cgagcgcgcagagagggagtggggtt (SEQ ID NO: 162) |
| Mouse U7 promoter | taacaacataggagctgtgattggctgttttcagccaatcagcactgActcatttgcatagcctttacaagcggtcacaaact |
| | caagaaacgagcggttttaatagtcttttagaatattgtttatcgaaccgaataaggaactgtgctttgtgattcacatatc |
| | agtggaggggtgtggaaatggcaccttgatctcaccctcatcgaaagtggagttgatgtcctTccctggctcgctacagacg |
| | cacttccgcaa (SEQ ID NO: 48) |
| Z38/z42 forward | TACCTCCAACATCAAGGAAGATGGCAGTAACCACAGGTTGTGTCACCAGAGTAAC |
| fusion spacer | A (SEQ ID NO: 150) |
| SMOPT | aATTTTTGGAGca (SEQ ID NO: 161) |
| Mouse stem loop | ggttttctgacttcggtcggaaaacccct (SEQ ID NO: 145) |
| (WT) short loop | |
| Mouse U7 | cccaatttcactggtctacaatgaaagcaaaacagttctcttccccgctccccggtgtgtgagaggggctttgatccttctct |
| terminator | ggtttcctaggaaacgcgtatgtg (SEQ ID NO: 156) |
| Mouse U7 promoter | taacaacataggagctgtgattggctgttttcagccaatcagcactgActcatttgcatagcctttacaagcggtcacaaac |
| | tcaagaaacgagcggttttaatagtcttttagaatattgtttatcgaaccgaataaggaactgtgctttgtgattcacatat |
| | cagtggaggggtgtggaaatggcaccttgatctcaccctcatcgaaagtggagttgatgtcctTccctggctcgctacagac |
| | gcacttccgcaa (SEQ ID NO: 48) |
| Z38/z42 forward | TACCTCCAACATCAAGGAAGATGGCAGTAACCACAGGTTGTGTCACCAGAGTAAC |
| fusion spacer | A (SEQ ID NO: 150) |
| SMOPT | aATTTTTGGAGca (SEQ ID NO: 161) |
| Mouse stem loop | ggttttctgacttcggtcggaaaacccct (SEQ ID NO: 145) |
| (WT) short loop | |
| Mouse U7 | cccaatttcactggtctacaatgaaagcaaaacagttctcttccccgctccccggtgtgtgagaggggctttgatccttctc |
| terminator | tggtttcctaggaaacgcgtatgtg (SEQ ID NO: 156) |
| 3′ ITR | Aggaacccctagtgatggagttggccactccctctctgcgcgctcgctcgctcactgaggccgggcgaccaaaggtcgcccgac |
| | gcgggctttgcccgggcggcctcagtgagcgagcgagcgcgcagctgcctgcagg (SEQ ID NO: 60) |

An exemplary AAV vector of the disclosure comprises from 5′ to 3′: a first inverted terminal repeat (ITR) sequence, a U7 promoter sequence, a DMD exon 51 targeting U7 esnRNA sequence, a U7 downstream terminator sequence, a U1 promoter sequence, a DMD exon 51 targeting U7 esnRNA sequence, a U1 downstream terminator sequence, and a second ITR sequence. In some aspects, a nucleic acid sequence encoding AAV vector A04527 encoding DMD exon 51 targeting snRNA sequences comprises SEQ ID NO: 172.

A04527: scAAV-mU7p-z38/42-mU7term_mU1p-z38/42-mU1term; Mouse Loop No ISD No eSL

| Plasmid Element | Nucleotide Sequence 5′ to 3′ |
|---|---|
| 5′ ITR | ctgcgcgctcgctcgctcactgaggccgcccgggcaaagcccgggcgtcgggcgacctttggtcgcccggcctcagtgagcgag |
| | cgagcgcgcagagagggagtggggtt (SEQ ID NO: 162) |
| Mouse U7 | taacaacataggagctgtgattggctgttttcagccaatcagcactgActcatttgcatagcctttacaagcggtcacaaactc |
| promoter | aagaaacgagcggttttaatagtcttttagaatattgtttatcgaaccgaataaggaactgtgctttgtgattcacatatcag |
| | tggaggggtgtggaaatggcaccttgatctcaccctcatcgaaagtggagttgatgtcctTccctggctcgctacagacgcact |
| | tccgcaa (SEQ ID NO: 48) |
| Z38/z42 forward | TACCTCCAACATCAAGGAAGATGGCAGTAACCACAGGTTGTGTCACCAGAGTAAC |
| fusion spacer | A (SEQ ID NO: 150) |
| SMOPT | aATTTTTGGAGca (SEQ ID NO: 161) |
| Mouse stem loop | ggttttctgacttcggtcggaaaacccct (SEQ ID NO: 145) |
| (WT) short loop | |
| Mouse U7 | cccaatttcactggtctacaatgaaagcaaaacagttctcttccccgctccccggtgtgtgagaggggctttgatccttctctg |
| terminator | gtttcctaggaaacgcgtatgtg (SEQ ID NO: 156) |
| Mouse U1 | TTGTTCCTCTTAGTGTTAATTCACACTAAAGACTGTGCATCCGACTCCTACATTTAT |
| promoter | GAAAGTAAATGCCTGTTGTTAGAACAAAAAAGGCTACAGAACAAAAAACAAAGC |
| | GAAATACCATCTGCTTTAGGTTCAGTGTGGTATTTTCCCGCTGACAGGGAGGCGG |
| | GTTTTTGGGTACAGGAAACGAGTCACTATGGAGGCGGTACTATGTAGATGAGAAT |
| | TCAGGTGCAAACTGGGAAAAGCAACTGCTTCCAAATATTTGTGATTTTTACAGTGT |
| | AGTTTTGGAAAAACTCTTAGCCTACCAATTCTTCTAAGTGTTTTAAAATGTGGGAG |
| | CCAGTACACATGAAGTTATAGAGTGTTTTAATGAGGCTTAAATATTTACCGTAACT |
| | ATGAAATGCTACGCATATCATGCTGTTCAGGCTCCGTGGCCACGCAACTC (SEQ ID |
| | NO: 47) |
| Z38/z42 forward | TACCTCCAACATCAAGGAAGATGGCAGTAACCACAGGTTGTGTCACCAGAGTAAC |
| fusion spacer | A (SEQ ID NO: 150) |
| SMOPT | aATTTTTGGAGca (SEQ ID NO: 161) |
| Mouse stem loop | ggttttctgacttcggtcggaaaacccct (SEQ ID NO: 145) |
| (WT) short loop | |
| Mouse U1 | GTTTACTTGGTTTTAAAAATAGCTTGCACTAGCGATACGGAATATGGTTATTAGGT |
| terminator | TTGTTAGGCATCATGTCGTGTCTTACTATAGAAAAATAACGTAGTGTTCATTTTAG |
| | CCTGCCTGTATGTGTTAATTTGTCCTTATTGCGCATTGTTCTTGTTAAGTCTTCTGT |
| | AAGGAGTTGCGGGTTTCAAACTGTCAGTCTGAGAGCA (SEQ ID NO: 57) |
| 3′ ITR | aggaacccctagtgatggagttggccactccctctctgcgcgctcgctcgctcactgaggccgggcgaccaaaggtcgcccga |
| | cgcccgggctttgcccgggggcctcagtgagcgagcgagcgcgcagctgcctgcagg (SEQ ID NO: 60) |

An exemplary AAV vector of the disclosure comprises from 5′ to 3′: a first inverted terminal repeat (ITR) sequence, a U7 promoter sequence, a DMD exon 51 targeting U7 esnRNA sequence, a U7 downstream terminator sequence, a U1 promoter sequence, a DMD exon 51 targeting U7 esnRNA sequence, a U1 downstream terminator sequence, and a second ITR sequence. In some aspects, a nucleic acid sequence encoding AAV vector A04528 encoding DMD exon 51 targeting snRNA sequences comprises SEQ ID NO: 173.

A04528: scAAV-mU7p-z38/42-mU7term_mU1p-z38/42-mU1term; Human Loop with ISD and eSL

| Plasmid Element | Nucleotide Sequence 5′ to 3′ |
|---|---|
| 5′ ITR | ctgcgcgctcgctcgctcactgaggccgcccgggcaaagcccgggcgtcgggcgacctttggtcgcccggcctcagtgagcgag |
| | cgagcgcgcagagagggagtggggtt (SEQ ID NO: 162) |
| Mouse U7 | taacaacataggagctgtgattggctgttttcagccaatcagcactgActcatttgcatagcctttacaagcggtcacaaactc |
| promoter | aagaaacgagcggttttaatagtcttttagaatattgtttatcgaaccgaataaggaactgtgctttgtgattcacatatcagt |
| | ggaggggtgtggaaatggcaccttgatctcaccctcatcgaaagtggagttgatgtcctTccctggctcgctacagacgcact |
| | tccgcaa (SEQ ID NO: 48) |
| ISD | ggagt (SEQ ID NO: 12) |
| Z38/z42 forward | TACCTCCAACATCAAGGAAGATGGCAGTAACCACAGGTTGTGTCACCAGAGTAA |
| fusion spacer | CA (SEQ ID NO: 150) |
| SMOPT | AATTTTTGGAGTA (SEQ ID NO: 164) |
| Human stem loop | ggctttctggctccttaccggaaagcc (SEQ ID NO: 1) |
| (eSL) | |
| Mouse U7 | cccaatttcactggtctacaatgaaagcaaaacagttctcttccccgctccccggtgtgtgagaggggctttgatccttctctg |
| terminator | gtttcctaggaaacgcgtatgtg (SEQ ID NO: 156) |
| Mouse U1 | TTGTTCCTCTTAGTGTTAATTCACACTAAAGACTGTGCATCCGACTCCTACATTTA |
| promoter | TGAAAGTAAATGCCTGTTGTTAGAACAAAAAAGGCTACAGAACAAAAAACAAA |
| | GCGAAATACCATCTGCTTTAGGTTCAGTGTGGTATTTTCCCGCTGACAGGGAGGC |
| | GGGTTTTTGGGTACAGGAAACGAGTCACTATGGAGGCGGTACTATGTAGATGAG |
| | AATTCAGGTGCAAACTGGGAAAAGCAACTGCTTCCAAATATTTGTGATTTTTACA |
| | GTGTAGTTTTGGAAAAACTCTTAGCCTACCAATTCTTCTAAGTGTTTTAAAATGT |
| | GGGAGCCAGTACACATGAAGTTATAGAGTGTTTTAATGAGGCTTAAATATTTACC |
| | GTAACTATGAAATGCTACGCATATCATGCTGTTCAGGCTCCGTGGCCACGCAACT |
| | C (SEQ ID NO: 47) |
| ISD | ggagt (SEQ ID NO: 12) |
| Z38/z42 forward | TACCTCCAACATCAAGGAAGATGGCAGTAACCACAGGTTGTGTCACCAGAGTAA |
| fusion spacer | CA (SEQ ID NO: 150) |
| SMOPT | AATTTTTGGAGTA (SEQ ID NO: 164) |
| Human stem loop | ggctttctggctccttaccggaaagcc (SEQ ID NO: 1) |
| (eSL) | |
| Mouse U1 | GTTTACTTGGTTTTAAAAATAGCTTGCACTAGCGATACGGAATATGGTTATTAGG |
| terminator | TTTGTTAGGCATCATGTCGTGTCTTACTATAGAAAAATAACGTAGTGTTCATTTT |
| | AGCCTGCCTGTATGTGTTAATTTGTCCTTATTGCGCATTGTTCTTGTTAAGTCTTC |
| | TGTAAGGAGTTGCGGGTTTCAAACTGTCAGTCTGAGAGCA (SEQ ID NO: |
| | 57) |
| 3′ ITR | aggaacccctagtgatggagttggccactccctctctgcgcgctcgctcgctcactgaggccgggcgaccaaaggtcgcccgac |
| | gcccgggctttgcccgggcggcctcagtgagcgagcgagcgcgcagctgcctgcagg (SEQ ID NO: 60) |

An exemplary AAV vector of the disclosure comprises from 5′ to 3′: a first inverted terminal repeat (ITR) sequence, a U7 promoter sequence, a DMD exon 51 targeting U7 esnRNA sequence, a U7 downstream terminator sequence, a U1 promoter sequence, a DMD exon 51 targeting U7 esnRNA sequence, a U1 downstream terminator sequence, and a second ITR sequence. In some aspects, a nucleic acid sequence encoding AAV vector A04529 encoding DMD exon 51 targeting snRNA sequences comprises SEQ ID NO: 174.

A04529: scAAV-mU7p-z38/42-mU7term_mU1p-z38/42-mU1term; Human Loop with ISD and eSL with Added CCU Tail

| Plasmid Element | Nucleotide Sequence 5′ to 3′ |
|---|---|
| 5′ ITR | ctgcgcgctcgctcgctcactgaggccgcccgggcaaagcccgggcgtcgggcgacctttggtcgcccggcctcagtgagcgag |
| | cgagcgcgcagagagggagtggggtt (SEQ ID NO: 162) |
| Mouse U7 | taacaacataggagctgtgattggctgttttcagccaatcagcactgActcatttgcatagcctttacaagcggtcacaaactc |
| promoter | aagaaacgagcggttttaatagtcttttagaatattgtttatcgaaccgaataaggaactgtgctttgtgattcacatatcagt |
| | ggaggggtgtggaaatggcaccttgatctcaccctcatcgaaagtggagttgatgtcctTccctggctcgctacagacgcac |
| | ttccgcaa (SEQ ID NO: 48) |
| ISD | ggagt (SEQ ID NO: 12) |
| Z38/z42 forward | TACCTCCAACATCAAGGAAGATGGCAGTAACCACAGGTTGTGTCACCAGAGTAA |
| fusion spacer | CA (SEQ ID NO: 150) |
| SMOPT | AATTTTTGGAGTA (SEQ ID NO: 164) |
| Human stem loop | ggctttctggctccttaccggaaagcccct (SEQ ID NO: 163) |
| (eSL) with | |
| CCU tail | |
| Mouse U7 | cccaatttcactggtctacaatgaaagcaaaacagttctcttccccgctccccggtgtgtgagaggggctttgatccttctct |
| terminator | ggtttcctaggaaacgcgtatgtg (SEQ ID NO: 156) |
| Mouse U1 | TTGTTCCTCTTAGTGTTAATTCACACTAAAGACTGTGCATCCGACTCCTACATTTA |
| promoter | TGAAAGTAAATGCCTGTTGTTAGAACAAAAAAGGCTACAGAACAAAAAACAAA |
| | GCGAAATACCATCTGCTTTAGGTTCAGTGTGGTATTTTCCCGCTGACAGGGAGGC |
| | GGGTTTTTGGGTACAGGAAACGAGTCACTATGGAGGCGGTACTATGTAGATGAG |
| | AATTCAGGTGCAAACTGGGAAAAGCAACTGCTTCCAAATATTTGTGATTTTTACA |
| | GTGTAGTTTTGGAAAAACTCTTAGCCTACCAATTCTTCTAAGTGTTTTAAAATGT |
| | GGGAGCCAGTACACATGAAGTTATAGAGTGTTTTAATGAGGCTTAAATATTTACC |
| | GTAACTATGAAATGCTACGCATATCATGCTGTTCAGGCTCCGTGGCCACGCAACT |
| | C (SEQ ID NO: 47) |
| ISD | ggagt (SEQ ID NO: 12) |
| Z38/z42 forward | TACCTCCAACATCAAGGAAGATGGCAGTAACCACAGGTTGTGTCACCAGAGTAA |
| fusion spacer | CA (SEQ ID NO: 150) |
| SMOPT | AATTTTTGGAGTA (SEQ ID NO: 164) |
| Human stem loop | ggctttctggctccttaccggaaagcccct (SEQ ID NO: 163) |
| (eSL) with | |
| CCU tail | |
| Mouse U1 | GTTTACTTGGTTTTAAAAATAGCTTGCACTAGCGATACGGAATATGGTTATTAGG |
| terminator | TTTGTTAGGCATCATGTCGTGTCTTACTATAGAAAAATAACGTAGTGTTCATTTT |
| | AGCCTGCCTGTATGTGTTAATTTGTCCTTATTGCGCATTGTTCTTGTTAAGTCTTC |
| | TGTAAGGAGTTGCGGGTTTCAAACTGTCAGTCTGAGAGCA (SEQ ID NO: |
| | 57) |
| 3′ ITR | aggaacccctagtgatggagttggccactccctctctgcgcgctcgctcgctcactgaggccgggcgaccaaaggtcgcccga |
| | cgcccgggctttgcccgggggcctcagtgagcgagcgagcgcgcagctgcctgcagg (SEQ ID NO: 60) |

An exemplary AAV vector of the disclosure comprises from 5′ to 3′: a first inverted terminal repeat (ITR) sequence, a U7 promoter sequence, a DMD exon 51 targeting U7 esnRNA sequence, a U7 downstream terminator sequence, a U1 promoter sequence, a DMD exon 51 targeting U7 esnRNA sequence, a U1 downstream terminator sequence, and a second ITR sequence. In some aspects, a nucleic acid sequence encoding AAV vector A04537 encoding DMD exon 51 targeting snRNA sequences comprises SEQ ID NO: 175.

A04537: scAAV-mU7p-z38/42-mU7term_mU1prom-p-z38/42-mU1term; Long Mouse Loop with 5′ ISD and eSL

| Plasmid Element | Nucleotide Sequence 5′ to 3′ |
|---|---|
| 5′ ITR | ctgcgcgctcgctcgctcactgaggccgcccgggcaaagcccgggcgtcgggcgacctttggtcgcccggcctcagtgagcgag |
| | cgagcgcgcagagagggagtggggtt (SEQ ID NO: 162) |
| Mouse U7 | taacaacataggagctgtgattggctgttttcagccaatcagcactgActcatttgcatagcctttacaagcggtcacaaactc |
| promoter | aagaaacgagcggttttaatagtcttttagaatattgtttatcgaaccgaataaggaactgtgctttgtgattcacatatcagt |
| | ggaggggtgtggaaatggcaccttgatctcaccctcatcgaaagtggagttgatgtcctTccctggctcgctacagacgcact |
| | tccgcaa (SEQ ID NO: 48) |
| ISD | ggagt (SEQ ID NO: 12) |
| Z38/z42 forward | TACCTCCAACATCAAGGAAGATGGCAGTAACCACAGGTTGTGTCACCAGAGTAA |
| fusion spacer | CA (SEQ ID NO: 150) |
| SMOPT | aATTTTTGGAGca (SEQ ID NO: 161) |
| mouse stem loop | Ggttttctgacctccttcggtcggaaaacccct (SEQ ID NO: 147) |
| (eSL) with CCU | |
| tail (long | |
| extended loop) | |
| Mouse U7 | Cccaatttcactggtctacaatgaaagcaaaacagttctcttccccgctccccggtgtgtgagaggggctttgatccttctctg |
| terminator | gtttcctaggaaacgcgtatgtg (SEQ ID NO: 156) |
| Mouse U1 | TTGTTCCTCTTAGTGTTAATTCACACTAAAGACTGTGCATCCGACTCCTACATTTA |
| promoter | TGAAAGTAAATGCCTGTTGTTAGAACAAAAAAGGCTACAGAACAAAAAACAAA |
| | GCGAAATACCATCTGCTTTAGGTTCAGTGTGGTATTTTCCCGCTGACAGGGAGGC |
| | GGGTTTTTGGGTACAGGAAACGAGTCACTATGGAGGCGGTACTATGTAGATGAG |
| | AATTCAGGTGCAAACTGGGAAAAGCAACTGCTTCCAAATATTTGTGATTTTTACA |
| | GTGTAGTTTTGGAAAAACTCTTAGCCTACCAATTCTTCTAAGTGTTTTAAAATGT |
| | GGGAGCCAGTACACATGAAGTTATAGAGTGTTTTAATGAGGCTTAAATATTTACC |
| | GTAACTATGAAATGCTACGCATATCATGCTGTTCAGGCTCCGTGGCCACGCAACT |
| | C (SEQ ID NO: 47) |
| ISD | ggagt (SEQ ID NO: 12) |
| Z38/z42 forward | TACCTCCAACATCAAGGAAGATGGCAGTAACCACAGGTTGTGTCACCAGAGTAA |
| fusion spacer | CA (SEQ ID NO: 150) |
| SMOPT | aATTTTTGGAGca (SEQ ID NO: 161) |
| mouse stem loop | ggttttctgacctccttcggtcggaaaacccct (SEQ ID NO: 147) |
| (eSL) with | |
| CCU tail (long | |
| extended loop) | |
| Mouse U1 | GTTTACTTGGTTTTAAAAATAGCTTGCACTAGCGATACGGAATATGGTTATTAGG |
| terminator | TTTGTTAGGCATCATGTCGTGTCTTACTATAGAAAAATAACGTAGTGTTCATTTT |
| | AGCCTGCCTGTATGTGTTAATTTGTCCTTATTGCGCATTGTTCTTGTTAAGTCTTC |
| | TGTAAGGAGTTGCGGGTTTCAAACTGTCAGTCTGAGAGCA (SEQ ID NO: 57) |
| 3′ ITR | aggaacccctagtgatggagttggccactccctctctgcgcgctcgctcgctcactgaggccgggcgaccaaaggtcgcccga |
| | cgcccgggctttgcccgggggcctcagtgagcgagcgagcgcgcagctgcctgcagg (SEQ ID NO: 60) |

An exemplary AAV vector of the disclosure comprises from 5′ to 3′: a first inverted terminal repeat (ITR) sequence, a U7 promoter sequence, a CUG repeat targeting U7 esnRNA sequence, a U7 downstream terminator sequence, a U1 promoter sequence, a CUG repeat targeting U7 esnRNA sequence, a U1 downstream terminator sequence, and a second ITR sequence. In some aspects, a nucleic acid sequence encoding AAV vector A04233 encoding CUG repeat targeting snRNA sequences comprises SEQ TD NO: 176.

A04233: scAAV-hU7-CUGx15-hU1-CUGx15 (3′mut ITR)

| Plasmid Element | Nucleotide Sequence 5′ to 3′ |
|---|---|
| 5′ ITR | cctgcaggcagctgcgcgctcgctcgctcactgaggccgcccgggcgtcgggcgacctttggtcgccc |
| | ggcctcagtgagcgagcgagcgcgcagagagggagtggccaactccatcactaggggttcct |
| | (SEQ ID NO: 59) |
| human U7 promoter | TACTGCCGAATCCAGGTCTCCGGGCTTAACAACAACGAAGGGGCTGTGACTGGC |
| | TGCTTTCTCAACCAATCAGCACCGAACTCATTTGCATGGGCTGAGAACAAATGTT |
| | CGCGAACTCTAGAAATGAATGACTTAAGTAAGTTCCTTAGAATATTATTTTTCCT |
| | ACTGAAAGTTACCACATGCGTCGTTGTTTATACAGTAATAGGAACAAGAAAAAA |
| | GTCACCTAAGCTCACCCTCATCAATTGTGGAGTTCCTTTATATCCCATCTTCTCTC |
| | CAAACACATACGCA (SEQ ID NO: 39) |
| ISD | ggagt (SEQ ID NO: 12) |
| CUG repeat | CAGCAGCAGCAGCAGCAGCAGCAGCAGCAGCAGCAGCAGCAGCAG (SEQ ID NO: |
| targeting sequence | 165) |
| SMOPT | AATTTTTGGAGTA (SEQ ID NO: 164) |
| Human stem loop | ggctttctggctccttaccggaaagcc (SEQ ID NO: 1) |
| (eSL) | |
| Human U7 terminator | CCTCTTATGATGTTTGTTGCCAATGATAGATTGTTTTCACTGTGCAAAAATTATGG |
| | GTAGTTTTGGTGGTCTTGATGCAGTTGTAAGCTTGGAG (SEQ ID NO: 49) |
| Human U1 promoter | TAAGGACCAGCTTCTTTGGGAGAGAACAGACGCAGGGGCGGGAGGGAAAAAGG |
| | GAGAGGCAGACGTCACTTCCCCTTGGCGGCTCTGGCAGCAGATTGGTCGGTTGA |
| | GTGGCAGAAAGGCAGACGGGGACTGGGCAAGGCACTGTCGGTGACATCACGGA |
| | CAGGGCGACTTCTATGTAGATGAGGCAGCGCAGAGGCTGACGTCTTCGCCACTT |
| | GCTGCTTCACCACGAAGGAGTTCCCGTGCCCTGGGAGCGGGTTCAGGACCGCTG |
| | ATCGGAAGTGAGAATCCCAGCTGTGTGTCAGGGCTGGAAAGGGCTCGGGAGTGC |
| | GCGGGGCAAGTGACCGTGTGTGTAAAGAGTGAGGCGTATGAGGCTGTGTCGGGG |
| | CAGAGGCacaacgtttc (SEQ ID NO: 40) |
| ISD | ggagt (SEQ ID NO: 12) |
| spacer | CAGCAGCAGCAGCAGCAGCAGCAGCAGCAGCAGCAGCAGCAGCAG (SEQ ID NO: |
| | 165) |
| SMOPT | AATTTTTGGAGTA (SEQ ID NO: 164) |
| Human stem loop | ggctttctggctccttaccggaaagcc (SEQ ID NO: 1) |
| (eSL) | |
| human U1 terminator | ACTTTCTGGAGTTTCAAAAACAGACTGTACGCCAAGGGTCATATCTTTTT (SEQ |
| | ID NO: 50) |
| 3′mut ITR | CCACTCCCTCTCTGCGCGCTCGCTCGCTCACTGAGGCCGGGCGACCAAAGGTCGC |
| | CCGACGCCCGGGCTTTGCCCGGGCGGCCTCAGTGAGCGAGCGAGCGCGCAGAGA |
| | GGGA (SEQ ID NO: 61) |

An exemplary AAV vector of the disclosure comprises from 5′ to 3′: a first inverted terminal repeat (ITR) sequence, a U7 promoter sequence, a CUG repeat targeting U7 esnRNA sequence, a U7 downstream terminator sequence, a U1 promoter sequence, a CUG repeat targeting U7 esnRNA sequence, a U1 downstream terminator sequence, and a second ITR sequence. In some aspects, a nucleic acid sequence encoding AAV vector A04234 encoding CUG repeat targeting snRNA sequences comprises SEQ TD NO: 177.

A04234: scAAV-hU7-CUGx15-hU1-CUGx15 (5′ mut ITR)

| Plasmid Element | Nucleotide Sequence 5′ to 3′ |
|---|---|
| 5′ mut ITR | ctgcgcgctcgctcgctcactgaggccgcccgggcaaagcccgggcgtcgggcgacctttggtcgcccggcctcag |
| | tgagcgagcgagcgcgcagagagggagtggggtt (SEQ ID NO: 162) |
| human U7 | TACTGCCGAATCCAGGTCTCCGGGCTTAACAACAACGAAGGGGCTGTGACTGGC |
| promoter | TGCTTTCTCAACCAATCAGCACCGAACTCATTTGCATGGGCTGAGAACAAATGTT |
| | CGCGAACTCTAGAAATGAATGACTTAAGTAAGTTCCTTAGAATATTATTTTTCCT |
| | ACTGAAAGTTACCACATGCGTCGTTGTTTATACAGTAATAGGAACAAGAAAAAA |
| | GTCACCTAAGCTCACCCTCATCAATTGTGGAGTTCCTTTATATCCCATCTTCTCTC |
| | CAAACACATACGCA (SEQ ID NO: 39) |
| ISD | ggagt (SEQ ID NO: 12) |
| spacer | CAGCAGCAGCAGCAGCAGCAGCAGCAGCAGCAGCAGCAGCAGCAG (SEQ ID NO: |
| | 165) |
| SMOPT | AATTTTTGGAGTA (SEQ ID NO: 164) |
| Human stem loop | ggctttctggctccttaccggaaagcc (SEQ ID NO: 1) |
| (eSL) | |
| Human U7 | CCTCTTATGATGTTTGTTGCCAATGATAGATTGTTTTCACTGTGCAAAAATTATGG |
| terminator | GTAGTTTTGGTGGTCTTGATGCAGTTGTAAGCTTGGAG (SEQ ID NO: 49) |
| Human U1 | TAAGGACCAGCTTCTTTGGGAGAGAACAGACGCAGGGGCGGGAGGGAAAAAGG |
| promoter | GAGAGGCAGACGTCACTTCCCCTTGGCGGCTCTGGCAGCAGATTGGTCGGTTGA |
| | GTGGCAGAAAGGCAGACGGGGACTGGGCAAGGCACTGTCGGTGACATCACGGA |
| | CAGGGCGACTTCTATGTAGATGAGGCAGCGCAGAGGCTGACGTCTTCGCCACTT |
| | GCTGCTTCACCACGAAGGAGTTCCCGTGCCCTGGGAGCGGGTTCAGGACCGCTG |
| | ATCGGAAGTGAGAATCCCAGCTGTGTGTCAGGGCTGGAAAGGGCTCGGGAGTGC |
| | GCGGGGCAAGTGACCGTGTGTGTAAAGAGTGAGGCGTATGAGGCTGTGTCGGGG |
| | CAGAGGCacaacgtttc (SEQ ID NO: 40) |
| ISD | ggagt (SEQ ID NO: 12) |
| spacer | CAGCAGCAGCAGCAGCAGCAGCAGCAGCAGCAGCAGCAGCAGCAG (SEQ ID NO: |
| | 165) |
| SMOPT | AATTTTTGGAGTA (SEQ ID NO: 164) |
| Human stem loop | ggctttctggctccttaccggaaagcc (SEQ ID NO: 1) |
| (eSL) | |
| human U1 | ACTTTCTGGAGTTTCAAAAACAGACTGTACGCCAAGGGTCATATCTTTTT (SEQ |
| terminator | ID NO: 50) |
| 3′ITR | aaggaacccctagtgatggagttggccactccctctctgcgcgctcgctcgctcactgaggccgggcgaccaaag |
| | gtcgcccgacgcccgggctttgcccgggggcctcagtgagcgagcgagcgcgccagc (SEQ ID NO: 166) |

An exemplary AAV vector of the disclosure comprises from 5′ to 3′: a first inverted terminal repeat (ITR) sequence, a U7 promoter sequence, a CAG repeat targeting U7 esnRNA sequence, a U7 downstream terminator sequence, a U1 promoter sequence, a CAG repeat targeting U7 esnRNA sequence, a U1 downstream terminator sequence, and a second ITR sequence. In some aspects, a nucleic acid sequence encoding AAV vector A04390 encoding CAG repeat targeting snRNA sequences comprises SEQ TD NO: 178.

A04390: scAAV: 5′mITR hU7-CAG15X-hU1-CAGx15

| Plasmid Element | Nucleotide Sequence 5′ to 3′ |
|---|---|
| 5′ mut ITR | ctgcgcgctcgctcgctcactgaggccgcccgggcaaagcccgggcgtcgggcgacctttggtcgcccggcctcag |
| | tgagcgagcgagcgcgcagagagggagtggggtt (SEQ ID NO: 162) |
| human U7 | TACTGCCGAATCCAGGTCTCCGGGCTTAACAACAACGAAGGGGCTGTGACTGGC |
| promoter | TGCTTTCTCAACCAATCAGCACCGAACTCATTTGCATGGGCTGAGAACAAATGTT |
| | CGCGAACTCTAGAAATGAATGACTTAAGTAAGTTCCTTAGAATATTATTTTTCCT |
| | ACTGAAAGTTACCACATGCGTCGTTGTTTATACAGTAATAGGAACAAGAAAAAA |
| | GTCACCTAAGCTCACCCTCATCAATTGTGGAGTTCCTTTATATCCCATCTTCTCTC |
| | CAAACACATACGCA (SEQ ID NO: 39) |
| ISD | ggagt (SEQ ID NO: 12) |
| CAG targeting | CTGCTGCTGCTGCTGCTGCTGCTGCTGCTGCTGCTGCTGCTGCTG (SEQ ID NO: |
| sequence | 167) |
| SMOPT | AATTTTTGGAGTA (SEQ ID NO: 164) |
| Human stem loop | ggctttctggctccttaccggaaagcc (SEQ ID NO: 1) |
| (eSL) | |
| Human U7 | CCTCTTATGATGTTTGTTGCCAATGATAGATTGTTTTCACTGTGCAAAAATTATGG |
| terminator | GTAGTTTTGGTGGTCTTGATGCAGTTGTAAGCTTGGAG (SEQ ID NO: 49) |
| Human U1 | TAAGGACCAGCTTCTTTGGGAGAGAACAGACGCAGGGGCGGGAGGGAAAAAGG |
| promoter | GAGAGGCAGACGTCACTTCCCCTTGGCGGCTCTGGCAGCAGATTGGTCGGTTGA |
| | GTGGCAGAAAGGCAGACGGGGACTGGGCAAGGCACTGTCGGTGACATCACGGA |
| | CAGGGCGACTTCTATGTAGATGAGGCAGCGCAGAGGCTGACGTCTTCGCCACTT |
| | GCTGCTTCACCACGAAGGAGTTCCCGTGCCCTGGGAGCGGGTTCAGGACCGCTG |
| | ATCGGAAGTGAGAATCCCAGCTGTGTGTCAGGGCTGGAAAGGGCTCGGGAGTGC |
| | GCGGGGCAAGTGACCGTGTGTGTAAAGAGTGAGGCGTATGAGGCTGTGTCGGGG |
| | CAGAGGCacaacgtttc (SEQ ID NO: 40) |
| ISD | ggagt (SEQ ID NO: 12) |
| CAG targeting | CTGCTGCTGCTGCTGCTGCTGCTGCTGCTGCTGCTGCTGCTGCTG (SEQ ID NO: |
| sequence | 167) |
| SMOPT | AATTTTTGGAGTA (SEQ ID NO: 164) |
| Human stem loop | ggctttctggctccttaccggaaagcc (SEQ ID NO: 1) |
| (eSL) | |
| human U1 | ACTTTCTGGAGTTTCAAAAACAGACTGTACGCCAAGGGTCATATCTTTTT (SEQ |
| terminator | ID NO: 50) |
| 3′ITR | aaggaacccctagtgatggagttggccactccctctctgcgcgctcgctcgctcactgaggccgggcgaccaaa |
| | ggtcgcccgacgcccgggctttgcccgggcggcctcagtgagcgagcgagcgcgccagc (SEQ ID NO: 166) |

An exemplary AAV vector of the disclosure comprises from 5′ to 3′: a first inverted terminal repeat (ITR) sequence, a U7 promoter sequence, a CUG repeat targeting U7 esnRNA sequence, a U7 downstream terminator sequence, a U1 promoter sequence, a CUG repeat targeting U7 esnRNA sequence, a U1 downstream terminator sequence, and a second ITR sequence. In some aspects, a nucleic acid sequence encoding AAV vector A04530 encoding CUG repeat targeting snRNA sequences comprises SEQ TD NO: 231.

A04530: scAAV-mU7p-CUGx15-mU1p-CUGx15: Mouseloop with ISD and eSL

| Plasmid | |
|---|---|
| Element | Nucleotide Sequence 5′ to 3′ |
| 5′ mut | ctgcgcgctcgctcgctcactgaggccgc |
| ITR | ccgggcaaagcccgggcgtcgggcgacct |
| | ttggtcgcccggcctcagtgagcgagcga |
| | gcgcgcagagagggagtggggtt |
| | (SEQ ID NO: 162) |
| mouse U7 | taacaacataggagctgtgattggctgtt |
| promoter | ttcagccaatcagcactgActcatttgca |
| | tagcctttacaagcggtcacaaactcaag |
| | aaacgagcggttttaatagtcttttagaa |
| | tattgtttatcgaaccgaataaggaactg |
| | tgctttgtgattcacatatcagtggaggg |
| | gtgtggaaatggcaccttgatctcaccct |
| | catcgaaagtggagttgatgtcctTccct |
| | ggctcgctacagacgcacttccgcaa |
| | (SEQ ID NO: 48) |
| ISD | ggagt (SEQ ID NO: 12) |
| spacer | CAGCAGCAGCAGCAGCAGCAGCAGCAGCA |
| | GCAGCAGCAGCAGCAG |
| | (SEQ ID NO: 165) |
| SMOPT | aATTTTTGGAGca |
| | (SEQ ID NO: 161) |
| Mouse stem | ggttttctgacctccgtcggaaaacccct |
| loop (eSL) | (SEQ ID NO: 12) |
| Mouse U7 | cccaatttcactggtctacaatgaaagca |
| terminator | aaacagttctcttccccgctccccggtgt |
| | gtgagaggggctttgatccttctctggtt |
| | tcctaggaaacgcgtatgtg |
| | (SEQ ID NO: 156) |
| Mouse U1 | TTGTTCCTCTTAGTGTTAATTCACACTAA |
| promoter | AGACTGTGCATCCGACTCCTACATTTATG |
| | AAAGTAAATGCCTGTTGTTAGAACAAAAA |
| | AGGCTACAGAACAAAAAACAAAGCGAAAT |
| | ACCATCTGCTTTAGGTTCAGTGTGGTATT |
| | TTCCCGCTGACAGGGAGGCGGGTTTTTGG |
| | GTACAGGAAACGAGTCACTATGGAGGCGG |
| | TACTATGTAGATGAGAATTCAGGTGCAAA |
| | CTGGGAAAAGCAACTGCTTCCAAATATTT |
| | GTGATTTTTACAGTGTAGTTTTGGAAAAA |
| | CTCTTAGCCTACCAATTCTTCTAAGTGTT |
| | TTAAAATGTGGGAGCCAGTACACATGAAG |
| | TTATAGAGTGTTTTAATGAGGCTTAAATA |
| | TTTACCGTAACTATGAAATGCTACGCATA |
| | TCATGCTGTTCAGGCTCCGTGGCCACGCA |
| | ACTC (SEQ ID NO: 47) |
| ISD | ggagt (SEQ ID NO: 12) |
| spacer | CAGCAGCAGCAGCAGCAGCAGCAGCAGCA |
| | GCAGCAGCAGCAGCAG |
| | (SEQ ID NO: 165) |
| SMOPT | aATTTTTGGAGca |
| | (SEQ ID NO: 161) |
| Mouse stem | ggttttctgacctccgtcggaaaacccct |
| loop (eSL) | (SEQ ID NO: 12) |
| Mouse U1 | GTTTACTTGGTTTTAAAAATAGCTTGCAC |
| terminator | TAGCGATACGGAATATGGTTATTAGGTTT |
| | GTTAGGCATCATGTCGTGTCTTACTATAG |
| | AAAAATAACGTAGTGTTCATTTTAGCCTG |
| | CCTGTATGTGTTAATTTGTCCTTATTGCG |
| | CATTGTTCTTGTTAAGTCTTCTGTAAGGA |
| | GTTGCGGGTTTCAAACTGTCAGTCTGAGA |
| | GCA (SEQ ID NO: 57) |
| 3′ITR | aggaacccctagtgatggagttggccact |
| | ccctctctgcgcgctcgctcgctcactga |
| | ggccgggcgaccaaaggtcgcccgacgcc |
| | cgggctttgcccgggcggcctcagtgagc |
| | gagcgagcgcgcagctgcctgcagg |
| | (SEQ ID NO: 60) |

An exemplary AAV vector of the disclosure comprises from 5′ to 3′: a first inverted terminal repeat (ITR) sequence, a U7 promoter sequence, a CUG repeat targeting U7 esnRNA sequence, a U7 downstream terminator sequence, a U1 promoter sequence, a CUG repeat targeting U7 esnRNA sequence, a U1 downstream terminator sequence, and a second ITR sequence. In some aspects, a nucleic acid sequence encoding AAV vector A04940 encoding CUG repeat targeting snRNA sequences comprises SEQ ID NO: 232.

A04940: scAAV-mU7p-CUGx10-mU1n-CUGx10: Mouseloop with ISD and eSL, No CCU

| Plasmid | |
|---|---|
| Element | Nucleotide Sequence 5′ to 3′ |
| 5′ mut ITR | ctgcgcgctcgctcgctcactgaggccgc |
| | ccgggcaaagcccgggcgtcgggcgacct |
| | ttggtcgcccggcctcagtgagcgagcga |
| | gcgcgcagagagggagtggggtt |
| | (SEQ ID NO: 162) |
| mouse U7 | taacaacataggagctgtgattggctgtt |
| promoter | ttcagccaatcagcactgActcatttgca |
| | tagcctttacaagcggtcacaaactcaag |
| | aaacgagcggttttaatagtcttttagaa |
| | tattgtttatcgaaccgaataaggaactg |
| | tgctttgtgattcacatatcagtggaggg |
| | gtgtggaaatggcaccttgatctcaccct |
| | catcgaaagtggagttgatgtcctTccct |
| | ggctcgctacagacgcacttccgcaa |
| | (SEQ ID NO: 48) |
| ISD | ggagt (SEQ ID NO: 12) |
| spacer | CAGCAGCAGCAGCAGCAGCAGCAGCAGCA |
| | G (SEQ ID NO: 165) |
| SMOPT | aATTTTTGGAGca |
| | (SEQ ID NO: 161) |
| Mouse stem | ggttttctgacctccgtcggaaaacc |
| loop (eSL) | (SEQ ID NO: 148) |
| Mouse U7 | cccaatttcactggtctacaatgaaagca |
| terminator | aaacagttctcttccccgctccccggtgt |
| | gtgagaggggctttgatccttctctggtt |
| | tcctaggaaacgcgtatgtg |
| | (SEQ ID NO: 156) |
| Mouse U1 | TTGTTCCTCTTAGTGTTAATTCACACTAA |
| promoter | AGACTGTGCATCCGACTCCTACATTTATG |
| | AAAGTAAATGCCTGTTGTTAGAACAAAAA |
| | AGGCTACAGAACAAAAAACAAAGCGAAAT |
| | ACCATCTGCTTTAGGTTCAGTGTGGTATT |
| | TTCCCGCTGACAGGGAGGCGGGTTTTTGG |
| | GTACAGGAAACGAGTCACTATGGAGGCGG |
| | TACTATGTAGATGAGAATTCAGGTGCAAA |
| | CTGGGAAAAGCAACTGCTTCCAAATATTT |
| | GTGATTTTTACAGTGTAGTTTTGGAAAAA |
| | CTCTTAGCCTACCAATTCTTCTAAGTGTT |
| | TTAAAATGTGGGAGCCAGTACACATGAAG |
| | TTATAGAGTGTTTTAATGAGGCTTAAATA |
| | TTTACCGTAACTATGAAATGCTACGCATA |
| | TCATGCTGTTCAGGCTCCGTGGCCACGCA |
| | ACTC (SEQ ID NO: 47) |
| ISD | ggagt (SEQ ID NO: 12) |
| spacer | CAGCAGCAGCAGCAGCAGCAGCAGCAGCA |
| | G (SEQ ID NO: 165) |
| SMOPT | aATTTTTGGAGca |
| | (SEQ ID NO: 161) |
| Mouse stem | ggttttctgacctccgtcggaaaacccct |
| loop (eSL) | (SEQ ID NO: 12) |
| Mouse U1 | GTTTACTTGGTTTTAAAAATAGCTTGCAC |
| terminator | TAGCGATACGGAATATGGTTATTAGGTTT |
| | GTTAGGCATCATGTCGTGTCTTACTATAG |
| | AAAAATAACGTAGTGTTCATTTTAGCCTG |
| | CCTGTATGTGTTAATTTGTCCTTATTGCG |
| | CATTGTTCTTGTTAAGTCTTCTGTAAGGA |
| | GTTGCGGGTTTCAAACTGTCAGTCTGAGA |
| | GCA (SEQ ID NO: 57) |
| 3′ITR | aggaacccctagtgatggagttggccact |
| | ccctctctgcgcgctcgctcgctcactga |
| | ggccgggcgaccaaaggtcgcccgacgcc |
| | cgggctttgcccgggcggcctcagtgagc |
| | gagcgagcgcgcagctgcctgcagg |
| | (SEQ ID NO: 60) |

An exemplary AAV vector of the disclosure comprises from 5′ to 3′: a first inverted terminal repeat (ITR) sequence, a U7 promoter sequence, a CAG repeat targeting U7 esnRNA sequence, a U7 downstream terminator sequence, a U1 promoter sequence, a CAG repeat targeting U7 esnRNA sequence, a U1 downstream terminator sequence, and a second ITR sequence. In some aspects, a nucleic acid sequence encoding AAV vector A04533 encoding CAG repeat targeting snRNA sequences comprises SEQ ID NO: 233.

A04533: scAAV-mU7p-CAGx15-mU1p-CAGx15; Mouseloop with ISD and eSL

| Plasmid | |
|---|---|
| Element | Nucleotide Sequence 5′ to 3′ |
| 5′ mut | ctgcgcgctcgctcgctcactgaggccgc |
| ITR | ccgggcaaagcccgggcgtcgggcgacct |
| | ttggtcgcccggcctcagtgagcgagcga |
| | gcgcgcagagagggagtggggtt |
| | (SEQ ID NO: 162) |
| mouse U7 | taacaacataggagctgtgattggctgtt |
| promoter | ttcagccaatcagcactgActcatttgca |
| | tagcctttacaagcggtcacaaactcaag |
| | aaacgagcggttttaatagtcttttagaa |
| | tattgtttatcgaaccgaataaggaactg |
| | tgctttgtgattcacatatcagtggaggg |
| | gtgtggaaatggcaccttgatctcaccct |
| | catcgaaagtggagttgatgtcctTccct |
| | ggctcgctacagacgcacttccgcaa |
| | (SEQ ID NO: 48) |
| ISD | ggagt (SEQ ID NO: 12) |
| spacer | CTGCTGCTGCTGCTGCTGCTGCTGCTGCT |
| | GCTGCTGCTGCTGCTG |
| | (SEQ ID NO: 167) |
| SMOPT | aATTTTTGGAGca |
| | (SEQ ID NO: 161) |
| Mouse stem | ggttttctgacctccgtcggaaaacccct |
| loop (eSL) | (SEQ ID NO: 12) |
| Mouse U7 | cccaatttcactggtctacaatgaaagca |
| terminator | aaacagttctcttccccgctccccggtgt |
| | gtgagaggggctttgatccttctctggtt |
| | tcctaggaaacgcgtatgtg |
| | (SEQ ID NO: 156) |
| Mouse U1 | TTGTTCCTCTTAGTGTTAATTCACACTAA |
| promoter | AGACTGTGCATCCGACTCCTACATTTATG |
| | AAAGTAAATGCCTGTTGTTAGAACAAAAA |
| | AGGCTACAGAACAAAAAACAAAGCGAAAT |
| | ACCATCTGCTTTAGGTTCAGTGTGGTATT |
| | TTCCCGCTGACAGGGAGGCGGGTTTTTGG |
| | GTACAGGAAACGAGTCACTATGGAGGCGG |
| | TACTATGTAGATGAGAATTCAGGTGCAAA |
| | CTGGGAAAAGCAACTGCTTCCAAATATTT |
| | GTGATTTTTACAGTGTAGTTTTGGAAAAA |
| | CTCTTAGCCTACCAATTCTTCTAAGTGTT |
| | TTAAAATGTGGGAGCCAGTACACATGAAG |
| | TTATAGAGTGTTTTAATGAGGCTTAAATA |
| | TTTACCGTAACTATGAAATGCTACGCATA |
| | TCATGCTGTTCAGGCTCCGTGGCCACGCA |
| | ACTC (SEQ ID NO: 47) |
| ISD | ggagt (SEQ ID NO: 12) |
| spacer | CTGCTGCTGCTGCTGCTGCTGCTGCTGCT |
| | GCTGCTGCTGCTGCTG |
| | (SEQ ID NO: 167) |
| SMOPT | aATTTTTGGAGca |
| | (SEQ ID NO: 161) |
| Mouse stem | ggttttctgacctccgtcggaaaacccct |
| loop (eSL) | (SEQ ID NO: 12) |
| Mouse U1 | GTTTACTTGGTTTTAAAAATAGCTTGCAC |
| terminator | TAGCGATACGGAATATGGTTATTAGGTTT |
| | GTTAGGCATCATGTCGTGTCTTACTATAG |
| | AAAAATAACGTAGTGTTCATTTTAGCCTG |
| | CCTGTATGTGTTAATTTGTCCTTATTGCG |
| | CATTGTTCTTGTTAAGTCTTCTGTAAGGA |
| | GTTGCGGGTTTCAAACTGTCAGTCTGAGA |
| | GCA (SEQ ID NO: 57) |
| 3′ ITR | Aggaacccctagtgatggagttggccact |
| | ccctctctgcgcgctcgctcgctcactga |
| | ggccgggcgaccaaaggtcgcccgacgcc |
| | cgggctttgcccgggcggcctcagtgagc |
| | gagcgagcgcgcagctgcctgcagg |
| | (SEQ ID NO: 60) |

An exemplary AAV vector of the disclosure comprises from 5′ to 3′: a first inverted terminal repeat (ITR) sequence, a U7 promoter sequence, a CUG repeat targeting U7 esnRNA sequence, a U7 downstream terminator sequence, a buffer sequence, a U7 promoter sequence, a CUG repeat targeting U7 esnRNA sequence, a U7 downstream terminator sequence, a buffer sequence, a U7 promoter sequence, a CUG repeat targeting U7 esnRNA sequence, a U7 downstream terminator sequence, a buffer sequence, a U7 promoter sequence, a CUG repeat targeting U7 esnRNA sequence, a U7 downstream terminator sequence, a buffer sequence, and a second ITR sequence. In some aspects, a nucleic acid sequence encoding AAV vector A02962 encoding CUG repeat targeting snRNA sequences comprises SEQ ID NO: 120.A02962 ss AAV-4×U7 (15XCUG) (derivative of P00369) targeting CUG repeats

ITR
(SEQ ID NO: 59)
Cctgcaggcagctgcgcgctcgctcgctcactgaggccgcccgggcgtcg
ggcgacctttggtcgcccggcctcagtgagcgagcgagcgcgcagagagg
gagtggccaactccatcactaggggttcct
U7 promoter
(SEQ ID NO: 39)
TACTGCCGAATCCAGGTCTCCGGGCTTAACAACAACGAAGGG
GCTGTGACTGGCTGCTTTCTCAACCAATCAGCACCGAACTCAT
TTGCATGGGCTGAGAACAAATGTTCGCGAACTCTAGAAATGA
ATGACTTAAGTAAGTTCCTTAGAATATTATTTTTCCTACTGAA
AGTTACCACATGCGTCGTTGTTTATACAGTAATAGGAACAAG
AAAAAAGTCACCTAAGCTCACCCTCATCAATTGTGGAGTTCC
TTTATATCCCATCTTCTCTCCAAACACATACGCA
U7 snRNA
(SEQ ID NO: 65)
ggagtCAGCAGCAGCAGCAGCAGCAGCAGCAGCAGCAGCAGCA
GCAGCAGAATTTTTGGAGTAggctttctggctccttaccggaaagcc
U7 Downstream (terminator)
(SEQ ID NO: 49)
CCTCTTATGATGTTTGTTGCCAATGATAGATTGTTTTCACTGT
GCAAAAATTATGGGTAGTTTTGGTGGTCTTGATGCAGTTGTA
AGCTTGGAG
30 bp buffer 1
(SEQ ID NO: 24)
CAAACTACAGAGCCAAGTGCTATCCACAGA
U7 promoter
(SEQ ID NO: 39)
TACTGCCGAATCCAGGTCTCCGGGCTTAACAACAACGAAGGG
GCTGTGACTGGCTGCTTTCTCAACCAATCAGCACCGAACTCAT
TTGCATGGGCTGAGAACAAATGTTCGCGAACTCTAGAAATGA
ATGACTTAAGTAAGTTCCTTAGAATATTATTTTTCCTACTGAA
AGTTACCACATGCGTCGTTGTTTATACAGTAATAGGAACAAG
AAAAAAGTCACCTAAGCTCACCCTCATCAATTGTGGAGTTCC
TTTATATCCCATCTTCTCTCCAAACACATACGCA
U7 snRNA
(SEQ ID NO: 65)
ggagtCAGCAGCAGCAGCAGCAGCAGCAGCAGCAGCAGCAGCA
GCAGCAGAATTTTTGGAGTAggctttctggctccttaccggaaagcc
U7 Downstream (terminator)
(SEQ ID NO: 49)
CCTCTTATGATGTTTGTTGCCAATGATAGATTGTTTTCACTGT
GCAAAAATTATGGGTAGTTTTGGTGGTCTTGATGCAGTTGTA
AGCTTGGAG
30 bp buffer 2
(SEQ ID NO: 25)
GAGCTTTCTGGGTTGCCATCTCAAGCAGAC
U7 promoter
(SEQ ID NO: 39)
TACTGCCGAATCCAGGTCTCCGGGCTTAACAACAACGAAGGG
GCTGTGACTGGCTGCTTTCTCAACCAATCAGCACCGAACTCAT
TTGCATGGGCTGAGAACAAATGTTCGCGAACTCTAGAAATGA
ATGACTTAAGTAAGTTCCTTAGAATATTATTTTTCCTACTGAA
AGTTACCACATGCGTCGTTGTTTATACAGTAATAGGAACAAG
AAAAAAGTCACCTAAGCTCACCCTCATCAATTGTGGAGTTCC
TTTATATCCCATCTTCTCTCCAAACACATACGCA
U7 snRNA
(SEQ ID NO: 65)
ggagtCAGCAGCAGCAGCAGCAGCAGCAGCAGCAGCAGCAGCA
GCAGCAGAATTTTTGGAGTAggctttctggctccttaccggaaagcc
U7 Downstream (terminator)
(SEQ ID NO: 49)
CCTCTTATGATGTTTGTTGCCAATGATAGATTGTTTTCACTGT
GCAAAAATTATGGGTAGTTTTGGTGGTCTTGATGCAGTTGTA
AGCTTGGAG
30 bp buffer 3
(SEQ ID NO: 26)
TACAAGGCCATCAGCTCATACTCACAATTG
U7 promoter
(SEQ ID NO: 39)
TACTGCCGAATCCAGGTCTCCGGGCTTAACAACAACGAAGGG
GCTGTGACTGGCTGCTTTCTCAACCAATCAGCACCGAACTCAT
TTGCATGGGCTGAGAACAAATGTTCGCGAACTCTAGAAATGA
ATGACTTAAGTAAGTTCCTTAGAATATTATTTTTCCTACTGAA
AGTTACCACATGCGTCGTTGTTTATACAGTAATAGGAACAAG
AAAAAAGTCACCTAAGCTCACCCTCATCAATTGTGGAGTTCC
TTTATATCCCATCTTCTCTCCAAACACATACGCA
U7 snRNA
(SEQ ID NO: 65)
ggagtCAGCAGCAGCAGCAGCAGCAGCAGCAGCAGCAGCAGCA
GCAGCAGAATTTTTGGAGTAggctttctggctccttaccggaaagcc
U7 Downstream (terminator)
(SEQ ID NO: 49)
CCTCTTATGATGTTTGTTGCCAATGATAGATTGTTTTCACTGT
GCAAAAATTATGGGTAGTTTTGGTGGTCTTGATGCAGTTGTA
AGCTTGGAG
ITR
(SEQ ID NO: 60)
Aggaacccctagtgatggagttggccactccctctctgcgcgctcgctcg
ctcactgaggccgggcgaccaaaggtcgcccgacgcccgggctttgcccg
ggcggcctcagtgagcgagcgagcgcgcagctgcctgcagg

An exemplary AAV vector of the disclosure comprises from 5′ to 3′: a first inverted terminal repeat (ITR) sequence, a U7 promoter sequence, a non-targeting U7 esnRNA sequence, a U7 downstream terminator sequence, a buffer sequence, a U7 promoter sequence, a non-targeting U7 esnRNA sequence, a U7 downstream terminator sequence, a buffer sequence, a U7 promoter sequence, a non-targeting U7 esnRNA sequence, a U7 downstream terminator sequence, a buffer sequence, a U7 promoter sequence, a non-targeting U7 esnRNA sequence, a U7 downstream terminator sequence, a buffer sequence, and a second ITR sequence. In some aspects, a nucleic acid sequence encoding AAV vector A02963 encoding a non-targeting snRNA sequences comprises SEQ ID NO: 121.

A02963 ss pAAV-4X U7(NT1) (derivative of P00369)

ITR
(SEQ ID NO: 59)
Cctgcaggcagctgcgcgctcgctcgctcactgaggccgcccgggcgtcg
ggcgacctttggtcgcccggcctcagtgagcgagcgagcgcgcagagagg
gagtggccaactccatcactaggggttcct
U7 promoter
(SEQ ID NO: 39)
TACTGCCGAATCCAGGTCTCCGGGCTTAACAACAACGAAGGGG
CTGTGACTGGCTGCTTTCTCAACCAATCAGCACCGAACTCATTT
GCATGGGCTGAGAACAAATGTTCGCGAACTCTAGAAATGAATG
ACTTAAGTAAGTTCCTTAGAATATTATTTTTCCTACTGAAAGTT
ACCACATGCGTCGTTGTTTATACAGTAATAGGAACAAGAAAAA
AGTCACCTAAGCTCACCCTCATCAATTGTGGAGTTCCTTTATAT
CCCATCTTCTCTCCAAACACATACGCA
U7 snRNA
(SEQ ID NO: 66)
ggagtTCACCAGAAGCGTACCATACTCACGAAATTTTTGGAGTAgg
ctttctggctccttaccggaaagcc
U7 Downstream (terminator)
(SEQ ID NO: 49)
CCTCTTATGATGTTTGTTGCCAATGATAGATTGTTTTCACTGTG
CAAAAATTATGGGTAGTTTTGGTGGTCTTGATGCAGTTGTAAGC
TTGGAG
30 bp buffer 1
(SEQ ID NO: 24)
CAAACTACAGAGCCAAGTGCTATCCACAGA
U7 promoter
(SEQ ID NO: 39)
TACTGCCGAATCCAGGTCTCCGGGCTTAACAACAACGAAGGGG
CTGTGACTGGCTGCTTTCTCAACCAATCAGCACCGAACTCATTT
GCATGGGCTGAGAACAAATGTTCGCGAACTCTAGAAATGAATG
ACTTAAGTAAGTTCCTTAGAATATTATTTTTCCTACTGAAAGTT
ACCACATGCGTCGTTGTTTATACAGTAATAGGAACAAGAAAAA
AGTCACCTAAGCTCACCCTCATCAATTGTGGAGTTCCTTTATAT
CCCATCTTCTCTCCAAACACATACGCA
U7 snRNA
(SEQ ID NO: 66)
ggagtTCACCAGAAGCGTACCATACTCACGAAATTTTTGGAGTAgg
ctttctggctccttaccggaaagcc
U7 Downstream (terminator)
(SEQ ID NO: 49)
CCTCTTATGATGITTGTTGCCAATGATAGATTGTTTTCACTGTG
CAAAAATTATGGGTAGTTTTGGTGGTCTTGATGCAGTTGTAAGC
TTGGAG
30 bp buffer 2
(SEQ ID NO: 25)
GAGCTTTCTGGGTTGCCATCTCAAGCAGAC
U7 promoter
(SEQ ID NO: 39)
TACTGCCGAATCCAGGTCTCCGGGCTTAACAACAACGAAGGGG
CTGTGACTGGCTGCTTTCTCAACCAATCAGCACCGAACTCATTT
GCATGGGCTGAGAACAAATGTTCGCGAACTCTAGAAATGAATG
ACTTAAGTAAGTTCCTTAGAATATTATTTTTCCTACTGAAAGTT
ACCACATGCGTCGTTGTTTATACAGTAATAGGAACAAGAAAAA
AGTCACCTAAGCTCACCCTCATCAATTGTGGAGTTCCTTTATAT
CCCATCTTCTCTCCAAACACATACGCA
U7 snRNA
(SEQ ID NO: 66)
ggagtTCACCAGAAGCGTACCATACTCACGAAATTTTTGGAGTAgg
ctttctggctccttaccggaaagcc
U7 Downstream (terminator)
(SEQ ID NO: 49)
CCTCTTATGATGTTTGTTGCCAATGATAGATTGTTTTCACTGTG
CAAAAATTATGGGTAGTTTTGGTGGTCTTGATGCAGTTGTAAGC
TTGGAG
30 bp buffer 3
(SEQ ID NO: 26)
TACAAGGCCATCAGCTCATACTCACAATTG
U7 promoter
(SEQ ID NO: 39)
TACTGCCGAATCCAGGTCTCCGGGCTTAACAACAACGAAGGGG
CTGTGACTGGCTGCTTTCTCAACCAATCAGCACCGAACTCATTT
GCATGGGCTGAGAACAAATGTTCGCGAACTCTAGAAATGAATG
ACTTAAGTAAGTTCCTTAGAATATTATTTTTCCTACTGAAAGTT
ACCACATGCGTCGTTGTTTATACAGTAATAGGAACAAGAAAAA
AGTCACCTAAGCTCACCCTCATCAATTGTGGAGTTCCTTTATAT
CCCATCTTCTCTCCAAACACATACGCA
U7 snRNA
(SEQ ID NO: 66)
ggagtTCACCAGAAGCGTACCATACTCACGAAATTTTTGGAGTAgg
ctttctggctccttaccggaaagcc
U7 Downstream (terminator)
(SEQ ID NO: 49)
CCTCTTATGATGTTTGTTGCCAATGATAGATTGTTTTCACTGTG
CAAAAATTATGGGTAGTTTTGGTGGTCTTGATGCAGTTGTAAGC
TTGGAG
ITR
(SEQ ID NO: 60)
Aggaacccctagtgatggagttggccactccctctctgcgcgctcgctcg
ctcactgaggccgggcgaccaaaggtcgcccgacgcccgggctttgcccc
gggggcctcagtgagcgagcgagcgcgcagctgctgcagg

An exemplary AAV vector of the disclosure comprises from 5′ to 3′: a first inverted terminal repeat (ITR) sequence, a U7 promoter sequence, a CAG repeat targeting U7 esnRNA sequence, a U7 downstream terminator sequence, a buffer sequence, a U7 promoter sequence, a CAG repeat targeting U7 esnRNA sequence, a U7 downstream terminator sequence, a buffer sequence, a U7 promoter sequence, a CAG repeat targeting U7 esnRNA sequence, a U7 downstream terminator sequence, a buffer sequence, a U7 promoter sequence, a CAG repeat targeting U7 esnRNA sequence, a U7 downstream terminator sequence, a buffer sequence, and a second ITR sequence. In some aspects, a nucleic acid sequence encoding AAV vector A02967 encoding a CAG repeat targeting snRNA sequences comprises SEQ ID NO: 122.

A02967 ss pAAV-4X U7(15XCAG) Targeting CAG Repeats

ITR
(SEQ ID NO: 59)
Cctgcaggcagctgcgcgctcgctcgctcactgaggccgcccgggcgtcg
ggcgacctttggtcgcccggcctcagtgagcgagcgagcgcgcagagagg
gagtggccaactccatcactaggggttcct
U7 promoter
(SEQ ID NO: 39)
TACTGCCGAATCCAGGTCTCCGGGCTTAACAACAACGAAGGG
GCTGTGACTGGCTGCTTTCTCAACCAATCAGCACCGAACTCAT
TTGCATGGGCTGAGAACAAATGTTCGCGAACTCTAGAAATGA
ATGACTTAAGTAAGTTCCTTAGAATATTATTTTTCCTACTGAAA
GTTACCACATGCGTCGTTGTTTATACAGTAATAGGAACAAGAA
AAAAGTCACCTAAGCTCACCCTCATCAATTGTGGAGTTCCTTT
ATATCCCATCTTCTCTCCAAACACATACGCA
U7 snRNA
(SEQ ID NO: 67)
ggagtCTGCTGCTGCTGCTGCTGCTGCTGCTGCTGCTGCTGCTGC
TGCTGAATTTTTGGAGTAggctttctggctccttaccggaaagcc
U7 Downstream (terminator)
(SEQ ID NO: 49)
CCTCTTATGATGTTTGTTGCCAATGATAGATTGTTTTCACTGTG
CAAAAATTATGGGTAGTTTTGGTGGTCTTGATGCAGTTGTAAG
CTTGGAG
30 bp buffer 1
(SEQ ID NO: 24)
CAAACTACAGAGCCAAGTGCTATCCACAGA
U7 promoter
(SEQ ID NO: 39)
TACTGCCGAATCCAGGTCTCCGGGCTTAACAACAACGAAGGG
GCTGTGACTGGCTGCTTTCTCAACCAATCAGCACCGAACTCAT
TTGCATGGGCTGAGAACAAATGTTCGCGAACTCTAGAAATGA
ATGACTTAAGTAAGTTCCTTAGAATATTATTTTTCCTACTGAAA
GTTACCACATGCGTCGTTGTTTATACAGTAATAGGAACAAGAA
AAAAGTCACCTAAGCTCACCCTCATCAATTGTGGAGTTCCTTT
ATATCCCATCTTCTCTCCAAACACATACGCA
U7 snRNA
(SEQ ID NO: 67)
ggagtCTGCTGCTGCTGCTGCTGCTGCTGCTGCTGCTGCTGCTGC
TGCTGAATTTTTGGAGTAggctttctggctccttaccggaaagcc
U7 Downstream (terminator)
(SEQ ID NO: 49)
CCTCTTATGATGTTTGTTGCCAATGATAGATTGTTTTCACTGTG
CAAAAATTATGGGTAGTTTTGGTGGTCTTGATGCAGTTGTAAG
CTTGGAG
30 bp buffer 2
(SEQ ID NO: 25)
GAGCTTTCTGGGTTGCCATCTCAAGCAGAC
U7 promoter
(SEQ ID NO: 39)
TACTGCCGAATCCAGGTCTCCGGGCTTAACAACAACGAAGGG
GCTGTGACTGGCTGCTTTCTCAACCAATCAGCACCGAACTCAT
TTGCATGGGCTGAGAACAAATGTTCGCGAACTCTAGAAATGA
ATGACTTAAGTAAGTTCCTTAGAATATTATTTTTCCTACTGAAA
GTTACCACATGCGTCGTTGTTTATACAGTAATAGGAACAAGAA
AAAAGTCACCTAAGCTCACCCTCATCAATTGTGGAGTTCCTTT
ATATCCCATCTTCTCTCCAAACACATACGCA
U7 snRNA
(SEQ ID NO: 67)
ggagtCTGCTGCTGCTGCTGCTGCTGCTGCTGCTGCTGCTGCTGC
TGCTGAATTTTTGGAGTAggctttctggctccttaccggaaagcc
U7 Downstream (terminator)
(SEQ ID NO: 49)
CCTCTTATGATGTTTGTTGCCAATGATAGATTGTTTTCACTGTG
CAAAAATTATGGGTAGTTTTGGTGGTCTTGATGCAGTTGTAAG
CTTGGAG
30bp buffer 3
(SEQ ID NO: 26)
TACAAGGCCATCAGCTCATACTCACAATTG
U7 promoter
(SEQ ID NO: 39)
TACTGCCGAATCCAGGTCTCCGGGCTTAACAACAACGAAGGG
GCTGTGACTGGCTGCTTTCTCAACCAATCAGCACCGAACTCAT
TTGCATGGGCTGAGAACAAATGTTCGCGAACTCTAGAAATGA
ATGACTTAAGTAAGTTCCTTAGAATATTATTTTTCCTACTGAAA
GTTACCACATGCGTCGTTGTTTATACAGTAATAGGAACAAGAA
AAAAGTCACCTAAGCTCACCCTCATCAATTGTGGAGTTCCTTT
ATATCCCATCTTCTCTCCAAACACATACGCA
U7 snRNA
(SEQ ID NO: 67)
ggagtCTGCTGCTGCTGCTGCTGCTGCTGCTGCTGCTGCTGCTGC
TGCTGAATTTTTGGAGTAggctttctggctccttaccggaaagcc
U7 Downstream (terminator)
(SEQ ID NO: 49)
CCTCTTATGATGTTTGTTGCCAATGATAGATTGTTTTCACTGTG
CAAAAATTATGGGTAGTTTTGGTGGTCTTGATGCAGTTGTAAG
CTTGGAG
ITR
(SEQ ID NO: 60)
Aggaacccctagtgatggagttggccactccctctctgcgcgctcgctcg
ctcactgaggccgggcgaccaaaggtcgcccgacgcccgggctttgcccg
ggcggcctcagtgagcgagcgagcgcgcagctgcctgcagg

An exemplary AAV vector of the disclosure comprises from 5′ to 3′: a first inverted terminal repeat (ITR) sequence, a U7 promoter sequence, a CUG repeat targeting U7 esnRNA sequence, a U7 downstream terminator sequence, a buffer sequence, a U7 promoter sequence, a CUG repeat targeting U7 esnRNA sequence, a U7 downstream terminator sequence, a buffer sequence, a U7 promoter sequence, a CUG repeat targeting U7 esnRNA sequence, a U7 downstream terminator sequence, a buffer sequence, a U7 promoter sequence, a CUG repeat targeting U7 esnRNA sequence, a U7 downstream terminator sequence, a buffer sequence, and a second ITR sequence. In some aspects, a nucleic acid sequence encoding AAV vector A03079 encoding a CUG repeat targeting snRNA sequences comprises SEQ ID NO: 123.

### A03079 sc AAV-4X U7(15XCUG) Derivative of P00369 Targeting CUG Repeats

ITR
(SEQ ID NO: 59)
Cctgcaggcagctgcgcgctcgctcgctcactgaggccgcccgggcgtcg
ggcgacctttggtcgcccggcctcagtgagcgagcgagcgcgcagagagg
gagtggccaactccatcactaggggttcct
U7 promoter
(SEQ ID NO: 39)
TACTGCCGAATCCAGGTCTCCGGGCTTAACAACAACGAAGGGG
CTGTGACTGGCTGCTTTCTCAACCAATCAGCACCGAACTCATTT
GCATGGGCTGAGAACAAATGTTCGCGAACTCTAGAAATGAATG
ACTTAAGTAAGTTCCTTAGAATATTATTTTTCCTACTGAAAGTT
ACCACATGCGTCGTTGTTTATACAGTAATAGGAACAAGAAAAA
AGTCACCTAAGCTCACCCTCATCAATTGTGGAGTTCCTTTATAT
CCCATCTTCTCTCCAAACACATACGCA
U7 snRNA
(SEQ ID NO: 65)
ggagtCAGCAGCAGCAGCAGCAGCAGCAGCAGCAGCAGCAGCAG
CAGCAGAATTTTTGGAGTAggctttctggctccttaccggaaagcc
U7 Downstream (terminator)
(SEQ ID NO: 49)
CCTCTTATGATGTTTGTTGCCAATGATAGATTGTTTTCACTGTGC
AAAAATTATGGGTAGTTTTGGTGGTCTTGATGCAGTTGTAAGCT
TGGAG
30 bp buffer 1
(SEQ ID NO: 24)
CAAACTACAGAGCCAAGTGCTATCCACAGA
U7 promoter
(SEQ ID NO: 39)
TACTGCCGAATCCAGGTCTCCGGGCTTAACAACAACGAAGGGG
CTGTGACTGGCTGCTTTCTCAACCAATCAGCACCGAACTCATTT
GCATGGGCTGAGAACAAATGTTCGCGAACTCTAGAAATGAATG
ACTTAAGTAAGTTCCTTAGAATATTATTTTTCCTACTGAAAGTT
ACCACATGCGTCGTTGTTTATACAGTAATAGGAACAAGAAAAA
AGTCACCTAAGCTCACCCTCATCAATTGTGGAGTTCCTTTATAT
CCCATCTTCTCTCCAAACACATACGCA
U7 snRNA
(SEQ ID NO: 65)
ggagtCAGCAGCAGCAGCAGCAGCAGCAGCAGCAGCAGCAGCAG
CAGCAGAATTTTTGGAGTAggctttctggctccttaccggaaagcc
U7 Downstream (terminator)
(SEQ ID NO: 49)
CCTCTTATGATGTTTGTTGCCAATGATAGATTGTTTTCACTGTGC
AAAAATTATGGGTAGTTTTGGTGGTCTTGATGCAGTTGTAAGCT
TGGAG
30 bp buffer 2
(SEQ ID NO: 25)
GAGCTTTCTGGGTTGCCATCTCAAGCAGAC
U7 promoter
(SEQ ID NO: 39)
TACTGCCGAATCCAGGTCTCCGGGCTTAACAACAACGAAGGGG
CTGTGACTGGCTGCTTTCTCAACCAATCAGCACCGAACTCATTT
GCATGGGCTGAGAACAAATGTTCGCGAACTCTAGAAATGAATG
ACTTAAGTAAGTTCCTTAGAATATTATTTTTCCTACTGAAAGTT
ACCACATGCGTCGTTGTTTATACAGTAATAGGAACAAGAAAAA
AGTCACCTAAGCTCACCCTCATCAATTGTGGAGTTCCTTTATAT
CCCATCTTCTCTCCAAACACATACGCA
U7 snRNA
(SEQ ID NO: 65)
ggagtCAGCAGCAGCAGCAGCAGCAGCAGCAGCAGCAGCAGCAG
CAGCAGAATTTTTGGAGTAggctttctggctccttaccggaaagcc
U7 Downstream (terminator)
(SEQ ID NO: 49)
CCTCTTATGATGTTTGTTGCCAATGATAGATTGTTTTCACTGTGC
AAAAATTATGGGTAGTTTTGGTGGTCTTGATGCAGTTGTAAGCT
TGGAG
30 bp buffer 3
(SEQ ID NO: 26)
TACAAGGCCATCAGCTCATACTCACAATTG
U7 promoter
(SEQ ID NO: 39)
TACTGCCGAATCCAGGTCTCCGGGCTTAACAACAACGAAGGGG
CTGTGACTGGCTGCTTTCTCAACCAATCAGCACCGAACTCATTT
GCATGGGCTGAGAACAAATGTTCGCGAACTCTAGAAATGAATG
ACTTAAGTAAGTTCCTTAGAATATTATTTTTCCTACTGAAAGTT
ACCACATGCGTCGTTGTTTATACAGTAATAGGAACAAGAAAAA
AGTCACCTAAGCTCACCCTCATCAATTGTGGAGTTCCTTTATAT
CCCATCTTCTCTCCAAACACATACGCA
U7 snRNA
(SEQ ID NO: 65)
ggagtCAGCAGCAGCAGCAGCAGCAGCAGCAGCAGCAGCAGCAG
CAGCAGAATTTTTGGAGTAggctttctggctccttaccggaaagcc
U7 Downstream (terminator)
(SEQ ID NO: 49)
CCTCTTATGATGTTTGTTGCCAATGATAGATTGTTTTCACTGTGC
AAAAATTATGGGTAGTTTTGGTGGTCTTGATGCAGTTGTAAGCT
TGGAG
ITR
(SEQ ID NO: 61)
CCACTCCCTCTCTGCGCGCTCGCTCGCTCACTGAGGCCGGGCGA
CCAAAGGTCGCCCGACGCCCGGGCTTTGCCCGGGCGGCCTCAG
TGAGCGAGCGAGCGCGCAGAGAGGGA

An exemplary AAV vector of the disclosure comprises from 5′ to 3′: a first inverted terminal repeat (ITR) sequence, a U7 promoter sequence, a non-targeting U7 esnRNA sequence, a U7 downstream terminator sequence, a buffer sequence, a U7 promoter sequence, a non-targeting U7 esnRNA sequence, a U7 downstream terminator sequence, a buffer sequence, a U7 promoter sequence, a non-targeting U7 esnRNA sequence, a U7 downstream terminator sequence, a buffer sequence, a U7 promoter sequence, a non-targeting U7 esnRNA sequence, a U7 downstream terminator sequence, a buffer sequence, and a second ITR sequence. In some aspects, a nucleic acid sequence encoding AAV vector A03080 encoding a non-repeat targeting snRNA sequences comprises SEQ ID NO: 124.

A03080 sc pAAV-4X U7(NT1) (Derivative of P00369)

ITR
(SEQ ID NO: 59)
Cctgcaggcagctgcgcgctcgctcgctcactgaggccgcccgggcgtcg
ggcgacctttggtcgcccggcctcagtgagcgagcgagcgcgcagagagg
gagtggccaactccatcactaggggttcct
U7 promoter
(SEQ ID NO: 39)
TACTGCCGAATCCAGGTCTCCGGGCTTAACAACAACGAAGGGG
CTGTGACTGGCTGCTTTCTCAACCAATCAGCACCGAACTCATTT
GCATGGGCTGAGAACAAATGTTCGCGAACTCTAGAAATGAATG
ACTTAAGTAAGTTCCTTAGAATATTATTTTTCCTACTGAAAGTTA
CCACATGCGTCGTTGTTTATACAGTAATAGGAACAAGAAAAAA
GTCACCTAAGCTCACCCTCATCAATTGTGGAGTTCCTTTATATCC
CATCTTCTCTCCAAACACATACGCA
U7 snRNA
(SEQ ID NO: 66)
ggagtTCACCAGAAGCGTACCATACTCACGAAATTTTTGGAGTAggc
tttctggctccttaccggaaagcc
U7 Downstream (terminator)
(SEQ ID NO: 49)
CCTCTTATGATGTTTGTTGCCAATGATAGATTGTTTTCACTGTGC
AAAAATTATGGGTAGTTTTGGTGGTCTTGATGCAGTTGTAAGCT
TGGAG
30 bp buffer 1
(SEQ ID NO: 24)
CAAACTACAGAGCCAAGTGCTATCCACAGA
U7 promoter
(SEQ ID NO: 39)
TACTGCCGAATCCAGGTCTCCGGGCTTAACAACAACGAAGGGG
CTGTGACTGGCTGCTTTCTCAACCAATCAGCACCGAACTCATTT
GCATGGGCTGAGAACAAATGTTCGCGAACTCTAGAAATGAATG
ACTTAAGTAAGTTCCTTAGAATATTATTTTTCCTACTGAAAGTTA
CCACATGCGTCGTTGTTTATACAGTAATAGGAACAAGAAAAAA
GTCACCTAAGCTCACCCTCATCAATTGTGGAGTTCCTTTATATCC
CATCTTCTCTCCAAACACATACGCA
U7 snRNA
(SEQ ID NO: 66)
ggagtTCACCAGAAGCGTACCATACTCACGAAATTTTTGGAGTAggc
tttctggctccttaccggaaagcc
U7 Downstream (terminator)
(SEQ ID NO: 49)
CCTCTTATGATGTTTGTTGCCAATGATAGATTGTTTTCACTGTGC
AAAAATTATGGGTAGTTTTGGTGGTCTTGATGCAGTTGTAAGCT
TGGAG
30 bp buffer 2
(SEQ ID NO: 25)
GAGCTTTCTGGGTTGCCATCTCAAGCAGAC
U7 promoter
(SEQ ID NO: 39)
TACTGCCGAATCCAGGTCTCCGGGCTTAACAACAACGAAGGGG
CTGTGACTGGCTGCTTTCTCAACCAATCAGCACCGAACTCATTT
GCATGGGCTGAGAACAAATGTTCGCGAACTCTAGAAATGAATG
ACTTAAGTAAGTTCCTTAGAATATTATTTTTCCTACTGAAAGTTA
CCACATGCGTCGTTGTTTATACAGTAATAGGAACAAGAAAAAA
GTCACCTAAGCTCACCCTCATCAATTGTGGAGTTCCTTTATATCC
CATCTTCTCTCCAAACACATACGCA
U7 snRNA
(SEQ ID NO: 66)
ggagtTCACCAGAAGCGTACCATACTCACGAAATTTTTGGAGTAggc
tttctggctccttaccggaaagcc
U7 Downstream (terminator)
(SEQ ID NO: 49)
CCTCTTATGATGTTTGTTGCCAATGATAGATTGTTTTCACTGTGC
AAAAATTATGGGTAGTTTTGGTGGTCTTGATGCAGTTGTAAGCT
TGGAG
30 bp buffer 3
(SEQ ID NO: 26)
TACAAGGCCATCAGCTCATACTCACAATTG
U7 promoter
(SEQ ID NO: 39)
TACTGCCGAATCCAGGTCTCCGGGCTTAACAACAACGAAGGGG
CTGTGACTGGCTGCTTTCTCAACCAATCAGCACCGAACTCATTT
GCATGGGCTGAGAACAAATGTTCGCGAACTCTAGAAATGAATG
ACTTAAGTAAGTTCCTTAGAATATTATTTTTCCTACTGAAAGTTA
CCACATGCGTCGTTGTTTATACAGTAATAGGAACAAGAAAAAA
GTCACCTAAGCTCACCCTCATCAATTGTGGAGTTCCTTTATATCC
CATCTTCTCTCCAAACACATACGCA
U7 snRNA
(SEQ ID NO: 66)
ggagtTCACCAGAAGCGTACCATACTCACGAAATTTTTGGAGTAggc
tttctggctccttaccggaaagcc
U7 Downstream (terminator)
(SEQ ID NO: 49)
CCTCTTATGATGTTTGTTGCCAATGATAGATTGTTTTCACTGTGC
AAAAATTATGGGTAGTTTTGGTGGTCTTGATGCAGTTGTAAGCT
TGGAG
ITR
(SEQ ID NO: 61)
CCACTCCCTCTCTGCGCGCTCGCTCGCTCACTGAGGCCGGGCGA
CCAAAGGTCGCCCGACGCCCGGGCTTTGCCCGGGCGGCCTCAGT
GAGCGAGCGAGCGCGCAGAGAGGGA

An exemplary AAV vector of the disclosure comprises from 5′ to 3′: a first inverted terminal repeat (ITR) sequence, a U7 promoter sequence, a CAG repeat targeting U7 esnRNA sequence, a U7 downstream terminator sequence, a buffer sequence, a U7 promoter sequence, a CAG repeat targeting U7 esnRNA sequence, a U7 downstream terminator sequence, a buffer sequence, a U7 promoter sequence, a CAG repeat targeting U7 esnRNA sequence, a U7 downstream terminator sequence, a buffer sequence, a U7 promoter sequence, a CAG repeat targeting U7 esnRNA sequence, a U7 downstream terminator sequence, a buffer sequence, and a second ITR sequence. In some aspects, a nucleic acid sequence encoding AAV vector A03081 encoding a CAG repeat targeting snRNA sequences comprises SEQ ID NO: 125.

A03081 sc pAAV-4X U7(15XCAG) Targeting CAG Repeats

ITR
(SEQ ID NO: 59)
Cctgcaggcagctgcgcgctcgctcgctcactgaggccgcccgggcgtcg
ggcgacctttggtcgcccggcctcagtgagcgagcgagcgcgcagagagg
gagtggccaactccatcactaggggttcct
U7 promoter
(SEQ ID NO: 39)
TACTGCCGAATCCAGGTCTCCGGGCTTAACAACAACGAAGGGG
CTGTGACTGGCTGCTTTCTCAACCAATCAGCACCGAACTCATTT
GCATGGGCTGAGAACAAATGTTCGCGAACTCTAGAAATGAATG
ACTTAAGTAAGTTCCTTAGAATATTATTTTTCCTACTGAAAGTTA
CCACATGCGTCGTTGTTTATACAGTAATAGGAACAAGAAAAAA
GTCACCTAAGCTCACCCTCATCAATTGTGGAGTTCCTTTATATCC
CATCTTCTCTCCAAACACATACGCA
U7 snRNA
(SEQ ID NO: 67)
ggagtCTGCTGCTGCTGCTGCTGCTGCTGCTGCTGCTGCTGCTGCTG
CTGAATTTTTGGAGTAggctttctggctccttaccggaaagcc
U7 Downstream (terminator)
(SEQ ID NO: 49)
CCTCTTATGATGTTTGTTGCCAATGATAGATTGTTTTCACTGTGC
AAAAATTATGGGTAGTTTTGGTGGTCTTGATGCAGTTGTAAGCT
TGGAG
30 bp buffer 1
(SEQ ID NO: 24)
CAAACTACAGAGCCAAGTGCTATCCACAGA
U7 promoter
(SEQ ID NO: 39)
TACTGCCGAATCCAGGTCTCCGGGCTTAACAACAACGAAGGGG
CTGTGACTGGCTGCTTTCTCAACCAATCAGCACCGAACTCATTT
GCATGGGCTGAGAACAAATGTTCGCGAACTCTAGAAATGAATG
ACTTAAGTAAGTTCCTTAGAATATTATTTTTCCTACTGAAAGTTA
CCACATGCGTCGTTGTTTATACAGTAATAGGAACAAGAAAAAA
GTCACCTAAGCTCACCCTCATCAATTGTGGAGTTCCTTTATATCC
CATCTTCTCTCCAAACACATACGCA
U7 snRNA
(SEQ ID NO: 67)
ggagtCTGCTGCTGCTGCTGCTGCTGCTGCTGCTGCTGCTGCTGCTG
CTGAATTTTTGGAGTAggctttctggctccttaccggaaagcc
U7 Downstream (terminator)
(SEQ ID NO: 49)
CCTCTTATGATGTTTGTTGCCAATGATAGATTGTTTTCACTGTGC
AAAAATTATGGGTAGTTTTGGTGGTCTTGATGCAGTTGTAAGCT
TGGAG
30 bp buffer 2
(SEQ ID NO: 25)
GAGCTTTCTGGGTTGCCATCTCAAGCAGAC
U7 promoter
(SEQ ID NO: 39)
TACTGCCGAATCCAGGTCTCCGGGCTTAACAACAACGAAGGGG
CTGTGACTGGCTGCTTTCTCAACCAATCAGCACCGAACTCATTT
GCATGGGCTGAGAACAAATGTTCGCGAACTCTAGAAATGAATG
ACTTAAGTAAGTTCCTTAGAATATTATTTTTCCTACTGAAAGTTA
CCACATGCGTCGTTGTTTATACAGTAATAGGAACAAGAAAAAA
GTCACCTAAGCTCACCCTCATCAATTGTGGAGTTCCTTTATATCC
CATCTTCTCTCCAAACACATACGCA
U7 snRNA
(SEQ ID NO: 67)
ggagtCTGCTGCTGCTGCTGCTGCTGCTGCTGCTGCTGCTGCTGCTG
CTGAATTTTTGGAGTAggctttctggctccttaccggaaagcc
U7 Downstream (terminator)
(SEQ ID NO: 49)
CCTCTTATGATGTTTGTTGCCAATGATAGATTGTTTTCACTGTGC
AAAAATTATGGGTAGTTTTGGTGGTCTTGATGCAGTTGTAAGCT
TGGAG
30 bp buffer 3
(SEQ ID NO: 26)
TACAAGGCCATCAGCTCATACTCACAATTG
U7 promoter
(SEQ ID NO: 39)
TACTGCCGAATCCAGGTCTCCGGGCTTAACAACAACGAAGGGG
CTGTGACTGGCTGCTTTCTCAACCAATCAGCACCGAACTCATTT
GCATGGGCTGAGAACAAATGTTCGCGAACTCTAGAAATGAATG
ACTTAAGTAAGTTCCTTAGAATATTATTTTTCCTACTGAAAGTTA
CCACATGCGTCGTTGTTTATACAGTAATAGGAACAAGAAAAAA
GTCACCTAAGCTCACCCTCATCAATTGTGGAGTTCCTTTATATCC
CATCTTCTCTCCAAACACATACGCA
U7 snRNA
(SEQ ID NO: 67)
ggagtCTGCTGCTGCTGCTGCTGCTGCTGCTGCTGCTGCTGCTGCTG
CTGAATTTTTGGAGTAggctttctggctccttaccggaaagcc
U7 Downstream (terminator)
(SEQ ID NO: 49)
CCTCTTATGATGTTTGTTGCCAATGATAGATTGTTTTCACTGTGC
AAAAATTATGGGTAGTTTTGGTGGTCTTGATGCAGTTGTAAGCT
TGGAG
ITR
(SEQ ID NO: 61)
CCACTCCCTCTCTGCGCGCTCGCTCGCTCACTGAGGCCGGGCGA
CCAAAGGTCGCCCGACGCCCGGGCTTTGCCCGGGCGGCCTCAGT
GAGCGAGCGAGCGCGCAGAGAGGGA

An exemplary AAV vector of the disclosure comprises from 5′ to 3′: a first inverted terminal repeat (ITR) sequence, a U7 promoter sequence, a CUG repeat targeting U7 esnRNA sequence, a U7 downstream terminator sequence, a CMV promoter sequence, a sequence encoding green fluorescent protein (GFP), a sv40 polyA sequence, and a second ITR sequence. In some aspects, a nucleic acid sequence encoding AAV vector A02888 encoding a CUG repeat targeting snRNA sequence comprises SEQ ID NO: 126.

### A02888 AAV_1xU7(15xCUG)_CMV_GFP (No Buffer) Targeting CUG Repeats

ITR
(SEQ ID NO: 59)
Cctgcaggcagctgcgcgctcgctcgctcactgaggccgcccgggcgtcg
ggcgacctttggtcgcccggcctcagtgagcgagcgagcgcgcagagagg
gagtggccaactccatcactaggggttcct
U7 promoter
(SEQ ID NO: 39)
TACTGCCGAATCCAGGTCTCCGGGCTTAACAACAACGAAGGGG
CTGTGACTGGCTGCTTTCTCAACCAATCAGCACCGAACTCATTT
GCATGGGCTGAGAACAAATGTTCGCGAACTCTAGAAATGAATG
ACTTAAGTAAGTTCCTTAGAATATTATTTTTCCTACTGAAAGTTA
CCACATGCGTCGTTGTTTATACAGTAATAGGAACAAGAAAAAA
GTCACCTAAGCTCACCCTCATCAATTGTGGAGTTCCTTTATATCC
CATCTTCTCTCCAAACACATACGCA
U7 snRNA
(SEQ ID NO: 65)
ggagtCAGCAGCAGCAGCAGCAGCAGCAGCAGCAGCAGCAGCAG
CAGCAGAATTTTTGGAGTAggctttctggctccttaccggaaagcc
U7 Downstream (terminator)
(SEQ ID NO: 49)
CCTCTTATGATGTTTGTTGCCAATGATAGATTGTTTTCACTGTGC
AAAAATTATGGGTAGTTTTGGTGGTCTTGATGCAGTTGTAAGCT
TGGAG
CMV promoter
(SEQ ID NO: 62)
Ccttacataacttacggtaaatggcccgcctggctgaccgcccaacgacc
cccgcccattgacgtcaataatgacgtatgttcccatagtaacgccaata
gggactttccattgacgtcaatgggtggagtatttacggtaaactgccca
cttggcagtacatcaagtgtatcatatgccaagtacgccccctattgacg
tcaatgacggtaaatggcccgcctggcattatgcccagtacatgacctta
tgggactttcctacttggcagtacatctacgtattagtcatcgctattac
catggtgatgcggttttggcagtacatcaatgggcgtggatagcggtttg
actcacggggatttccaagtctccaccccattgacgtcaatgggagtttg
ttttggcaccaaaatcaacgggactttccaaaatgtcgtaacaactccgc
cccattgacgcaaatgggcggtaggcgtgtacggtgggaggtctatataa
gcagagct
eGFP
(SEQ ID NO: 63)
ATGgtgagcaagggcgaggagctgttcaccggggtggtgcccatcctggt
cgagctggacggcgacgtaaacggccacaagttcagcgtgtccggcgagg
gcgagggcgatgccacctacggcaagctgaccctgaagttcatctgcacc
accggcaagctgcccgtgccctggcccaccctcgtgaccaccctgaccta
cggcgtgcagtgcttcagccgctaccccgaccacatgaagcagcacgact
tcttcaagtccgccatgcccgaaggctacgtccaggagcgcaccatcttc
ttcaaggacgacggcaactacaagacccgcgccgaggtgaagttcgaggg
cgacaccctggtgaaccgcatcgagctgaagggcatcgacttcaaggagg
acggcaacatcctggggcacaagctggagtacaactacaacagccacaac
gtctatatcatggccgacaagcagaagaacggcatcaaggtgaacttcaa
gatccgccacaacatcgaggacggcagcgtgcagctcgccgaccactacc
agcagaacacccccatcggcgacggccccgtgctgctgcccgacaaccac
tacctgagcacccagtccgccctgagcaaagaccccaacgagaagcgcga
tcacatggtcctgctggagttcgtgaccgccgccgggatcactctcggca
tggacgagctgtacaagTAA
SV40 Poly A
(SEQ ID NO: 64)
Aacttgtttattgcagcttataatggttacaaataaagcaatagcatcac
aaatttcacaaataaagcatttttttcactgcattctagttgtggtttgt
ccaaactcatcaatgtatctta
ITR
(SEQ ID NO: 60)
Aggaacccctagtgatggagttggccactccctctctgcgcgctcgctcg
ctcactgaggccgggcgaccaaaggtcgcccgacgcccgggctttgcccg
ggggcctcagtgagcgagcgagcgcgcagctgcctgcagg

An exemplary AAV vector of the disclosure comprises from 5′ to 3′: a first inverted terminal repeat (ITR) sequence, a U7 promoter sequence, a non-targeting U7 esnRNA sequence, a U7 downstream terminator sequence, a CMV promoter sequence, a sequence encoding green fluorescent protein (GFP), a sv40 polyA sequence, and a second ITR sequence. In some aspects, a nucleic acid sequence encoding AAV vector A02889 encoding a non-targeting repeat targeting snRNA sequence comprises SEQ ID NO: 127.

A02889 pAAV_1xU7(NT1)_CMV_GFP (No Buffer)

ITR
(SEQ ID NO: 59)
Cctgcaggcagctgcgcgctcgctcgctcactgaggccgcccgggcgtcg
ggcgacctttggtcgcccggcctcagtgagcgagcgagcgcgcagagagg
gagtggccaactccatcactaggggttcct
U7 promoter
(SEQ ID NO: 39)
TACTGCCGAATCCAGGTCTCCGGGCTTAACAACAACGAAGGGG
CTGTGACTGGCTGCTTTCTCAACCAATCAGCACCGAACTCATTT
GCATGGGCTGAGAACAAATGTTCGCGAACTCTAGAAATGAATG
ACTTAAGTAAGTTCCTTAGAATATTATTTTTCCTACTGAAAGTTA
CCACATGCGTCGTTGTTTATACAGTAATAGGAACAAGAAAAAA
GTCACCTAAGCTCACCCTCATCAATTGTGGAGTTCCTTTATATCC
CATCTTCTCTCCAAACACATACGCA
U7 snRNA
(SEQ ID NO: 66)
ggagtTCACCAGAAGCGTACCATACTCACGAAATTTTTGGAGTAggc
tttctggctccttaccggaaagcc
U7 Downstream (terminator)
(SEQ ID NO: 49)
CCTCTTATGATGTTTGTTGCCAATGATAGATTGTTTTCACTGTGC
AAAAATTATGGGTAGTTTTGGTGGTCTTGATGCAGTTGTAAGCT
TGGAG
CMV promoter
(SEQ ID NO. 62)
Cgttacataacttacggtaaatggcccgcctggctgaccgcccaacgacc
cccgcccattgacgtcaataatgacgtatgttcccatagtaacgccaata
gggactttccattgacgtcaatgggtggagtatttacggtaaactgccca
cttggcagtacatcaagtgtatcatatgccaagtacgccccctattgacg
tcaatgacggtaaatggcccgcctggcattatgcccagtacatgacctta
tgggactttcctacttggcagtacatctacgtattagtcatcgctattac
catggtgatgcggttttggcagtacatcaatgggcgtggatagcggtttg
actcacggggatttccaagtctccaccccattgacgtcaatgggagtttg
ttttggcaccaaaatcaacgggactttccaaaatgtcgtaacaactccgc
cccattgacgcaaatgggcggtaggcgtgtacggtgggaggtctatataa
gcagagct
eGFP
(SEQ ID NO: 63)
ATGgtgagcaagggcgaggagctgttcaccggggtggtgcccatcctggt
cgagctggacggcgacgtaaacggccacaagttcagcgtgtccggcgagg
gcgagggcgatgccacctacggcaagctgaccctgaagttcatctgcacc
accggcaagctgcccgtgccctggcccaccctcgtgaccaccctgaccta
cggcgtgcagtgcttcagccgctaccccgaccacatgaagcagcacgact
tcttcaagtccgccatgcccgaaggctacgtccaggagcgcaccatcttc
ttcaaggacgacggcaactacaagacccgcgccgaggtgaagttcgaggg
cgacaccctggtgaaccgcatcgagctgaagggcatcgacttcaaggagg
acggcaacatcctggggcacaagctggagtacaactacaacagccacaac
gtctatatcatggccgacaagcagaagaacggcatcaaggtgaacttcaa
gatccgccacaacatcgaggacggcagcgtgcagctcgccgaccactacc
agcagaacacccccatcggcgacggccccgtgctgctgcccgacaaccac
tacctgagcacccagtccgccctgagcaaagaccccaacgagaagcgcga
tcacatggtcctgctggagttcgtgaccgccgccgggatcactctcggca
tggacgagctgtacaagTAA
SV40 Poly A
(SEQ ID NO: 64)
Aacttgtttattgcagcttataatggttacaaataaagcaatagcatcac
aaatttcacaaataaagcatttttttcactgcattctagttgtggtttgt
ccaaactcatcaatgtatctta
ITR
(SEQ ID NO: 60)
Aggaacccctagtgatggagttggccactccctctctgcgcgctcgctcg
ctcactgaggccgggcgaccaaaggtcgcccgacgcccgggctttgcccg
ggcggcctcagtgagcgagcgagcgcgcagctgcctgcagg

An exemplary AAV vector of the disclosure comprises from 5′ to 3′: a first inverted terminal repeat (ITR) sequence, a U7 promoter sequence, a CAG repeat targeting U7 esnRNA sequence, a U7 downstream terminator sequence, a CMV promoter sequence, a sequence encoding green fluorescent protein (GFP), a sv40 polyA sequence, and a second ITR sequence. In some aspects, a nucleic acid sequence encoding AAV vector A02925 encoding a CAG repeat targeting repeat targeting snRNA sequence comprises SEQ ID NO: 128.

A02925 pAAV_1xU7(15xCAG)_CMV_GFP (no buffer) targeting CAG repeats

ITR
(SEQ ID NO: 59)
Cctgcaggcagctgcgcgctcgctcgctcactgaggccgcccgggcgtcg
ggcgacctttggtcgcccggcctcagtgagcgagcgagcgcgcagagagg
gagtggccaactccatcactaggggttcct
U7 promoter
(SEQ ID NO: 39)
TACTGCCGAATCCAGGTCTCCGGGCTTAACAACAACGAAGGGG
CTGTGACTGGCTGCTTTCTCAACCAATCAGCACCGAACTCATTT
GCATGGGCTGAGAACAAATGTTCGCGAACTCTAGAAATGAATG
ACTTAAGTAAGTTCCTTAGAATATTATTTTTCCTACTGAAAGTTA
CCACATGCGTCGTTGTTTATACAGTAATAGGAACAAGAAAAAA
GTCACCTAAGCTCACCCTCATCAATTGTGGAGTTCCTTTATATCC
CATCTTCTCTCCAAACACATACGCA
U7 snRNA
(SEQ ID NO: 67)
ggagtCTGCTGCTGCTGCTGCTGCTGCTGCTGCTGCTGCTGCTGCTG
CTGAATTTTTGGAGTAggctttctggctccttaccggaaagcc
U7 Downstream (terminator)
(SEQ ID NO: 49)
CCTCTTATGATGTTTGTTGCCAATGATAGATTGTTTTCACTGTGC
AAAAATTATGGGTAGTTTTGGTGGTCTTGATGCAGTTGTAAGCT
TGGAG
CMV promoter
(SEQ ID NO: 62)
Cgttacataacttacggtaaatggcccgcctggctgaccgcccaacgacc
cccgcccattgacgtcaataatgacgtatgttcccatagtaacgccaata
gggactttccattgacgtcaatgggtggagtatttacggtaaactgccca
cttggcagtacatcaagtgtatcatatgccaagtacgccccctattgacg
tcaatgacggtaaatggcccgcctggcattatgcccagtacatgacctta
tgggactttcctacttggcagtacatctacgtattagtcatcgctattac
catggtgatgcggttttggcagtacatcaatgggcgtggatagcggtttg
actcacggggatttccaagtctccaccccattgacgtcaatgggagtttg
ttttggcaccaaaatcaacgggactttccaaaatgtcgtaacaactccgc
cccattgacgcaaatgggcggtaggcgtgtacggtgggaggtctatataa
gcagagct
eGFP
(SEQ ID NO: 63)
ATGgtgagcaagggcgaggagctgttcaccggggtggtgcccatcctggt
cgagctggacggcgacgtaaacggccacaagttcagcgtgtccggcgagg
gcgagggcgatgccacctacggcaagctgaccctgaagttcatctgcacc
accggcaagctgcccgtgccctggcccaccctcgtgaccaccctgaccta
cggcgtgcagtgcttcagccgctaccccgaccacatgaagcagcacgact
tcttcaagtccgccatgcccgaaggctacgtccaggagcgcaccatcttc
ttcaaggacgacggcaactacaagacccgcgccgaggtgaagttcgaggg
cgacaccctggtgaaccgcatcgagctgaagggcatcgacttcaaggagg
acggcaacatcctggggcacaagctggagtacaactacaacagccacaac
gtctatatcatggccgacaagcagaagaacggcatcaaggtgaacttcaa
gatccgccacaacatcgaggacggcagcgtgcagctcgccgaccactacc
agcagaacacccccatcggcgacggccccgtgctgctgcccgacaaccac
tacctgagcacccagtccgccctgagcaaagaccccaacgagaagcgcga
tcacatggtcctgctggagttcgtgaccgccgccgggatcactctcggca
tggacgagctgtacaagTAA
SV40 Poly A
(SEQ ID NO: 64)
Aacttgtttattgcagcttataatggttacaaataaagcaatagcatcac
aaatttcacaaataaagcatttttttcactgcattctagttgtggtttgt
ccaaactcatcaatgtatctta
ITR
(SEQ ID NO: 60)
Aggaacccctagtgatggagttggccactccctctctgcgcgctcgctcg
ctcactgaggccgggcgaccaaaggtcgcccgacgcccgggctttgcccg
ggggcctcagtgagcgagcgagcgcgcagctgcctgcagg

An exemplary AAV vector of the disclosure comprises from 5′ to 3′: a first inverted terminal repeat (ITR) sequence, a U7 promoter sequence, a CUG repeat targeting U7 esnRNA sequence, a U7 downstream terminator sequence, a buffer sequence, a U7 promoter sequence, a CUG repeat targeting U7 esnRNA sequence, a U7 downstream terminator sequence, a buffer sequence, a U7 promoter sequence, a CUG repeat targeting U7 esnRNA sequence, a U7 downstream terminator sequence, a CMV promoter sequence, a sequence encoding green fluorescent protein (GFP), a sv40 polyA sequence, and a second ITR sequence. In some aspects, a nucleic acid sequence encoding AAV vector A02894 encoding a CUG repeat targeting repeat targeting snRNA sequence comprises SEQ ID NO: 129.

A02894 pAAV_3xU7(15xCUG)_CMV_GFP (100 bp buffer) targeting CUG repeats

ITR
(SEQ ID NO: 59)
Cctgcaggcagctgcgcgctcgctcgctcactgaggccgcccgggcgtcg
ggcgacctttggtcgcccggcctcagtgagcgagcgagcgcgcagagagg
gagtggccaactccatcactaggggttcct
U7 promoter
(SEQ ID NO: 39)
TACTGCCGAATCCAGGTCTCCGGGCTTAACAACAACGAAGGGG
CTGTGACTGGCTGCTTTCTCAACCAATCAGCACCGAACTCATTT
GCATGGGCTGAGAACAAATGTTCGCGAACTCTAGAAATGAATG
ACTTAAGTAAGTTCCTTAGAATATTATTTTTCCTACTGAAAGTTA
CCACATGCGTCGTTGTTTATACAGTAATAGGAACAAGAAAAAA
GTCACCTAAGCTCACCCTCATCAATTGTGGAGTTCCTTTATATCC
CATCTTCTCTCCAAACACATACGCA
U7 snRNA
(SEQ ID NO: 65)
ggagtCAGCAGCAGCAGCAGCAGCAGCAGCAGCAGCAGCAGCAG
CAGCAGAATTTTTGGAGTAggctttctggctccttaccggaaagcc
U7 Downstream (terminator)
(SEQ ID NO: 49)
CCTCTTATGATGTTTGTTGCCAATGATAGATTGTTTTCACTGTGC
AAAAATTATGGGTAGTTTTGGTGGTCTTGATGCAGTTGTAAGCT
TGGAG
100 bp buffer 1
(SEQ ID NO: 31)
CAAACTACAGAGCCAAGTGCTATCCACAGAGAGCTTTCTGGGTT
GCCATCTCAAGCAGACTACAAGGCCATCAGCTCATACTCACAAT
TGACTTTGAGAG
U7 promoter
(SEQ ID NO: 39)
TACTGCCGAATCCAGGTCTCCGGGCTTAACAACAACGAAGGGG
CTGTGACTGGCTGCTTTCTCAACCAATCAGCACCGAACTCATTT
GCATGGGCTGAGAACAAATGTTCGCGAACTCTAGAAATGAATG
ACTTAAGTAAGTTCCTTAGAATATTATTTTTCCTACTGAAAGTTA
CCACATGCGTCGTTGTTTATACAGTAATAGGAACAAGAAAAAA
GTCACCTAAGCTCACCCTCATCAATTGTGGAGTTCCTTTATATCC
CATCTTCTCTCCAAACACATACGCA
U7 snRNA
(SEQ ID NO: 65)
ggagtCAGCAGCAGCAGCAGCAGCAGCAGCAGCAGCAGCAGCAG
CAGCAGAATTTTTGGAGTAggctttctggctccttaccggaaagcc
U7 Downstream (terminator)
(SEQ ID NO: 49)
CCTCTTATGATGTTTGTTGCCAATGATAGATTGTTTTCACTGTGC
AAAAATTATGGGTAGTTTTGGTGGTCTTGATGCAGTTGTAAGCT
TGGAG
100 bp buffer 2
(SEQ ID NO: 28)
TTGACCACATACGTGCTCTTTCAAAGTTCTGTGTTTGAAGTTATG
TTAGTAACAACTGATGCCCATCCTGCAATGACAAATCCAATTCT
CAGTGCAGCTC
U7 promoter
(SEQ ID NO: 39)
TACTGCCGAATCCAGGTCTCCGGGCTTAACAACAACGAAGGGG
CTGTGACTGGCTGCTTTCTCAACCAATCAGCACCGAACTCATTT
GCATGGGCTGAGAACAAATGTTCGCGAACTCTAGAAATGAATG
ACTTAAGTAAGTTCCTTAGAATATTATTTTTCCTACTGAAAGTTA
CCACATGCGTCGTTGTTTATACAGTAATAGGAACAAGAAAAAA
GTCACCTAAGCTCACCCTCATCAATTGTGGAGTTCCTTTATATCC
CATCTTCTCTCCAAACACATACGCA
U7 snRNA
(SEQ ID NO: 65)
ggagtCAGCAGCAGCAGCAGCAGCAGCAGCAGCAGCAGCAGCAG
CAGCAGAATTTTTGGAGTAggctttctggctccttaccggaaagcc
U7 Downstream (terminator)
(SEQ ID NO: 49)
CCTCTTATGATGTTTGTTGCCAATGATAGATTGTTTTCACTGTGC
AAAAATTATGGGTAGTTTTGGTGGTCTTGATGCAGTTGTAAGCT
TGGAG
CMV promoter
(SEQ ID NO: 62)
Cgttacataacttacggtaaatggcccgcctggctgaccgcccaacgacc
cccgcccattgacgtcaataatgacgtatgttcccatagtaacgccaata
gggactttccattgacgtcaatgggtggagtatttacggtaaactgccca
cttggcagtacatcaagtgtatcatatgccaagtacgccccctattgacg
tcaatgacggtaaatggcccgcctggcattatgcccagtacatgacctta
tgggactttcctacttggcagtacatctacgtattagtcatcgctattac
catggtgatgcggttttggcagtacatcaatgggcgtggatagcggtttg
actcacggggatttccaagtctccaccccattgacgtcaatgggagtttg
ttttggcaccaaaatcaacgggactttccaaaatgtcgtaacaactccgc
cccattgacgcaaatgggcggtaggcgtgtacggtgggaggtctatataa
gcagagct
eGFP
(SEQ ID NO: 63)
ATGgtgagcaagggcgaggagctgttcaccggggtggtgcccatcctggt
cgagctggacggcgacgtaaacggccacaagttcagcgtgtccggcgagg
gcgagggcgatgccacctacggcaagctgaccctgaagttcatctgcacc
accggcaagctgcccgtgccctggcccaccctcgtgaccaccctgaccta
cggcgtgcagtgcttcagccgctaccccgaccacatgaagcagcacgact
tcttcaagtccgccatgcccgaaggctacgtccaggagcgcaccatcttc
ttcaaggacgacggcaactacaagacccgcgccgaggtgaagttcgaggg
cgacaccctggtgaaccgcatcgagctgaagggcatcgacttcaaggagg
acggcaacatcctggggcacaagctggagtacaactacaacagccacaac
gtctatatcatggccgacaagcagaagaacggcatcaaggtgaacttcaa
gatccgccacaacatcgaggacggcagcgtgcagctcgccgaccactacc
agcagaacacccccatcggcgacggccccgtgctgctgcccgacaaccac
tacctgagcacccagtccgccctgagcaaagaccccaacgagaagcgcga
tcacatggtcctgctggagttcgtgaccgccgccgggatcactctcggca
tggacgagctgtacaagTAA
SV40 Poly A
(SEQ ID NO: 64)
Aacttgtttattgcagcttataatggttacaaataaagcaatagcatcac
aaatttcacaaataaagcatttttttcactgcattctagttgtggtttgt
ccaaactcatcaatgtatctta
ITR
(SEQ ID NO: 60)
Aggaacccctagtgatggagttggccactccctctctgcgcgctcgctcg
ctcactgaggccgggcgaccaaaggtcgcccgacgcccgggctttgcccg
ggcggcctcagtgagcgagcgagcgcgcagctgcctgcagg

An exemplary AAV vector of the disclosure comprises from 5′ to 3′: a first inverted terminal repeat (ITR) sequence, a U7 promoter sequence, a non-targeting U7 esnRNA sequence, a U7 downstream terminator sequence, a buffer sequence, a U7 promoter sequence, a non-targeting U7 esnRNA sequence, a U7 downstream terminator sequence, a buffer sequence, a U7 promoter sequence, a non-targeting U7 esnRNA sequence, a U7 downstream terminator sequence, a CMV promoter sequence, a sequence encoding green fluorescent protein (GFP), a sv40 polyA sequence, and a second ITR sequence. In some aspects, a nucleic acid sequence encoding AAV vector A02895 encoding a non-targeting snRNA sequence comprises SEQ ID NO: 130.

A02895 pAAV_3xU7(NT1)_CMV_GFP (100 bp buffer)

ITR
(SEQ ID NO: 59)
Cctgcaggcagctgcgcgctcgctcgctcactgaggccgcccgggcgtcg
ggcgacctttggtcgcccggcctcagtgagcgagcgagcgcgcagagagg
gagtggccaactccatcactaggggttcct
U7 promoter
(SEQ ID NO: 39)
TACTGCCGAATCCAGGTCTCCGGGCTTAACAACAACGAAGGGG
CTGTGACTGGCTGCTTTCTCAACCAATCAGCACCGAACTCATTT
GCATGGGCTGAGAACAAATGTTCGCGAACTCTAGAAATGAATG
ACTTAAGTAAGTTCCTTAGAATATTATTTTTCCTACTGAAAGTTA
CCACATGCGTCGTTGTTTATACAGTAATAGGAACAAGAAAAAA
GTCACCTAAGCTCACCCTCATCAATTGTGGAGTTCCTTTATATCC
CATCTTCTCTCCAAACACATACGCA
U7 snRNA
(SEQ ID NO: 66)
ggagtTCACCAGAAGCGTACCATACTCACGAAATTTTTGGAGTAggc
tttctggctccttaccggaaagcc
U7 Downstream (terminator)
(SEQ ID NO: 49)
CCTCTTATGATGTTTGTTGCCAATGATAGATTGTTTTCACTGTGC
AAAAATTATGGGTAGTTTTGGTGGTCTTGATGCAGTTGTAAGCT
TGGAG
100 bp buffer 1
(SEQ ID NO: 27)
CAAACTACAGAGCCAAGTGCTATCCACAGAGAGCTTTCTGGGTT
GCCATCTCAAGCAGACTACAAGGCCATCAGCTCATACTCACAAT
TGACTTTGAGAG
U7 promoter
(SEQ ID NO: 39)
TACTGCCGAATCCAGGTCTCCGGGCTTAACAACAACGAAGGGG
CTGTGACTGGCTGCTTTCTCAACCAATCAGCACCGAACTCATTT
GCATGGGCTGAGAACAAATGTTCGCGAACTCTAGAAATGAATG
ACTTAAGTAAGTTCCTTAGAATATTATTTTTCCTACTGAAAGTTA
CCACATGCGTCGTTGTTTATACAGTAATAGGAACAAGAAAAAA
GTCACCTAAGCTCACCCTCATCAATTGTGGAGTTCCTTTATATCC
CATCTTCTCTCCAAACACATACGCA
U7 snRNA
(SEQ ID NO: 66)
ggagtTCACCAGAAGCGTACCATACTCACGAAATTTTTGGAGTAggc
tttctggctccttaccggaaagcc
U7 Downstream (terminator)
(SEQ ID NO: 49)
CCTCTTATGATGTTTGTTGCCAATGATAGATTGTTTTCACTGTGC
AAAAATTATGGGTAGTTTTGGTGGTCTTGATGCAGTTGTAAGCT
TGGAG
100 bp buffer 2
(SEQ ID NO: 28)
TTGACCACATACGTGCTCTTTCAAAGTTCTGTGTTTGAAGTTATG
TTAGTAACAACTGATGCCCATCCTGCAATGACAAATCCAATTCT
CAGTGCAGCTC
U7 promoter
(SEQ ID NO: 39)
TACTGCCGAATCCAGGTCTCCGGGCTTAACAACAACGAAGGGG
CTGTGACTGGCTGCTTTCTCAACCAATCAGCACCGAACTCATTT
GCATGGGCTGAGAACAAATGTTCGCGAACTCTAGAAATGAATG
ACTTAAGTAAGTTCCTTAGAATATTATTTTTCCTACTGAAAGTTA
CCACATGCGTCGTTGTTTATACAGTAATAGGAACAAGAAAAAA
GTCACCTAAGCTCACCCTCATCAATTGTGGAGTTCCTTTATATCC
CATCTTCTCTCCAAACACATACGCA
U7 snRNA
(SEQ ID NO: 66)
ggagtTCACCAGAAGCGTACCATACTCACGAAATTTTTGGAGTAggc
tttctggctccttaccggaaagcc
U7 Downstream (terminator)
(SEQ ID NO: 49)
CCTCTTATGATGTTTGTTGCCAATGATAGATTGTTTTCACTGTGC
AAAAATTATGGGTAGTTTTGGTGGTCTTGATGCAGTTGTAAGCT
TGGAG
CMV promoter
(SEQ ID NO: 62)
Cgttacataacttacggtaaatggcccgcctggctgaccgcccaacgacc
cccgcccattgacgtcaataatgacgtatgttcccatagtaacgccaata
gggactttccattgacgtcaatgggtggagtatttacggtaaactgccca
cttggcagtacatcaagtgtatcatatgccaagtacgccccctattgacg
tcaatgacggtaaatggcccgcctggcattatgcccagtacatgacctta
tgggactttcctacttggcagtacatctacgtattagtcatcgctattac
catggtgatgcggttttggcagtacatcaatgggcgtggatagcggtttg
actcacggggatttccaagtctccaccccattgacgtcaatgggagtttg
ttttggcaccaaaatcaacgggactttccaaaatgtcgtaacaactccgc
cccattgacgcaaatgggcggtaggcgtgtacggtgggaggtctatataa
gcagagct
eGFP
(SEQ ID NO: 63)
ATGgtgagcaagggcgaggagctgttcaccggggtggtgcccatcctggt
cgagctggacggcgacgtaaacggccacaagttcagcgtgtccggcgagg
gcgagggcgatgccacctacggcaagctgaccctgaagttcatctgcacc
accggcaagctgcccgtgccctggcccaccctcgtgaccaccctgaccta
cggcgtgcagtgcttcagccgctaccccgaccacatgaagcagcacgact
tcttcaagtccgccatgcccgaaggctacgtccaggagcgcaccatcttc
ttcaaggacgacggcaactacaagacccgcgccgaggtgaagttcgaggg
cgacaccctggtgaaccgcatcgagctgaagggcatcgacttcaaggagg
acggcaacatcctggggcacaagctggagtacaactacaacagccacaac
gtctatatcatggccgacaagcagaagaacggcatcaaggtgaacttcaa
gatccgccacaacatcgaggacggcagcgtgcagctcgccgaccactacc
agcagaacacccccatcggcgacggccccgtgctgctgcccgacaaccac
tacctgagcacccagtccgccctgagcaaagaccccaacgagaagcgcga
tcacatggtcctgctggagttcgtgaccgccgccgggatcactctcggca
tggacgagctgtacaagTAA
SV40 Poly A
(SEQ ID NO: 64)
Aacttgtttattgcagcttataatggttacaaataaagcaatagcatcac
aaatttcacaaataaagcatttttttcactgcattctagttgtggtttgt
ccaaactcatcaatgtatctta
ITR
(SEQ ID NO: 60)
Aggaacccctagtgatggagttggccactccctctctgcgcgctcgctcg
ctcactgaggccgggcgaccaaaggtcgcccgacgcccgggctttgcccg
ggcggcctcagtgagcgagcgagcgcgcagctgcctgcagg

An exemplary AAV vector of the disclosure comprises from 5′ to 3′: a first inverted terminal repeat (ITR) sequence, a U7 promoter sequence, a CAG repeat targeting U7 esnRNA sequence, a U7 downstream terminator sequence, a buffer sequence, a U7 promoter sequence, a CAG repeat targeting U7 esnRNA sequence, a U7 downstream terminator sequence, a buffer sequence, a U7 promoter sequence, a CAG repeat targeting U7 esnRNA sequence, a U7 downstream terminator sequence, a CMV promoter sequence, a sequence encoding green fluorescent protein (GFP), a sv40 polyA sequence, and a second ITR sequence. In some aspects, a nucleic acid sequence encoding AAV vector A02968 encoding a CAG repeat targeting repeat targeting snRNA sequence comprises SEQ ID NO: 131.

A02968 pAAV_3xU7(15XCAG)_CMV_GFP (100 bp Buffer) Targeting CAG Repeats

ITR
(SEQ ID NO: 59)
Cctgcaggcagctgcgcgctcgctcgctcactgaggccgcccgggcgtcg
ggcgacctttggtcgcccggcctcagtgagcgagcgagcgcgcagagagg
gagtggccaactccatcactaggggttcct
U7 promoter
(SEQ ID NO: 39)
TACTGCCGAATCCAGGTCTCCGGGCTTAACAACAACGAAGGGG
CTGTGACTGGCTGCTTTCTCAACCAATCAGCACCGAACTCATTT
GCATGGGCTGAGAACAAATGTTCGCGAACTCTAGAAATGAATG
ACTTAAGTAAGTTCCTTAGAATATTATTTTTCCTACTGAAAGTT
ACCACATGCGTCGTTGTTTATACAGTAATAGGAACAAGAAAAA
AGTCACCTAAGCTCACCCTCATCAATTGTGGAGTTCCTTTATAT
CCCATCTTCTCTCCAAACACATACGCA
U7 snRNA
(SEQ ID NO: 67)
ggagtCTGCTGCTGCTGCTGCTGCTGCTGCTGCTGCTGCTGCTGCT
GCTGAATTTTTGGAGTAggctttctggctccttaccggaaagcc
U7 Downstream (terminator)
(SEQ ID NO: 49)
CCTCTTATGATGTTTGTTGCCAATGATAGATTGTTTTCACTGTGC
AAAAATTATGGGTAGTTTTGGTGGTCTTGATGCAGTTGTAAGCT
TGGAG
100 bp buffer 1
(SEQ ID NO: 27)
CAAACTACAGAGCCAAGTGCTATCCACAGAGAGCTTTCTGGGT
TGCCATCTCAAGCAGACTACAAGGCCATCAGCTCATACTCACA
ATTGACTTTGAGAG
U7 promoter
(SEQ ID NO: 39)
TACTGCCGAATCCAGGTCTCCGGGCTTAACAACAACGAAGGGG
CTGTGACTGGCTGCTTTCTCAACCAATCAGCACCGAACTCATTT
GCATGGGCTGAGAACAAATGTTCGCGAACTCTAGAAATGAATG
ACTTAAGTAAGTTCCTTAGAATATTATTTTTCCTACTGAAAGTT
ACCACATGCGTCGTTGTTTATACAGTAATAGGAACAAGAAAAA
AGTCACCTAAGCTCACCCTCATCAATTGTGGAGTTCCTTTATAT
CCCATCTTCTCTCCAAACACATACGCA
U7 snRNA
(SEQ ID NO: 67)
ggagtCTGCTGCTGCTGCTGCTGCTGCTGCTGCTGCTGCTGCTGCT
GCTGAATTTTTGGAGTAggctttctggctccttaccggaaagcc
U7 Downstream (terminator)
(SEQ ID NO: 49)
CCTCTTATGATGTTTGTTGCCAATGATAGATTGTTTTCACTGTGC
AAAAATTATGGGTAGTTTTGGTGGTCTTGATGCAGTTGTAAGCT
TGGAG
100 bp buffer 2
(SEQ ID NO: 28)
TTGACCACATACGTGCTCTTTCAAAGTTCTGTGTTTGAAGTTAT
GTTAGTAACAACTGATGCCCATCCTGCAATGACAAATCCAATT
CTCAGTGCAGCTC
U7 promoter
(SEQ ID NO: 39)
TACTGCCGAATCCAGGTCTCCGGGCTTAACAACAACGAAGGGG
CTGTGACTGGCTGCTTTCTCAACCAATCAGCACCGAACTCATTT
GCATGGGCTGAGAACAAATGTTCGCGAACTCTAGAAATGAATG
ACTTAAGTAAGTTCCTTAGAATATTATTTTTCCTACTGAAAGTT
ACCACATGCGTCGTTGTTTATACAGTAATAGGAACAAGAAAAA
AGTCACCTAAGCTCACCCTCATCAATTGTGGAGTTCCTTTATAT
CCCATCTTCTCTCCAAACACATACGCA
U7 snRNA
(SEQ ID NO: 67)
ggagtCTGCTGCTGCTGCTGCTGCTGCTGCTGCTGCTGCTGCTGCT
GCTGAATTTTTGGAGTAggctttctggctccttaccggaaagcc
U7 Downstream (terminator)
(SEQ ID NO: 49)
CCTCTTATGATGTTTGTTGCCAATGATAGATTGTTTTCACTGTGC
AAAAATTATGGGTAGTTTTGGTGGTCTTGATGCAGTTGTAAGCT
TGGAG
CMV promoter
(SEQ ID NO: 62)
Cgttacataacttacggtaaatggcccgcctggctgaccgcccaacgacc
cccgcccattgacgtcaataatgacgtatgttcccatagtaacgccaata
gggactttccattgacgtcaatgggtggagtatttacggtaaactgccca
cttggcagtacatcaagtgtatcatatgccaagtacgccccctattgacg
tcaatgacggtaaatggcccgcctggcattatgcccagtacatgacctta
tgggactttcctacttggcagtacatctacgtattagtcatcgctattac
catggtgatgcggttttggcagtacatcaatgggcgtggatagcggtttg
actcacggggatttccaagtctccaccccattgacgtcaatgggagtttg
ttttggcaccaaaatcaacgggactttccaaaatgtcgtaacaactccgc
cccattgacgcaaatgggcggtaggcgtgtacggtgggaggtctatataa
gcagagct
eGFP
(SEQ ID NO: 63)
ATGgtgagcaagggcgaggagctgttcaccggggtggtgcccatcctggt
cgagctggacggcgacgtaaacggccacaagttcagcgtgtccggcgagg
gcgagggcgatgccacctacggcaagctgaccctgaagttcatctgcacc
accggcaagctgcccgtgccctggcccaccctcgtgaccaccctgaccta
cggcgtgcagtgcttcagccgctaccccgaccacatgaagcagcacgact
tcttcaagtccgccatgcccgaaggctacgtccaggagcgcaccatcttc
ttcaaggacgacggcaactacaagacccgcgccgaggtgaagttcgaggg
cgacaccctggtgaaccgcatcgagctgaagggcatcgacttcaaggagg
acggcaacatcctggggcacaagctggagtacaactacaacagccacaac
gtctatatcatggccgacaagcagaagaacggcatcaaggtgaacttcaa
gatccgccacaacatcgaggacggcagcgtgcagctcgccgaccactacc
agcagaacacccccatcggcgacggccccgtgctgctgcccgacaaccac
tacctgagcacccagtccgccctgagcaaagaccccaacgagaagcgcga
tcacatggtcctgctggagttcgtgaccgccgccgggatcactctcggca
tggacgagctgtacaagTAA
SV40 Poly A
(SEQ ID NO: 64)
Aacttgtttattgcagcttataatggttacaaataaagcaatagcatcac
aaatttcacaaataaagcatttttttcactgcattctagttgtggtttgt
ccaaactcatcaatgtatctta
ITR
(SEQ ID NO: 60)
Aggaacccctagtgatggagttggccactccctctctgcgcgctcgctcg
ctcactgaggccgggcgaccaaaggtcgcccgacgcccgggctttgcccg
ggggcctcagtgagcgagcgagcgcgcagctgcctgcagg

An exemplary AAV vector of the disclosure comprises from 5′ to 3′: a first inverted terminal repeat (ITR) sequence, a U7 promoter sequence, a CUG repeat targeting U7 esnRNA sequence, a U7 downstream terminator sequence, a buffer sequence, a U7 promoter sequence, a CUG repeat targeting U7 esnRNA sequence, a U7 downstream terminator sequence, a buffer Attorney Docket No.: LOCN-020/C)2US (330675-2202) sequence, a U7 promoter sequence, a CUG repeat targeting U7 esnRNA sequence, a U7 downstream terminator sequence, a CMV promoter sequence, a sequence encoding green fluorescent protein (GFP), a sv40 polyA sequence, and a second ITR sequence. In some aspects, a nucleic acid sequence encoding AAV vector A02896 encoding a CUG repeat targeting repeat targeting snRNA sequence comprises SEQ ID NO: 132.

A02896 pAAV_3xU7(15xCUG)_CMV_GFP (500 bp Buffer)

ITR
(SEQ ID NO: 59)
Cctgcaggcagctgcgcgctcgctcgctcactgaggccgcccgggcgtcg
ggcgacctttggtcgcccggcctcagtgagcgagcgagcgcgcagagagg
gagtggccaactccatcactaggggttcct
U7 promoter
(SEQ ID NO: 39)
TACTGCCGAATCCAGGTCTCCGGGCTTAACAACAACGAAGGGG
CTGTGACTGGCTGCTTTCTCAACCAATCAGCACCGAACTCATTT
GCATGGGCTGAGAACAAATGTTCGCGAACTCTAGAAATGAATG
ACTTAAGTAAGTTCCTTAGAATATTATTTTTCCTACTGAAAGTTA
CCACATGCGTCGTTGTTTATACAGTAATAGGAACAAGAAAAAA
GTCACCTAAGCTCACCCTCATCAATTGTGGAGTTCCTTTATATCC
CATCTTCTCTCCAAACACATACGCA
U7 snRNA
(SEQ ID NO: 65)
ggagtCAGCAGCAGCAGCAGCAGCAGCAGCAGCAGCAGCAGCAG
CAGCAGAATTTTTGGAGTAggctttctggctccttaccggaaagcc
U7 Downstream (terminator)
(SEQ ID NO: 49)
CCTCTTATGATGTTTGTTGCCAATGATAGATTGTTTTCACTGTGC
AAAAATTATGGGTAGTTTTGGTGGTCTTGATGCAGTTGTAAGCT
TGGAG
500 bp buffer 1
(SEQ ID NO: 29)
CAAACTACAGAGCCAAGTGCTATCCACAGAGAGCTTTCTGGGTT
GCCATCTCAAGCAGACTACAAGGCCATCAGCTCATACTCACAAT
TGACTTTGAGAGTCATTTTCCAATGCTCCTACACACCCCTTCTTC
ACAATCCCCAACAAATCTGAGGCTGGAACTTGGTACCATAACAA
TCATTACATTATTTCACCAGAAGTACACCTTGCCTGGAAGATTG
GCATTATAGCATCTTCTAACATTGTGAAAGTTAGTGACCAATGA
GGAGATCCAAGTCAGTTCCAGTTGGATTTCTCTATACTCTATAAT
AAATATATATGGTGTCTTCAACAATAGGACTTTGCCATCCAGTG
ATGCTAAAAATCAATAACAATGGCAATAACCTGCCCTGTTTGGA
AAGCCTCTGGCTTCCATGACTAACAATTCAAGGCAGGTCTCCTA
TACCTAGTACTGAGATTTTTATTTGATAAACTATATCTTCTGGGA
GGAGAAGCATTGT
U7 promoter
(SEQ ID NO: 39)
TACTGCCGAATCCAGGTCTCCGGGCTTAACAACAACGAAGGGG
CTGTGACTGGCTGCTTTCTCAACCAATCAGCACCGAACTCATTT
GCATGGGCTGAGAACAAATGTTCGCGAACTCTAGAAATGAATG
ACTTAAGTAAGTTCCTTAGAATATTATTTTTCCTACTGAAAGTTA
CCACATGCGTCGTTGTTTATACAGTAATAGGAACAAGAAAAAA
GTCACCTAAGCTCACCCTCATCAATTGTGGAGTTCCTTTATATCC
CATCTTCTCTCCAAACACATACGCA
U7 snRNA
(SEQ ID NO: 65)
ggagtCAGCAGCAGCAGCAGCAGCAGCAGCAGCAGCAGCAGCAG
CAGCAGAATTTTTGGAGTAggctttctggctccttaccggaaagcc
U7 Downstream (terminator)
(SEQ ID NO: 49)
CCTCTTATGATGTTTGTTGCCAATGATAGATTGTTTTCACTGTGC
AAAAATTATGGGTAGTTTTGGTGGTCTTGATGCAGTTGTAAGCT
TGGAG
500 bp buffer 2
(SEQ ID NO: 30)
TTGACCACATACGTGCTCTTTCAAAGTTCTGTGTTTGAAGTTATG
TTAGTAACAACTGATGCCCATCCTGCAATGACAAATCCAATTCT
CAGTGCAGCTCTCTGAAATAGTTTTGCTTTCTCTCTCTAGGTCTG
TTCTATACTCCTAACTCTCCAGGAGTTTACAAGGAATAAAATCT
CTTCCAAATGCTTTCTGTTGCAACAACTGGACCATACTGAAAGC
TGAGGCCCACAATTGCAATCTAGGTTAGCAGGTAATCATTGTTG
GTGAGGTCCTCCCTTTCCCCAGGCTCGTGTTTGTATTGGGGAGC
AGGAAATTTTTGCTAGAGCAGCACTGCCATCTCTCTACACTCCA
CCTGATTGGTGGGATGGACCAGAGAAATGGACATTCCCAACAC
AGTCCCTCCTTTCACATCTGCTCACCTGCCCACAGGATACTTTCC
ACCATGCATACTGGGCTCTGCACCAACCATTCAGCAGTGATGAA
GAGGAAACTTGAAC
U7 promoter
(SEQ ID NO: 39)
TACTGCCGAATCCAGGTCTCCGGGCTTAACAACAACGAAGGGG
CTGTGACTGGCTGCTTTCTCAACCAATCAGCACCGAACTCATTT
GCATGGGCTGAGAACAAATGTTCGCGAACTCTAGAAATGAATG
ACTTAAGTAAGTTCCTTAGAATATTATTTTTCCTACTGAAAGTTA
CCACATGCGTCGTTGTTTATACAGTAATAGGAACAAGAAAAAA
GTCACCTAAGCTCACCCTCATCAATTGTGGAGTTCCTTTATATCC
CATCTTCTCTCCAAACACATACGCA
U7 snRNA
(SEQ ID NO: 65)
ggagtCAGCAGCAGCAGCAGCAGCAGCAGCAGCAGCAGCAGCAG
CAGCAGAATTTTTGGAGTAggctttctggctccttaccggaaagcc
U7 Downstream (terminator)
(SEQ ID NO: 49)
CCTCTTATGATGTTTGTTGCCAATGATAGATTGTTTTCACTGTGC
AAAAATTATGGGTAGTTTTGGTGGTCTTGATGCAGTTGTAAGCT
TGGAG
CMV promoter
(SEQ ID NO: 62)
Cgttacataacttacggtaaatggcccgcctggctgaccgcccaacgacc
cccgcccattgacgtcaataatgacgtatgttcccatagtaacgccaata
gggactttccattgacgtcaatgggtggagtatttacggtaaactgccca
cttggcagtacatcaagtgtatcatatgccaagtacgccccctattgacg
tcaatgacggtaaatggcccgcctggcattatgcccagtacatgacctta
tgggactttcctacttggcagtacatctacgtattagtcatcgctattac
catggtgatgcggttttggcagtacatcaatgggcgtggatagcggtttg
actcacggggatttccaagtctccaccccattgacgtcaatgggagtttg
ttttggcaccaaaatcaacgggactttccaaaatgtcgtaacaactccgc
cccattgacgcaaatgggcggtaggcgtgtacggtgggaggtctatataa
gcagagct
eGFP
(SEQ ID NO: 63)
ATGgtgagcaagggcgaggagctgttcaccggggtggtgcccatcctggt
cgagctggacggcgacgtaaacggccacaagttcagcgtgtccggcgagg
gcgagggcgatgccacctacggcaagctgaccctgaagttcatctgcacc
accggcaagctgcccgtgccctggcccaccctcgtgaccaccctgaccta
cggcgtgcagtgcttcagccgctaccccgaccacatgaagcagcacgact
tcttcaagtccgccatgcccgaaggctacgtccaggagcgcaccatcttc
ttcaaggacgacggcaactacaagacccgcgccgaggtgaagttcgaggg
cgacaccctggtgaaccgcatcgagctgaagggcatcgacttcaaggagg
acggcaacatcctggggcacaagctggagtacaactacaacagccacaac
gtctatatcatggccgacaagcagaagaacggcatcaaggtgaacttcaa
gatccgccacaacatcgaggacggcagcgtgcagctcgccgaccactacc
agcagaacacccccatcggcgacggccccgtgctgctgcccgacaaccac
tacctgagcacccagtccgccctgagcaaagaccccaacgagaagcgcga
tcacatggtcctgctggagttcgtgaccgccgccgggatcactctcggca
tggacgagctgtacaagTAA
SV40 Poly A
(SEQ ID NO: 64)
Aacttgtttattgcagcttataatggttacaaataaagcaatagcatcac
aaatttcacaaataaagcatttttttcactgcattctagttgtggtttgt
ccaaactcatcaatgtatctta
ITR
(SEQ ID NO: 60)
Aggaacccctagtgatggagttggccactccctctctgcgcgctcgctcg
ctcactgaggccgggcgaccaaaggtcgcccgacgcccgggctttgcccg
ggcggcctcagtgagcgagcgagcgcgcagctgcctgcagg

An exemplary AAV vector of the disclosure comprises from 5′ to 3′: a first inverted terminal repeat (ITR) sequence, a U7 promoter sequence, a non-targeting U7 esnRNA sequence, a U7 downstream terminator sequence, a buffer sequence, a U7 promoter sequence, a non-targeting U7 esnRNA sequence, a U7 downstream terminator sequence, a buffer sequence, a U7 promoter sequence, a non-targeting U7 esnRNA sequence, a U7 downstream terminator sequence, a CMV promoter sequence, a sequence encoding green fluorescent protein (GFP), a sv40 polyA sequence, and a second ITR sequence. In some aspects, a nucleic acid sequence encoding AAV vector A02897 encoding a non-targeting repeat targeting snRNA sequence comprises SEQ ID NO: 133.

A02897 pAAV_3xU7(NT1)_CMV_GFP (500 bp Buffer)

ITR
(SEQ ID NO: 59)
Cctgcaggcagctgcgcgctcgctcgctcactgaggccgcccgggcgtcg
ggcgacctttggtcgcccggcctcagtgagcgagcgagcgcgcagagagg
gagtggccaactccatcactaggggttcct
U7 promoter
(SEQ ID NO: 39)
TACTGCCGAATCCAGGTCTCCGGGCTTAACAACAACGAAGGGG
CTGTGACTGGCTGCTTTCTCAACCAATCAGCACCGAACTCATTT
GCATGGGCTGAGAACAAATGTTCGCGAACTCTAGAAATGAATG
ACTTAAGTAAGTTCCTTAGAATATTATTTTTCCTACTGAAAGTT
ACCACATGCGTCGTTGTTTATACAGTAATAGGAACAAGAAAAA
AGTCACCTAAGCTCACCCTCATCAATTGTGGAGTTCCTTTATAT
CCCATCTTCTCTCCAAACACATACGCA
U7 snRNA
(SEQ ID NO: 66)
ggagtTCACCAGAAGCGTACCATACTCACGAAATTTTTGGAGTAgg
ctttctggctccttaccggaaagcc
U7 (terminator) Downstream
(SEQ ID NO: 49)
CCTCTTATGATGTTTGTTGCCAATGATAGATTGTTTTCACTGTGC
AAAAATTATGGGTAGTTTTGGTGGTCTTGATGCAGTTGTAAGCT
TGGAG
500 bp buffer 1
(SEQ ID NO: 29)
CAAACTACAGAGCCAAGTGCTATCCACAGAGAGCTTTCTGGGT
TGCCATCTCAAGCAGACTACAAGGCCATCAGCTCATACTCACA
ATTGACTTTGAGAGTCATTTTCCAATGCTCCTACACACCCCTTC
TTCACAATCCCCAACAAATCTGAGGCTGGAACTTGGTACCATA
ACAATCATTACATTATTTCACCAGAAGTACACCTTGCCTGGAAG
ATTGGCATTATAGCATCTTCTAACATTGTGAAAGTTAGTGACCA
ATGAGGAGATCCAAGTCAGTTCCAGTTGGATTTCTCTATACTCT
ATAATAAATATATATGGTGTCTTCAACAATAGGACTTTGCCATC
CAGTGATGCTAAAAATCAATAACAATGGCAATAACCTGCCCTG
TTTGGAAAGCCTCTGGCTTCCATGACTAACAATTCAAGGCAGG
TCTCCTATACCTAGTACTGAGATTTTTATTTGATAAACTATATCT
TCTGGGAGGAGAAGCATTGT
U7 promoter
(SEQ ID NO: 39)
TACTGCCGAATCCAGGTCTCCGGGCTTAACAACAACGAAGGGG
CTGTGACTGGCTGCTTTCTCAACCAATCAGCACCGAACTCATTT
GCATGGGCTGAGAACAAATGTTCGCGAACTCTAGAAATGAATG
ACTTAAGTAAGTTCCTTAGAATATTATTTTTCCTACTGAAAGTT
ACCACATGCGTCGTTGTTTATACAGTAATAGGAACAAGAAAAA
AGTCACCTAAGCTCACCCTCATCAATTGTGGAGTTCCTTTATAT
CCCAT
U7 snRNA
(SEQ ID NO: 66)
ggagtTCACCAGAAGCGTACCATACTCACGAAATTTTTGGAGTAgg
ctttctggctccttaccggaaagcc
U7 Downstream (terminator)
(SEQ ID NO: 49)
CCTCTTATGATGTTTGTTGCCAATGATAGATTGTTTTCACTGTGC
AAAAATTATGGGTAGTTTTGGTGGTCTTGATGCAGTTGTAAGCT
TGGAG
500 bp buffer 2
(SEQ ID NO: 30)
TTGACCACATACGTGCTCTTTCAAAGTTCTGTGTTTGAAGTTAT
GTTAGTAACAACTGATGCCCATCCTGCAATGACAAATCCAATT
CTCAGTGCAGCTCTCTGAAATAGTTTTGCTTTCTCTCTCTAGGTC
TGTTCTATACTCCTAACTCTCCAGGAGTTTACAAGGAATAAAAT
CTCTTCCAAATGCTTTCTGTTGCAACAACTGGACCATACTGAAA
GCTGAGGCCCACAATTGCAATCTAGGTTAGCAGGTAATCATTG
TTGGTGAGGTCCTCCCTTTCCCCAGGCTCGTGTTTGTATTGGGG
AGCAGGAAATTTTTGCTAGAGCAGCACTGCCATCTCTCTACACT
CCACCTGATTGGTGGGATGGACCAGAGAAATGGACATTCCCAA
CACAGTCCCTCCTTTCACATCTGCTCACCTGCCCACAGGATACT
TTCCACCATGCATACTGGGCTCTGCACCAACCATTCAGCAGTGA
TGAAGAGGAAACTTGAAC
U7 promoter
(SEQ ID NO: 39)
TACTGCCGAATCCAGGTCTCCGGGCTTAACAACAACGAAGGGG
CTGTGACTGGCTGCTTTCTCAACCAATCAGCACCGAACTCATTT
GCATGGGCTGAGAACAAATGTTCGCGAACTCTAGAAATGAATG
ACTTAAGTAAGTTCCTTAGAATATTATTTTTCCTACTGAAAGTT
ACCACATGCGTCGTTGTTTATACAGTAATAGGAACAAGAAAAA
AGTCACCTAAGCTCACCCTCATCAATTGTGGAGTTCCTTTATAT
CCCATCTTCTCTCCAAACACATACGCA
U7 snRNA
(SEQ ID NO: 66)
ggagtTCACCAGAAGCGTACCATACTCACGAAATTTTTGGAGTAgg
ctttctggctccttaccggaaagcc
U7 Downstream (terminator)
(SEQ ID NO: 49)
CCTCTTATGATGTTTGTTGCCAATGATAGATTGTTTTCACTGTGC
AAAAATTATGGGTAGTTTTGGTGGTCTTGATGCAGTTGTAAGCT
TGGAG
CMV promoter
(SEQ ID NO: 62)
Cgttacataacttacggtaaatggcccgcctggctgaccgcccaacgacc
cccgcccattgacgtcaataatgacgtatgttcccatagtaacgccaata
gggactttccattgacgtcaatgggtggagtatttacggtaaactgccca
cttggcagtacatcaagtgtatcatatgccaagtacgccccctattgacg
tcaatgacggtaaatggcccgcctggcattatgcccagtacatgacctta
tgggactttcctacttggcagtacatctacgtattagtcatcgctattac
catggtgatgcggttttggcagtacatcaatgggcgtggatagcggtttg
actcacggggatttccaagtctccaccccattgacgtcaatgggagtttg
ttttggcaccaaaatcaacgggactttccaaaatgtcgtaacaactccgc
cccattgacgcaaatgggcggtaggcgtgtacggtgggaggtctatataa
gcagagct
eGFP
(SEQ ID NO: 63)
ATGgtgagcaagggcgaggagctgttcaccggggtggtgcccatcctggt
cgagctggacggcgacgtaaacggccacaagttcagcgtgtccggcgagg
gcgagggcgatgccacctacggcaagctgaccctgaagttcatctgcacc
accggcaagctgcccgtgccctggcccaccctcgtgaccaccctgaccta
cggcgtgcagtgcttcagccgctaccccgaccacatgaagcagcacgact
tcttcaagtccgccatgcccgaaggctacgtccaggagcgcaccatcttc
ttcaaggacgacggcaactacaagacccgcgccgaggtgaagttcgaggg
cgacaccctggtgaaccgcatcgagctgaagggcatcgacttcaaggagg
acggcaacatcctggggcacaagctggagtacaactacaacagccacaac
gtctatatcatggccgacaagcagaagaacggcatcaaggtgaacttcaa
gatccgccacaacatcgaggacggcagcgtgcagctcgccgaccactacc
agcagaacacccccatcggcgacggccccgtgctgctgcccgacaaccac
tacctgagcacccagtccgccctgagcaaagaccccaacgagaagcgcga
tcacatggtcctgctggagttcgtgaccgccgccgggatcactctcggca
tggacgagctgtacaagTAA
SV40 Poly A
(SEQ ID NO: 64)
Aacttgtttattgcagcttataatggttacaaataaagcaatagcatcac
aaatttcacaaataaagcatttttttcactgcattctagttgtggtttgt
ccaaactcatcaatgtatctta
ITR
(SEQ ID NO: 60)
Aggaacccctagtgatggagttggccactccctctctgcgcgctcgctcg
ctcactgaggccgggcgaccaaaggtcgcccgacgcccgggctttgcccg
ggcggcctcagtgagcgagcgagcgcgcagctgcctgcagg

An exemplary AAV vector of the disclosure comprises from 5′ to 3′: a first inverted terminal repeat (ITR) sequence, a U7 promoter sequence, a CAG repeat targeting U7 esnRNA sequence, a U7 downstream terminator sequence, a buffer sequence, a U7 promoter sequence, a CAG repeat targeting U7 esnRNA sequence, a U7 downstream terminator sequence, a buffer sequence, a U7 promoter sequence, a CAG repeat targeting U7 esnRNA sequence, a U7 downstream terminator sequence, a CMV promoter sequence, a sequence encoding green fluorescent protein (GFP), a sv40 polyA sequence, and a second ITR sequence. In some aspects, a nucleic acid sequence encoding AAV vector A02969 encoding a CAG repeat targeting repeat targeting snRNA sequence comprises SEQ ID NO: 134.

A02969 pAAV_3xU7(15xCAG)_CMV_GFP (500 bp Buffer) Targeting CAG Repeats

ITR
(SEQ ID NO: 59)
Cctgcaggcagctgcgcgctcgctcgctcactgaggccgcccgggcgtcg
ggcgacctttggtcgcccggcctcagtgagcgagcgagcgcgcagagagg
gagtggccaactccatcactaggggttcct
U7 promoter
(SEQ ID NO: 39)
TACTGCCGAATCCAGGTCTCCGGGCTTAACAACAACGAAGGGG
CTGTGACTGGCTGCTTTCTCAACCAATCAGCACCGAACTCATTT
GCATGGGCTGAGAACAAATGTTCGCGAACTCTAGAAATGAATG
ACTTAAGTAAGTTCCTTAGAATATTATTTTTCCTACTGAAAGTTA
CCACATGCGTCGTTGTTTATACAGTAATAGGAACAAGAAAAAA
GTCACCTAAGCTCACCCTCATCAATTGTGGAGTTCCTTTATATCC
CATCTTCTCTCCAAACACATACGCA
U7 snRNA
(SEQ ID NO: 67)
ggagtCTGCTGCTGCTGCTGCTGCTGCTGCTGCTGCTGCTGCTGCTG
CTGAATTTTTGGAGTAggctttctggctccttaccggaaagcc
U7 Downstream (terminator)
(SEQ ID NO: 49)
CCTCTTATGATGTTTGTTGCCAATGATAGATTGTTTTCACTGTGC
AAAAATTATGGGTAGTTTTGGTGGTCTTGATGCAGTTGTAAGCT
TGGAG
500 bp buffer 1
(SEQ ID NO: 29)
CAAACTACAGAGCCAAGTGCTATCCACAGAGAGCTTTCTGGGTT
GCCATCTCAAGCAGACTACAAGGCCATCAGCTCATACTCACAAT
TGACTTTGAGAGTCATTTTCCAATGCTCCTACACACCCCTTCTTC
ACAATCCCCAACAAATCTGAGGCTGGAACTTGGTACCATAACAA
TCATTACATTATTTCACCAGAAGTACACCTTGCCTGGAAGATTG
GCATTATAGCATCTTCTAACATTGTGAAAGTTAGTGACCAATGA
GGAGATCCAAGTCAGTTCCAGTTGGATTTCTCTATACTCTATAAT
AAATATATATGGTGTCTTCAACAATAGGACTTTGCCATCCAGTG
ATGCTAAAAATCAATAACAATGGCAATAACCTGCCCTGTTTGGA
AAGCCTCTGGCTTCCATGACTAACAATTCAAGGCAGGTCTCCTA
TACCTAGTACTGAGATTTTTATTTGATAAACTATATCTTCTGGGA
GGAGAAGCATTGT
U7 promoter
(SEQ ID NO: 39)
TACTGCCGAATCCAGGTCTCCGGGCTTAACAACAACGAAGGGG
CTGTGACTGGCTGCTTTCTCAACCAATCAGCACCGAACTCATTT
GCATGGGCTGAGAACAAATGTTCGCGAACTCTAGAAATGAATG
ACTTAAGTAAGTTCCTTAGAATATTATTTTTCCTACTGAAAGTTA
CCACATGCGTCGTTGTTTATACAGTAATAGGAACAAGAAAAAA
GTCACCTAAGCTCACCCTCATCAATTGTGGAGTTCCTTTATATCC
CATCTTCTCTCCAAACACATACGCA
U7 snRNA
(SEQ ID NO: 67)
ggagtCTGCTGCTGCTGCTGCTGCTGCTGCTGCTGCTGCTGCTGCTG
CTGAATTTTTGGAGTAggctttctggctccttaccggaaagcc
U7 Downstream (terminator)
(SEQ ID NO: 49)
CCTCTTATGATGTTTGTTGCCAATGATAGATTGTTTTCACTGTGC
AAAAATTATGGGTAGTTTTGGTGGTCTTGATGCAGTTGTAAGCT
TGGAG
500 bp buffer 2
(SEQ ID NO: 30)
TTGACCACATACGTGCTCTTTCAAAGTTCTGTGTTTGAAGTTATG
TTAGTAACAACTGATGCCCATCCTGCAATGACAAATCCAATTCT
CAGTGCAGCTCTCTGAAATAGTTTTGCTTTCTCTCTCTAGGTCTG
TTCTATACTCCTAACTCTCCAGGAGTTTACAAGGAATAAAATCT
CTTCCAAATGCTTTCTGTTGCAACAACTGGACCATACTGAAAGC
TGAGGCCCACAATTGCAATCTAGGTTAGCAGGTAATCATTGTTG
GTGAGGTCCTCCCTTTCCCCAGGCTCGTGTTTGTATTGGGGAGC
AGGAAATTTTTGCTAGAGCAGCACTGCCATCTCTCTACACTCCA
CCTGATTGGTGGGATGGACCAGAGAAATGGACATTCCCAACAC
AGTCCCTCCTTTCACATCTGCTCACCTGCCCACAGGATACTTTCC
ACCATGCATACTGGGCTCTGCACCAACCATTCAGCAGTGATGAA
GAGGAAACTTGAAC
U7 promoter
(SEQ ID NO: 39)
TACTGCCGAATCCAGGTCTCCGGGCTTAACAACAACGAAGGGG
CTGTGACTGGCTGCTTTCTCAACCAATCAGCACCGAACTCATTT
GCATGGGCTGAGAACAAATGTTCGCGAACTCTAGAAATGAATG
ACTTAAGTAAGTTCCTTAGAATATTATTTTTCCTACTGAAAGTTA
CCACATGCGTCGTTGTTTATACAGTAATAGGAACAAGAAAAAA
GTCACCTAAGCTCACCCTCATCAATTGTGGAGTTCCTTTATATCC
CATCTTCTCTCCAAACACATACGCA
U7 snRNA
(SEQ ID NO: 67)
ggagtCTGCTGCTGCTGCTGCTGCTGCTGCTGCTGCTGCTGCTGCTG
CTGAATTTTTGGAGTAggctttctggctccttaccggaaagcc
U7 Downstream (terminator)
(SEQ ID NO: 49)
CCTCTTATGATGTTTGTTGCCAATGATAGATTGTTTTCACTGTGC
AAAAATTATGGGTAGTTTTGGTGGTCTTGATGCAGTTGTAAGCT
TGGAG
CMV promoter
(SEQ ID NO: 62)
Cgttacataacttacggtaaatggcccgcctggctgaccgcccaacgacc
cccgcccattgacgtcaataatgacgtatgttcccatagtaacgccaata
gggactttccattgacgtcaatgggtggagtatttacggtaaactgccca
cttggcagtacatcaagtgtatcatatgccaagtacgccccctattgacg
tcaatgacggtaaatggcccgcctggcattatgcccagtacatgacctta
tgggactttcctacttggcagtacatctacgtattagtcatcgctattac
catggtgatgcggttttggcagtacatcaatgggcgtggatagcggtttg
actcacggggatttccaagtctccaccccattgacgtcaatgggagtttg
ttttggcaccaaaatcaacgggactttccaaaatgtcgtaacaactccgc
cccattgacgcaaatgggcggtaggcgtgtacggtgggaggtctatataa
gcagagct
eGFP
(SEQ ID NO: 63)
ATGgtgagcaagggcgaggagctgttcaccggggtggtgcccatcctggt
cgagctggacggcgacgtaaacggccacaagttcagcgtgtccggcgagg
gcgagggcgatgccacctacggcaagctgaccctgaagttcatctgcacc
accggcaagctgcccgtgccctggcccaccctcgtgaccaccctgaccta
cggcgtgcagtgcttcagccgctaccccgaccacatgaagcagcacgact
tcttcaagtccgccatgcccgaaggctacgtccaggagcgcaccatcttc
ttcaaggacgacggcaactacaagacccgcgccgaggtgaagttcgaggg
cgacaccctggtgaaccgcatcgagctgaagggcatcgacttcaaggagg
acggcaacatcctggggcacaagctggagtacaactacaacagccacaac
gtctatatcatggccgacaagcagaagaacggcatcaaggtgaacttcaa
gatccgccacaacatcgaggacggcagcgtgcagctcgccgaccactacc
agcagaacacccccatcggcgacggccccgtgctgctgcccgacaaccac
tacctgagcacccagtccgccctgagcaaagaccccaacgagaagcgcga
tcacatggtcctgctggagttcgtgaccgccgccgggatcactctcggca
tggacgagctgtacaagTAA
SV40 Poly A
(SEQ ID NO: 64)
Aacttgtttattgcagcttataatggttacaaataaagcaatagcatcac
aaatttcacaaataaagcatttttttcactgcattctagttgtggtttgt
ccaaactcatcaatgtatctta
ITR
(SEQ ID NO: 60)
Aggaacccctagtgatggagttggccactccctctctgcgcgctcgctcg
ctcactgaggccgggcgaccaaaggtcgcccgacgcccgggctttgcccg
ggggcctcagtgagcgagcgagcgcgcagctgcctgcagg

An exemplary AAV vector of the disclosure comprises from 5′ to 3′: a first inverted terminal repeat (ITR) sequence, a U7 promoter sequence, a DMD exon 51 targeting U7 esnRNA sequence, a U7 downstream terminator sequence, a U1 promoter sequence, a DMD exon 51 targeting U7 esnRNA sequence, a U1 downstream terminator sequence, and a second ITR sequence. In some aspects, a nucleic acid sequence encoding AAV vector A03981 encoding a DMD exon 51 targeting snRNA sequences comprises SEQ ID NO: 136.

A03981: pAAV(ssAAV)_U7promoter-esnRNA-z38/42-U7termination-U1promoter-esnRNAz38/z42-U1 Termination

ITR
(SEQ ID NO: 59)
cctgcaggcagctgcgcgctcgctcgctcactgaggccgcccgggcgtcg
ggcgacctttggtcgcccggcctcagtgagcgagcgagcgcgcagagagg
gagtggccaactccatcactaggggttcct
U7 promoter
(SEQ ID NO: 39)
TACTGCCGAATCCAGGTCTCCGGGCTTAACAACAACGAAGGGGCTG
TGACTGGCTGCTTTCTCAACCAATCAGCACCGAACTCATTTGCATGG
GCTGAGAACAAATGTTCGCGAACTCTAGAAATGAATGACTTAAGTA
AGTTCCTTAGAATATTATTTTTCCTACTGAAAGTTACCACATGCGTC
GTTGTTTATACAGTAATAGGAACAAGAAAAAAGTCACCTAAGCTCA
CCCTCATCAATTGTGGAGTTCCTTTATATCCCATCTTCTCTCCAAAC
ACATACGCA
z38/42 esnRNA (DMD exon 51)
(SEQ ID NO: 68)
ggagtTACCTCCAACATCAAGGAAGATGGCAGTAACCACAGGTTGTGT
CACCAGAGTAACAAATTTTTGGAGTAggctttctggctccttaccggaaa
gcc
U7 termination
(SEQ ID NO: 49)
CCTCTTATGATGTTTGTTGCCAATGATAGATTGTTTTCACTGTGCAA
AAATTATGGGTAGTTTTGGTGGTCTTGATGCAGTTGTAAGCTTGGAG
U1 promoter
(SEQ ID NO: 40)
TAAGGACCAGCTTCTTTGGGAGAGAACAGACGCAGGGGCGGGAGG
GAAAAAGGGAGAGGCAGACGTCACTTCCCCTTGGCGGCTCTGGCAG
CAGATTGGTCGGTTGAGTGGCAGAAAGGCAGACGGGGACTGGGCA
AGGCACTGTCGGTGACATCACGGACAGGGCGACTTCTATGTAGATG
AGGCAGCGCAGAGGCTGACGTCTTCGCCACTTGCTGCTTCACCACG
AAGGAGTTCCCGTGCCCTGGGAGCGGGTTCAGGACCGCTGATCGGA
AGTGAGAATCCCAGCTGTGTGTCAGGGCTGGAAAGGGCTCGGGAGT
GCGCGGGGCAAGTGACCGTGTGTGTAAAGAGTGAGGCGTATGAGG
CTGTGTCGGGGCAGAGGCacaacgtttc
z38/42 esnRNA (DMD exon 51)
(SEQ ID NO: 68)
ggagtTACCTCCAACATCAAGGAAGATGGCAGTAACCACAGGTTGTGT
CACCAGAGTAACAAATTTTTGGAGTAggctttctggctccttaccgga
aagcc
U1 termination
(SEQ ID NO: 50)
ACTTTCTGGAGTTTCAAAAACAGACTGTACGCCAAGGGTCATATCTTTTT
ITR
(SEQ ID NO: 60)
aggaacccctagtgatggagttggccactccctctctgcgcgctcgctcg
ctcactgaggccgggcgaccaaaggtcgcccgacgcccgggctttgcccg
ggggcctcagtgagcgagcgagcgcgcagctgcctgcagg

An exemplary AAV vector of the disclosure comprises from 5′ to 3′: a first inverted terminal repeat (ITR) sequence, a U7 promoter sequence, a DMD exon 51 targeting U7 esnRNA sequence, a U7 downstream terminator sequence, a U1 promoter sequence, a DMD exon 51 targeting U7 esnRNA sequence, a U1 downstream terminator sequence, and a second ITR sequence. In some aspects, a nucleic acid sequence encoding AAV vector A03981 encoding a DMD exon 51 targeting snRNA sequences comprises SEQ ID NO: 136.

A03981: pAAV(ssAAV)_U7promoter-esnRNA-z42-U7termination-U1promoter-esnRNA-z38-U1 Termination

ITR
(SEQ ID NO: 59)
cctgcaggcagctgcgcgctcgctcgctcactgaggccgcccgggcgtcg
ggcgacctttggtcgcccggcctcagtgagcgagcgagcgcgcagagagg
gagtggccaactccatcactaggggttcct
U7 promoter
(SEQ ID NO: 39)
TACTGCCGAATCCAGGTCTCCGGGCTTAACAACAACGAAGGGGCTG
TGACTGGCTGCTTTCTCAACCAATCAGCACCGAACTCATTTGCATGG
GCTGAGAACAAATGTTCGCGAACTCTAGAAATGAATGACTTAAGTA
AGTTCCTTAGAATATTATTTTTCCTACTGAAAGTTACCACATGCGTC
GTTGTTTATACAGTAATAGGAACAAGAAAAAAGTCACCTAAGCTCA
CCCTCATCAATTGTGGAGTTCCTTTATATCCCATCTTCTCTCCAAAC
ACATACGCA
z42 esnRNA (DMD exon 51)
(SEQ ID NO: 69)
ggagtGTAACCACAGGTTGTGTCACCAGAGTAACAAATTTTTGGAGTA
ggctttctggctccttaccggaaagcc
U7 termination
(SEQ ID NO: 49)
CCTCTTATGATGTTTGTTGCCAATGATAGATTGTTTTCACTGTGCAA
AAATTATGGGTAGTTTTGGTGGTCTTGATGCAGTTGTAAGCTTGGAG
U1 promoter
(SEQ ID NO: 40)
TAAGGACCAGCTTCTTTGGGAGAGAACAGACGCAGGGGCGGGAGG
GAAAAAGGGAGAGGCAGACGTCACTTCCCCTTGGCGGCTCTGGCAG
CAGATTGGTCGGTTGAGTGGCAGAAAGGCAGACGGGGACTGGGCA
AGGCACTGTCGGTGACATCACGGACAGGGCGACTTCTATGTAGATG
AGGCAGCGCAGAGGCTGACGTCTTCGCCACTTGCTGCTTCACCACG
AAGGAGTTCCCGTGCCCTGGGAGCGGGTTCAGGACCGCTGATCGGA
AGTGAGAATCCCAGCTGTGTGTCAGGGCTGGAAAGGGCTCGGGAGT
GCGCGGGGCAAGTGACCGTGTGTGTAAAGAGTGAGGCGTATGAGG
CTGTGTCGGGGCAGAGGCacaacgtttc
z38 esnRNA (DMD exon 51)
(SEQ ID NO: 70)
ggagtTACCTCCAACATCAAGGAAGATGGCAAATTTTTGGAGTAggcttt
ctggctccttaccggaaagcc
U1 termination
(SEQ ID NO: 50)
ACTTTCTGGAGTTTCAAAAACAGACTGTACGCCAAGGGTCATATCTTTTT
ITR
(SEQ ID NO: 60)
aggaacccctagtgatggagttggccactccctctctgcgcgctcgctcg
ctcactgaggccgggcgaccaaaggtcgcccgacgcccgggctttgcccg
ggggcctcagtgagcgagcgagcgcgcagctgcctgcagg

An exemplary AAV vector of the disclosure comprises from 5′ to 3′: a first inverted terminal repeat (ITR) sequence, a U7 promoter sequence, a DMD exon 51 targeting U7 esnRNA sequence, a U7 downstream terminator sequence, a U1 promoter sequence, a DMD exon 51 targeting U7 esnRNA sequence, a U1 downstream terminator sequence, and a second ITR sequence. In some aspects, a nucleic acid sequence encoding AAV vector A04229 encoding a DMD exon 51 targeting snRNA sequences comprises SEQ ID NO: 137.

A04229: pAAV(scAAV) U7promoter-esnRNA-z38/42-U7 Termination-U1promoter-esnRNAz38/z42-U1 Termination-Mut ITR

ITR
(SEQ ID NO: 59)
cctgcaggcagctgcgcgctcgctcgctcactgaggccgcccgggcgtcg
ggcgacctttggtcgcccggcctcagtgagcgagcgagcgcgcagagagg
gagtggccaactccatcactaggggttcct
U7 promoter
(SEQ ID NO: 39)
TACTGCCGAATCCAGGTCTCCGGGCTTAACAACAACGAAGGGGCT
GTGACTGGCTGCTTTCTCAACCAATCAGCACCGAACTCATTTGCAT
GGGCTGAGAACAAATGTTCGCGAACTCTAGAAATGAATGACTTAA
GTAAGTTCCTTAGAATATTATTTTTCCTACTGAAAGTTACCACATGC
GTCGTTGTTTATACAGTAATAGGAACAAGAAAAAAGTCACCTAAG
CTCACCCTCATCAATTGTGGAGTTCCTTTATATCCCATCTTCTCTCC
AAACACATACGCA
z38/42 esnRNA (DMD exon 51)
(SEQ ID NO: 68)
ggagtTACCTCCAACATCAAGGAAGATGGCAGTAACCACAGGTTGTG
TCACCAGAGTAACAAATTTTTGGAGTAggctttctggctccttaccggaa
agcc
U7 termination
(SEQ ID NO: 49)
CCTCTTATGATGTTTGTTGCCAATGATAGATTGTTTTCACTGTGCAA
AAATTATGGGTAGTTTTGGTGGTCTTGATGCAGTTGTAAGCTTGGA
G
U1 promoter
(SEQ ID NO: 40)
TAAGGACCAGCTTCTTTGGGAGAGAACAGACGCAGGGGCGGGAGG
GAAAAAGGGAGAGGCAGACGTCACTTCCCCTTGGCGGCTCTGGCA
GCAGATTGGTCGGTTGAGTGGCAGAAAGGCAGACGGGGACTGGGC
AAGGCACTGTCGGTGACATCACGGACAGGGCGACTTCTATGTAGA
TGAGGCAGCGCAGAGGCTGACGTCTTCGCCACTTGCTGCTTCACCA
CGAAGGAGTTCCCGTGCCCTGGGAGCGGGTTCAGGACCGCTGATC
GGAAGTGAGAATCCCAGCTGTGTGTCAGGGCTGGAAAGGGCTCGG
GAGTGCGCGGGGCAAGTGACCGTGTGTGTAAAGAGTGAGGCGTAT
GAGGCTGTGTCGGGGCAGAGGCacaacgtttc
z38/42 esnRNA (DMD exon 51)
(SEQ ID NO: 68)
TCACCAGAGTAACAAATTTTTGGAGTAggctttctggctccttaccggaa
agccggagtTACCTCCAACATCAAGGAAGATGGCAGTAACCACAGGTTGT
G
U1 termination
(SEQ ID NO: 50)
ACTTTCTGGAGTTTCAAAAACAGACTGTACGCCAAGGGTCATATCTTTTT
mITR
(SEQ ID NO: 61)
CCACTCCCTCTCTGCGCGCTCGCTCGCTCACTGAGGCCGGGCGACC
AAAGGTCGCCCGACGCCCGGGCTTTGCCCGGGCGGCCTCAGTGAG
CGAGCGAGCGCGCAGAGAGGGA

An exemplary AAV vector of the disclosure comprises from 5′ to 3′: a first inverted terminal repeat (ITR) sequence, a U7 promoter sequence, a DMD exon 51 targeting U7 esnRNA sequence, a U7 downstream terminator sequence, a buffer sequence, a U1 promoter sequence, a DMD exon 51 targeting U7 esnRNA sequence, a U1 downstream terminator sequence, a buffer sequence, and a second ITR sequence. In some aspects, a nucleic acid sequence encoding AAV vector A04184 encoding a DMD exon 51 targeting snRNA sequences comprises SEQ ID NO: 138.

A04184: pAAV(ssAAV)-U7promoter-z38/42-U7term_U1promoter-z38/42-U1term_Buffer Combination Sequence

ITR
(SEQ ID NO: 59)
cctgcaggcagctgcgcgctcgctcgctcactgaggccgcccgggcgtcg
ggcgacctttggtcgcccggcctcagtgagcgagcgagcgcgcagagagg
gagtggccaactccatcactaggggttcct
U7 promoter
(SEQ ID NO: 39)
TACTGCCGAATCCAGGTCTCCGGGCTTAACAACAACGAAGGGG
CTGTGACTGGCTGCTTTCTCAACCAATCAGCACCGAACTCATTT
GCATGGGCTGAGAACAAATGTTCGCGAACTCTAGAAATGAATG
ACTTAAGTAAGTTCCTTAGAATATTATTTTTCCTACTGAAAGTT
ACCACATGCGTCGTTGTTTATACAGTAATAGGAACAAGAAAAA
AGTCACCTAAGCTCACCCTCATCAATTGTGGAGTTCCTTTATAT
CCCATCTTCTCTCCAAACACATACGCA
z38/42 esnRNA (DMD exon 51)
(SEQ ID NO: 68)
ggagtTACCTCCAACATCAAGGAAGATGGCAGTAACCACAGGTTG
TGTCACCAGAGTAACAAATTTTTGGAGTAggctttctggctccttaccgg
aaagcc
U7 termination
(SEQ ID NO: 49)
CCTCTTATGATGTTTGTTGCCAATGATAGATTGTTTTCACTGTGC
AAAAATTATGGGTAGTTTTGGTGGTCTTGATGCAGTTGTAAGCT
TGGAG
U1 promoter
(SEQ ID NO: 40)
TAAGGACCAGCTTCTTTGGGAGAGAACAGACGCAGGGGCGGGA
GGGAAAAAGGGAGAGGCAGACGTCACTTCCCCTTGGCGGCTCT
GGCAGCAGATTGGTCGGTTGAGTGGCAGAAAGGCAGACGGGG
ACTGGGCAAGGCACTGTCGGTGACATCACGGACAGGGCGACTT
CTATGTAGATGAGGCAGCGCAGAGGCTGACGTCTTCGCCACTT
GCTGCTTCACCACGAAGGAGTTCCCGTGCCCTGGGAGCGGGTT
CAGGACCGCTGATCGGAAGTGAGAATCCCAGCTGTGTGTCAGG
GCTGGAAAGGGCTCGGGAGTGCGCGGGGCAAGTGACCGTGTGT
GTAAAGAGTGAGGCGTATGAGGCTGTGTCGGGGCAGAGGCacaa
cgtttc
z38/42 esnRNA (DMD exon 51)
(SEQ ID NO: 68)
ggagtTACCTCCAACATCAAGGAAGATGGCAGTAACCACAGGTTG
TGTCACCAGAGTAACAAATTTTTGGAGTAggctttctggctccttaccgg
aaagcc
U1 termination
(SEQ ID NO: 50)
ACTTTCTGGAGTTTCAAAAACAGACTGTACGCCAAGGGTCATAT
CTTTTT
Buffer combo sequence
(SEQ ID NO: 144)
CAAACTACAGAGCCAAGTGCTATCCACAGAGAGCTTTCTGGGT
TGCCATCTCAAGCAGACTACAAGGCCATCAGCTCATACTCACA
ATTGACTTTGAGAGTCATTTTCCAATGCTCCTACACACCCCTTCT
TCACAATCCCCAACAAATCTGAGGCTGGAACTTGGTACCATAA
CAATCATTACATTATTTCACCAGAAGTACACCTTGCCTGGAAGA
TTGGCATTATAGCATCTTCTAACATTGTGAAAGTTAGTGACCAA
TGAGGAGATCCAAGTCAGTTCCAGTTGGATTTCTCTATACTCTA
TAATAAATATATATGGTGTCTTCAACAATAGGACTTTGCCATCC
AGTGATGCTAAAAATCAATAACAATGGCAATAACCTGCCCTGT
TTGGAAAGCCTCTGGCTTCCATGACTAACAATTCAAGGCAGGTC
TCCTATACCTAGTACTGAGATTTTTATTTGATAAACTATATCTTC
TGGGAGGAGAAGCATTGTTTGACCACATACGTGCTCTTTCAAA
GTTCTGTGTTTGAAGTTATGTTAGTAACAACTGATGCCCATCCT
GCAATGACAAATCCAATTCTCAGTGCAGCTCTCTGAAATAGTTT
TGCTTTCTCTCTCTAGGTCTGTTCTATACTCCTAACTCTCCAGGA
GTTTACAAGGAATAAAATCTCTTCCAAATGCTTTCTGTTGCAAC
AACTGGACCATACTGAAAGCTGAGGCCCACAATTGCAATCTAG
GTTAGCAGGTAATCATTGTTGGTGAGGTCCTCCCTTTCCCCAGG
CTCGTGTTTGTATTGGGGAGCAGGAAATTTTTGCTAGAGCAGCA
CTGCCATCTCTCTACACTCCACCTGATTGGTGGGATGGACCAGA
GAAATGGACATTCCCAACACAGTCCCTCCTTTCACATCTGCTCA
CCTGCCCACAGGATACTTTCCACCATGCATACTGGGCTCTGCAC
CAACCATTCAGCAGTGATGAAGAGGAAACTTGAACCCATGCAG
AGTACCTCTAGAGAAAAGTTTCTAAAGTTGGTTTTGCCTTGCTT
AGAAAGGAGGTCAAAGGTGAAGCCTAGGAGAAACATTCAGCA
ATGTTGCACAAAGACATGAGAATAAATGATTTTAGCATCCTGA
AATGAAGAGCATAGTGATCTCACCATAGCTACCCACCTACCCA
GAGAACTTCCAGGCTTTCACTGTCTCTTTTCTGAATCACCCACA
TTACAGAATATCAGTCATTGTGTAATTAGATAACAGATTGTGAG
GTGCCCAAACAGAAACAAACTTTACAGGAGGAGAGAGAAAAT
CAAGGTGATGACTATGGAGAACAAAATGAGAAGGAGAGGTGG
AGTTCCTGCTGCATGAACACCTATCCTGCTATAGAGCCCATTTC
TTCCATAAATAATACAACTCTGTAACTCCATAGGTCAGAAAGA
AACAGTGTTCTGTGTCTTCCCATCTAGCACCACAAACCCCCACA
AGATTAATTTGTTTCTAGGGACCCTTAAATCTCTATCAAAATTC
TGGAAACCTCTACTTTAGAAAATCTTATATTTATTATTCAGGCT
ACTTTCCAGAGTGATAAGCTACTGAGTCTCCTAAGTGTCATCTA
TGGTACACAGGGATAAGATCAGGAATAAACCTGGG
ITR
(SEQ ID NO: 60)
aggaacccctagtgatggagttggccactccctctctgcgcgctcgctcg
ctcactgaggccgggcgaccaaaggtcgcccgacgcccgggctttgcccg
ggggcctcagtgagcgagcgagcgcgcagctgcctgcagg

An exemplary AAV vector of the disclosure comprises from 5′ to 3′: a first inverted terminal repeat (ITR) sequence, a U7 promoter sequence, a CUG repeat targeting U7 esnRNA sequence, a U7 downstream terminator sequence, a buffer sequence, a U1 promoter sequence, a CUG repeat targeting U7 esnRNA sequence, a U1 downstream terminator sequence, an eCMV promoter sequence, a sequence encoding GFP, a polyA sequence, and a second ITR sequence. In some aspects, a nucleic acid sequence encoding AAV vector A03681 encoding a CUG repeat targeting snRNA sequences comprises SEQ ID NO: 139.

A03681: pAAV(ssAAV)-U7 promoter-zCUGx15-U7 term_U1 promoter-zCUGx15-U1 Term-eCMV-eGFP

ITR
(SEQ ID NO: 59)
cctgcaggcagctgcgcgctcgctcgctcactgaggccgcccgggcgtcg
ggcgacctttggtcgcccggcctcagtgagcgagcgagcgcgcagagagg
gagtggccaactccatcactaggggttcct
U7 promoter
(SEQ ID NO: 39)
TACTGCCGAATCCAGGTCTCCGGGCTTAACAACAACGAAGGGGCTG
TGACTGGCTGCTTTCTCAACCAATCAGCACCGAACTCATTTGCATGG
GCTGAGAACAAATGTTCGCGAACTCTAGAAATGAATGACTTAAGTA
AGTTCCTTAGAATATTATTTTTCCTACTGAAAGTTACCACATGCGTCG
TTGTTTATACAGTAATAGGAACAAGAAAAAAGTCACCTAAGCTCAC
CCTCATCAATTGTGGAGTTCCTTTATATCCCATCTTCTCTCCAAACAC
ATACGCA
zCUGx15 esnRNA
(SEQ ID NO: 65)
ggagtCAGCAGCAGCAGCAGCAGCAGCAGCAGCAGCAGCAGCAGCAG
CAGAATTTTTGGAGTAggctttctggctccttaccggaaagcc
U7 termination
(SEQ ID NO: 49)
CCTCTTATGATGTTTGTTGCCAATGATAGATTGTTTTCACTGTGCAAA
AATTATGGGTAGTTTTGGTGGTCTTGATGCAGTTGTAAGCTTGGAG
U1 promoter
(SEQ ID NO: 40)
TAAGGACCAGCTTCTTTGGGAGAGAACAGACGCAGGGGCGGGAGGG
AAAAAGGGAGAGGCAGACGTCACTTCCCCTTGGCGGCTCTGGCAGC
AGATTGGTCGGTTGAGTGGCAGAAAGGCAGACGGGGACTGGGCAAG
GCACTGTCGGTGACATCACGGACAGGGCGACTTCTATGTAGATGAG
GCAGCGCAGAGGCTGACGTCTTCGCCACTTGCTGCTTCACCACGAAG
GAGTTCCCGTGCCCTGGGAGCGGGTTCAGGACCGCTGATCGGAAGT
GAGAATCCCAGCTGTGTGTCAGGGCTGGAAAGGGCTCGGGAGTGCG
CGGGGCAAGTGACCGTGTGTGTAAAGAGTGAGGCGTATGAGGCTGT
GTCGGGGCAGAGGCacaacgtttc
ZCUGx15 esnRNA
(SEQ ID NO: 65)
ggagtCAGCAGCAGCAGCAGCAGCAGCAGCAGCAGCAGCAGCAGCAG
CAGAATTTTTGGAGTAggctttctggctccttaccggaaagcc
U1 termination
(SEQ ID NO: 50)
ACTTTCTGGAGTTTCAAAAACAGACTGTACGCCAAGGGTCATATCTT
TTT
eCMV promoter
(SEQ ID NO: 62)
cgttacataacttacggtaaatggcccgcctggctgaccgcccaacgacc
cccgcccattgacgtcaataatgacgtatgttcccatagtaacgccaata
gggactttccattgacgtcaatgggtggagtatttacggtaaactgccca
cttggcagtacatcaagtgtatcatatgccaagtacgccccctattgacg
tcaatgacggtaaatggcccgcctggcattatgcccagtacatgacctta
tgggactttcctacttggcagtacatctacgtattagtcatcgctattac
catg
eGFP
(SEQ ID NO: 63)
ATGgtgagcaagggcgaggagctgttcaccggggtggtgcccatcctggt
cgagctggacggcgacgtaaacggccacaagttcagcgtgtccggcgagg
gcgagggcgatgccacctacggcaagctgaccctgaagttcatctgcacc
accggcaagctgcccgtgccctggcccaccctcgtgaccaccctgaccta
cggcgtgcagtgcttcagccgctaccccgaccacatgaagcagcacgact
tcttcaagtccgccatgcccgaaggctacgtccaggagcgcaccatcttc
ttcaaggacgacggcaactacaagacccgcgccgaggtgaagttcgaggg
cgacaccctggtgaaccgcatcgagctgaagggcatcgacttcaaggagg
acggcaacatcctggggcacaagctggagtacaactacaacagccacaac
gtctatatcatggccgacaagcagaagaacggcatcaaggtgaacttcaa
gatccgccacaacatcgaggacggcagcgtgcagctcgccgaccactacc
agcagaacacccccatcggcgacggccccgtgctgctgcccgacaaccac
tacctgagcacccagtccgccctgagcaaagaccccaacgagaagcgcga
tcacatggtcctgctggagttcgtgaccgccgccgggatcactctcggca
tggacgagctgtacaagTAA
SV40 pA
(SEQ ID NO: 64)
aacttgtttattgcagcttataatggttacaaataaagcaatagcatcac
aaatttcacaaataaagcatttttttcactgcattctagttgtggtttgt
ccaaactcatcaatgtatctta
ITR
(SEQ ID NO: 60)
aggaacccctagtgatggagttggccactccctctctgcgcgctcgctcg
ctcactgaggccgggcgaccaaaggtcgcccgacgcccgggctttgcccg
ggggcctcagtgagcgagcgagcgcgcagctgcctgcagg

An exemplary AAV vector of the disclosure comprises from 5′ to 3′: a first inverted terminal repeat (ITR) sequence, a U1 promoter sequence, a CUG repeat targeting U7 esnRNA sequence, a U1 downstream terminator sequence, a buffer sequence, a U7 promoter sequence, a CUG repeat targeting U7 esnRNA sequence, a U7 downstream terminator sequence, an eCMV promoter sequence, a sequence encoding GFP, a polyA sequence, and a second ITR sequence. In some aspects, a nucleic acid sequence encoding AAV vector A03682 encoding a CUG repeat targeting snRNA sequences comprises SEQ ID NO: 140.

A03682: pAAV(ssAAV)-U1 Promoter-zCUGx15-U1 Term_U7 Promoter-zCUGx15-U7 Term-eCMV-eGFP

ITR
(SEQ ID NO: 59)
cctgcaggcagctgcgcgctcgctcgctcactgaggccgcccgggcgtcg
ggcgacctttggtcgcccggcctcagtgagcgagcgagcgcgcagagagg
gagtggccaactccatcactaggggttcct
U1 promoter
(SEQ ID NO: 40)
TAAGGACCAGCTTCTTTGGGAGAGAACAGACGCAGGGGCGGGAGG
GAAAAAGGGAGAGGCAGACGTCACTTCCCCTTGGCGGCTCTGGCAG
CAGATTGGTCGGTTGAGTGGCAGAAAGGCAGACGGGGACTGGGCA
AGGCACTGTCGGTGACATCACGGACAGGGCGACTTCTATGTAGATG
AGGCAGCGCAGAGGCTGACGTCTTCGCCACTTGCTGCTTCACCACG
AAGGAGTTCCCGTGCCCTGGGAGCGGGTTCAGGACCGCTGATCGGA
AGTGAGAATCCCAGCTGTGTGTCAGGGCTGGAAAGGGCTCGGGAGT
GCGCGGGGCAAGTGACCGTGTGTGTAAAGAGTGAGGCGTATGAGG
CTGTGTCGGGGCAGAGGCacaacgtttc
ZCUGx15 esnRNA
(SEQ ID NO: 65)
ggagtCAGCAGCAGCAGCAGCAGCAGCAGCAGCAGCAGCAGCAGCAG
CAGAATTTTTGGAGTAggctttctggctccttaccggaaagcc
U1 termination
(SEQ ID NO: 50)
ACTTTCTGGAGTTTCAAAAACAGACTGTACGCCAAGGGTCATATCTT
TTT
U7 promoter
(SEQ ID NO: 39)
AATCCAGGTCTCCGGGCTTAACAACAACGAAGGGGCTGTGACTGGC
TGCTTTCTCAACCAATCAGCACCGAACTCATTTGCATGGGCTGAGA
ACAAATGTTCGCGAACTCTAGAAATGAATGACTTAAGTAAGTTCCT
TAGAATATTATTTTTCCTACTGAAAGTTACCACATGCGTCGTTGTTT
ATACAGTAATAGGAACAAGAAAAAAGTCACCTAAGCTCACCCTCAT
CAATTGTGGAGTTCCTTTATATCCCATCTTCTCTCCAAACACATACG
CA
zCUGx15 esnRNA
(SEQ ID NO: 65)
ggagtCAGCAGCAGCAGCAGCAGCAGCAGCAGCAGCAGCAGCAGCAG
CAGAATTTTTGGAGTAggctttctggctccttaccggaaagcc
U7 termination
(SEQ ID NO: 49)
CCTCTTATGATGTTTGTTGCCAATGATAGATTGTTTTCACTGTGCAA
AAATTATGGGTAGTTTTGGTGGTCTTGATGCAGTTGTAAGCTTGGAG
eCMV promoter
(SEQ ID NO: 62)
cgttacataacttacggtaaatggcccgcctggctgaccgcccaacgacc
cccgcccattgacgtcaataatgacgtatgttcccatagtaacgccaata
gggactttccattgacgtcaatgggtggagtatttacggtaaactgccca
cttggcagtacatcaagtgtatcatatgccaagtacgccccctattgacg
tcaatgacggtaaatggcccgcctggcattatgcccagtacatgacctta
tgggactttcctacttggcagtacatctacgtattagtcatcgctattac
catg
eGFP
(SEQ ID NO: 63)
ATGgtgagcaagggcgaggagctgttcaccggggtggtgcccatcctggt
cgagctggacggcgacgtaaacggccacaagttcagcgtgtccggcgagg
gcgagggcgatgccacctacggcaagctgaccctgaagttcatctgcacc
accggcaagctgcccgtgccctggcccaccctcgtgaccaccctgaccta
cggcgtgcagtgcttcagccgctaccccgaccacatgaagcagcacgact
tcttcaagtccgccatgcccgaaggctacgtccaggagcgcaccatcttc
ttcaaggacgacggcaactacaagacccgcgccgaggtgaagttcgaggg
cgacaccctggtgaaccgcatcgagctgaagggcatcgacttcaaggagg
acggcaacatcctggggcacaagctggagtacaactacaacagccacaac
gtctatatcatggccgacaagcagaagaacggcatcaaggtgaacttcaa
gatccgccacaacatcgaggacggcagcgtgcagctcgccgaccactacc
agcagaacacccccatcggcgacggccccgtgctgctgcccgacaaccac
tacctgagcacccagtccgccctgagcaaagaccccaacgagaagcgcga
tcacatggtcctgctggagttcgtgaccgccgccgggatcactctcggca
tggacgagctgtacaagTAA
SV40 pA
(SEQ ID NO: 64)
aacttgtttattgcagcttataatggttacaaataaagcaatagcatcac
aaatttcacaaataaagcatttttttcactgcattctagttgtggtttgt
ccaaactcatcaatgtatctta
ITR
(SEQ ID NO: 60)
aggaacccctagtgatggagttggccactccctctctgcgcgctcgctcg
ctcactgaggccgggcgaccaaaggtcgcccgacgcccgggctttgcccg
ggggcctcagtgagcgagcgagcgcgcagctgcctgcagg

An exemplary AAV vector of the disclosure comprises from 5′ to 3′: a first inverted terminal repeat (ITR) sequence, a U7 promoter sequence, a non-targeting U7 esnRNA sequence, a U7 downstream terminator sequence, a buffer sequence, a U1 promoter sequence, a non-targeting U7 esnRNA sequence, a U1 downstream terminator sequence, an eCMV promoter sequence, a sequence encoding GFP, a polyA sequence, and a second ITR sequence. In some aspects, a nucleic acid sequence encoding AAV vector A03683 encoding a non-targeting snRNA sequences comprises SEQ ID NO: 141.

A03683: pAAV(ssAAV)-U7 Promoter-Znt-U7 Term_U1 Promoter-Znt-U1 Term-eCMV-eGFP

ITR
(SEQ ID NO: 59)
cctgcaggcagctgcgcgctcgctcgctcactgaggccgcccgggcgtcg
ggcgacctttggtcgcccggcctcagtgagcgagcgagcgcgcagagagg
gagtggccaactccatcactaggggttcct
U7 promoter
(SEQ ID NO: 39)
TACTGCCGAATCCAGGTCTCCGGGCTTAACAACAACGAAGGGGCTG
TGACTGGCTGCTTTCTCAACCAATCAGCACCGAACTCATTTGCATGG
GCTGAGAACAAATGTTCGCGAACTCTAGAAATGAATGACTTAAGTA
AGTTCCTTAGAATATTATTTTTCCTACTGAAAGTTACCACATGCGTC
GTTGTTTATACAGTAATAGGAACAAGAAAAAAGTCACCTAAGCTCA
CCCTCATCAATTGTGGAGTTCCTTTATATCCCATCTTCTCTCCAAAC
ACATACGCA
z-nt esnRNA
(SEQ ID NO: 66)
ggagtTCACCAGAAGCGTACCATACTCACGAAATTTTTGGAGTAggcttt
ctggctccttaccggaaagcc
U7 termination
(SEQ ID NO: 49)
CCTCTTATGATGTTTGTTGCCAATGATAGATTGTTTTCACTGTGCAA
AAATTATGGGTAGTTTTGGTGGTCTTGATGCAGTTGTAAGCTTGGAG
U1 promoter
(SEQ ID NO: 40)
TAAGGACCAGCTTCTTTGGGAGAGAACAGACGCAGGGGCGGGAGG
GAAAAAGGGAGAGGCAGACGTCACTTCCCCTTGGCGGCTCTGGCAG
CAGATTGGTCGGTTGAGTGGCAGAAAGGCAGACGGGGACTGGGCA
AGGCACTGTCGGTGACATCACGGACAGGGCGACTTCTATGTAGATG
AGGCAGCGCAGAGGCTGACGTCTTCGCCACTTGCTGCTTCACCACG
AAGGAGTTCCCGTGCCCTGGGAGCGGGTTCAGGACCGCTGATCGGA
AGTGAGAATCCCAGCTGTGTGTCAGGGCTGGAAAGGGCTCGGGAGT
GCGCGGGGCAAGTGACCGTGTGTGTAAAGAGTGAGGCGTATGAGG
CTGTGTCGGGGCAGAGGCacaacgtttc
Z-nt esnRNA
(SEQ ID NO: 66)
ggagtTCACCAGAAGCGTACCATACTCACGAAATTTTTGGAGTAggcttt
ctggctccttaccggaaagcc
U1 termination
(SEQ ID NO: 50)
ACTTTCTGGAGTTTCAAAAACAGACTGTACGCCAAGGGTCATATCTTTTT
eCMV promoter
(SEQ ID NO: 62)
cgttacataacttacggtaaatggcccgcctggctgaccgcccaacgacc
cccgcccattgacgtcaataatgacgtatgttcccatagtaacgccaata
gggactttccattgacgtcaatgggtggagtatttacggtaaactgccca
cttggcagtacatcaagtgtatcatatgccaagtacgccccctattgacg
tcaatgacggtaaatggcccgcctggcattatgcccagtacatgacctta
tgggactttcctacttggcagtacatctacgtattagtcatcgctattac
catg
eGFP
(SEQ ID NO: 63)
ATGgtgagcaagggcgaggagctgttcaccggggtggtgcccatcctggt
cgagctggacggcgacgtaaacggccacaagttcagcgtgtccggcgagg
gcgagggcgatgccacctacggcaagctgaccctgaagttcatctgcacc
accggcaagctgcccgtgccctggcccaccctcgtgaccaccctgaccta
cggcgtgcagtgcttcagccgctaccccgaccacatgaagcagcacgact
tcttcaagtccgccatgcccgaaggctacgtccaggagcgcaccatcttc
ttcaaggacgacggcaactacaagacccgcgccgaggtgaagttcgaggg
cgacaccctggtgaaccgcatcgagctgaagggcatcgacttcaaggagg
acggcaacatcctggggcacaagctggagtacaactacaacagccacaac
gtctatatcatggcegacaagcagaagaacggcatcaaggtgaacttcaa
gatccgccacaacatcgaggacggcagegtgcagctcgccgaccactacc
agcagaacacccccatcggcgacggccccgtgctgctgcccgacaaccac
tacctgagcacccagtccgccctgagcaaagaccccaacgagaagcgcga
tcacatggtcctgctggagttcgtgaccgccgccgggatcactctcggca
tggacgagctgtacaagTAA
SV40 pA
(SEQ ID NO: 64)
aacttgtttattgcagcttataatggttacaaataaagcaatagcatcac
aaatttcacaaataaagcatttttttcactgcattctagttgtggtttgt
ccaaactcatcaatgtatctta
ITR
(SEQ ID NO: 60)
Aggaacccctagtgatggagttggccactccctctctgcgcgctcgctcg
ctcactgaggccgggcgaccaaaggtcgcccgacgcccgggctttgcccg
ggggcctcagtgagcgagcgagcgcgcagctgcctgcagg

An exemplary AAV vector of the disclosure comprises from 5′ to 3′: a first inverted terminal repeat (ITR) sequence, a U7 promoter sequence, a CUG repeat targeting U7 esnRNA sequence, a U7 downstream terminator sequence, a buffer sequence, a U1 promoter sequence, a CUG repeat targeting U7 esnRNA sequence, a U1 downstream terminator sequence, and a second ITR sequence. In some aspects, a nucleic acid sequence encoding AAV vector A03684 encoding a CUG repeat targeting snRNA sequences comprises SEQ ID NO: 142.

A03864: pAAV(ssAAV)-U7 Promoter-zCUGx15-U7 Term_U1 Promoter-zCUGx15-U1 Term (Minus GFP—Derivative of 3681)

ITR
(SEQ ID NO: 59)
cctgcaggcagctgcgcgctcgctcgctcactgaggccgcccgggcgtcg
ggcgacctttggtcgcccggcctcagtgagcgagcgagcgcgcagagagg
gagtggccaactccatcactaggggttcct
U7 promoter
(SEQ ID NO: 39)
TACTGCCGAATCCAGGTCTCCGGGCTTAACAACAACGAAGGGGCTG
TGACTGGCTGCTTTCTCAACCAATCAGCACCGAACTCATTTGCATGG
GCTGAGAACAAATGTTCGCGAACTCTAGAAATGAATGACTTAAGTA
AGTTCCTTAGAATATTATTTTTCCTACTGAAAGTTACCACATGCGTC
GTTGTTTATACAGTAATAGGAACAAGAAAAAAGTCACCTAAGCTCA
CCCTCATCAATTGTGGAGTTCCTTTATATCCCATCTTCTCTCCAAAC
ACATACGCA
ZCUGx15 esnRNA
(SEQ ID NO: 65)
ggagtCAGCAGCAGCAGCAGCAGCAGCAGCAGCAGCAGCAGCAGCAG
CAGAATTTTTGGAGTAggctttctggctccttaccggaaagcc
U7 termination
(SEQ ID NO: 49)
CCTCTTATGATGTTTGTTGCCAATGATAGATTGTTTTCACTGTGCAA
AAATTATGGGTAGTTTTGGTGGTCTTGATGCAGTTGTAAGCTTGGAG
U1 promoter
(SEQ ID NO: 40)
TAAGGACCAGCTTCTTTGGGAGAGAACAGACGCAGGGGCGGGAGG
GAAAAAGGGAGAGGCAGACGTCACTTCCCCTTGGCGGCTCTGGCAG
CAGATTGGTCGGTTGAGTGGCAGAAAGGCAGACGGGGACTGGGCA
AGGCACTGTCGGTGACATCACGGACAGGGCGACTTCTATGTAGATG
AGGCAGCGCAGAGGCTGACGTCTTCGCCACTTGCTGCTTCACCACG
AAGGAGTTCCCGTGCCCTGGGAGCGGGTTCAGGACCGCTGATCGGA
AGTGAGAATCCCAGCTGTGTGTCAGGGCTGGAAAGGGCTCGGGAGT
GCGCGGGGCAAGTGACCGTGTGTGTAAAGAGTGAGGCGTATGAGG
CTGTGTCGGGGCAGAGGCacaacgtttc
ZCUGx15 esnRNA
(SEQ ID NO: 65)
ggagtCAGCAGCAGCAGCAGCAGCAGCAGCAGCAGCAGCAGCAGCAG
CAGAATTTTTGGAGTAggctttctggctccttaccggaaagcc
U1 termination
(SEQ ID NO: 50)
ACTTTCTGGAGTTTCAAAAACAGACTGTACGCCAAGGGTCATATCTT
TTT
ITR
(SEQ ID NO: 60)
aggaacccctagtgatggagttggccactccctctctgcgcgctcgctcg
ctcactgaggccgggcgaccaaaggtcgcccgacgcccgggctttgcccg
ggggcctcagtgagcgagcgagcgcgcagctgcctgcagg

An exemplary AAV vector of the disclosure comprises from 5′ to 3′: a first inverted terminal repeat (ITR) sequence, a U7 promoter sequence, a CUG repeat targeting U7 esnRNA sequence, a U7 downstream terminator sequence, a buffer sequence, a U1 promoter sequence, a CUG repeat targeting U7 esnRNA sequence, a U1 downstream terminator sequence, and a second ITR sequence. In some aspects, a nucleic acid sequence encoding AAV vector A04233 encoding a CUG repeat targeting snRNA sequences comprises SEQ ID NO: 143.

A04233: pAAV(scAAV)-U7promoter-zCUGx15-U7termination-U1promoter-zCUGx15-U1termination (derivative of A03681 no GFP in scAAV)

ITR
(SEQ ID NO: 59)
cctgcaggcagctgcgcgctcgctcgctcactgaggccgcccgggcgtc
gggcgacctttggtcgcccggcctcagtgagcgagcgagcgcgcagaga
gggagtggccaactccatcactaggggttcct
U7 promoter
(SEQ ID NO: 39)
TACTGCCGAATCCAGGTCTCCGGGCTTAACAACAACGAAGGGGCTG
TGACTGGCTGCTTTCTCAACCAATCAGCACCGAACTCATTTGCATGG
GCTGAGAACAAATGTTCGCGAACTCTAGAAATGAATGACTTAAGTA
AGTTCCTTAGAATATTATTTTTCCTACTGAAAGTTACCACATGCGTC
GTTGTTTATACAGTAATAGGAACAAGAAAAAAGTCACCTAAGCTCA
CCCTCATCAATTGTGGAGTTCCTTTATATCCCATCTTCTCTCCAAAC
ACATACGCA
zCUGx15 esnRNA
(SEQ ID NO: 65)
ggagtCAGCAGCAGCAGCAGCAGCAGCAGCAGCAGCAGCAGCAGCAG
CAGAATTTTTGGAGTAggctttctggctccttaccggaaagcc
U7 termination
(SEQ ID NO: 49)
CCTCTTATGATGTTTGTTGCCAATGATAGATTGTTTTCACTGTGCAA
AAATTATGGGTAGTTTTGGTGGTCTTGATGCAGTTGTAAGCTTGGAG
U1 promoter
(SEQ ID NO: 40)
TAAGGACCAGCTTCTTTGGGAGAGAACAGACGCAGGGGCGGGAGG
GAAAAAGGGAGAGGCAGACGTCACTTCCCCTTGGCGGCTCTGGCAG
CAGATTGGTCGGTTGAGTGGCAGAAAGGCAGACGGGGACTGGGCA
AGGCACTGTCGGTGACATCACGGACAGGGCGACTTCTATGTAGATG
AGGCAGCGCAGAGGCTGACGTCTTCGCCACTTGCTGCTTCACCACG
AAGGAGTTCCCGTGCCCTGGGAGCGGGTTCAGGACCGCTGATCGGA
AGTGAGAATCCCAGCTGTGTGTCAGGGCTGGAAAGGGCTCGGGAGT
GCGCGGGGCAAGTGACCGTGTGTGTAAAGAGTGAGGCGTATGAGG
CTGTGTCGGGGCAGAGGCacaacgtttc
ZCUGx15 esnRNA
(SEQ ID NO: 65)
ggagtCAGCAGCAGCAGCAGCAGCAGCAGCAGCAGCAGCAGCAGCAG
CAGAATTTTTGGAGTAggctttctggctccttaccggaaagcc
U1 termination
(SEQ ID NO: 50)
ACTTTCTGGAGTTTCAAAAACAGACTGTACGCCAAGGGTCATATCTT
TTT
mITR
(SEQ ID NO: 61)
CCACTCCCTCTCTGCGCGCTCGCTCGCTCACTGAGGCCGGGCGACCA
AAGGTCGCCCGACGCCCGGGCTTTGCCCGGGCGGCCTCAGTGAGCG
AGCGAGCGCGCAGAGAGGGA

### Promoter Sequences

Gene therapy and RNA-targeting snRNA gene therapy compositions of the disclosure comprise promoter sequences derived from an snRNA.

A “promoter” is a control sequence that is a region of a polynucleotide sequence at which initiation and rate of transcription are controlled. It may contain genetic elements at which regulatory proteins and molecules may bind such as RNA polymerase and other transcription factors.

In some embodiments of the compositions and methods of the disclosure, an expression vector, viral vector or non-viral vector provided herein, includes without limitation, an expression control element. An “expression control element” as used herein refers to any sequence that regulates the expression of a coding sequence, such as a gene. Exemplary expression control elements include but are not limited to promoters, enhancers, microRNAs, post-transcriptional regulatory elements, polyadenylation signal sequences, and introns. Expression control elements may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors. An “enhancer” is a region of DNA that can be bound by activating proteins to increase the likelihood or frequency of transcription.

In some embodiments of the compositions and methods of the disclosure, an expression vector, viral vector or non-viral vector provided herein, includes without limitation, vector elements such as a buffer sequence derived human genomic sequences downstream from an snRNA and as such will have the capability to encoding multiple snRNAs from a single construct.

In some embodiments, with the incorporation of coding sequences into the compositions disclosed herein, multicistronic vectors can simultaneously express two or more separate proteins from the same mRNA. The two strategies most widely used for constructing multicistronic configurations are through the use of an IRES or a 2A self-cleaving site. An “IRES” refers to an internal ribosome entry site or portion thereof of viral, prokaryotic, or eukaryotic origin which are used within polycistronic vector constructs. In some embodiments, an IRES is an RNA element that allows for translation initiation in a cap-independent manner. The term “self-cleaving peptides” or “sequences encoding self-cleaving peptides” or “2A self-cleaving site” refer to linking sequences which are used within vector constructs to incorporate sites to promote ribosomal skipping and thus to generate two polypeptides from a single promoter, such self-cleaving peptides include without limitation, T2A, and P2A peptides or other sequences encoding the self-cleaving peptides.

In another embodiment, the vector configurations can comprise linker(s), signal sequence(s), and/or tag(s).

In some embodiments, the vector is a viral vector. In some embodiments, the vector is an adenoviral vector, an adeno-associated viral (AAV) vector, or a lentiviral vector. In some embodiments, the vector is a retroviral vector, an adenoviral/retroviral chimera vector, a herpes simplex viral I or II vector, a parvoviral vector, a reticuloendotheliosis viral vector, a polioviral vector, a papillomaviral vector, a vaccinia viral vector, or any hybrid or chimeric vector incorporating favorable aspects of two or more viral vectors. In some embodiments, the vector further comprises one or more expression control elements operably linked to the polynucleotide. In some embodiments, the vector further comprises one or more selectable markers. In some embodiments, the AAV vector has low toxicity. In some embodiments, the AAV vector does not incorporate into the host genome, thereby having a low probability of causing insertional mutagenesis. In some embodiments, the AAV vector can encode a range of total polynucleotides from 4.5 kb to 4.75 kb. In some embodiments, exemplary AAV vectors that may be used in any of the herein described compositions, systems, methods, and kits can include an AAV1 vector, a modified AAV1 vector, an AAV2 vector, a modified AAV2 vector, an AAV2-Tyr mutant vector, an AAV3 vector, a modified AAV3 vector, an AAV4 vector, a modified AAV4 vector, an AAV5 vector, a modified AAV5 vector, an AAV6 vector, a modified AAV6 vector, an AAV7 vector, a modified AAV7 vector, an AAV8 vector, an AAVrh8 vector, an AAV9 vector, an AAV.rh10 vector, a modified AAV.rh10 vector, an AAVrh.74, an AAV.rh32/33 vector, a modified AAV.rh32/33 vector, an AAV.rh43 vector, a modified AAV.rh43 vector, an AAV.rh64R1 vector, and a modified AAV.rh64R1 vector, an AAV-Tyr mutant vector, AAV-Tyr-Ser mutant vector, AAV-Tyr-Ser-Thr mutant vector and any combinations or equivalents thereof. In some embodiments, the lentiviral vector is an integrase-competent lentiviral vector (ICLV). In some embodiments, the lentiviral vector can refer to the transgene plasmid vector as well as the transgene plasmid vector in conjunction with related plasmids (e.g., a packaging plasmid, a rev expressing plasmid, an envelope plasmid) as well as a lentiviral-based particle capable of introducing exogenous nucleic acid into a cell through a viral or viral-like entry mechanism.

Lentiviral vectors are well-known in the art (see, e.g., Trono D. (2002) Lentiviral vectors, New York: Spring-Verlag Berlin Heidelberg and Durand et al. (2011) Viruses 3(2):132-159 doi: 10.3390/v3020132). In some embodiments, exemplary lentiviral vectors that may be used in any of the herein described compositions, systems, methods, and kits can include a human immunodeficiency virus (HIV) 1 vector, a modified human immunodeficiency virus (HIV) 1 vector, a human immunodeficiency virus (HIV) 2 vector, a modified human immunodeficiency virus (HIV) 2 vector, a sooty mangabey simian immunodeficiency virus (SIV_{SM}) vector, a modified sooty mangabey simian immunodeficiency virus (SIV_{SM}) vector, a African green monkey simian immunodeficiency virus (SIV_{AGM}) vector, a modified African green monkey simian immunodeficiency virus (SIV_{AGM}) vector, an equine infectious anemia virus (EIAV) vector, a modified equine infectious anemia virus (EIAV) vector, a feline immunodeficiency virus (FIV) vector, a modified feline immunodeficiency virus (FIV) vector, a Visna/maedi virus (VNV/VMV) vector, a modified Visna/maedi virus (VNV/VMV) vector, a caprine arthritis-encephalitis virus (CAEV) vector, a modified caprine arthritis-encephalitis virus (CAEV) vector, a bovine immunodeficiency virus (BIV), or a modified bovine immunodeficiency virus (BIV).

### Nucleic Acids

An NOI (nucleotide sequence of interest) includes, without limitation, any nucleotide sequence or transgene capable of being delivered by a vector. NOIs can be synthetic, derived from naturally occurring DNA or RNA, codon optimized, recombinant RNA/DNA, cDNA, partial genomic DNA, and/or combinations thereof. The NOI can be a coding region or partial coding region, but need not be a coding region. An NOI can be RNA/DNA in a sense or anti-sense orientation. An NOI can be an snRNA. NOIs are also referred herein, without limitation, as transgenes, heterologous sequences, genes, therapeutic genes. An NOI may also encode an RNA (ribonucleoprotein complex) a POI (protein of interest), a partial POI, a mutated version or variant of a POI. A POI may be analogous to or correspond to a wild-type protein. A POI may also be a fusion protein or ribonucleoprotein complex such as an snRNP. In some aspects, RNA sequences disclosed herein may be represented as DNA sequences and it is within the ability of the skilled artisan to derive the sequence of an RNA sequence from a DNA sequence.

### Codon Optimization

In some embodiments, NOIs or transgenes or GOIs such as nucleic acid sequences encoding RNA-targeting snRNAs of the disclosure are codon optimized nucleic acid sequences. In some embodiments, the codon optimized sequence exhibits at least 5%, at least 10%, at least 20%, at least 30%, at least 50%, at least 75%, at least 100%, at least 200%, at least 300%, at least 500%, or at least 1000% increased transcription or translation in a human subject relative to a wild-type or non-codon optimized nucleic acid sequence.

In some aspects, a codon optimized nucleic acid sequence exhibits increased stability. In some aspects, a codon optimized nucleic acid sequence exhibits increased stability through increased resistance to hydrolysis. In some embodiments, the codon optimized sequence exhibits at least 5%, at least 10%, at least 20%, at least 30%, at least 50%, at least 75%, at least 100%, at least 200%, at least 300%, at least 500%, or at least 1000% increased stability relative to a wild-type or non-codon optimized nucleic acid sequence. In some embodiments, the codon optimized sequence exhibits at least 5%, at least 10%, at least 20%, at least 30%, at least 50%, at least 75%, at least 100%, at least 200%, at least 300%, at least 500%, or at least 1000% increased resistance to hydrolysis in a human subject relative to a wild-type or non-codon optimized nucleic acid sequence.

In some aspects, a codon optimized nucleic acid sequence can comprise no donor splice sites. In some aspects, a codon optimized nucleic acid sequence can comprise no more than about one, or about two, or about three, or about four, or about five, or about six, or about seven, or about eight, or about nine, or about ten donor splice sites. In some aspects, a codon optimized nucleic acid sequence comprises at least one, or at least two, or at least three, or at least four, or at least five, or at least six, or at least seven, or at least eight, or at least nine, or at least ten fewer donor splice sites as compared to a non-codon optimized nucleic acid sequence.

Without wishing to be bound by theory, the removal of donor splice sites in the codon optimized nucleic acid sequence can unexpectedly and unpredictably increase expression of protein of interest in vivo, as cryptic splicing is prevented. Moreover, cryptic splicing may vary between different subjects, meaning that the expression level of a protein comprising donor splice sites may unpredictably vary between different subjects. Such unpredictability is unacceptable in the context of human therapy. Accordingly, the codon optimized nucleic acid sequences which lacks donor splice sites, unexpectedly and surprisingly allows for increased expression of the protein in human subjects and regularizes expression of the protein across different human subjects.

In some aspects, a codon optimized nucleic acid sequence can have a GC content that differs from the GC content of the non-codon optimized nucleic acid sequence encoding the RNA-targeting snRNA. In some aspects, the GC content of a codon optimized nucleic acid sequence is more evenly distributed across the entire nucleic acid sequence, as compared to the non-codon optimized nucleic acid sequence.

Without wishing to be bound by theory, by more evenly distributing the GC content across the entire nucleic acid sequence, the codon optimized nucleic acid sequence exhibits a more uniform melting temperature (“Tm”) across the length of the transcript. The uniformity of melting temperature results unexpectedly in increased expression of the codon optimized nucleic acid in a human subject, as transcription and/or translation of the nucleic acid sequence occurs with less stalling of the polymerase and/or ribosome.

In some aspects, a codon optimized nucleic acid sequence can have fewer repressive microRNA target binding sites as compared to the non-codon optimized nucleic acid sequence. In some aspects, a codon optimized nucleic acid sequence can have at least one, or at least two, or at least three, or at least four, or at least five, or at least six, or at least seven, or at least eight, or at least nine, or at least ten, or at least ten fewer repressive microRNA target binding sites as compared to the non-codon optimized nucleic acid sequence.

Without wishing to be bound by theory, by having fewer repressive microRNA target binding sites, the codon optimized nucleic acid sequence unexpectedly exhibits increased expression in a human subject.

Provided herein are the nucleic acid sequences encoding the gene therapy compositions or RNA-targeting snRNA systems for use in gene transfer and expression techniques described herein. It should be understood, although not always explicitly stated that the sequences provided herein can be used to provide the expression product as well as substantially identical sequences that encode an RNA or express and produce a protein that has the same biological properties. These “biologically equivalent” or “biologically active” or “equivalent” polypeptides are encoded by equivalent polynucleotides as described herein. They may possess at least 60%, or alternatively, at least 65%, or alternatively, at least 70%, or alternatively, at least 75%, or alternatively, at least 80%, or alternatively at least 85%, or alternatively at least 90%, or alternatively at least 95% or alternatively at least 98%, identical primary amino acid sequence to the reference polypeptide when compared using sequence identity methods run under default conditions. Specific polypeptide sequences are provided as examples of particular embodiments. Modifications to the sequences to amino acids with alternate amino acids that have similar charge. Additionally, an equivalent polynucleotide is one that hybridizes under stringent conditions to the reference polynucleotide or its complement or in reference to a polypeptide, a polypeptide encoded by a polynucleotide that hybridizes to the reference encoding polynucleotide under stringent conditions or its complementary strand. Alternatively, an equivalent polypeptide or protein is one that is expressed from an equivalent polynucleotide.

The NOIs or nucleic acid sequences (e.g., polynucleotide sequences) disclosed herein may be codon-optimized which is a technique well known in the art. Codon optimization refers to the fact that different cells differ in their usage of particular codons. This codon bias corresponds to a bias in the relative abundance of particular tRNAs in the cell type. By altering the codons in the sequence to match with the relative abundance of corresponding tRNAs, it is possible to increase expression. It is also possible to decrease expression by deliberately choosing codons for which the corresponding tRNAs are known to be rare in a particular cell type. Codon usage tables are known in the art for mammalian cells, as well as for a variety of other organisms. Based on the genetic code, nucleic acid sequences coding for, e.g., an esnRNA, esnRNA can be generated. In some embodiments, such a sequence is optimized for expression in a host or target cell, such as a host cell used to express the esnRNA or a cell in which the disclosed methods are practiced (such as in a mammalian cell, e.g., a human cell). Codon preferences and codon usage tables for a particular species can be used to engineer isolated nucleic acid molecules encoding an esnRNA that takes advantage of the codon usage preferences of that particular species. For example, the esnRNA disclosed herein can be designed to have codons that are preferentially used by a particular organism of interest. In one example, an esnRNA nucleic acid sequence is optimized for expression in human cells, such as one having at least 70%, at least 80%, at least 85%, at least 90%, at least 92%, at least 95%, at least 98%, or at least 99% sequence identity to its corresponding wild-type or originating nucleic acid sequence. In some embodiments, an isolated nucleic acid molecule encoding at least one esnRNA (which can be part of a vector) includes at least one esnRNA coding sequence that is codon optimized for expression in a eukaryotic cell, or at least one esnRNA coding sequence codon optimized for expression in a human cell. In one embodiment, such a codon optimized esnRNA coding sequence has at least 80%, at least 85%, at least 90%, at least 92%, at least 95%, at least 96%, at least 97%, at least 98%, at least 99%, or 100% sequence identity to its corresponding wild-type or originating sequence. In another embodiment, a eukaryotic cell codon optimized nucleic acid sequence encodes esnRNA having at least 85%, at least 90%, at least 92%, at least 95%, at least 96%, at least 97%, at least 98%, at least 99%, or 100% sequence identity to its corresponding wild-type or originating sequence. In another embodiment, a variety of clones containing functionally equivalent nucleic acids may be routinely generated, such as nucleic acids which differ in sequence but which encode the same esnRNA sequence. Silent mutations in the coding sequence result from the degeneracy (i.e., redundancy) of the genetic code, whereby more than one codon can encode the same amino acid residue. Thus, for example, leucine can be encoded by CTT, CTC, CTA, CTG, TTA, or TTG; serine can be encoded by TCT, TCC, TCA, TCG, AGT, or AGC; asparagine can be encoded by AAT or AAC; aspartic acid can be encoded by GAT or GAC; cysteine can be encoded by TGT or TGC; alanine can be encoded by GCT, GCC, GCA, or GCG; glutamine can be encoded by CAA or CAG; tyrosine can be encoded by TAT or TAC; and isoleucine can be encoded by ATT, ATC, or ATA. Tables showing the standard genetic code can be found in various sources (see, for example, Stryer, 1988, Biochemistry, 3.sup.rd Edition, W.H. 5 Freeman and Co., NY).

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PC reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10× SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous”sequence shares less than 40% identity, or alternatively less than 25% identity, with one of the sequences of the present invention.

### Cells

In some embodiments of the compositions and methods of the disclosure, a cell of the disclosure is a prokaryotic cell.

In some embodiments of the compositions and methods of the disclosure, a cell of the disclosure is a eukaryotic cell. In some embodiments, the cell is a mammalian cell. In some embodiments, the cell is a bovine, murine, feline, equine, porcine, canine, simian, or human cell. In some embodiments, the cell is a non-human mammalian cell such as a non-human primate cell.

In some embodiments, a cell of the disclosure is a somatic cell. In some embodiments, a cell of the disclosure is a germline cell. In some embodiments, a germline cell of the disclosure is not a human cell.

In some embodiments of the compositions and methods of the disclosure, a cell of the disclosure is a stem cell. In some embodiments, a cell of the disclosure is an embryonic stem cell. In some embodiments, an embryonic stem cell of the disclosure is not a human cell. In some embodiments, a cell of the disclosure is a multipotent stem cell or a pluripotent stem cell. In some embodiments, a cell of the disclosure is an adult stem cell. In some embodiments, a cell of the disclosure is an induced pluripotent stem cell (iPSC). In some embodiments, a cell of the disclosure is a hematopoietic stem cell (HSC).

In some embodiments of the compositions and methods of the disclosure, a somatic cell of the disclosure is a neuronal cell. In one embodiment, a cell or cells of a patient treated with compositions disclosed herein include, without limitation, central nervous system (neurons), peripheral nervous system (neurons), peripheral motor neurons, and/or sensory neurons. In one embodiment, a neuronal cell is a glial cell.

In some embodiments of the compositions and methods of the disclosure, a somatic cell of the disclosure is a fibroblast or an epithelial cell. In some embodiments, an epithelial cell of the disclosure forms a squamous cell epithelium, a cuboidal cell epithelium, a columnar cell epithelium, a stratified cell epithelium, a pseudostratified columnar cell epithelium or a transitional cell epithelium. In some embodiments, an epithelial cell of the disclosure forms a gland including, but not limited to, a pineal gland, a thymus gland, a pituitary gland, a thyroid gland, an adrenal gland, an apocrine gland, a holocrine gland, a merocrine gland, a serous gland, a mucous gland and a sebaceous gland. In some embodiments, an epithelial cell of the disclosure contacts an outer surface of an organ including, but not limited to, a lung, a spleen, a stomach, a pancreas, a bladder, an intestine, a kidney, a gallbladder, a liver, a larynx or a pharynx. In some embodiments, an epithelial cell of the disclosure contacts an outer surface of a blood vessel or a vein.

In some embodiments of the disclosure, a somatic cell is an ocular cell. An ocular cell includes, without limitation, corneal epithelial cells, keratyocytes, retinal pigment epithelial (RPE) cells, lens epithelial cells, iris pigment epithelial cells, conjunctival fibroblasts, non-pigmented ciliary epithelial cells, trabecular meshwork cells, ocular choroid fibroblasts, conjunctival epithelial cells, In some embodiments, an ocular cell is a retinal cell or a corneal cell. In one embodiment, a retinal cell is a photoreceptor cell or a retinal pigment epithelial cell. In another embodiment, a retinal cell is a ganglion cell, an amacrine cell, a bipolar cell, a horizontal cell, a Müller glial cell, a rod cell, or a cone cell. In some embodiments of the compositions and methods of the disclosure, a somatic cell of the disclosure is a primary cell.

In some embodiments of the compositions and methods of the disclosure, a somatic cell of the disclosure is a cultured cell.

In some embodiments of the compositions and methods of the disclosure, a somatic cell of the disclosure is in vivo, in vitro, ex vivo or in situ.

In some embodiments of the compositions and methods of the disclosure, a somatic cell of the disclosure is autologous or allogeneic.

### Methods of Use

The disclosure provides a method of encoding an RNA or expressing an NOI in a cell using the snRNA systems disclosed herein. In one embodiment, the disclosure provides a method of modifying an RNA or the activity of a protein encoded by an RNA molecule comprising contacting the composition of the disclosure and the target RNA molecule under conditions suitable for binding to the RNA molecule.

The disclosure provides a method of modifying the level of expression of an RNA molecule of the disclosure or a protein encoded by the RNA molecule comprising contacting the composition of the disclosure and a cell comprising the RNA molecule under conditions suitable for binding to the RNA molecule. In some embodiments, the cell is in vivo, in vitro, ex vivo or in situ. In some embodiments, the composition of the disclosure comprises a vector comprising snRNA sequences. In some embodiments, the vector is an AAV.

The disclosure provides a method of modifying the level of expression of an RNA molecule of the disclosure or a protein encoded by the RNA molecule comprising contacting the composition of the disclosure and the RNA molecule under conditions suitable for knocking down, blocking, splicing, multi-targeting, or editing the target RNA. In some embodiments, the vector is an AAV.

The disclosure provides a method of modifying a target RNA or an activity of a protein encoded by an RNA molecule comprising contacting the composition and a cell comprising the RNA molecule under conditions suitable knocking down, blocking, splicing, multi-targeting, or editing the target RNA. In some embodiments, the cell is in vivo, in vitro, ex vivo or in situ. In some embodiments, the composition comprises a vector comprising the snRNA sequences disclosed herein. In some embodiments, the vector is an AAV.

The disclosure provides a method of treating a disease or disorder comprising administering to a subject a therapeutically effective amount of an snRNA composition of the disclosure.

The disclosure provides a method of treating a disease in a patient in need of such treatment comprising administering to the patient a therapeutically effective amount of an snRNA composition of the disclosure, wherein the composition comprises a vector comprising snRNA sequences disclosed herein, wherein the composition modifies, reduces, destroys, knocks down or ablates a level of expression of a toxic repeat RNA (compared to the level of expression of a toxic repeat RNA treated with a non-targeting (NT) control or compared to no treatment). In another embodiment, the level of reduction is 1-fold or greater. In another embodiment, the level of reduction is 2-fold, 3-fold, 4-fold, 5-fold, 6-fold, 7-fold, 8-fold, 9-fold or 10-fold. In another embodiment, the level of reduction is 10-fold or greater. In another embodiment, the level of reduction is between 10-fold and 20-fold. In another embodiment, the level of reduction is 11-fold, 12-fold, 13-fold, 14-fold, 15-fold, 16-fold, 17-fold, 18-fold, 19-fold, or 20-fold. In another embodiment, the gene therapy compositions disclosed herein when administered to a patient lead to 20%-100% destruction of the toxic repeat RNA. In one embodiment, the % elimination of the toxic repeat RNA is any of 20-99%, 25%-99%, 50%-99%, 80%-99%, 90%-99%, 95%-99%. In one embodiment, the % elimination is 90%, 91%, 92%, 93%, 94%, 95%, 96%, 97%, 98%, or 99%. In another embodiment, % elimination is complete elimination or 100% elimination of the toxic repeat RNA.

The disclosure provides a method of treating a disease or disorder in a subject comprising administering an RNA-targeting nucleic acid molecule (i.e. an esnRNA of the disclosure) or an AAV vector comprising an esnRNA of the disclosure.

In some aspects, the disease or disorder is associated with a toxic repeat RNA sequence. In some aspects, the toxic repeat RNA sequence is a CAG, CUG, GGCCCC, CCGGG, or GGCCC+CCGGGG RNA repeat.

In some aspects, the disease or disorder is myotonic dystrophy (DM1) or Huntington's disease (HD).

In some aspects, the disease or disorder is Duchenne Muscular Dystrophy. In some aspects, the RNA-targeting nucleic acid molecule or AAV vector targets an RNA sequence encoding dystrophin (DMD). In some aspects, the RNA sequence encoding DMD comprises an intronic or exonic sequence. In some aspects, the exonic sequence comprises exon 51, or a flanking region thereof, of DMD.

In some embodiments of the methods of the disclosure, a subject of the disclosure has been diagnosed with a disease to be treated. In some embodiments, the subject of the disclosure presents at least one sign or symptom of a disorder or disease to be treated. In some embodiments, the subject of the disclosure presents at least one sign or symptom of a disease.

In some embodiments of the methods of the disclosure, a subject of the disclosure is female. In some embodiments of the methods of the disclosure, a subject of the disclosure is male. In some embodiments, a subject of the disclosure has two XX or XY chromosomes. In some embodiments, a subject of the disclosure has two XX or XY chromosomes and a third chromosome, either an X or a Y.

In some embodiments of the methods of the disclosure, a subject of the disclosure is a neonate, an infant, a child, an adult, a senior adult, or an elderly adult. In some embodiments of the methods of the disclosure, a subject of the disclosure is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 days old. In some embodiments of the methods of the disclosure, a subject of the disclosure is at least 1, 2, 3, 4, 5, 6, 7, 8, 9, 10, 11 or 12 months old. In some embodiments of the methods of the disclosure, a subject of the disclosure is at least 1, 2, 3, 4, 5, 6, 7, 8, 9, 10, 15, 20, 25, 30, 35, 40, 45, 50, 55, 60, 65, 70, 75, 80, 85, 90, 95, 100 or any number of years or partial years in between of age.

In some embodiments of the methods of the disclosure, a subject of the disclosure is a mammal. In some embodiments, a subject of the disclosure is a non-human mammal.

In some embodiments of the methods of the disclosure, a subject of the disclosure is a human.

In some embodiments of the methods of the disclosure, a therapeutically effective amount comprises a single dose of a composition of the disclosure. In some embodiments, a therapeutically effective amount comprises a therapeutically effective amount comprises at least one dose of a composition of the disclosure. In some embodiments, a therapeutically effective amount comprises a therapeutically effective amount comprises one or more dose(s) of a composition of the disclosure.

In some embodiments of the methods of the disclosure, a therapeutically effective amount eliminates a sign or symptom of the disease or disorder. In some embodiments, a therapeutically effective amount reduces a severity of a sign or symptom of the disease or disorder.

In some embodiments of the methods of the disclosure, a therapeutically effective amount eliminates the disease or disorder.

In some embodiments of the methods of the disclosure, a therapeutically effective amount prevents an onset of a disease or disorder. In some embodiments, a therapeutically effective amount delays the onset of a disease or disorder. In some embodiments, a therapeutically effective amount reduces the severity of a sign or symptom of the disease or disorder. In some embodiments, a therapeutically effective amount improves a prognosis for the subject.

In some embodiments of the methods of the disclosure, a composition of the disclosure is administered to the subject via intracerebral administration. In some embodiments, the composition of the disclosure is administered to the subject by an intrastriatal route. In some embodiments, the composition of the disclosure is administered to the subject by a stereotaxic injection or an infusion. In some embodiments, the composition is administered to the brain. In some embodiments of the methods of the disclosure, a composition of the disclosure is administered to the subject locally.

In some embodiments, the compositions disclosed herein are formulated as pharmaceutical compositions. Briefly, pharmaceutical compositions for use as disclosed herein may comprise a protein(s) or a polynucleotide encoding the protein(s), optionally comprised in an AAV, which is optionally also immune orthogonal, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the disclosure may be formulated for routes of administration, such as e.g., oral, enteral, topical, transdermal, intranasal, and/or inhalation; and for routes of administration via injection or infusion such as, e.g., intravenous, intramuscular, subpial, intrathecal, intraparenchymal, intrathecal, intrastriatal, subcutaneous, intradermal, intraperitoneal, intratumoral, intravenous, intraocular, and/or parenteral administration. In certain embodiments, the compositions of the present disclosure are formulated for intracerebral or intrastriatal administration.

### EXAMPLES

### Example 1: esnRNA Targeting CUG Repeats in DM1 Cell Model

### Materials and Methods:

CTG480 HeLa cells were seeded at 100,000 cells/ml in 12 well plate. Next day snRNAs were transfected using lipofectamine (2 μL P3000 and 2.5 μl lipofectamine per sample). 24 h our later cells were trypsinized and ½ of the cells seeded in chamber slides for FISH and the other ½ in a 24-well plate for RNA analysis.

Next day RNA was extracted for RNA analysis and cells were fixed for FISH. For FISH CUG foci was labeled with CUG fluorescent probe, and the cell nuclei was stained with DAPI.

Purpose: To determine whether engineered snRNPs comprising the engineered stem loop are capable of knocking down CUG expansions and therefore decrease the number of RNA foci in a DM1 cell model.

Rationale: The U7 snRNA can be programmed to target mRNAs by replacing its histone mRNA annealing sequence with a sequence complementary to a target of interest. U7 with 15×CAG repeats has been shown by others to reduce CUG repeat expansions, which cause DM1 (Frangois et al NSMB 2011). We optimized this snRNA system by engineering the stem loop (SEQ ID NO: 1) to better stabilize the secondary structure of U7 snRNA in cells. We compared our engineered U7 stem loop to the U7 stem loop known in the art (Frangois et al NSMB 2011). The constructs contain 15×CAG repeats and an optimized Sm binding domain, which further helps to stabilize the programmed U7. The CUG-targeting constructs were tested to determine whether they can deplete CUG foci by binding to and blocking CUG repeats in an appropriate cell model and compared to U7s that contained a non-targeting (NT) sequence. See FIGS. 1A-D and 2A-C.

Constructs and controls were transiently transfected into HeLa(CTG480) which has 480 CTG repeats inserted into its genome by CRISPR to model DM1 and express CUG foci as shown by in-situ hybridization. Cells were seeded and fixed onto chamber slides, and RNA in-situ hybridization was performed on the cells using a probe for CUG and dapi for nuclear staining. The co-localization of the CUG probe and DAPI signal was quantified using Halo.

Treatment with 15×CAG esnRNA constructs (to target/block CUG repeats) significantly decreased CUG RNA foci.

Compared to their respective non-targeting control, the non-engineered snRNA and esnRNA constructs led to a 39% and 68% reduction in average #foci/cell, respectively.

### Example 2: esnRNA Targeting CAG Repeats

### Materials and Methods:

HEK293T cells were seeded at 150,000 cells/ml in a 12 well plate. Next day snRNAs were transfected using lipofectamine (2 μL P3000 and 2.5 μl lipofectamine per sample). Cells were transfected with the CAG reporter construct (containing 80 CAG repeats) and increasing doses (from 100 to 1000 ng) of the snRNA plasmids targeting CAG repeats (referred as CUG-15) or the control non-targeting (nt).

RNA was extracted for RNA analysis 48 h post transfection using the QiaCube Connect. qRT-PCR was performed to detect the expression levels of the CAG reporter and normalized to the expression levels of the GAPDH reference gene.

Another set of cells was used for Western blot analysis with a myc tag antibody to detect the protein levels of Poly-Q CAG containing reporter. GAPDH was used as loading control.

Purpose: Mutant HTT is caused by the existence of microsatellite repeats in exon one of HTT. Here, we explored whether engineered snRNPs comprising the engineered stem loop are capable of knocking down CAG microsatellite repeat RNA in a CAG80 reporter assay.

### Rationale:

The U7 snRNA can be programmed to target mRNAs by replacing its histone mRNA annealing sequence with a sequence complementary to a target of interest. The esnRNA system reprogrammed to target CAG repeats comprises an eSL of SEQ ID NO: 1 to better stabilize the secondary structure of U7 snRNA in cells. The constructs contain 15×CUG repeats to target CAG expansions. These constructs also contained an optimized Sm binding domain, which further helps to stabilize the programmed U7. The CAG-targeting constructs were tested to determine whether they can knock down CAG repeats and poly-Q containing protein compared to eSLs that contained a non-targeting sequence.

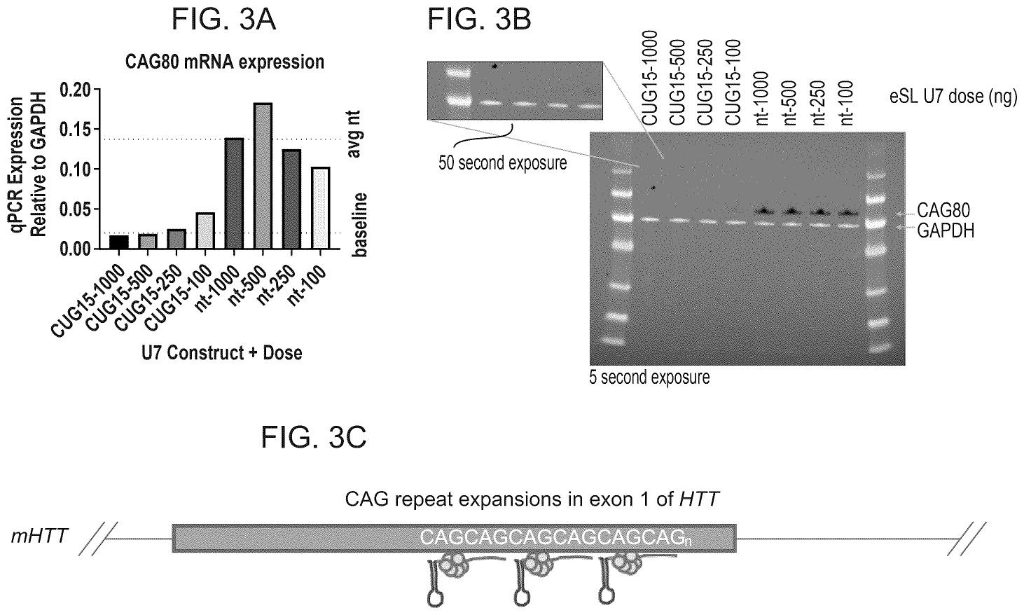

Complete knockdown of detectable PolyQ protein was achieved by the highest 3 doses of U7. An almost complete knockdown was achieved at the lowest U7 dose. Accordingly, we show herein a dose-dependent knockdown of CAG80 reporter mRNA to baseline levels. See FIGS. 3A-3C.

### Example 3: esnRNA Modulation of USH2A Minigene Exon 13 Splicing

### Materials and Methods:

HEK293T cells were seeded at 150,000/ml in 12 well plate. Next day cells were transfected with 50 ng of USH2A minigene with a total of 250 ng, 500 ng, 1 μg of pcDNA expressing non-targeting esnRNA or USH2A exon 13-targeting esnRNA with lipofectamine.

Cells were harvested 48 h post-transfection. RNA was extracted using Qiacube connect, reverse transcribed, and semiquantitative PCR performed with primers that anneal to USH2A exon 12 and 14.

Quantitation of U7 was performed by PCR with primers annealing to the non-targeting esnRNA.

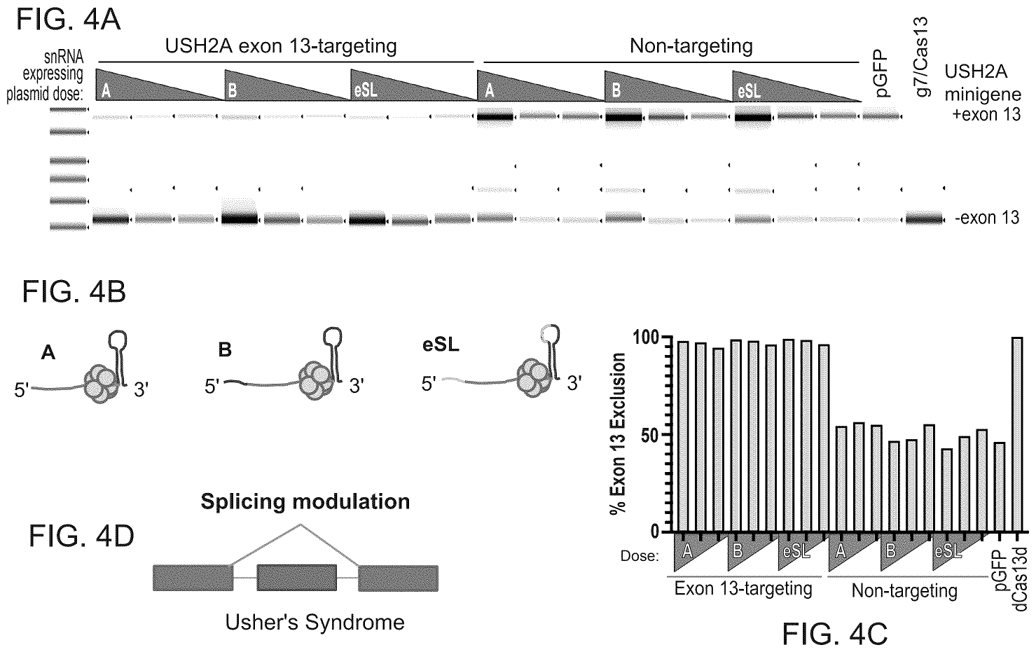

Rationale: USH2A exon 13 mutations lead to Usher Syndrome Type 2A. Exon 13 is an in-frame exon and a therapeutic strategy includes skipping exon 13. We designed esnRNAs comprising an eSL disclosed herein that target and anneal to exon splicing enhancers on exon 13 of USH2A and block the inclusion of exon 13. Our programmed esnRNA system to target USH2A exon 13 results in efficient exon 13 skipping on minigene. PCR results suggest higher steady state levels of the esnRNA system compared to non-engineered SL. See FIGS. 4A-4D. See also FIG. 1A.

### Example 4: Splicing Correction by esnRNA-Mediated CUG-Repeat Blocking in DM1 Patient Myotubes

### Materials and Methods:

DM1 patient myotubes containing 2600 CUG repeats in the 3′UTR of DMPK were differentiated for 13 days before introduction of esnRNA comprising eSL disclosed herein targeting 15×CUG repeats. RNA was extracted from cells 24 hours after treatment, RNA was reverse transcribed and semiquantitative PCR was performed to DM1 disease splicing biomarkers LDB3 exon 11, BIN1 exon 11 and DMD exon 78. Analysis of splicing isoforms was performed by calculating the percentage of the healthy splice isoform (LDB3 exon 11 excluded, BIN1 exon 11 included and DMD exon 78 included) over the total signal from both the disease and healthy isoforms.

### Rationale:

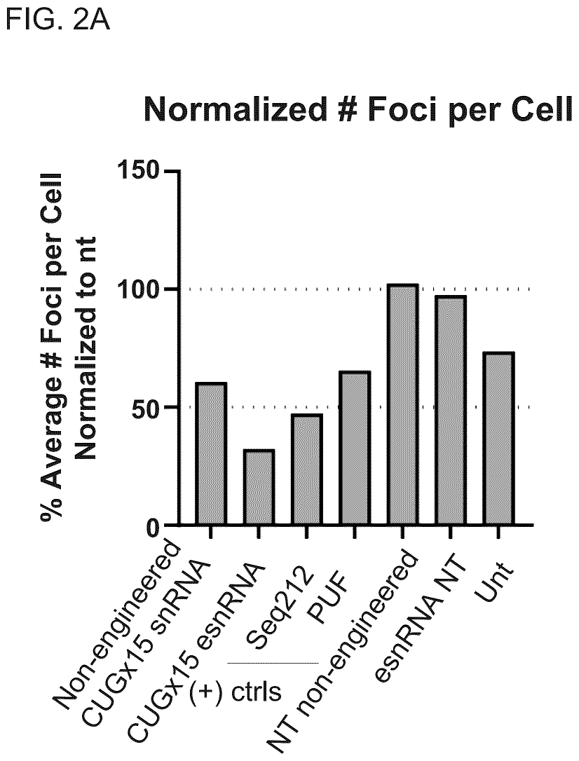

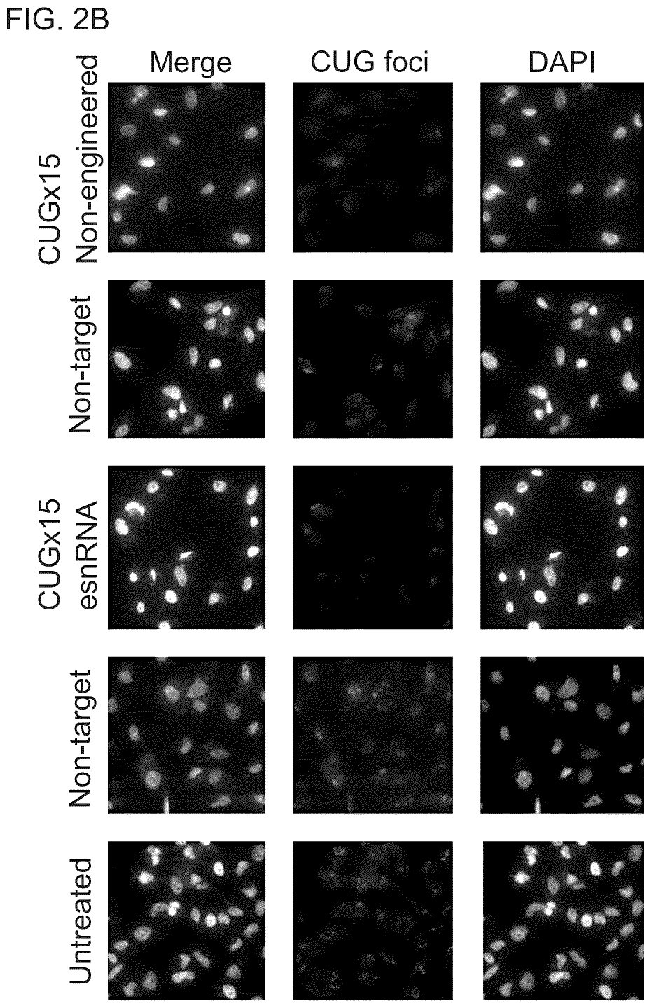

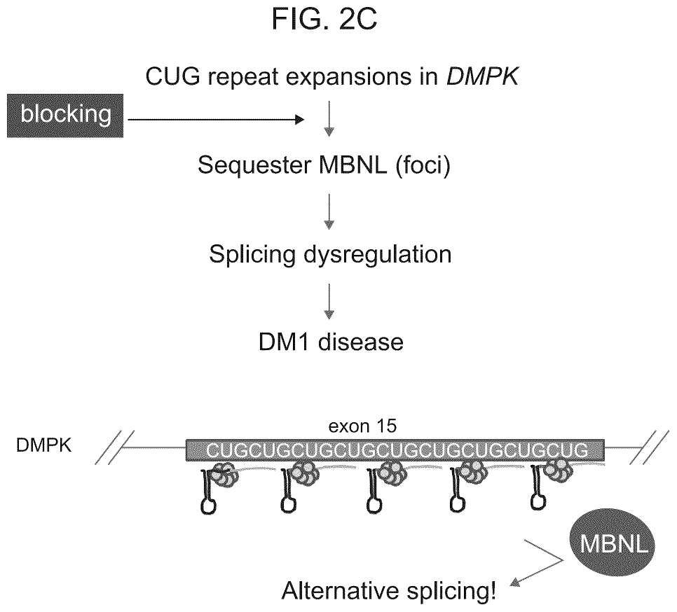

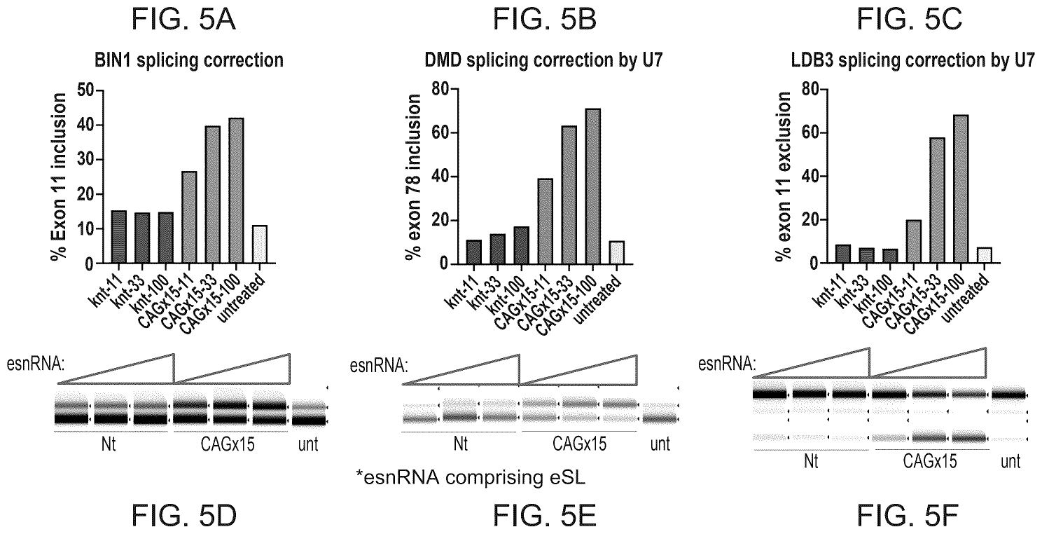

DM1 is caused by CUG repeat expansions in the 3′UTR of the DMPK gene that sequesters MBNL protein and as a result lead to dysregulation of alternative splicing. esnRNAs comprising an eSL disclosed herein were programmed to target CUG (15×CUGs) and this resulted in a dose dependent increase in healthy splice isoforms for all three tested disease splicing biomarkers compared to the non-targeting control. See FIGS. 5A-5F. See also FIG. 2C.

### Example 5: Testing Alternative 5′ Stabilizing Motifs in U7-Based eSLs and ISDs

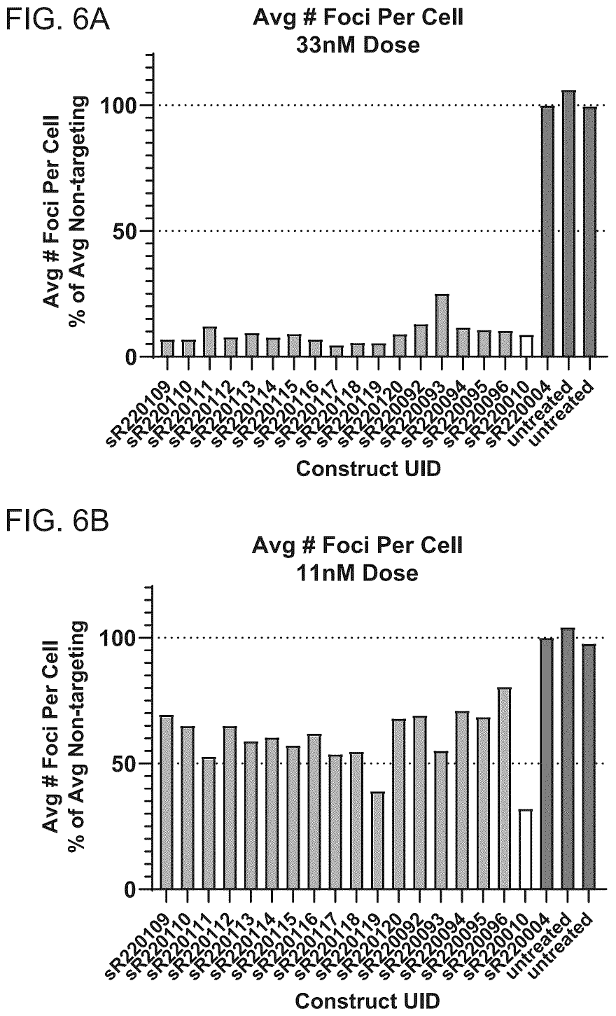

Efficacy of CUGx15 targeting snRNA with alternative 5′ interaction stabilizing domain (ISD) in reducing RNA foci was shown and determined by synthetic snRNA transfection in CTG480 HeLa cells followed by fluorescent in-situ hybridization for CUG RNA foci. See FIG. 6A-6B. (sR220109, sR220110, sR220111, sR220112, sR220113, sR220114, sR220115, sR220116, sR220117, sR220118, sR220119, sR220120, sR2200092, sR2200093, sR2200094, sR2200095, and sR2200096=alternative engineered stem loops with corresponding 5′ ISDs. sR220010=selected engineered stem loop and corresponding 5′ ISD. sR220004=non-targeting snRNA.)

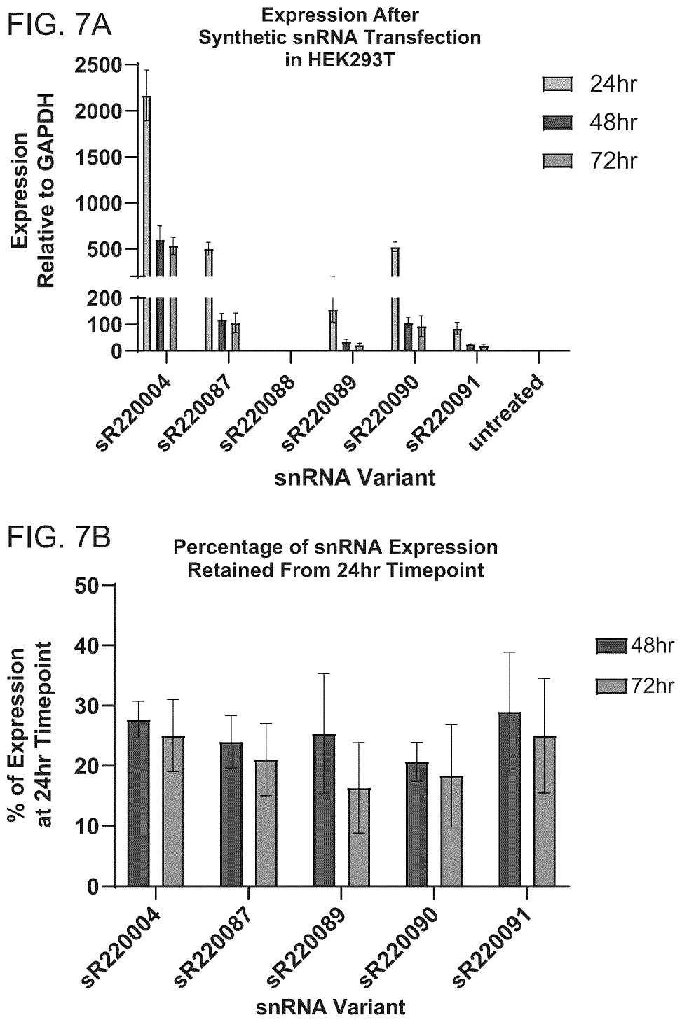

Expression levels of non-targeting snRNAs with alternative 5′ stabilizing domains at 24 hr, 48 hr, and 72 hr time points after transfection of synthetic snRNA were evaluated. See FIG. 7A-7B. sR220004 had the highest expression at all time points. sR220088 had no detectable expression. eSL/5′ISD found in 004 was selected for future constructs. sR220004 and sR220091 had the highest retention of expression at 48 hr and 72 hr time points.

### Example 6: U7 snRNA Efficacy and Expression by PolIII and PolII Promoters

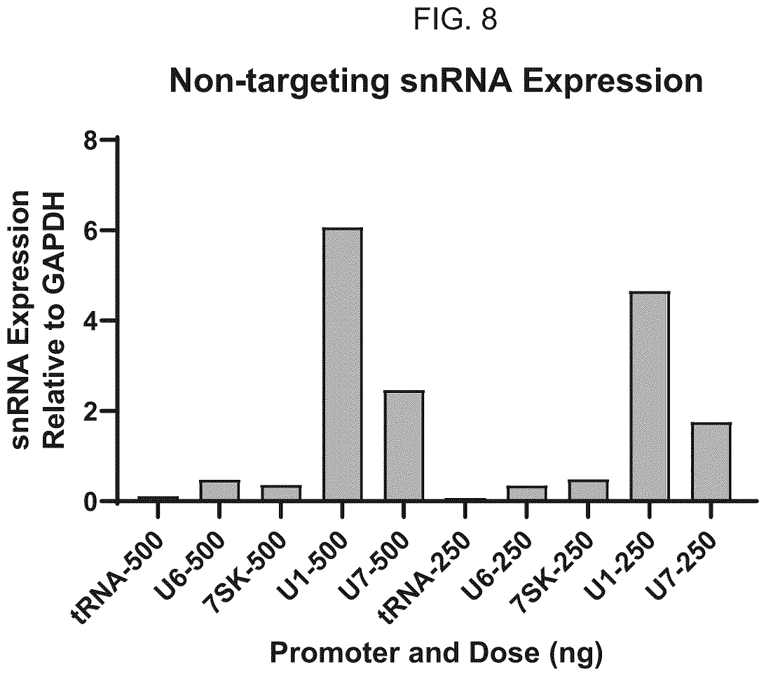

snRNA expression was evaluated by qPCR in CTG480 HeLa cells when snRNA expression was driven by different human PolIII and PolII promoters. See FIG. 8. The U1 promoter drives the highest level of snRNAs, followed by the U7 promoter. The PolIII promoters U6 and 7SK had low expression, and the PolIII tRNA promoter had lowest expression.

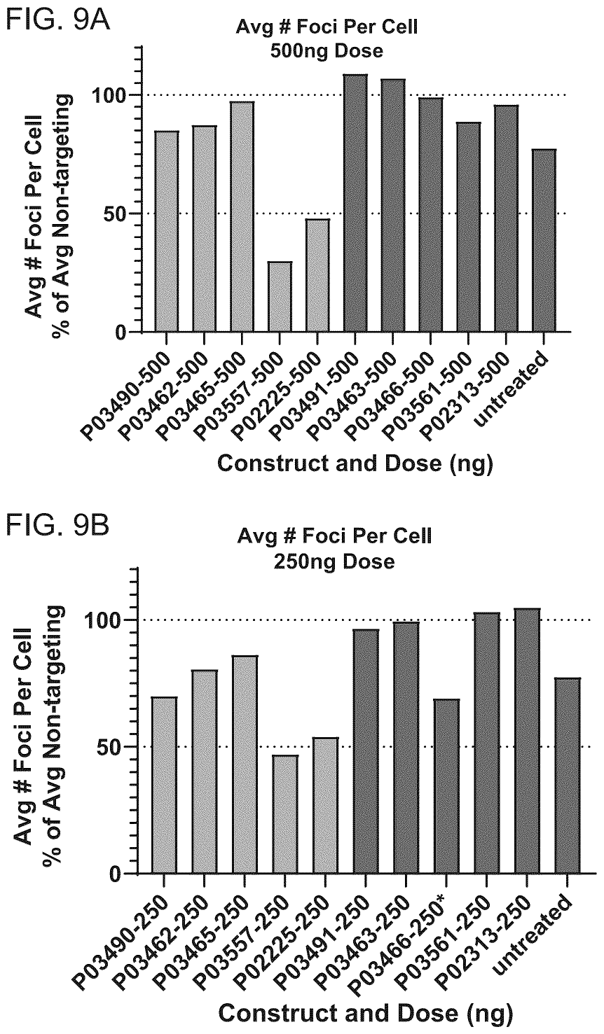

CUG foci quantification was evaluated by RNA FISH in CTG480 HeLa cells. See FIGS. 9A-9B. Only U1 and U7 promoters were clearly efficacious in reducing the number of CUG foci, with U1 being more efficacious than U7 (P03490, P03462, P03465, P03557, and P02225=CUG-targeting; P03491, P03463, P03466, P03561, and P02313=non-targeting).

| UID | Promoter, target | |
|---|---|---|
| P03490 | tRNA | (CUGx15) |
| P03462 | U6 | (CUGx15) |
| P03465 | 7SK | (CUGx15) |
| P03557 | U1 | (CUGx15) |
| P02225 | U7 | (CUGx15) |
| P03491 | tRNA | (nt) |
| P03463 | U6 | (nt) |
| P03466 | 7SK | (nt) |
| P03561 | U1 | (nt) |
| P02313 | U7 | (nt) |

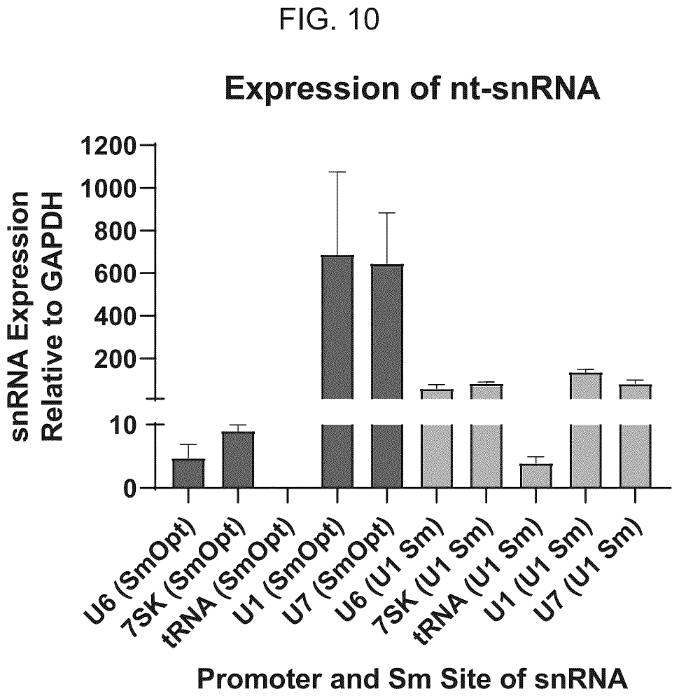

snRNA expression was determined by qPCR in HEK293T cells (see FIG. 10). Expression of U7 snRNAs under PolIII promoters increased up to 25× when SmOpt was converted to U1 Sm binding site. Expression by PolII U1 and U7 promoters decreased when Sm site was converted to U1 Sm. Expression by U1 and U7 promoters are superior to Pol III promoters.

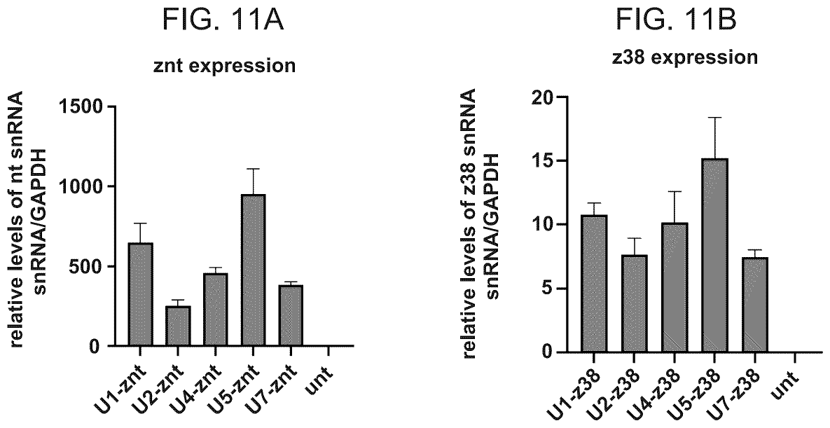

snRNA transfection into HEK293T cells was conducted. See FIG. 11A-11B. Here znt esnRNA and z38 esnRNA expression was driven by distinct snRNA promoters. Those constructs were transfected into HEK293T cells. 48 h post-transfection RNA was extracted and cDNA was made. Primers specific for each snRNA (z) were used for qPCR to determine snRNA expression and relative promoter strength.

### Example 7: esnRNA Constructs Result in DMD Exon Skipping of Exon 51 in Patient Myotubes

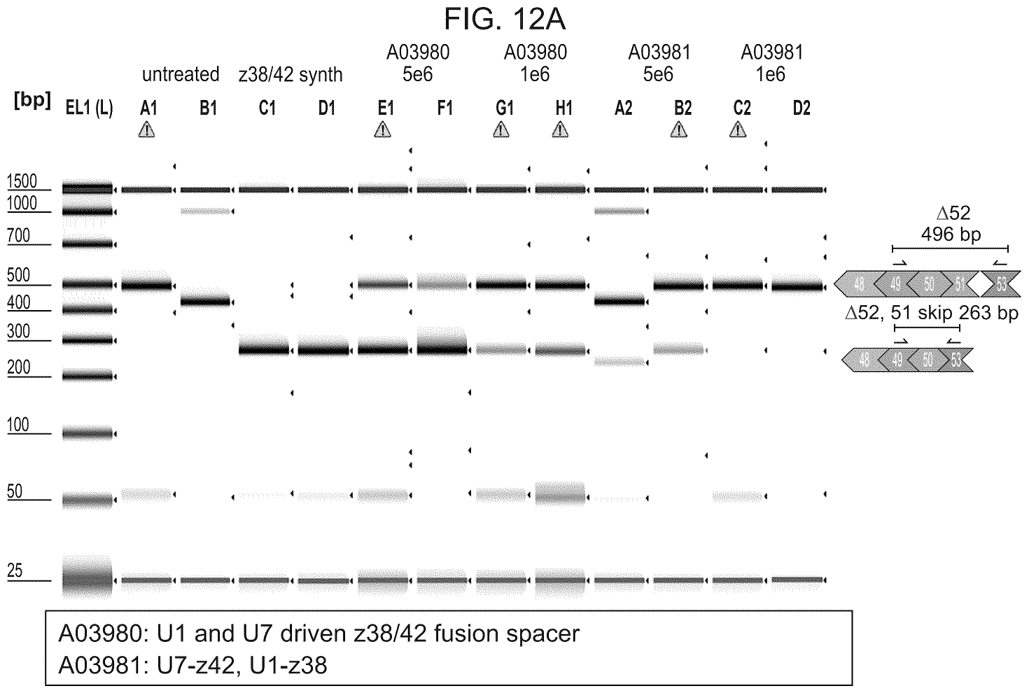

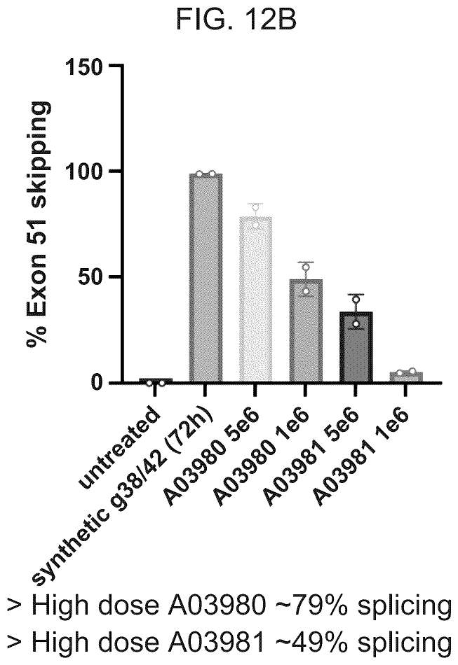

Transduction of human delta exon 52 myotubes with snRNA disclosed herein was conducted. See FIG. 12A-12B. Quantification of the band intensities using amplification primers determined % exon 51 skipping.

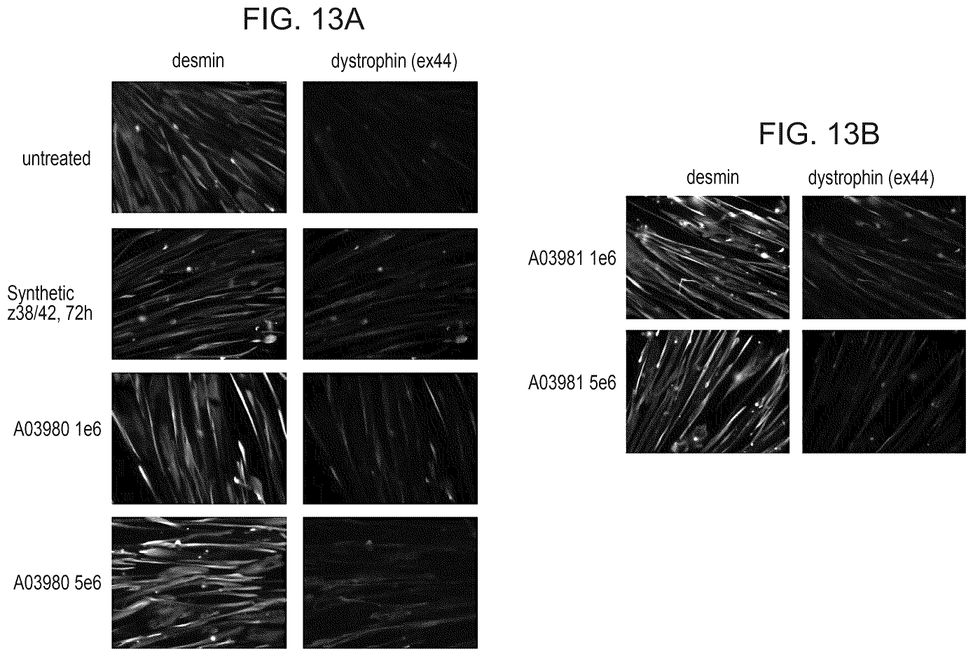

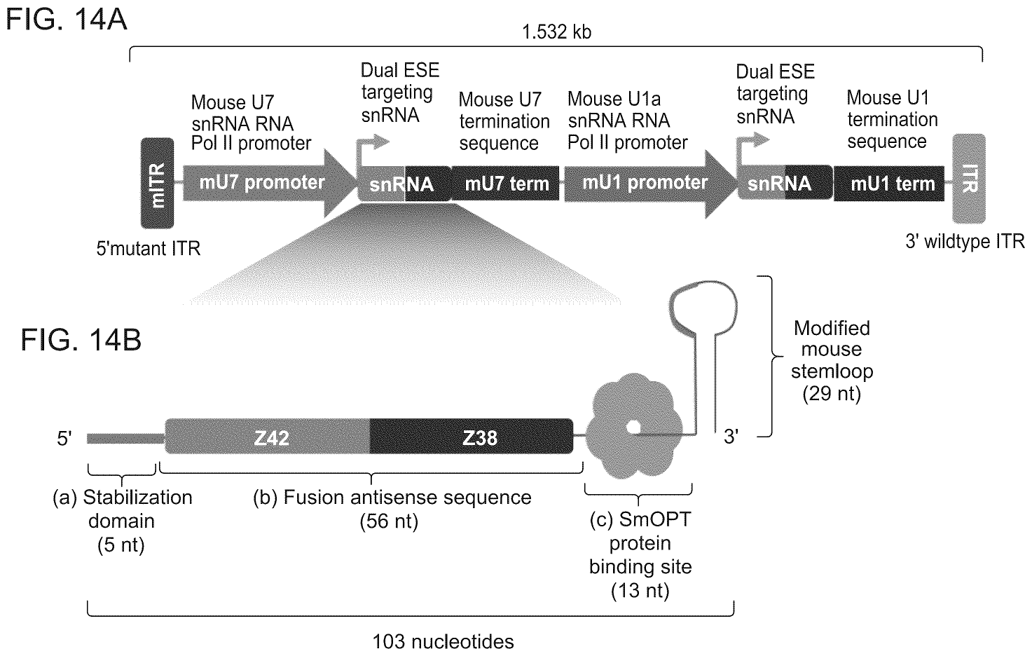

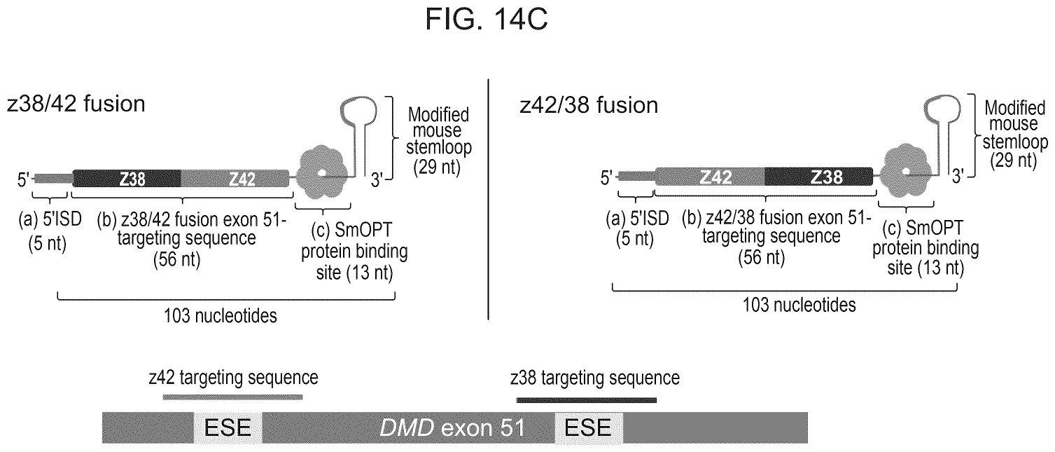

Human delta exon 52 myotubes were transduced with AAV9 carrying snRNAs disclosed herein. Immunofluorescence was performed to desmin and dystrophin. Dystrophin can be detected after treatment with exon 51-targeting snRNAs. See FIG. 13A-13B.

### Example 8: Exon 51 Skipping Skeletal Myotubes and a DMD Mouse Model

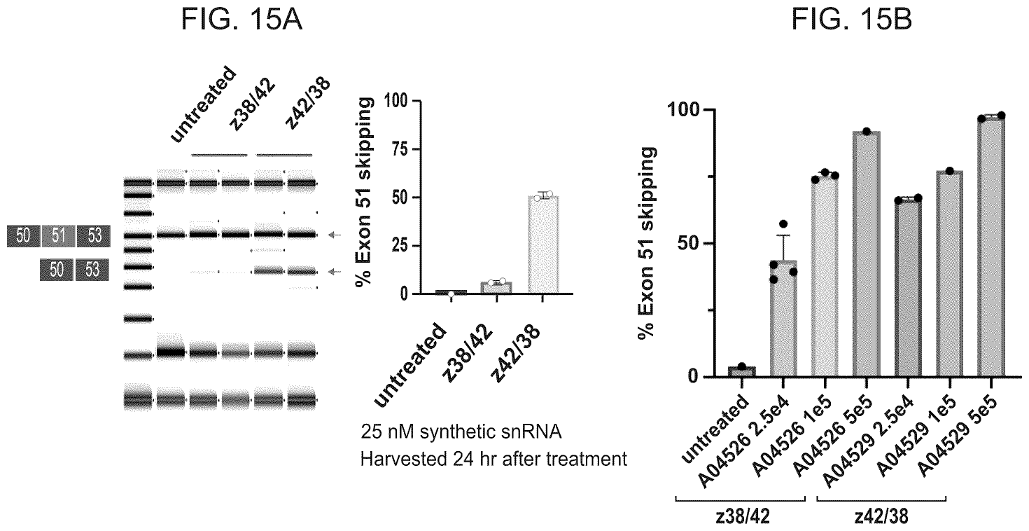

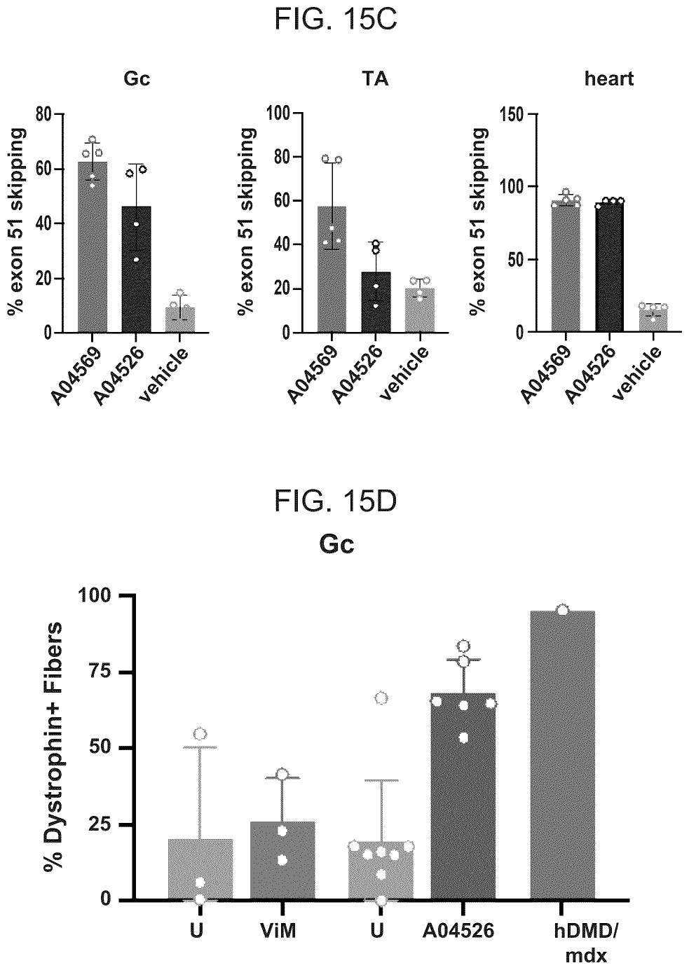

Human skeletal myotubes with exon 52 deleted were transfected with exon 51 RNA-targeting snRNAs carrying fusion antisense sequences (z73=z38/z42) and z187-z42/z38) which target exon splicing enhancers (ESEs). 24 hours after transfection of a low dose of the snRNA s exon 51 skipping levels were quantified (tape station image FIG. 15A). Human skeletal myotubes (with DMD exon 52 deleted) were transduced with AAV9-A04526 (2×z73) and A04569 (2×z187). The myotubes were transduced for 7 days with MO N of 2.5e4, 1e5 and 5e5. AAV9-based exon 51 skipping was quantified (FIG. 15B). Mice carrying hDMD with exon 52 deleted (del52hDMD/mdx) were injected retro-orbitally with 3e12 vector genomes of A04526 and A04569. Following 3-week survival, RNA was extracted from the tibialis anterior muscle (TA), the Gastrocnemius (Gc) and the heart. Semi-quantitative RT-PCR was performed to detect exon 51 included and excluded bands. (FIG. 150 shows quantification of exon 51 skipping in the humanized DMD mouse model. Intramuscular delivery of a 3e11 vg/muscle dose of A04526 or an exon 51 ESE-targeting Morpholino (ViM) to the Gastrocnemius of del52hDMD/mdx mice. FIG. 15D shows the quantification of dystrophin positive fibers after 4 weeks. hDMD/mdx mice (gray bar) which expresses wildtype human dystrophin serves as a positive control for dystrophin expression. U=untreated.

### Example 9: AAV9-Based Splicing Reversal and CUG Repeat Knockdown in DM1 Patient-Derived Myotubes

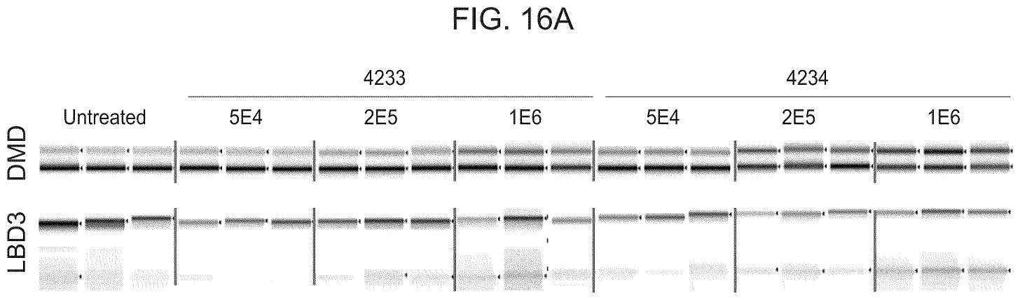

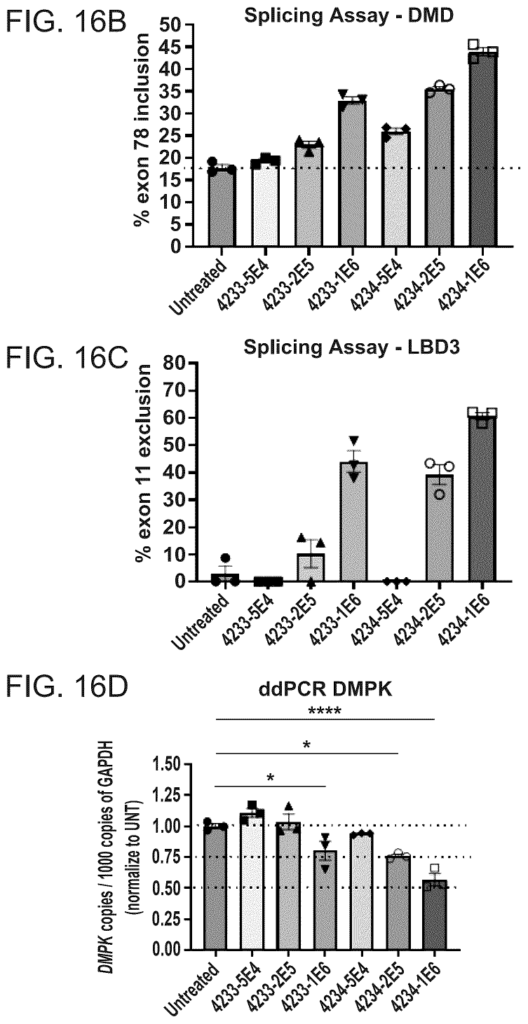

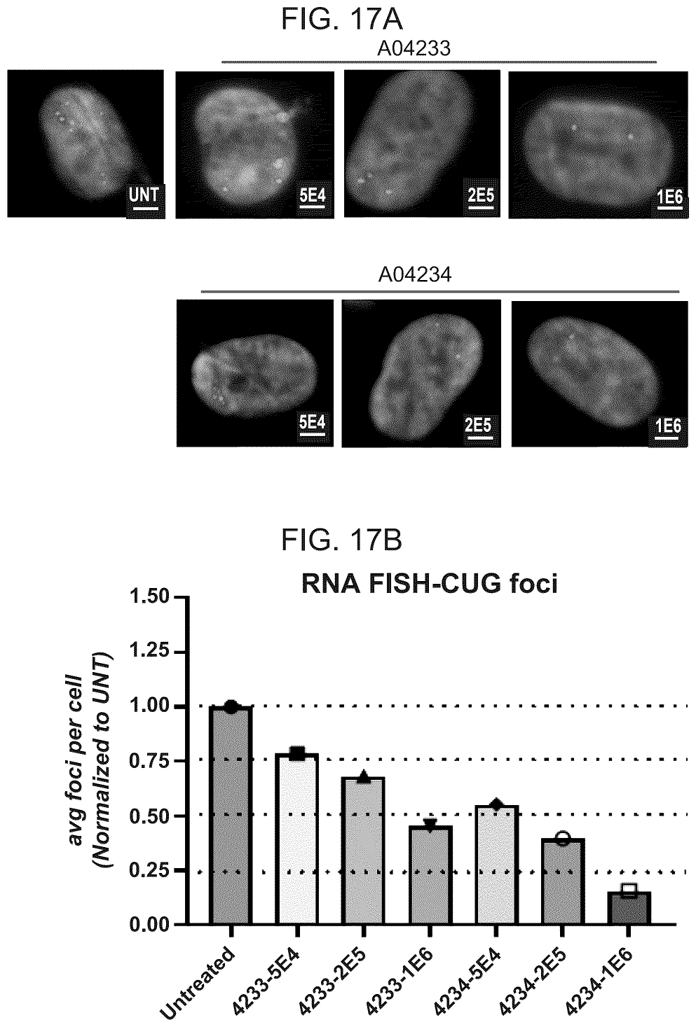

DM1 patient fibroblasts were differentiated into myotubes by transduction with myoD Adenovirus vector (for 5 days). Myotubes were transduced with MOIs of 5e4, 2e5 and 1e6 of A04233 and A04234 (scAAV9 with dual snRNA cassettes targeting CUG repeats) and harvested 7 days post-transduction. Splicing reversal of DMD and LBD3 RNAs and knockdown of DMPK after treatment with U7 snRNA targeting CUG repeats in patient derived myotubes (containing 1700 CUG repeats). See FIGS. 16A-D. Cell nuclei were stained with DAPI and CUG FISH was performed with a CUG labeled probe. Cells were processed 7 days post-transduction for quantification of average number of CUG foci per cell. See FIGS. 17A-B.

### Example 10: AAV9-Based HTT Knockdown in HD Patient Cells and HD Mouse Model

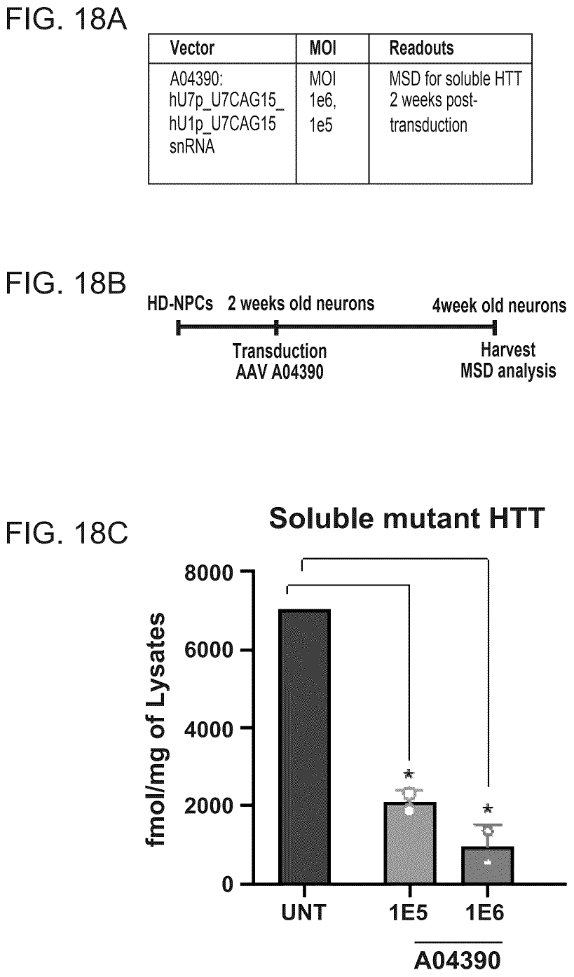

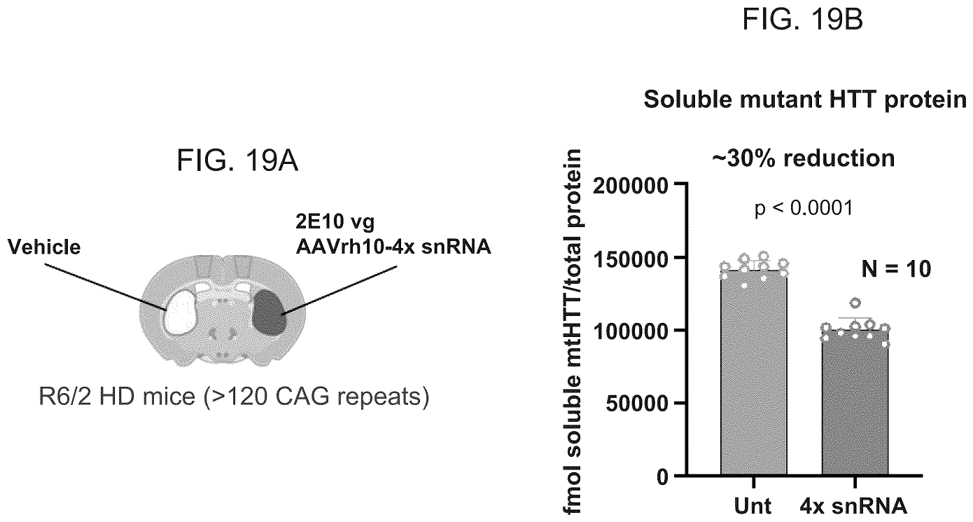

HD patient iPSc (containing 66 CAG repeats) were differentiated into cortical neurons for 2 weeks and then transduced with 2 different MOIs of AAV-A04390 snRNA vector (2×snRNA cassette targeting CAG repeats) for an additional 2 weeks. Then cortical neurons were harvested and mutant soluble HTT quantified by Meso scale discovery immunoassay. See FIGS. 18A-18C. Then, an in vivo study was designed to target CAG repeats in an R6/2 HD mouse model. An AAV-snRNA vector targeting CAG repeats (A03081: AAVrh10-snRNA (4×snRNAs with CAGx15 TS)) was administered intrastriatal at 2e10 vector genomes and post-treatment mutant soluble HTT protein was quantified via Meso scale discovery immunoassay (MSD) on untreated and snRNA treated mice (n=10). See FIGS. 19A-B.

### Example 11: Higher Levels of Expression with Engineered Stem Loop snRNA Vectors Compared to Native Stem Loop snRNA Vectors

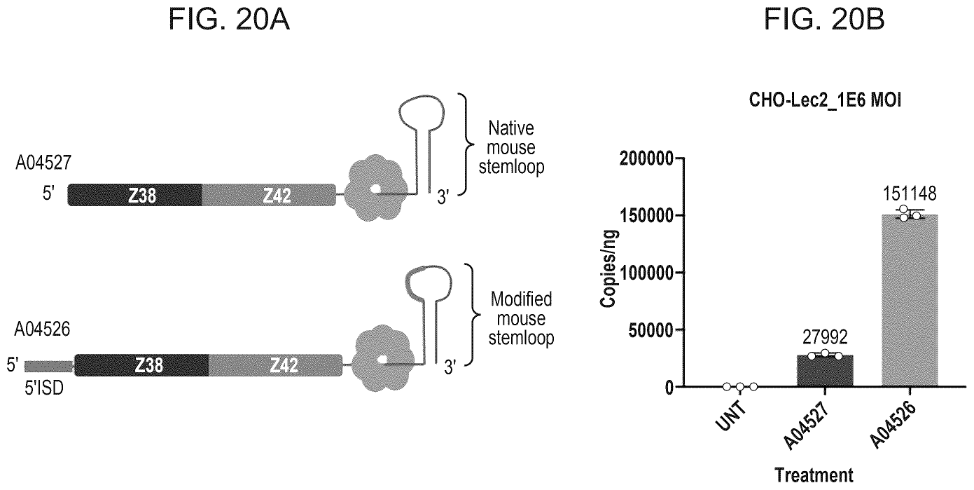

CHO-Lec2 cells were transduced at an MOI of 1e6 with AAV9-A04527 vector (native stem loop expressing z38/42 targeting DMD exon 51 ESEs) or AAV9-A04526 vector (engineered stem loop expressing z38/42 targeting DMD exon 51 ESEs). Expression of z38/42 is higher in cells transduced with engineered snRNA vector compared to expression of z38/42 in cells transduced with native stem loop snRNA vector. See FIGS. 20A-B.

### INCORPORATION BY REFERENCE

Every document cited herein, including any cross referenced or related patent or application is hereby incorporated herein by reference in its entirety unless expressly excluded or otherwise limited. The citation of any document is not an admission that it is prior art with respect to any invention disclosed or embodimented herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

### Other Embodiments

While particular embodiments of the disclosure have been illustrated and described, various other changes and modifications can be made without departing from the spirit and scope of the disclosure. The scope of the appended claims includes all such changes and modifications that are within the scope of this disclosure.

1-20. (canceled)

21. An RNA-targeting nucleic acid molecule comprising an engineered small nuclear RNA (esnRNA), wherein the esnRNA comprises an engineered stem loop (eSL) comprising one or more nucleic acid sequences selected from the group consisting of SEQ ID NOS: 1-11, 147-148, 163, and 186-205.

22. The RNA-targeting nucleic acid molecule of claim 21, wherein the esnRNA comprises a targeting sequence (TS) that targets a target RNA of interest.

23. The RNA-targeting nucleic acid molecule of claim 22, wherein the target RNA of interest comprises pre-mRNA or mRNA sequence.

24. The RNA-targeting nucleic acid molecule of claim 22, wherein the target RNA of interest comprises a microsatellite repeat RNA.

25. The RNA-targeting nucleic acid molecule of claim 24, wherein the microsatellite repeat RNA is selected from the group consisting of CUG, CAG, and GGGGCC+CCCCGG.

26. The RNA-targeting nucleic acid molecule of claim 21, wherein the esnRNA comprises two targeting sequences (TSs) that target two RNAs of interest.

27. The RNA-targeting nucleic acid molecule of claim 26, wherein the two TSs are a fusion sequence.

28. The RNA-targeting nucleic acid molecule of claim 21, wherein the esnRNA comprises an Sm binding domain (SmBD) selected from the group consisting of a U1, U2, U4, and U5 SmBD.

29. The RNA-targeting nucleic acid molecule of claim 28, wherein the SmBD comprises a nucleic acid sequence selected from the group consisting of ATTTTT, AATTTTTGG, AATTTGTGG, AATTTGTGG, AATTTCTGG, GATTTTTGG, AATTTTTGA, AATTTTTTG, SEQ ID NO: 161 (AATTTTTGGAGCA), and SEQ ID NO: 164 (AATTTTTGGAGTA).

30. The RNA-targeting nucleic acid molecule of claim 21, wherein the esnRNA comprises a 5′ interaction stabilizer domain (5′ISD) comprising a nucleotide sequence selected from the group consisting of GGAGT, CCTCT, GGAGGT, CCTCCT, AGCCAG, GGAAG, GAAGAAG, GTTG, CCGAA, TAAGGAG, GAAG, OR GGCTT.

31. A vector comprising one or more RNA-targeting nucleic acid molecules of claim 21.

32. The vector of claim 31, wherein the viral vector is an AAV vector.

33. The vector of claim 31, wherein the one or more of RNA-targeting nucleic acid molecules are operably linked to a promoter.

34. The vector of claim 33, wherein the promoter comprises a U7 promoter or a U1 promoter.

35. The vector of claim 31, wherein the esnRNA is operably linked to a downstream terminator (DT).

36. The vector of claim 35, wherein the DT is a U7 downstream terminator or a U1 downstream terminator.

37. The vector of claim 31, wherein the vector comprises at least one, at least two, at least three, at least four, or at least five esnRNA.

38. The vector of claim 37, wherein each esnRNA is separated by a buffer sequence.

39. The vector of claim 38, wherein the buffer sequence comprises a nucleic acid sequence selected from the group consisting of SEQ ID NOS: 24-30.

40. A method of targeting one or more target RNAs of interest, comprising contacting a cell comprising the one or more target RNAs of interest with an RNA-targeting nucleic acid molecule comprising (i) a targeting sequence (TS) that targets the target RNA of interest and (ii) an engineered small nuclear RNA (esnRNA), wherein the esnRNA comprises an engineered stem loop (eSL) comprising one or more nucleic acid sequences selected from the group consisting of SEQ ID NOS: 1-11, 147-148, 163, and 186-205, and wherein the contacting results in blocking, knocking down, editing, exon-skipping or splicing the one or more target RNAs of interest.

41. A method of treating a disease or disorder in a subject comprising administering to the subject an RNA-targeting nucleic acid molecule comprising (i) a targeting sequence (TS) that targets the target RNA of interest and (ii) an engineered small nuclear RNA (esnRNA), wherein the esnRNA comprises an engineered stem loop (eSL) comprising one or more nucleic acid sequences selected from the group consisting of SEQ ID NOS: 1-11, 147-148, 163, and 186-205, or administering an AAV vector comprising a sequence encoding the RNA-targeting nucleic acid molecule.

42. The method of claim 41, wherein the disease or disorder is associated with a toxic repeat RNA sequence.

43. The method of claim 41, wherein the disease or disorder is myotonic dystrophy (DM1) or Huntington's disease (HD).


SEQUENCE LISTING (233 sequences)

SEQ ID NO: 1 - DNA, 27 bp, synthetic construct
  source 1..27: mol_type = other DNA
ggctttctggctccttaccggaaagcc

SEQ ID NO: 2 - DNA, 27 bp, synthetic construct
  source 1..27: mol_type = other DNA
ggctttctgggaggttaccggaaagcc

SEQ ID NO: 3 - DNA, 28 bp, synthetic construct
  source 1..28: mol_type = other DNA
ggctttctggcctccttaccggaaagcc

SEQ ID NO: 4 - DNA, 28 bp, synthetic construct
  source 1..28: mol_type = other DNA
ggctttctggggaggttaccggaaagcc

SEQ ID NO: 5 - DNA, 27 bp, synthetic construct
  source 1..27: mol_type = other DNA
ggctttctggctggctaccggaaagcc

SEQ ID NO: 6 - DNA, 25 bp, synthetic construct
  source 1..25: mol_type = other DNA
ggctttctggcttccccggaaagcc

SEQ ID NO: 7 - DNA, 27 bp, synthetic construct
  source 1..27: mol_type = other DNA
ggctttctggcttcttcccggaaagcc

SEQ ID NO: 8 - DNA, 27 bp, synthetic construct
  source 1..27: mol_type = other DNA
ggctttctggcaacttaccggaaagcc

SEQ ID NO: 9 - DNA, 27 bp, synthetic construct
  source 1..27: mol_type = other DNA
ggctttctggttcggtaccggaaagcc

SEQ ID NO: 10 - DNA, 28 bp, synthetic construct
  source 1..28: mol_type = other DNA
ggctttctggaagccttaccggaaagcc

SEQ ID NO: 11 - DNA, 27 bp, synthetic construct
  source 1..27: mol_type = other DNA
ggctttctggcttcttaccggaaagcc

SEQ ID NO: 12-23 - skipped (12 entries)

SEQ ID NO: 24 - DNA, 30 bp, synthetic construct
  source 1..30: mol_type = other DNA
caaactacagagccaagtgctatccacaga

SEQ ID NO: 25 - DNA, 30 bp, synthetic construct
  source 1..30: mol_type = other DNA
gagctttctgggttgccatctcaagcagac

SEQ ID NO: 26 - DNA, 30 bp, synthetic construct
  source 1..30: mol_type = other DNA
tacaaggccatcagctcatactcacaattg

SEQ ID NO: 27 - DNA, 100 bp, synthetic construct
  source 1..100: mol_type = other DNA
caaactacagagccaagtgctatccacagagagctttctgggttgccatctcaagcagactacaaggccatcagctcatactcacaattgactttgagag

SEQ ID NO: 28 - DNA, 100 bp, synthetic construct
  source 1..100: mol_type = other DNA
ttgaccacatacgtgctctttcaaagttctgtgtttgaagttatgttagtaacaactgatgcccatcctgcaatgacaaatccaattctcagtgcagctc

SEQ ID NO: 29 - DNA, 500 bp, synthetic construct
  source 1..500: mol_type = other DNA
caaactacagagccaagtgctatccacagagagctttctgggttgccatctcaagcagactacaaggccatcagctcatactcacaattgactttgagagtcattttccaatgctcctacacaccccttcttcacaatccccaacaaatctgaggctggaacttggtaccataacaatcattacattatttcaccagaagtacaccttgcctggaagattggcattatagcatcttctaacattgtgaaagttagtgaccaatgaggagatccaagtcagttccagttggatttctctatactctataataaatatatatggtgtcttcaacaataggactttgccatccagtgatgctaaaaatcaataacaatggcaataacctgccctgtttggaaagcctctggcttccatgactaacaattcaaggcaggtctcctatacctagtactgagatttttatttgataaactatatcttctgggaggagaagcattgt

SEQ ID NO: 30 - DNA, 500 bp, synthetic construct
  source 1..500: mol_type = other DNA
ttgaccacatacgtgctctttcaaagttctgtgtttgaagttatgttagtaacaactgatgcccatcctgcaatgacaaatccaattctcagtgcagctctctgaaatagttttgctttctctctctaggtctgttctatactcctaactctccaggagtttacaaggaataaaatctcttccaaatgctttctgttgcaacaactggaccatactgaaagctgaggcccacaattgcaatctaggttagcaggtaatcattgttggtgaggtcctccctttccccaggctcgtgtttgtattggggagcaggaaatttttgctagagcagcactgccatctctctacactccacctgattggtgggatggaccagagaaatggacattcccaacacagtccctcctttcacatctgctcacctgcccacaggatactttccaccatgcatactgggctctgcaccaaccattcagcagtgatgaagaggaaacttgaac

SEQ ID NO: 31-38 - skipped (8 entries)

SEQ ID NO: 39 - DNA, 288 bp, synthetic construct
  source 1..288: mol_type = other DNA
tactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgca

SEQ ID NO: 40 - DNA, 392 bp, synthetic construct
  source 1..392: mol_type = other DNA
aaggaccagcttctttgggagagaacagacgcaggggcgggagggaaaaagggagaggcagacgtcacttccccttggcggctctggcagcagattggtcggttgagtggcagaaaggcagacggggactgggcaaggcactgtcggtgacatcacggacagggcgacttctatgtagatgaggcagcgcagaggctgacgtcttcgccacttgctgcttcaccacgaaggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagaggcacaacgtttc

SEQ ID NO: 41 - DNA, 247 bp, synthetic construct
  source 1..247: mol_type = other DNA
ccacgccctctgtgaaagggcggggcatgcaaattcgaaatgaaagcccgggaacgccggaagaagcacgggtgtaagatttcccttttcaaaggcagagaataagaaatcagcccgagagtgtaagggcgtcaatagcgctgtggacgagacagagggaatggggcaaggagcgaggctggggctctcaccgcgacttgaatgtggatgagagtgggacggtgacggcgggcgcgaaggcgagcgc

SEQ ID NO: 42 - DNA, 250 bp, synthetic construct
  source 1..250: mol_type = other DNA
ttcgcagtctctgaattaagtctattagcatgttcctcccatagtgctttgcttcatatcaacaaaaacctagctaagtgaaatcagcaacgatatgcagaaaccacctacgcaggtcacaaacatctttctatgattgtataattttcaagcaagcaataagtgaagatttttccataggccctaaactcacctttgcgaaataggaagctggtttattgggagtgatgagcagggggcgtaacaaatt

SEQ ID NO: 43 - DNA, 300 bp, synthetic construct
  source 1..300: mol_type = other DNA
gcagcaaggcctccacttcaccccctaaaggttgccccaagagcaccgtgtgactgctaaggtatttccggagtctaaagacgattattcaggtctcatttgcatacccataatacactgcaaacagtatttttttcggaaaaacatttatatattgcttgacatttttaagtatgagaattttgcatgcagaatttttttgtataaactttctcaggtagtaacccttgggattagtagacaccatcagtgtactaggaattgcagttacccgaaaattgagttacagaagtaactggt

SEQ ID NO: 44 - DNA, 296 bp, synthetic construct
  source 1..296: mol_type = other DNA
gtttattacagggacagcagagatccagtttggttaattaaggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 45 - DNA, 243 bp, synthetic construct
  source 1..243: mol_type = other DNA
ctgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctc

SEQ ID NO: 46 - DNA, 106 bp, synthetic construct
  source 1..106: mol_type = other DNA
caggactagtcttttaggtcaaaaagaagaagctttgtaaccgttggtttccgtagtgtagtggttatcacgttcgcctaacacgcgaaaggtccccggttcgaag

SEQ ID NO: 47 - DNA, 439 bp, synthetic construct
  source 1..439: mol_type = other DNA
ttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactc

SEQ ID NO: 48 - DNA, 258 bp, synthetic construct
  source 1..258: mol_type = other DNA
taacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaa

SEQ ID NO: 49 - DNA, 94 bp, synthetic construct
  source 1..94: mol_type = other DNA
cctcttatgatgtttgttgccaatgatagattgttttcactgtgcaaaaattatgggtagttttggtggtcttgatgcagttgtaagcttggag

SEQ ID NO: 50 - DNA, 50 bp, synthetic construct
  source 1..50: mol_type = other DNA
actttctggagtttcaaaaacagactgtacgccaagggtcatatcttttt

SEQ ID NO: 51 - DNA, 50 bp, synthetic construct
  source 1..50: mol_type = other DNA
ccggggatacaacgtgtttcctaaaagtagagggaggtaagagacggtag

SEQ ID NO: 52 - DNA, 50 bp, synthetic construct
  source 1..50: mol_type = other DNA
ctgaattttcttgcagttgaacaacagaggctttttttgtgtgtgtgggg

SEQ ID NO: 53 - DNA, 50 bp, synthetic construct
  source 1..50: mol_type = other DNA
atatgtggtaatccaacaatagaaattatttttaagtttgtgtgttcctt

SEQ ID NO: 54-56 - skipped (3 entries)

SEQ ID NO: 57 - DNA, 206 bp, synthetic construct
  source 1..206: mol_type = other DNA
gtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagca

SEQ ID NO: 58 - DNA, 110 bp, synthetic construct
  source 1..110: mol_type = other DNA
cccaatttcactggtctacaatgaaagcaaaacagttctcttccccgctccccggtgtgtgagaggggctttgatccttctctggtttcctaggaaacgcgtatgtgtac

SEQ ID NO: 59 - DNA, 130 bp, synthetic construct
  source 1..130: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 60 - DNA, 141 bp, synthetic construct
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 61 - DNA, 113 bp, synthetic construct
  source 1..113: mol_type = other DNA
ccactccctctctgcgcgctcgctcgctcactgaggccgggcgaccaaaggtcgcccgacgcccgggctttgcccgggcggcctcagtgagcgagcgagcgcgcagagaggga

SEQ ID NO: 62 - DNA, 508 bp, synthetic construct
  source 1..508: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 63 - DNA, 720 bp, synthetic construct
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 64 - DNA, 122 bp, synthetic construct
  source 1..122: mol_type = other DNA
aacttgtttattgcagcttataatggttacaaataaagcaatagcatcacaaatttcacaaataaagcatttttttcactgcattctagttgtggtttgtccaaactcatcaatgtatctta

SEQ ID NO: 65 - DNA, 90 bp, synthetic construct
  source 1..90: mol_type = other DNA
ggagtcagcagcagcagcagcagcagcagcagcagcagcagcagcagcagaatttttggagtaggctttctggctccttaccggaaagcc

SEQ ID NO: 66 - DNA, 71 bp, synthetic construct
  source 1..71: mol_type = other DNA
ggagttcaccagaagcgtaccatactcacgaaatttttggagtaggctttctggctccttaccggaaagcc

SEQ ID NO: 67 - DNA, 90 bp, synthetic construct
  source 1..90: mol_type = other DNA
ggagtctgctgctgctgctgctgctgctgctgctgctgctgctgctgctgaatttttggagtaggctttctggctccttaccggaaagcc

SEQ ID NO: 68 - DNA, 101 bp, synthetic construct
  source 1..101: mol_type = other DNA
ggagttacctccaacatcaaggaagatggcagtaaccacaggttgtgtcaccagagtaacaaatttttggagtaggctttctggctccttaccggaaagcc

SEQ ID NO: 69 - DNA, 75 bp, synthetic construct
  source 1..75: mol_type = other DNA
ggagtgtaaccacaggttgtgtcaccagagtaacaaatttttggagtaggctttctggctccttaccggaaagcc

SEQ ID NO: 70 - DNA, 71 bp, synthetic construct
  source 1..71: mol_type = other DNA
ggagttacctccaacatcaaggaagatggcaaatttttggagtaggctttctggctccttaccggaaagcc

SEQ ID NO: 71 - DNA, 90 bp, synthetic construct
  source 1..90: mol_type = other DNA
ggagtcagcagcagcagcagcagcagcagcagcagcagcagcagcagcagaatttgtggagtaggctttctggctccttaccggaaagcc

SEQ ID NO: 72 - DNA, 90 bp, synthetic construct
  source 1..90: mol_type = other DNA
ggagtcagcagcagcagcagcagcagcagcagcagcagcagcagcagcagtcgagttggagtaggctttctggctccttaccggaaagcc

SEQ ID NO: 73 - DNA, 81 bp, synthetic construct
  source 1..81: mol_type = other DNA
ggagtcagcagcagcagcagcagcagcagcagcagcagcagcagcagcagagtaggctttctggctccttaccggaaagcc

SEQ ID NO: 74 - DNA, 71 bp, synthetic construct
  source 1..71: mol_type = other DNA
ggagttcaccagaagcgtaccatactcacgaaatttgtggagtaggctttctggctccttaccggaaagcc

SEQ ID NO: 75 - DNA, 71 bp, synthetic construct
  source 1..71: mol_type = other DNA
ggagttcaccagaagcgtaccatactcacgatcgagttggagtaggctttctggctccttaccggaaagcc

SEQ ID NO: 76 - DNA, 62 bp, synthetic construct
  source 1..62: mol_type = other DNA
ggagttcaccagaagcgtaccatactcacgaagtaggctttctggctccttaccggaaagcc

SEQ ID NO: 77 - DNA, 90 bp, synthetic construct
  source 1..90: mol_type = other DNA
ggagtctgctgctgctgctgctgctgctgctgctgctgctgctgctgctgaatttgtggagtaggctttctggctccttaccggaaagcc

SEQ ID NO: 78 - DNA, 90 bp, synthetic construct
  source 1..90: mol_type = other DNA
ggagtctgctgctgctgctgctgctgctgctgctgctgctgctgctgctgtcgagttggagtaggctttctggctccttaccggaaagcc

SEQ ID NO: 79 - DNA, 81 bp, synthetic construct
  source 1..81: mol_type = other DNA
ggagtctgctgctgctgctgctgctgctgctgctgctgctgctgctgctgagtaggctttctggctccttaccggaaagcc

SEQ ID NO: 80 - DNA, 60 bp, synthetic construct
  source 1..60: mol_type = other DNA
ggagtctgctgctgctgctgaatttgtggagtaggctttctggctccttaccggaaagcc

SEQ ID NO: 81 - DNA, 60 bp, synthetic construct
  source 1..60: mol_type = other DNA
ggagtctgctgctgctgctgtcgagttggagtaggctttctggctccttaccggaaagcc

SEQ ID NO: 82 - DNA, 51 bp, synthetic construct
  source 1..51: mol_type = other DNA
ggagtctgctgctgctgctgagtaggctttctggctccttaccggaaagcc

SEQ ID NO: 83 - DNA, 58 bp, synthetic construct
  source 1..58: mol_type = other DNA
ggagttacctccaacatcaaggaagatggcaggctttctggctccttaccggaaagcc

SEQ ID NO: 84 - DNA, 96 bp, synthetic construct
  source 1..96: mol_type = other DNA
gttcccgaagtaacccttcgggaacggagttcaccagaagcgtaccatactcacgaaatttttggagtaggctttctggctccttaccggaaagcc

SEQ ID NO: 85 - DNA, 91 bp, synthetic construct
  source 1..91: mol_type = other DNA
gttcccgaagtaacccttcgggaactcaccagaagcgtaccatactcacgaaatttttggagtaggctttctggctccttaccggaaagcc

SEQ ID NO: 86 - DNA, 91 bp, synthetic construct
  source 1..91: mol_type = other DNA
gttcccgaagggagtcttcgggaactcaccagaagcgtaccatactcacgaaatttttggagtaggctttctggctccttaccggaaagcc

SEQ ID NO: 87 - DNA, 72 bp, synthetic construct
  source 1..72: mol_type = other DNA
gggccctcaccagaagcgtaccatactcacgaaatttttggagtaggctttctggctccttaccggaaagcc

SEQ ID NO: 88 - DNA, 96 bp, synthetic construct
  source 1..96: mol_type = other DNA
gagaagattcgtcttctctctctctcttcttcaccagaagcgtaccatactcacgaaatttttggagtaggctttctggctccttaccggaaagcc

SEQ ID NO: 89 - DNA, 115 bp, synthetic construct
  source 1..115: mol_type = other DNA
gttcccgaagtaacccttcgggaacggagtcagcagcagcagcagcagcagcagcagcagcagcagcagcagcagaatttttggagtaggctttctggctccttaccggaaagcc

SEQ ID NO: 90 - DNA, 110 bp, synthetic construct
  source 1..110: mol_type = other DNA
gttcccgaagtaacccttcgggaaccagcagcagcagcagcagcagcagcagcagcagcagcagcagcagaatttttggagtaggctttctggctccttaccggaaagcc

SEQ ID NO: 91 - DNA, 110 bp, synthetic construct
  source 1..110: mol_type = other DNA
gttcccgaagggagtcttcgggaaccagcagcagcagcagcagcagcagcagcagcagcagcagcagcagaatttttggagtaggctttctggctccttaccggaaagcc

SEQ ID NO: 92 - DNA, 91 bp, synthetic construct
  source 1..91: mol_type = other DNA
gggccccagcagcagcagcagcagcagcagcagcagcagcagcagcagcagaatttttggagtaggctttctggctccttaccggaaagcc

SEQ ID NO: 93 - DNA, 115 bp, synthetic construct
  source 1..115: mol_type = other DNA
gagaagattcgtcttctctctctctcttctcagcagcagcagcagcagcagcagcagcagcagcagcagcagcagaatttttggagtaggctttctggctccttaccggaaagcc

SEQ ID NO: 94 - DNA, 71 bp, synthetic construct; identical to SEQ ID NO: 66
  source 1..71: mol_type = other DNA

SEQ ID NO: 95 - DNA, 71 bp, synthetic construct
  source 1..71: mol_type = other DNA
cctcttcaccagaagcgtaccatactcacgaaatttttggagtaggctttctgggaggttaccggaaagcc

SEQ ID NO: 96 - DNA, 73 bp, synthetic construct
  source 1..73: mol_type = other DNA
ggaggttcaccagaagcgtaccatactcacgaaatttttggagtaggctttctggcctccttaccggaaagcc

SEQ ID NO: 97 - DNA, 73 bp, synthetic construct
  source 1..73: mol_type = other DNA
cctccttcaccagaagcgtaccatactcacgaaatttttggagtaggctttctggggaggttaccggaaagcc

SEQ ID NO: 98 - DNA, 72 bp, synthetic construct
  source 1..72: mol_type = other DNA
agccagtcaccagaagcgtaccatactcacgaaatttttggagtaggctttctggctggctaccggaaagcc

SEQ ID NO: 99 - DNA, 69 bp, synthetic construct
  source 1..69: mol_type = other DNA
ggaagtcaccagaagcgtaccatactcacgaaatttttggagtaggctttctggcttccccggaaagcc

SEQ ID NO: 100 - DNA, 73 bp, synthetic construct
  source 1..73: mol_type = other DNA
gaagaagtcaccagaagcgtaccatactcacgaaatttttggagtaggctttctggcttcttcccggaaagcc

SEQ ID NO: 101 - DNA, 70 bp, synthetic construct
  source 1..70: mol_type = other DNA
gttgtcaccagaagcgtaccatactcacgaaatttttggagtaggctttctggcaacttaccggaaagcc

SEQ ID NO: 102 - DNA, 71 bp, synthetic construct
  source 1..71: mol_type = other DNA
ccgaatcaccagaagcgtaccatactcacgaaatttttggagtaggctttctggttcggtaccggaaagcc

SEQ ID NO: 103 - DNA, 73 bp, synthetic construct
  source 1..73: mol_type = other DNA
taaggagtcaccagaagcgtaccatactcacgaaatttttggagtaggctttctggctccttaccggaaagcc

SEQ ID NO: 104 - DNA, 70 bp, synthetic construct
  source 1..70: mol_type = other DNA
gaagtcaccagaagcgtaccatactcacgaaatttttggagtaggctttctggcttcttcccggaaagcc

SEQ ID NO: 105 - DNA, 72 bp, synthetic construct
  source 1..72: mol_type = other DNA
ggctttcaccagaagcgtaccatactcacgaaatttttggagtaggctttctggaagccttaccggaaagcc

SEQ ID NO: 106 - DNA, 70 bp, synthetic construct
  source 1..70: mol_type = other DNA
gaagtcaccagaagcgtaccatactcacgaaatttttggagtaggctttctggcttcttaccggaaagcc

SEQ ID NO: 107 - DNA, 90 bp, synthetic construct; identical to SEQ ID NO: 65
  source 1..90: mol_type = other DNA

SEQ ID NO: 108 - DNA, 90 bp, synthetic construct
  source 1..90: mol_type = other DNA
cctctcagcagcagcagcagcagcagcagcagcagcagcagcagcagcagaatttttggagtaggctttctgggaggttaccggaaagcc

SEQ ID NO: 109 - DNA, 92 bp, synthetic construct
  source 1..92: mol_type = other DNA
ggaggtcagcagcagcagcagcagcagcagcagcagcagcagcagcagcagaatttttggagtaggctttctggcctccttaccggaaagcc

SEQ ID NO: 110 - DNA, 92 bp, synthetic construct
  source 1..92: mol_type = other DNA
cctcctcagcagcagcagcagcagcagcagcagcagcagcagcagcagcagaatttttggagtaggctttctggggaggttaccggaaagcc

SEQ ID NO: 111 - DNA, 91 bp, synthetic construct
  source 1..91: mol_type = other DNA
agccagcagcagcagcagcagcagcagcagcagcagcagcagcagcagcagaatttttggagtaggctttctggctggctaccggaaagcc

SEQ ID NO: 112 - DNA, 88 bp, synthetic construct
  source 1..88: mol_type = other DNA
ggaagcagcagcagcagcagcagcagcagcagcagcagcagcagcagcagaatttttggagtaggctttctggcttccccggaaagcc

SEQ ID NO: 113 - DNA, 92 bp, synthetic construct
  source 1..92: mol_type = other DNA
gaagaagcagcagcagcagcagcagcagcagcagcagcagcagcagcagcagaatttttggagtaggctttctggcttcttcccggaaagcc

SEQ ID NO: 114 - DNA, 89 bp, synthetic construct
  source 1..89: mol_type = other DNA
gttgcagcagcagcagcagcagcagcagcagcagcagcagcagcagcagaatttttggagtaggctttctggcaacttaccggaaagcc

SEQ ID NO: 115 - DNA, 90 bp, synthetic construct
  source 1..90: mol_type = other DNA
ccgaacagcagcagcagcagcagcagcagcagcagcagcagcagcagcagaatttttggagtaggctttctggttcggtaccggaaagcc

SEQ ID NO: 116 - DNA, 92 bp, synthetic construct
  source 1..92: mol_type = other DNA
taaggagcagcagcagcagcagcagcagcagcagcagcagcagcagcagcagaatttttggagtaggctttctggctccttaccggaaagcc

SEQ ID NO: 117 - DNA, 89 bp, synthetic construct
  source 1..89: mol_type = other DNA
gaagcagcagcagcagcagcagcagcagcagcagcagcagcagcagcagaatttttggagtaggctttctggcttcttcccggaaagcc

SEQ ID NO: 118 - DNA, 91 bp, synthetic construct
  source 1..91: mol_type = other DNA
ggcttcagcagcagcagcagcagcagcagcagcagcagcagcagcagcagaatttttggagtaggctttctggaagccttaccggaaagcc

SEQ ID NO: 119 - DNA, 89 bp, synthetic construct
  source 1..89: mol_type = other DNA
gaagcagcagcagcagcagcagcagcagcagcagcagcagcagcagcagaatttttggagtaggctttctggcttcttaccggaaagcc

SEQ ID NO: 120 - DNA, 2469 bp, synthetic construct
  source 1..2469: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccggtcgcgtctagtaacttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagtcagcagcagcagcagcagcagcagcagcagcagcagcagcagcagaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaagcaaactacagagccaagtgctatccacagacttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagtcagcagcagcagcagcagcagcagcagcagcagcagcagcagcagaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaaggagctttctgggttgccatctcaagcagaccttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagtcagcagcagcagcagcagcagcagcagcagcagcagcagcagcagaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaagtacaaggccatcagctcatactcacaattgcttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagtcagcagcagcagcagcagcagcagcagcagcagcagcagcagcagaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaaggtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 121 - DNA, 2393 bp, synthetic construct
  source 1..2393: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccggtcgcgtctagtaacttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagttcaccagaagcgtaccatactcacgaaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaagcaaactacagagccaagtgctatccacagacttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagttcaccagaagcgtaccatactcacgaaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaaggagctttctgggttgccatctcaagcagaccttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagttcaccagaagcgtaccatactcacgaaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaagtacaaggccatcagctcatactcacaattgcttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagttcaccagaagcgtaccatactcacgaaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaaggtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 122 - DNA, 2469 bp, synthetic construct
  source 1..2469: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccggtcgcgtctagtaacttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagtctgctgctgctgctgctgctgctgctgctgctgctgctgctgctgaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaagcaaactacagagccaagtgctatccacagacttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagtctgctgctgctgctgctgctgctgctgctgctgctgctgctgctgaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaaggagctttctgggttgccatctcaagcagaccttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagtctgctgctgctgctgctgctgctgctgctgctgctgctgctgctgaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaagtacaaggccatcagctcatactcacaattgcttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagtctgctgctgctgctgctgctgctgctgctgctgctgctgctgctgaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaaggtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 123 - DNA, 2451 bp, synthetic construct
  source 1..2451: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccggtcgcgtctagtaaccggtcttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagtcagcagcagcagcagcagcagcagcagcagcagcagcagcagcagaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaagcaaactacagagccaagtgctatccacagacttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagtcagcagcagcagcagcagcagcagcagcagcagcagcagcagcagaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaaggagctttctgggttgccatctcaagcagaccttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagtcagcagcagcagcagcagcagcagcagcagcagcagcagcagcagaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaagtacaaggccatcagctcatactcacaattgcttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagtcagcagcagcagcagcagcagcagcagcagcagcagcagcagcagaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaaggtgcggacccaacggccgcctagtccactccctctctgcgcgctcgctcgctcactgaggccgggcgaccaaaggtcgcccgacgcccgggctttgcccgggcggcctcagtgagcgagcgagcgcgcagagaggga

SEQ ID NO: 124 - DNA, 2375 bp, synthetic construct
  source 1..2375: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccggtcgcgtctagtaaccggtcttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagttcaccagaagcgtaccatactcacgaaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaagcaaactacagagccaagtgctatccacagacttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagttcaccagaagcgtaccatactcacgaaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaaggagctttctgggttgccatctcaagcagaccttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagttcaccagaagcgtaccatactcacgaaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaagtacaaggccatcagctcatactcacaattgcttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagttcaccagaagcgtaccatactcacgaaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaaggtgcggacccaacggccgcctagtccactccctctctgcgcgctcgctcgctcactgaggccgggcgaccaaaggtcgcccgacgcccgggctttgcccgggcggcctcagtgagcgagcgagcgcgcagagaggga

SEQ ID NO: 125 - DNA, 2451 bp, synthetic construct
  source 1..2451: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccggtcgcgtctagtaaccggtcttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagtctgctgctgctgctgctgctgctgctgctgctgctgctgctgctgaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaagcaaactacagagccaagtgctatccacagacttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagtctgctgctgctgctgctgctgctgctgctgctgctgctgctgctgaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaaggagctttctgggttgccatctcaagcagaccttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagtctgctgctgctgctgctgctgctgctgctgctgctgctgctgctgaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaagtacaaggccatcagctcatactcacaattgcttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagtctgctgctgctgctgctgctgctgctgctgctgctgctgctgctgaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaaggtgcggacccaacggccgcctagtccactccctctctgcgcgctcgctcgctcactgaggccgggcgaccaaaggtcgcccgacgcccgggctttgcccgggcggcctcagtgagcgagcgagcgcgcagagaggga

SEQ ID NO: 126 - DNA, 2516 bp, synthetic construct
  source 1..2516: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagtcagcagcagcagcagcagcagcagcagcagcagcagcagcagcagaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaagctagtcgcgtcgacggatccgaattcgatatcgctagcataatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaagatatctagatctcgaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 127 - DNA, 2497 bp, synthetic construct
  source 1..2497: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagttcaccagaagcgtaccatactcacgaaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaagctagtcgcgtcgacggatccgaattcgatatcgctagcataatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaagatatctagatctcgaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 128 - DNA, 2516 bp, synthetic construct
  source 1..2516: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagtctgctgctgctgctgctgctgctgctgctgctgctgctgctgctgaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaagctagtcgcgtcgacggatccgaattcgatatcgctagcataatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaagatatctagatctcgaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 129 - DNA, 3756 bp, synthetic construct
  source 1..3756: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtcttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagtcagcagcagcagcagcagcagcagcagcagcagcagcagcagcagaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaagcaaactacagagccaagtgctatccacagagagctttctgggttgccatctcaagcagactacaaggccatcagctcatactcacaattgactttgagagcttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagtcagcagcagcagcagcagcagcagcagcagcagcagcagcagcagaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaagttgaccacatacgtgctctttcaaagttctgtgtttgaagttatgttagtaacaactgatgcccatcctgcaatgacaaatccaattctcagtgcagctccttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagtcagcagcagcagcagcagcagcagcagcagcagcagcagcagcagaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaagactagtcgcgtcgacggatccgaattcgatatcgctagcataatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaagatatctagatctcgaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 130 - DNA, 3699 bp, synthetic construct
  source 1..3699: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtcttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagttcaccagaagcgtaccatactcacgaaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaagcaaactacagagccaagtgctatccacagagagctttctgggttgccatctcaagcagactacaaggccatcagctcatactcacaattgactttgagagcttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagttcaccagaagcgtaccatactcacgaaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaagttgaccacatacgtgctctttcaaagttctgtgtttgaagttatgttagtaacaactgatgcccatcctgcaatgacaaatccaattctcagtgcagctccttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagttcaccagaagcgtaccatactcacgaaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaagactagtcgcgtcgacggatccgaattcgatatcgctagcataatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaagatatctagatctcgaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 131 - DNA, 3756 bp, synthetic construct
  source 1..3756: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtcttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagtctgctgctgctgctgctgctgctgctgctgctgctgctgctgctgaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaagcaaactacagagccaagtgctatccacagagagctttctgggttgccatctcaagcagactacaaggccatcagctcatactcacaattgactttgagagcttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagtctgctgctgctgctgctgctgctgctgctgctgctgctgctgctgaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaagttgaccacatacgtgctctttcaaagttctgtgtttgaagttatgttagtaacaactgatgcccatcctgcaatgacaaatccaattctcagtgcagctccttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagtctgctgctgctgctgctgctgctgctgctgctgctgctgctgctgaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaagactagtcgcgtcgacggatccgaattcgatatcgctagcataatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaagatatctagatctcgaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 132 - DNA, 4556 bp, synthetic construct
  source 1..4556: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtcttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagtcagcagcagcagcagcagcagcagcagcagcagcagcagcagcagaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaagcaaactacagagccaagtgctatccacagagagctttctgggttgccatctcaagcagactacaaggccatcagctcatactcacaattgactttgagagtcattttccaatgctcctacacaccccttcttcacaatccccaacaaatctgaggctggaacttggtaccataacaatcattacattatttcaccagaagtacaccttgcctggaagattggcattatagcatcttctaacattgtgaaagttagtgaccaatgaggagatccaagtcagttccagttggatttctctatactctataataaatatatatggtgtcttcaacaataggactttgccatccagtgatgctaaaaatcaataacaatggcaataacctgccctgtttggaaagcctctggcttccatgactaacaattcaaggcaggtctcctatacctagtactgagatttttatttgataaactatatcttctgggaggagaagcattgtcttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagtcagcagcagcagcagcagcagcagcagcagcagcagcagcagcagaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaagttgaccacatacgtgctctttcaaagttctgtgtttgaagttatgttagtaacaactgatgcccatcctgcaatgacaaatccaattctcagtgcagctctctgaaatagttttgctttctctctctaggtctgttctatactcctaactctccaggagtttacaaggaataaaatctcttccaaatgctttctgttgcaacaactggaccatactgaaagctgaggcccacaattgcaatctaggttagcaggtaatcattgttggtgaggtcctccctttccccaggctcgtgtttgtattggggagcaggaaatttttgctagagcagcactgccatctctctacactccacctgattggtgggatggaccagagaaatggacattcccaacacagtccctcctttcacatctgctcacctgcccacaggatactttccaccatgcatactgggctctgcaccaaccattcagcagtgatgaagaggaaacttgaaccttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagtcagcagcagcagcagcagcagcagcagcagcagcagcagcagcagaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaagactagtcgcgtcgacggatccgaattcgatatcgctagcataatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaagatatctagatctcgaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 133 - DNA, 4499 bp, synthetic construct
  source 1..4499: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtcttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagttcaccagaagcgtaccatactcacgaaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaagcaaactacagagccaagtgctatccacagagagctttctgggttgccatctcaagcagactacaaggccatcagctcatactcacaattgactttgagagtcattttccaatgctcctacacaccccttcttcacaatccccaacaaatctgaggctggaacttggtaccataacaatcattacattatttcaccagaagtacaccttgcctggaagattggcattatagcatcttctaacattgtgaaagttagtgaccaatgaggagatccaagtcagttccagttggatttctctatactctataataaatatatatggtgtcttcaacaataggactttgccatccagtgatgctaaaaatcaataacaatggcaataacctgccctgtttggaaagcctctggcttccatgactaacaattcaaggcaggtctcctatacctagtactgagatttttatttgataaactatatcttctgggaggagaagcattgtcttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagttcaccagaagcgtaccatactcacgaaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaagttgaccacatacgtgctctttcaaagttctgtgtttgaagttatgttagtaacaactgatgcccatcctgcaatgacaaatccaattctcagtgcagctctctgaaatagttttgctttctctctctaggtctgttctatactcctaactctccaggagtttacaaggaataaaatctcttccaaatgctttctgttgcaacaactggaccatactgaaagctgaggcccacaattgcaatctaggttagcaggtaatcattgttggtgaggtcctccctttccccaggctcgtgtttgtattggggagcaggaaatttttgctagagcagcactgccatctctctacactccacctgattggtgggatggaccagagaaatggacattcccaacacagtccctcctttcacatctgctcacctgcccacaggatactttccaccatgcatactgggctctgcaccaaccattcagcagtgatgaagaggaaacttgaaccttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagttcaccagaagcgtaccatactcacgaaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaagactagtcgcgtcgacggatccgaattcgatatcgctagcataatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaagatatctagatctcgaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 134 - DNA, 4556 bp, synthetic construct
  source 1..4556: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtcttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagtctgctgctgctgctgctgctgctgctgctgctgctgctgctgctgaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaagcaaactacagagccaagtgctatccacagagagctttctgggttgccatctcaagcagactacaaggccatcagctcatactcacaattgactttgagagtcattttccaatgctcctacacaccccttcttcacaatccccaacaaatctgaggctggaacttggtaccataacaatcattacattatttcaccagaagtacaccttgcctggaagattggcattatagcatcttctaacattgtgaaagttagtgaccaatgaggagatccaagtcagttccagttggatttctctatactctataataaatatatatggtgtcttcaacaataggactttgccatccagtgatgctaaaaatcaataacaatggcaataacctgccctgtttggaaagcctctggcttccatgactaacaattcaaggcaggtctcctatacctagtactgagatttttatttgataaactatatcttctgggaggagaagcattgtcttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagtctgctgctgctgctgctgctgctgctgctgctgctgctgctgctgaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaagttgaccacatacgtgctctttcaaagttctgtgtttgaagttatgttagtaacaactgatgcccatcctgcaatgacaaatccaattctcagtgcagctctctgaaatagttttgctttctctctctaggtctgttctatactcctaactctccaggagtttacaaggaataaaatctcttccaaatgctttctgttgcaacaactggaccatactgaaagctgaggcccacaattgcaatctaggttagcaggtaatcattgttggtgaggtcctccctttccccaggctcgtgtttgtattggggagcaggaaatttttgctagagcagcactgccatctctctacactccacctgattggtgggatggaccagagaaatggacattcccaacacagtccctcctttcacatctgctcacctgcccacaggatactttccaccatgcatactgggctctgcaccaaccattcagcagtgatgaagaggaaacttgaaccttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagtctgctgctgctgctgctgctgctgctgctgctgctgctgctgctgaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaagactagtcgcgtcgacggatccgaattcgatatcgctagcataatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaagatatctagatctcgaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 135 - DNA, 1424 bp, synthetic construct
  source 1..1424: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagttacctccaacatcaaggaagatggcagtaaccacaggttgtgtcaccagagtaacaaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaagctagtcgcgtaaggaccagcttctttgggagagaacagacgcaggggcgggagggaaaaagggagaggcagacgtcacttccccttggcggctctggcagcagattggtcggttgagtggcagaaaggcagacggggactgggcaaggcactgtcggtgacatcacggacagggcgacttctatgtagatgaggcagcgcagaggctgacgtcttcgccacttgctgcttcaccacgaaggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagaggcacaacgtttcggagttacctccaacatcaaggaagatggcagtaaccacaggttgtgtcaccagagtaacaaatttttggagtaggctttctggctccttaccggaaagccactttctggagtttcaaaaacagactgtacgccaagggtcatatctttttcgtcgacggatccgaattcgatatctagatctcgaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 136 - DNA, 1368 bp, synthetic construct
  source 1..1368: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagtgtaaccacaggttgtgtcaccagagtaacaaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaagctagtcgcgtaaggaccagcttctttgggagagaacagacgcaggggcgggagggaaaaagggagaggcagacgtcacttccccttggcggctctggcagcagattggtcggttgagtggcagaaaggcagacggggactgggcaaggcactgtcggtgacatcacggacagggcgacttctatgtagatgaggcagcgcagaggctgacgtcttcgccacttgctgcttcaccacgaaggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagaggcacaacgtttcggagttacctccaacatcaaggaagatggcaaatttttggagtaggctttctggctccttaccggaaagccactttctggagtttcaaaaacagactgtacgccaagggtcatatctttttcgtcgacggatccgaattcgatatctagatctcgaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 137 - DNA, 1407 bp, synthetic construct
  source 1..1407: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccggtcgcgtctagtaacttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagttacctccaacatcaaggaagatggcagtaaccacaggttgtgtcaccagagtaacaaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaagctagtcgcgtaaggaccagcttctttgggagagaacagacgcaggggcgggagggaaaaagggagaggcagacgtcacttccccttggcggctctggcagcagattggtcggttgagtggcagaaaggcagacggggactgggcaaggcactgtcggtgacatcacggacagggcgacttctatgtagatgaggcagcgcagaggctgacgtcttcgccacttgctgcttcaccacgaaggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagaggcacaacgtttcggagttacctccaacatcaaggaagatggcagtaaccacaggttgtgtcaccagagtaacaaatttttggagtaggctttctggctccttaccggaaagccactttctggagtttcaaaaacagactgtacgccaagggtcatatctttttcgtcgacggatccgaattcgatatctagatctcgaggtaaccacgtgcggaccgagcctagtccactccctctctgcgcgctcgctcgctcactgaggccgggcgaccaaaggtcgcccgacgcccgggctttgcccgggcggcctcagtgagcgagcgagcgcgcagagaggga

SEQ ID NO: 138 - DNA, 3074 bp, synthetic construct
  source 1..3074: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagttacctccaacatcaaggaagatggcagtaaccacaggttgtgtcaccagagtaacaaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaagctagtcgcgtaaggaccagcttctttgggagagaacagacgcaggggcgggagggaaaaagggagaggcagacgtcacttccccttggcggctctggcagcagattggtcggttgagtggcagaaaggcagacggggactgggcaaggcactgtcggtgacatcacggacagggcgacttctatgtagatgaggcagcgcagaggctgacgtcttcgccacttgctgcttcaccacgaaggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagaggcacaacgtttcggagttacctccaacatcaaggaagatggcagtaaccacaggttgtgtcaccagagtaacaaatttttggagtaggctttctggctccttaccggaaagccactttctggagtttcaaaaacagactgtacgccaagggtcatatctttttcgtcgacggatccgcaaactacagagccaagtgctatccacagagagctttctgggttgccatctcaagcagactacaaggccatcagctcatactcacaattgactttgagagtcattttccaatgctcctacacaccccttcttcacaatccccaacaaatctgaggctggaacttggtaccataacaatcattacattatttcaccagaagtacaccttgcctggaagattggcattatagcatcttctaacattgtgaaagttagtgaccaatgaggagatccaagtcagttccagttggatttctctatactctataataaatatatatggtgtcttcaacaataggactttgccatccagtgatgctaaaaatcaataacaatggcaataacctgccctgtttggaaagcctctggcttccatgactaacaattcaaggcaggtctcctatacctagtactgagatttttatttgataaactatatcttctgggaggagaagcattgtttgaccacatacgtgctctttcaaagttctgtgtttgaagttatgttagtaacaactgatgcccatcctgcaatgacaaatccaattctcagtgcagctctctgaaatagttttgctttctctctctaggtctgttctatactcctaactctccaggagtttacaaggaataaaatctcttccaaatgctttctgttgcaacaactggaccatactgaaagctgaggcccacaattgcaatctaggttagcaggtaatcattgttggtgaggtcctccctttccccaggctcgtgtttgtattggggagcaggaaatttttgctagagcagcactgccatctctctacactccacctgattggtgggatggaccagagaaatggacattcccaacacagtccctcctttcacatctgctcacctgcccacaggatactttccaccatgcatactgggctctgcaccaaccattcagcagtgatgaagaggaaacttgaacccatgcagagtacctctagagaaaagtttctaaagttggttttgccttgcttagaaaggaggtcaaaggtgaagcctaggagaaacattcagcaatgttgcacaaagacatgagaataaatgattttagcatcctgaaatgaagagcatagtgatctcaccatagctacccacctacccagagaacttccaggctttcactgtctcttttctgaatcacccacattacagaatatcagtcattgtgtaattagataacagattgtgaggtgcccaaacagaaacaaactttacaggaggagagagaaaatcaaggtgatgactatggagaacaaaatgagaaggagaggtggagttcctgctgcatgaacacctatcctgctatagagcccatttcttccataaataatacaactctgtaactccataggtcagaaagaaacagtgttctgtgtcttcccatctagcaccacaaacccccacaagattaatttgtttctagggacccttaaatctctatcaaaattctggaaacctctactttagaaaatcttatatttattattcaggctactttccagagtgataagctactgagtctcctaagtgtcatctatggtacacagggataagatcaggaataaacctgggaattcgatatctagatctcgaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 139 - DNA, 3051 bp, synthetic construct
  source 1..3051: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagtcagcagcagcagcagcagcagcagcagcagcagcagcagcagcagaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaagctagtcgcgtaaggaccagcttctttgggagagaacagacgcaggggcgggagggaaaaagggagaggcagacgtcacttccccttggcggctctggcagcagattggtcggttgagtggcagaaaggcagacggggactgggcaaggcactgtcggtgacatcacggacagggcgacttctatgtagatgaggcagcgcagaggctgacgtcttcgccacttgctgcttcaccacgaaggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagaggcacaacgtttcggagtcagcagcagcagcagcagcagcagcagcagcagcagcagcagcagaatttttggagtaggctttctggctccttaccggaaagccactttctggagtttcaaaaacagactgtacgccaagggtcatatctttttcgtcgacggatccgaattcgatatcgctagcataatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaagatatctagatctcgaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 140 - DNA, 3055 bp, synthetic construct
  source 1..3055: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacttcgcgatgtacgggccagatatatactgccgactagttaaggaccagcttctttgggagagaacagacgcaggggcgggagggaaaaagggagaggcagacgtcacttccccttggcggctctggcagcagattggtcggttgagtggcagaaaggcagacggggactgggcaaggcactgtcggtgacatcacggacagggcgacttctatgtagatgaggcagcgcagaggctgacgtcttcgccacttgctgcttcaccacgaaggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagaggcacaacgtttcggagtcagcagcagcagcagcagcagcagcagcagcagcagcagcagcagaatttttggagtaggctttctggctccttaccggaaagccactttctggagtttcaaaaacagactgtacgccaagggtcatatctttttaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagtcagcagcagcagcagcagcagcagcagcagcagcagcagcagcagaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaagctagtcgcgtcgacggatccgaattcgatatcgctagcataatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaagatatctagatctcgaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 141 - DNA, 3013 bp, synthetic construct
  source 1..3013: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagttcaccagaagcgtaccatactcacgaaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaagctagtcgcgtaaggaccagcttctttgggagagaacagacgcaggggcgggagggaaaaagggagaggcagacgtcacttccccttggcggctctggcagcagattggtcggttgagtggcagaaaggcagacggggactgggcaaggcactgtcggtgacatcacggacagggcgacttctatgtagatgaggcagcgcagaggctgacgtcttcgccacttgctgcttcaccacgaaggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagaggcacaacgtttcggagttcaccagaagcgtaccatactcacgaaatttttggagtaggctttctggctccttaccggaaagccactttctggagtttcaaaaacagactgtacgccaagggtcatatctttttcgtcgacggatccgaattcgatatcgctagcataatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaagatatctagatctcgaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 142 - DNA, 1402 bp, synthetic construct
  source 1..1402: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagtcagcagcagcagcagcagcagcagcagcagcagcagcagcagcagaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaagctagtcgcgtaaggaccagcttctttgggagagaacagacgcaggggcgggagggaaaaagggagaggcagacgtcacttccccttggcggctctggcagcagattggtcggttgagtggcagaaaggcagacggggactgggcaaggcactgtcggtgacatcacggacagggcgacttctatgtagatgaggcagcgcagaggctgacgtcttcgccacttgctgcttcaccacgaaggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagaggcacaacgtttcggagtcagcagcagcagcagcagcagcagcagcagcagcagcagcagcagaatttttggagtaggctttctggctccttaccggaaagccactttctggagtttcaaaaacagactgtacgccaagggtcatatctttttcgtcgacggatccgaattcgatatctagatctcgaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 143 - DNA, 1385 bp, synthetic construct
  source 1..1385: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccggtcgcgtctagtaacttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagtcagcagcagcagcagcagcagcagcagcagcagcagcagcagcagaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaagctagtcgcgtaaggaccagcttctttgggagagaacagacgcaggggcgggagggaaaaagggagaggcagacgtcacttccccttggcggctctggcagcagattggtcggttgagtggcagaaaggcagacggggactgggcaaggcactgtcggtgacatcacggacagggcgacttctatgtagatgaggcagcgcagaggctgacgtcttcgccacttgctgcttcaccacgaaggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagaggcacaacgtttcggagtcagcagcagcagcagcagcagcagcagcagcagcagcagcagcagaatttttggagtaggctttctggctccttaccggaaagccactttctggagtttcaaaaacagactgtacgccaagggtcatatctttttcgtcgacggatccgaattcgatatctagatctcgaggtaaccacgtgcggaccgagcctagtccactccctctctgcgcgctcgctcgctcactgaggccgggcgaccaaaggtcgcccgacgcccgggctttgcccgggcggcctcagtgagcgagcgagcgcgcagagaggga

SEQ ID NO: 144 - DNA, 1650 bp, synthetic construct
  source 1..1650: mol_type = other DNA
caaactacagagccaagtgctatccacagagagctttctgggttgccatctcaagcagactacaaggccatcagctcatactcacaattgactttgagagtcattttccaatgctcctacacaccccttcttcacaatccccaacaaatctgaggctggaacttggtaccataacaatcattacattatttcaccagaagtacaccttgcctggaagattggcattatagcatcttctaacattgtgaaagttagtgaccaatgaggagatccaagtcagttccagttggatttctctatactctataataaatatatatggtgtcttcaacaataggactttgccatccagtgatgctaaaaatcaataacaatggcaataacctgccctgtttggaaagcctctggcttccatgactaacaattcaaggcaggtctcctatacctagtactgagatttttatttgataaactatatcttctgggaggagaagcattgtttgaccacatacgtgctctttcaaagttctgtgtttgaagttatgttagtaacaactgatgcccatcctgcaatgacaaatccaattctcagtgcagctctctgaaatagttttgctttctctctctaggtctgttctatactcctaactctccaggagtttacaaggaataaaatctcttccaaatgctttctgttgcaacaactggaccatactgaaagctgaggcccacaattgcaatctaggttagcaggtaatcattgttggtgaggtcctccctttccccaggctcgtgtttgtattggggagcaggaaatttttgctagagcagcactgccatctctctacactccacctgattggtgggatggaccagagaaatggacattcccaacacagtccctcctttcacatctgctcacctgcccacaggatactttccaccatgcatactgggctctgcaccaaccattcagcagtgatgaagaggaaacttgaacccatgcagagtacctctagagaaaagtttctaaagttggttttgccttgcttagaaaggaggtcaaaggtgaagcctaggagaaacattcagcaatgttgcacaaagacatgagaataaatgattttagcatcctgaaatgaagagcatagtgatctcaccatagctacccacctacccagagaacttccaggctttcactgtctcttttctgaatcacccacattacagaatatcagtcattgtgtaattagataacagattgtgaggtgcccaaacagaaacaaactttacaggaggagagagaaaatcaaggtgatgactatggagaacaaaatgagaaggagaggtggagttcctgctgcatgaacacctatcctgctatagagcccatttcttccataaataatacaactctgtaactccataggtcagaaagaaacagtgttctgtgtcttcccatctagcaccacaaacccccacaagattaatttgtttctagggacccttaaatctctatcaaaattctggaaacctctactttagaaaatcttatatttattattcaggctactttccagagtgataagctactgagtctcctaagtgtcatctatggtacacagggataagatcaggaataaacctggg

SEQ ID NO: 145 - DNA, 29 bp, synthetic construct
  source 1..29: mol_type = other DNA
ggttttctgacttcggtcggaaaacccct

SEQ ID NO: 146 - DNA, 29 bp, synthetic construct
  source 1..29: mol_type = other DNA
ggttttctgacctccgtcggaaaacccct

SEQ ID NO: 147 - DNA, 33 bp, synthetic construct
  source 1..33: mol_type = other DNA
ggttttctgacctccttcggtcggaaaacccct

SEQ ID NO: 148 - DNA, 26 bp, synthetic construct
  source 1..26: mol_type = other DNA
ggttttctgacctccgtcggaaaacc

SEQ ID NO: 149 - DNA, 56 bp, synthetic construct
  source 1..56: mol_type = other DNA
gtaaccacaggttgtgtcaccagagtaacatacctccaacatcaaggaagatggca

SEQ ID NO: 150 - DNA, 56 bp, synthetic construct
  source 1..56: mol_type = other DNA
tacctccaacatcaaggaagatggcagtaaccacaggttgtgtcaccagagtaaca

SEQ ID NO: 151 - DNA, 256 bp, synthetic construct
  source 1..256: mol_type = other DNA
taacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgc

SEQ ID NO: 152 - DNA, 440 bp, synthetic construct
  source 1..440: mol_type = other DNA
tgccaacagctttgaaatcctctgtgctcttgtgccaatcccaccctcagaacagggagtctgtcggttatgttttcgtcctttacagttcttcgccgtctaatccattggaaacctcctttatttaaaggaaaccgtgtccaaattcagtgtacagaggtgcaaacctccgccccgctccttgtaggaagcccacgggaactgggccaatcggacggcgcttactcggctcatttacatacccataacacaccgcggctaatgcaaatattttcgtgaaaagatcttccattactcgaagttttaaaactaaaaacaaaatattatttcaaaagatcatgaagaaatttctgtcggtaacagcagtttcaattgataaatcaccatcacttatctaggaggttctgttactctagaagtgaattaagcaggacagctgt

SEQ ID NO: 153 - DNA, 231 bp, synthetic construct
  source 1..231: mol_type = other DNA
gggcggggcatgcaaataactgctctgtggaactctgggagcaaaaacaaaaaactgcaaccaaaacttctcggcctccttgaatcttacaggcttttcgtggcgtaaaggtggtgtactcaatgaagaggagagtctgtgttggctgcatgtttgagtcggttggttggtgactgtgaattaaaggtgtggtcggtgttgagtgtatggggcgtgtgggcgtagttcggt

SEQ ID NO: 154 - DNA, 311 bp, synthetic construct
  source 1..311: mol_type = other DNA
cgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgttt

SEQ ID NO: 155 - DNA, 97 bp, synthetic construct
  source 1..97: mol_type = other DNA
catgcaaattacgcgctgtgctttgtgggaaatcaccctaaacgtaaaatttattcctctttcgagccttatagtggcggccggtctacaccctaaa

SEQ ID NO: 156 - DNA, 107 bp, synthetic construct
  source 1..107: mol_type = other DNA
cccaatttcactggtctacaatgaaagcaaaacagttctcttccccgctccccggtgtgtgagaggggctttgatccttctctggtttcctaggaaacgcgtatgtg

SEQ ID NO: 157 - DNA, 200 bp, synthetic construct
  source 1..200: mol_type = other DNA
ccacattttgtgtttaaaaatagaaatatttaagtgagatcagtttaaatctgctttatctagggtgtctaactgcttgcatctttttaacttttccttatttgtgagaaggtctgtaaaacttattatatgttagtacactgtagctgtcttcagacactccagaagagggattcagatcaccttacagatggttttga

SEQ ID NO: 158 - DNA, 78 bp, synthetic construct
  source 1..78: mol_type = other DNA
ccctctggggagtaaagttggttttaaagtcagagcatggtgattgtagggcagtccaacttttttaaatatgctgtg

SEQ ID NO: 159-160 - skipped (2 entries)

SEQ ID NO: 161 - DNA, 13 bp, synthetic construct
  source 1..13: mol_type = other DNA
aatttttggagca

SEQ ID NO: 162 - DNA, 110 bp, synthetic construct
  source 1..110: mol_type = other DNA
ctgcgcgctcgctcgctcactgaggccgcccgggcaaagcccgggcgtcgggcgacctttggtcgcccggcctcagtgagcgagcgagcgcgcagagagggagtggggtt

SEQ ID NO: 163 - DNA, 30 bp, synthetic construct
  source 1..30: mol_type = other DNA
ggctttctggctccttaccggaaagcccct

SEQ ID NO: 164 - DNA, 13 bp, synthetic construct
  source 1..13: mol_type = other DNA
aatttttggagta

SEQ ID NO: 165 - DNA, 45 bp, synthetic construct
  source 1..45: mol_type = other DNA
cagcagcagcagcagcagcagcagcagcagcagcagcagcagcag

SEQ ID NO: 166 - DNA, 133 bp, synthetic construct
  source 1..133: mol_type = other DNA
aaggaacccctagtgatggagttggccactccctctctgcgcgctcgctcgctcactgaggccgggcgaccaaaggtcgcccgacgcccgggctttgcccgggcggcctcagtgagcgagcgagcgcgccagc

SEQ ID NO: 167 - DNA, 45 bp, synthetic construct
  source 1..45: mol_type = other DNA
ctgctgctgctgctgctgctgctgctgctgctgctgctgctgctg

SEQ ID NO: 168 - DNA, 1532 bp, synthetic construct
  source 1..1532: mol_type = other DNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaggagtgtaaccacaggttgtgtcaccagagtaacatacctccaacatcaaggaagatggcaaatttttggagcaggttttctgacctccgtcggaaaacccctcccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcggagtgtaaccacaggttgtgtcaccagagtaacatacctccaacatcaaggaagatggcaaatttttggagcaggttttctgacctccgtcggaaaacccctgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 169 - DNA, 1532 bp, synthetic construct
  source 1..1532: mol_type = other DNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaggagttacctccaacatcaaggaagatggcagtaaccacaggttgtgtcaccagagtaacaaatttttggagcaggttttctgacctccgtcggaaaacccctcccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcggagttacctccaacatcaaggaagatggcagtaaccacaggttgtgtcaccagagtaacaaatttttggagcaggttttctgacctccgtcggaaaacccctgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 170 - DNA, 1526 bp, synthetic construct
  source 1..1526: mol_type = other DNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaggagtgtaaccacaggttgtgtcaccagagtaacatacctccaacatcaaggaagatggcaaatttttggagcaggttttctgacctccgtcggaaaacccccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcggagtgtaaccacaggttgtgtcaccagagtaacatacctccaacatcaaggaagatggcaaatttttggagcaggttttctgacctccgtcggaaaaccgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 171 - DNA, 1242 bp, synthetic construct
  source 1..1242: mol_type = other DNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaatacctccaacatcaaggaagatggcagtaaccacaggttgtgtcaccagagtaacaaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtgtaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaatacctccaacatcaaggaagatggcagtaaccacaggttgtgtcaccagagtaacaaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtggaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 172 - DNA, 1522 bp, synthetic construct
  source 1..1522: mol_type = other DNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaatacctccaacatcaaggaagatggcagtaaccacaggttgtgtcaccagagtaacaaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactctacctccaacatcaaggaagatggcagtaaccacaggttgtgtcaccagagtaacaaatttttggagcaggttttctgacttcggtcggaaaacccctgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 173 - DNA, 1528 bp, synthetic construct
  source 1..1528: mol_type = other DNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaggagttacctccaacatcaaggaagatggcagtaaccacaggttgtgtcaccagagtaacaaatttttggagtaggctttctggctccttaccggaaagcccccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcggagttacctccaacatcaaggaagatggcagtaaccacaggttgtgtcaccagagtaacaaatttttggagtaggctttctggctccttaccggaaagccgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 174 - DNA, 1534 bp, synthetic construct
  source 1..1534: mol_type = other DNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaggagttacctccaacatcaaggaagatggcagtaaccacaggttgtgtcaccagagtaacaaatttttggagtaggctttctggctccttaccggaaagcccctcccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcggagttacctccaacatcaaggaagatggcagtaaccacaggttgtgtcaccagagtaacaaatttttggagtaggctttctggctccttaccggaaagcccctgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 175 - DNA, 1540 bp, synthetic construct
  source 1..1540: mol_type = other DNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaggagttacctccaacatcaaggaagatggcagtaaccacaggttgtgtcaccagagtaacaaatttttggagcaggttttctgacctccttcggtcggaaaacccctcccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcggagttacctccaacatcaaggaagatggcagtaaccacaggttgtgtcaccagagtaacaaatttttggagcaggttttctgacctccttcggtcggaaaacccctgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 176 - DNA, 1385 bp, synthetic construct; identical to SEQ ID NO: 143
  source 1..1385: mol_type = other DNA

SEQ ID NO: 177 - DNA, 1393 bp, synthetic construct
  source 1..1393: mol_type = other DNA
ctgcgcgctcgctcgctcactgaggccgcccgggcaaagcccgggcgtcgggcgacctttggtcgcccggcctcagtgagcgagcgagcgcgcagagagggagtggggttcttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagtcagcagcagcagcagcagcagcagcagcagcagcagcagcagcagaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaagctagtcgcgtaaggaccagcttctttgggagagaacagacgcaggggcgggagggaaaaagggagaggcagacgtcacttccccttggcggctctggcagcagattggtcggttgagtggcagaaaggcagacggggactgggcaaggcactgtcggtgacatcacggacagggcgacttctatgtagatgaggcagcgcagaggctgacgtcttcgccacttgctgcttcaccacgaaggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagaggcacaacgtttcggagtcagcagcagcagcagcagcagcagcagcagcagcagcagcagcagaatttttggagtaggctttctggctccttaccggaaagccactttctggagtttcaaaaacagactgtacgccaagggtcatatctttttcgtcgacggatccgaattcgatatctagatctcgaggtaaccacgtgcggaccgagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagc

SEQ ID NO: 178 - DNA, 1390 bp, synthetic construct
  source 1..1390: mol_type = other DNA
ctgcgcgctcgctcgctcactgaggccgcccgggcaaagcccgggcgtcgggcgacctttggtcgcccggcctcagtgagcgagcgagcgcgcagagagggagtggggttgtacagtgaggatctatttcaattcctcgagtactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagtctgctgctgctgctgctgctgctgctgctgctgctgctgctgctgaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaagctagtcgcgtaaggaccagcttctttgggagagaacagacgcaggggcgggagggaaaaagggagaggcagacgtcacttccccttggcggctctggcagcagattggtcggttgagtggcagaaaggcagacggggactgggcaaggcactgtcggtgacatcacggacagggcgacttctatgtagatgaggcagcgcagaggctgacgtcttcgccacttgctgcttcaccacgaaggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagaggcacaacgtttcggagtctgctgctgctgctgctgctgctgctgctgctgctgctgctgctgaatttttggagtaggctttctggctccttaccggaaagccactttctggagtttcaaaaacagactgtacgccaagggtcatatctttttcgtcgacggatccgaattcgatatcgctagcctgcagtcgagatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagc

SEQ ID NO: 179 - DNA, 103 bp, synthetic construct
  source 1..103: mol_type = other DNA
ggagtgtaaccacaggttgtgtcaccagagtaacatacctccaacatcaaggaagatggcaaatttttggagcaggttttctgacctccgtcggaaaacccct

SEQ ID NO: 180 - DNA, 103 bp, synthetic construct
  source 1..103: mol_type = other DNA
ggagttacctccaacatcaaggaagatggcagtaaccacaggttgtgtcaccagagtaacaaatttttggagcaggttttctgacctccgtcggaaaacccct

SEQ ID NO: 181 - DNA, 100 bp, synthetic construct
  source 1..100: mol_type = other DNA
ggagtgtaaccacaggttgtgtcaccagagtaacatacctccaacatcaaggaagatggcaaatttttggagcaggttttctgacctccgtcggaaaacc

SEQ ID NO: 182 - DNA, 98 bp, synthetic construct
  source 1..98: mol_type = other DNA
tacctccaacatcaaggaagatggcagtaaccacaggttgtgtcaccagagtaacaaatttttggagcaggttttctgacttcggtcggaaaacccct

SEQ ID NO: 183 - DNA, 101 bp, synthetic construct; identical to SEQ ID NO: 68
  source 1..101: mol_type = other DNA

SEQ ID NO: 184 - DNA, 104 bp, synthetic construct
  source 1..104: mol_type = other DNA
ggagttacctccaacatcaaggaagatggcagtaaccacaggttgtgtcaccagagtaacaaatttttggagtaggctttctggctccttaccggaaagcccct

SEQ ID NO: 185 - DNA, 107 bp, synthetic construct
  source 1..107: mol_type = other DNA
ggagttacctccaacatcaaggaagatggcagtaaccacaggttgtgtcaccagagtaacaaatttttggagcaggttttctgacctccttcggtcggaaaacccct

SEQ ID NO: 186 - DNA, 29 bp, synthetic construct
  source 1..29: mol_type = other DNA
ggctttctggcctccgccggaaagcccct

SEQ ID NO: 187 - DNA, 30 bp, synthetic construct
  source 1..30: mol_type = other DNA
ggttttctgacactccgtcggaaaacccct

SEQ ID NO: 188 - DNA, 29 bp, synthetic construct
  source 1..29: mol_type = other DNA
ggttttctgatctccatcggaaaacccct

SEQ ID NO: 189 - DNA, 29 bp, synthetic construct
  source 1..29: mol_type = other DNA
ggttttccgacctccgtcggaaaacccct

SEQ ID NO: 190 - DNA, 30 bp, synthetic construct
  source 1..30: mol_type = other DNA
ggctttctggcactccaccggaaagcccct

SEQ ID NO: 191 - DNA, 30 bp, synthetic construct
  source 1..30: mol_type = other DNA
ggctttctggcactccgccggaaagcccct

SEQ ID NO: 192 - DNA, 29 bp, synthetic construct
  source 1..29: mol_type = other DNA
ggctttctggcctccaccggaaagcccct

SEQ ID NO: 193 - DNA, 29 bp, synthetic construct
  source 1..29: mol_type = other DNA
ggttttccggtctccaccggaaagccccc

SEQ ID NO: 194 - DNA, 29 bp, synthetic construct
  source 1..29: mol_type = other DNA
ggctttccggtctccaccggaaagcccct

SEQ ID NO: 195 - DNA, 29 bp, synthetic construct
  source 1..29: mol_type = other DNA
ggctttccggcctccgccggaaagcccct

SEQ ID NO: 196 - DNA, 29 bp, synthetic construct
  source 1..29: mol_type = other DNA
ggttttccggtctccaccggaaaaccctt

SEQ ID NO: 197 - DNA, 29 bp, synthetic construct
  source 1..29: mol_type = other DNA
ggttttccgtgctcccacggaaaaccctt

SEQ ID NO: 198 - DNA, 29 bp, synthetic construct
  source 1..29: mol_type = other DNA
ggttttccggcctccgccggaaaaccctt

SEQ ID NO: 199 - DNA, 30 bp, synthetic construct
  source 1..30: mol_type = other DNA
ggttttccgtgactcccacggaaaaccctt

SEQ ID NO: 200 - DNA, 30 bp, synthetic construct
  source 1..30: mol_type = other DNA
ggttttccggcactccgccggaaaaccctt

SEQ ID NO: 201 - DNA, 29 bp, synthetic construct
  source 1..29: mol_type = other DNA
ggtcttccggtctcctccggaaggccccc

SEQ ID NO: 202 - DNA, 27 bp, synthetic construct
  source 1..27: mol_type = other DNA
ggtcttccggctccccggaaggccccc

SEQ ID NO: 203 - DNA, 29 bp, synthetic construct
  source 1..29: mol_type = other DNA
ggctttccgtgctcccacggaaagcccct

SEQ ID NO: 204 - DNA, 30 bp, synthetic construct
  source 1..30: mol_type = other DNA
ggctttccgtgactcccacggaaagcccct

SEQ ID NO: 205 - DNA, 30 bp, synthetic construct
  source 1..30: mol_type = other DNA
ggctttccggcactccgccggaaagcccct

SEQ ID NO: 206 - DNA, 26 bp, synthetic construct
  source 1..26: mol_type = other DNA
tacctccaacatcaaggaagatggca

SEQ ID NO: 207 - DNA, 30 bp, synthetic construct
  source 1..30: mol_type = other DNA
gtaaccacaggttgtgtcaccagagtaaca

SEQ ID NO: 208 - DNA, 58 bp, synthetic construct
  source 1..58: mol_type = other DNA
cataccttctgcttgatgatcatctcgcaccagagtaacagtctgagtaggagctaaa

SEQ ID NO: 209 - DNA, 53 bp, synthetic construct
  source 1..53: mol_type = other DNA
cataccttctgcttgatgatcatctcgtgccgctgcccaatgccatcctggag

SEQ ID NO: 210 - DNA, 52 bp, synthetic construct
  source 1..52: mol_type = other DNA
cataccttctgcttgatgatcatctcggcccaatgccatcctggagttcctg

SEQ ID NO: 211 - DNA, 56 bp, synthetic construct; identical to SEQ ID NO: 150
  source 1..56: mol_type = other DNA

SEQ ID NO: 212 - DNA, 56 bp, synthetic construct
  source 1..56: mol_type = other DNA
aacatcaaggaagatggcatttctaggtaaccacaggttgtgtcaccagagtaaca

SEQ ID NO: 213 - DNA, 52 bp, synthetic construct
  source 1..52: mol_type = other DNA
aacatcaaggaagatggcatttctagtgccgctgcccaatgccatcctggag

SEQ ID NO: 214 - DNA, 52 bp, synthetic construct
  source 1..52: mol_type = other DNA
tttttctcataccttctgcttgatgtctgagtaggagctaaaatattttggg

SEQ ID NO: 215 - DNA, 56 bp, synthetic construct; identical to SEQ ID NO: 149
  source 1..56: mol_type = other DNA

SEQ ID NO: 216 - DNA, 58 bp, synthetic construct
  source 1..58: mol_type = other DNA
cgagatgatcatcaagcagaaggtatgcaccagagtaacagtctgagtaggagctaaa

SEQ ID NO: 217 - DNA, 57 bp, synthetic construct
  source 1..57: mol_type = other DNA
caccagagtaacagtctgagtaggagctaaatacctccaacatcaaggaagatggca

SEQ ID NO: 218 - DNA, 57 bp, synthetic construct
  source 1..57: mol_type = other DNA
tacctccaacatcaaggaagatggcacaccagagtaacagtctgagtaggagctaaa

SEQ ID NO: 219 - DNA, 54 bp, synthetic construct
  source 1..54: mol_type = other DNA
gtctgagtaggagctaaaatattttgggtacctccaacatcaaggaagatggca

SEQ ID NO: 220 - DNA, 54 bp, synthetic construct
  source 1..54: mol_type = other DNA
tacctccaacatcaaggaagatggcagtctgagtaggagctaaaatattttggg

SEQ ID NO: 221 - DNA, 52 bp, synthetic construct
  source 1..52: mol_type = other DNA
gtctgagtaggagctaaaatattttgggtttttctcataccttctgcttgat

SEQ ID NO: 222 - DNA, 45 bp, synthetic construct
  source 1..45: mol_type = other DNA
ccttctgcttgatgatcatctcggagtaacagtctgagtaggagc

SEQ ID NO: 223 - DNA, 48 bp, synthetic construct
  source 1..48: mol_type = other DNA
ccttctgcttgatgatcatctcgccagagtaacagtctgagtaggagc

SEQ ID NO: 224 - DNA, 43 bp, synthetic construct
  source 1..43: mol_type = other DNA
ccttctgcttgatgatcatctcgcagtctgagtaggagctaaa

SEQ ID NO: 225 - DNA, 42 bp, synthetic construct
  source 1..42: mol_type = other DNA
ctcataccttctgcttgatggagtaacagtctgagtaggagc

SEQ ID NO: 226 - DNA, 45 bp, synthetic construct
  source 1..45: mol_type = other DNA
ctcataccttctgcttgatgccagagtaacagtctgagtaggagc

SEQ ID NO: 227 - DNA, 40 bp, synthetic construct
  source 1..40: mol_type = other DNA
ctcataccttctgcttgatgcagtctgagtaggagctaaa

SEQ ID NO: 228 - DNA, 30 bp, synthetic construct; identical to SEQ ID NO: 187
  source 1..30: mol_type = other DNA

SEQ ID NO: 229 - DNA, 29 bp, synthetic construct
  source 1..29: mol_type = other DNA
ggttttctgatctccatcggaaaacccct

SEQ ID NO: 230 - DNA, 29 bp, synthetic construct
  source 1..29: mol_type = other DNA
ggttttccgacctccgtcggaaaacccct

SEQ ID NO: 231 - DNA, 1510 bp, synthetic construct
  source 1..1510: mol_type = other DNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaggagtcagcagcagcagcagcagcagcagcagcagcagcagcagcagcagaatttttggagcaggttttctgacctccgtcggaaaacccctcccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcggagtcagcagcagcagcagcagcagcagcagcagcagcagcagcagcagaatttttggagcaggttttctgacctccgtcggaaaacccctgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 232 - DNA, 1474 bp, synthetic construct
  source 1..1474: mol_type = other DNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaggagtcagcagcagcagcagcagcagcagcagcagaatttttggagcaggttttctgacctccgtcggaaaacccccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcggagtcagcagcagcagcagcagcagcagcagcagaatttttggagcaggttttctgacctccgtcggaaaaccgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 233 - DNA, 1510 bp, synthetic construct
  source 1..1510: mol_type = other DNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaggagtctgctgctgctgctgctgctgctgctgctgctgctgctgctgctgaatttttggagcaggttttctgacctccgtcggaaaacccctcccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcggagtctgctgctgctgctgctgctgctgctgctgctgctgctgctgctgaatttttggagcaggttttctgacctccgtcggaaaacccctgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg